(19) United States
(12) Patent Application Publication
(10) Pub. No.: US 2003/0068301 A1
(43) Pub. Date: Apr. 10, 2003

(54) Method and reagent for inhibiting hepatitis B virus replication

(76) Inventors: Kenneth Draper, Reno, NV (US); Lawrence Blatt, Boulder, CO (US); James A. McSwiggen, Boulder, CO (US); David Morrissey, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/877,478

(22) Filed: Jun. 8, 2001

Related U.S. Application Data
(63) Continuation-in-part of application No. 09/696,347, filed on Oct. 24, 2000.
Application No. 09/696,347 is a continuation-in-part of application No. 09/636,385, filed on Aug. 9, 2000.
Application No. 09/636,385 is a continuation-in-part of application No. 09/531,025, filed on Mar. 20, 2000.
Application No. 09/531,025 is a continuation-in-part of application No. 09/436,430, filed on Nov. 8, 1999.
Application No. 09/436,430 is a continuation of application No. 08/193,627, filed on Feb. 7, 1994, now Pat. No. 6,017,756.
Application No. 08/193,627 is a continuation of application No. 07/882,712, filed on May 14, 1992, now abandoned.

(51) Int. Cl. A61K 38/21; C07H 21/02; A61K 48/00

(52) U.S. Cl. 424/85.6; 514/44; 424/85.7; 536/23.1

(57) ABSTRACT

Nucleic acid molecules, including antisense and enzymatic nucleic acid molecules, such as hammerhead ribozymes, DNAzymes, Inozymes, Zinzymes, Amberzymes, and G-cleaver ribozymes, which modulate the synthesis, expression and/or stability of an RNA encoding one or more protein components of Hepatitis B virus (HBV), and methods for their use alone or in combination with other therapies, such as 3TC® (Lamivudine) and Interferons are disclosed.

This patent application is a continuation-in-part of Draper et al, U.S. Ser. No. (09/696,347), filed Oct. 24, 2000, entitled “METHOD AND REAGENT FOR INHIBITING HEPATITIS B VIRUS REPLICATION”, which is a continuation-in-part of Draper et al, U.S. Ser. No. (09/636,385), filed Aug. 9, 2000, entitled “METHOD AND REAGENT FOR INHIBITING HEPATITIS B VIRUS REPLICATION”, which is a continuation in part of Draper et al., U.S. Ser. No. (09/531,025), filed Mar. 20, 2000, entitled “METHOD AND REAGENT FOR INHIBITING HEPATITIS B VIRUS REPLICATION”, which is a continuation in part of Draper, U.S. Ser. No. (09/436,430), filed Nov. 8, 1999, entitled “METHOD AND REAGENT FOR INHIBITING HEPATITIS B VIRUS REPLICATION”, which is a continuation of U.S. Ser. No. (08/193,627), filed Feb. 7, 1994, now U.S. Pat. No. 6,017,756, which is a continuation of U.S. Ser. No. (07/882,712), filed May 14, 1992, entitled “METHOD AND REAGENT FOR INHIBITING HEPATITIS B VIRUS REPLICATION”, now abandoned. These applications are hereby incorporated by reference herein in their entireties, including the drawings.

### BACKGROUND OF THE INVENTION

The present invention concerns compounds, compositions, and methods for the study, diagnosis, and treatment of degenerative and disease states related to hepatitis B virus (HBV) replication and gene expression. Specifically, the invention relates to nucleic acid molecules used to inhibit expression of HBV.

The following is a discussion of relevant art pertaining to hepatitis B virus (HBV). The discussion is not meant to be complete and is provided only for understanding of the invention that follows. The summary is not an admission that any of the work described below is prior art to the claimed invention.

Chronic hepatitis B is caused by an enveloped virus, commonly known as the hepatitis B virus or HBV. HBV is transmitted via infected blood or other body fluids, especially saliva and semen, during delivery, sexual activity, or sharing of needles contaminated by infected blood. Individuals may be “carriers” and transmit the infection to others without ever having experienced symptoms of the disease. Persons at highest risk are those with multiple sex partners, those with a history of sexually transmitted diseases, parenteral drug users, infants born to infected mothers, “close” contacts or sexual partners of infected persons, and healthcare personnel or other service employees who have contact with blood. Transmission is also possible via tattooing, ear or body piercing, and acupuncture; the virus is also stable on razors, toothbrushes, baby bottles, eating utensils, and some hospital equipment such as respirators, scopes and instruments. There is no evidence that HBsAg positive food handlers pose a health risk in an occupational setting, nor should they be excluded from work. Hepatitis B has never been documented as being a food-borne disease. The average incubation period is 60 to 90 days, with a range of 45 to 180; the number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of severity include: (1) The size of the dose to which the person was exposed; (2) the person's age with younger patients experiencing a milder form of the disease; (3) the status of the immune system with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and tiredness/loss of energy. Jaundice is not experienced in all cases, however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement may occur accompanied by right upper-quadrant pain. The acute phase of the disease may be accompanied by severe depression, meningitis, Guillain-Barré syndrome, myelitis, encephalitis, agranulocytosis, and/or thrombocytopenia.

Hepatitis B is generally self-limiting and will resolve in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life do so.

Hepatitis B virus (HBV) infects over 300 million people worldwide (Imperial, 1999, Gastroenterol. Hepatol., 14 (suppl), S1-5). In the United States approximately 1.25 million individuals are chronic carriers of HBV as evidenced by the fact that they have measurable hepatitis B virus surface antigen HBsAg in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation is the only treatment option for patients with end-stage liver disease from HBV.

The natural progression of chronic HBV infection over a 10 to 20 year period leads to cirrhosis in 20-to-50% of patients and progression of HBV infection to hepatocellular carcinoma has been well documented. There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al, 1993, American Journal of Gastroenterology, 88, 240-243). Treatment of hepatocellular carcinoma with chemotherapeutic agents has not proven effective and only 10% of patients will benefit from surgery due to extensive tumor invasion of the liver (Trinchet et al., 1994, Presse Medicine, 23, 831-833). Given the aggressive nature of primary hepatocellular carcinoma, the only viable treatment alternative to surgery is liver transplantation (Pichlmayr et al., 1994, Hepatology., 20, 33S-40S).

Upon progression to cirrhosis, patients with chronic HCV infection present with clinical features, which are common to clinical cirrhosis regardless of the initial cause (D'Amico et al., 1986, Digestive Diseases and Sciences, 31, 468-475). These clinical features may include: bleeding esophageal varices, ascites, jaundice, and encephalopathy (Zakim D, Boyer T D. Hepatology a textbook of liver disease, Second Edition Volume 1. 1990 W. B. Saunders Company. Philadelphia). In the early stages of cirrhosis, patients are classified as compensated, meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Hepatitis B virus is a double-stranded circular DNA virus. It is a member of the Hepadnaviridae family. The virus consists of a central core that contains a core antigen (HBcAg) surrounded by an envelope containing a surface protein/surface antigen (HBsAg) and is 42 nm in diameter. It also contains an e antigen (HBeAg) which, along with HBcAg and HBsAg, is helpful in identifying this disease

In HBV virions, the genome is found in an incomplete double-stranded form. HBV uses a reverse transcriptase to transcribe a positive-sense full length RNA version of its genome back into DNA. This reverse transcriptase also contains DNA polymerase activity and thus begins replicating the newly synthesized minus-sense DNA strand. However, it appears that the core protein encapsidates the reverse-transcriptase/polymerase before it completes replication.

From the free-floating form, the virus must first attach itself specifically to a host cell membrane. Viral attachment is one of the crucial steps which determines host and tissue specificity. However, currently there are no in vitro cell-lines that can be infected by HBV. There are some cells lines, such as HepG2, which can support viral replication only upon transient or stable transfection using HBV DNA.

After attachment, fusion of the viral envelope and host membrane must occur to allow the viral core proteins containing the genome and polymerase to enter the cell. Once inside, the genome is translocated to the nucleus where it is repaired and cyclized.

The complete closed circular DNA genome of HBV remains in the nucleus and gives rise to four transcripts. These transcripts initiate at unique sites but share the same 3′-ends. The 3.5-kb pregenomic RNA serves as a template for reverse transcription and also encodes the nucleocapsid protein and polymerase. A subclass of this transcript with a 5′-end extension codes for the precore protein that, after processing, is secreted as HBV e antigen. The 2.4-kb RNA encompasses the pre-Sl open reading frame (ORF) that encodes the large surface protein. The 2.1-kb RNA encompasses the pre-S2 and S ORFs that encode the middle and small surface proteins, respectively. The smallest transcript (˜0.8-kb) codes for the X protein, a transcriptional activator.

Multiplication of the HBV genome begins within the nucleus of an infected cell. RNA polymerase II transcribes the circular HBV DNA into greater-than-full length mRNA. Since the mRNA is longer than the actual complete circular DNA, redundant ends are formed. Once produced, the pregenomic RNA exits the nucleus and enters the cytoplasm.

The packaging of pregenomic RNA into core particles is triggered by the binding of the HBV polymerase to the 5′ epsilon stem-loop. RNA encapsidation is believed to occur as soon as binding occurs. The HBV polymerase also appears to require associated core protein in order to function. The HBV polymerase initiates reverse transcription from the 5′ epsilon stem-loop three to four base pairs at which point the polymerase and attached nascent DNA are transferred to the 3′ copy of the DR1 region. Once there, the (−)DNA is extended by the HBV polymerase while the RNA template is degraded by the HBV polymerase RNAse H activity. When the HBV polymerase reaches the 5′ end, a small stretch of RNA is left undigested by the RNAse H activity. This segment of RNA is comprised of a small sequence just upstream and including the DR1 region. The RNA oligomer is then translocated and annealed to the DR2 region at the 5′ end of the (−)DNA. It is used as a primer for the (+)DNA synthesis which is also generated by the HBV polymerase. It appears that the reverse transcription as well as plus strand synthesis may occur in the completed core particle.

Since the pregenomic RNA is required as a template for DNA synthesis, this RNA is an excellent target for ribozyme cleavage. Nucleoside analogues that have been documented to inhibit HBV replication target the reverse transcriptase activity needed to convert the pregenomic RNA into DNA. Ribozyme cleavage of the pregenomic RNA template would be expected to result in a similar inhibition of HBV replication. Further, targeting the 3′-end of the pregenomic RNA that is common to all HBV transcripts could result in reduction of all HBV gene products and an additional level of inhibition of HBV replication.

### Cell Culture Models

As previously mentioned HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA would be co-transfected with ribozymes in cell culture. Such an approach has been used to report intracellular ribozyme activity against HBV (zu Putlitz, et al., 1999, J Virol., 73, 5381-5387, and Kim et al., 1999, Biochem. Biophys. Res. Commun., 257, 759-765). In addition, stable hepatocyte cell lines have been generated that express HBV. In these cells only ribozyme would need to be delivered; however, a delivery screen would need to be performed.

### Phenotypic Assays

Intracellular HBV gene expression can be assayed by a Taqman® assay for HBV RNA or by ELISA for HBV protein. Extracellular virus can be assayed by PCR for DNA or ELISA for protein. Antibodies are commercially available for HBV surface antigen and core protein. A secreted alkaline phosphatase expression plasmid can be used to normalize for differences in transfection efficiency and sample recovery.

### Animal Models

There are several small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

### Therapeutic Approaches

Current therapeutic goals of treatment are three-fold: to eliminate infectivity and transmission of HBV to others, to arrest the progression of liver disease and improve the clinical prognosis, and to prevent the development of hepatocellular carcinoma (HCC).

Interferon alpha use is the most common therapy for HBV; however, recently Lamivudine (3TC®) has been approved by the FDA. Interferon alpha (IFN-alpha) is one treatment for chronic hepatitis B. The standard duration of IFN-alpha therapy is 16 weeks, however, the optimal treatment length is still poorly defined. A complete response (HBV DNA negative HBeAg negative) occurs in approximately 25% of patients. Several factors have been identified that predict a favorable response to therapy including: High ALT, low HBV DNA , being female, and heterosexual orientation.

There is also a risk of reactivation of the hepatitis B virus even after a successful response, this occurs in around 5% of responders and normally occurs within 1 year.

Side effects resulting from treatment with type 1 interferons can be divided into four general categories including: Influenza-like symptoms, neuropsychiatric, laboratory abnormalities, and other miscellaneous side effects. Examples of influenza-like symptoms include, fatigue, fever; myalgia, malaise, appetite loss, tachycardia, rigors, headache and arthralgias. The influenza-like symptoms are usually short-lived and tend to abate after the first four weeks of dosing (Dusheiko et al., 1994, Journal of Viral Hepatitis, 1, 3-5). Neuropsychiatric side effects include irritability, apathy, mood changes, insomnia, cognitive changes, and depression. Laboratory abnormalities include the reduction of myeloid cells, including granulocytes, platelets and to a lesser extent, red blood cells. These changes in blood cell counts rarely lead to any significant clinical sequellae. In addition, increases in triglyceride concentrations and elevations in serum alaine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea, diarrhea, abdominal and back pain, pruritus, alopecia, and rhinorrhea. In general, most side effects will abate after 4 to 8 weeks of therapy (Dushieko et al., supra ).

Lamivudine (3TC®) is a nucleoside analogue, which is a very potent and specific inhibitor of HBV DNA synthesis. Lamivudine has recently been approved for the treatment of chronic Hepatitis B. Unlike treatment with interferon, treatment with 3TC® does not eliminate the HBV from the patient. Rather, viral replication is controlled and chronic administration results in improvements in liver histology in over 50% of patients. Phase III studies with 3TC®, showed that treatment for one year was associated with reduced liver inflammation and a delay in scarring of the liver. In addition, patients treated with Lamivudine (100 mg per day) had a 98 percent reduction in hepatitis B DNA and a significantly higher rate of seroconversion, suggesting disease improvements after completion of therapy. However, stopping of therapy resulted in a reactivation of HBV replication in most patients. In addition recent reports have documented 3TC® resistance in approximately 30% of patients.

Current therapies for treating HBV infection, including interferon and nucleoside analogues, are only partially effective. In addition, drug resistance to nucleoside analogues is now emerging, making treatment of chronic Hepatitis B more difficult. Thus, a need exists for effective treatment of this disease which utilizes antiviral inhibitors which work by mechanisms other than those currently utilized in the treatment of both acute and chronic hepatitis B infections.

Draper, U.S. Pat. No. 6,017,756, describes the use of ribozymes for the inhibition of Hepatitis B Virus.

Passman et al., 2000, Biochem. Biophys. Res. Commun., 268(3), 728-733.; Gan et al., 1998, J. Med. Coll. PLA, 13(3), 157-159.; Li et al., 1999, Jiefangiun Yixue Zazhi, 24(2), 99-101.; Putlitz et al, 1999, J. Virol., 73(7), 5381-5387.; Kim et al., 1999, Biochem. Biophys. Res. Commun., 257(3), 759-765.; Xu et al., 1998, Bingdu Xuebao, 14(4), 365-369.; Welch et al., 1997, Gene Ther., 4(7), 736-743.; Goldenberg et al., 1997, International PCT publication No. WO 97/08309, Wands et al., 1997, J. of Gastroenterology and Hepatology, 12(suppl.), S354-S369.; Ruiz et al., 1997, BioTechniques, 22(2), 338-345.; Gan et al., 1996, J. Med. Coll. PLA, 11(3), 171-175.; Beck and Nassal, 1995, Nucleic Acids Res., 23(24), 4954-62.; Goldenberg, 1995, International PCT publication No. WO 95/22600.; Xu et al, 1993, Bingdu Xuebao, 9(4), 331-6.; Wang et al., 1993, Bingdu Xuebao, 9(3), 278-80, all describe ribozymes that are targeted to cleave a specific HBV target site.

### SUMMARY OF THE INVENTION

This invention relates to enzymatic nucleic acid molecules directed to disrupt the function of RNA species of hepatitis B virus (HBV) and/or encoded by the HBV. In particular, applicant describes the selection and function of enzymatic nucleic acid molecules capable of specifically cleaving HBV RNA. Such enzymatic nucleic acid molecules may be used to treat diseases and disorders associated with HBV infection.

In one embodiment, the invention features an enzymatic nucleic acid molecule that specifically cleaves RNA derived from hepatitis B virus (HBV), wherein the enzymatic nucleic acid molecule comprises sequence defined as Seq. ID No. 6346.

In another embodiment, the invention features a pharmaceutical composition comprising an enzymatic nucleic acid molecule of the invention in a pharmaceutically acceptable carrier.

In another embodiment, the invention features a mammalian cell, for example a human cell, including an enzymatic nucleic acid molecule contemplated by the invention.

In one embodiment, the invention features a method for treatment of cirrhosis, liver failure or hepatocellular carcinoma comprising administering to a patient an enzymatic nucleic acid molecule the invention under conditions suitable for the treatment.

In another embodiment, the invention features a method of treatment of a patient having a condition associated with HBV infection, comprising contacting cells of said patient with an enzymatic nucleic acid molecule of the invention, and further comprising the use of one or more drug therapies, for example type I interferon or 3TC® (lamivudine), under conditions suitable for said treatment. In another embodiment, the other therapy is administered simultaneously with or separately from the enzymatic nucleic acid molecule.

In another embodiment, the invention features a method for inhibiting HBV replication in a mammalian cell comprising administering to the cell an enzymatic nucleic acid molecule of the invention under conditions suitable for the inhibition.

In yet another embodiment, the invention features a method of cleaving a separate RNA molecule comprising, contacting an enzymatic nucleic acid molecule of the invention with the separate RNA molecule under conditions suitable for the cleavage of the separate RNA molecule.

In one embodiment, cleavage by an enzymatic nucleic acid molecule of the invention is carried out in the presence of a divalent cation, for example Mg2+.

In another embodiment, an enzymatic nucleic acid molecule of the invention is chemically synthesized.

In another embodiment, the type I interferon contemplated by the invention is interferon alpha, interferon beta, polyethylene glycol interferon, polyethylene glycol interferon alpha 2a, polyethylene glycol interferon alpha 2b, polyethylene glycol consensus interferon.

In one embodiment, the invention features a pharmaceutical composition comprising type I interferon and an enzymatic nucleic acid molecule of the invention, in a pharmaceutically acceptable carrier.

In another embodiment, the invention features a method of administering to a cell, for example a mammalian cell or human cell, an enzymatic nucleic acid molecule of the invention independently or in conjunction with other therapeutic compounds such as type I interferon or 3TC® (lamivudine), comprising contacting the cell with the enzymatic nucleic acid molecule under conditions suitable for the administration.

In another embodiment, administration of an enzymatic nucleic acid molecule of the invention is in the presence of a delivery reagent, for example a lipid, cationic lipid, phospholipid, or liposome.

In a preferred embodiment, the invention features novel nucleic acid-based techniques such as enzymatic nucleic acid molecules and antisense molecules and methods for their use to down regulate or inhibit the expression of HBV RNA and/or replication of HBV.

In a preferred embodiment, the invention features the use of one or more of the enzymatic nucleic acid-based techniques to inhibit the expression of the genes encoding HBV viral proteins. Specifically, the invention features the use of enzymatic nucleic acid-based techniques to specifically inhibit the expression of the HBV viral genome.

In another preferred embodiment, the invention features nucleic acid-based inhibitors (e.g., enzymatic nucleic acid molecules (ribozymes), antisense nucleic acids, triplex DNA, antisense nucleic acids containing RNA cleaving chemical groups) and methods for their use to down regulate or inhibit the expression of RNA (e.g., HBV) capable of progression and/or maintenance of hepatitis, hepatocellular carcinoma, cirrhosis, and/or liver failure.

In one embodiment, nucleic acid molecules of the invention are used to treat HBV infected cells or a HBV infected patient wherein the HBV is resistant or the patient does not respond to treatment with 3TC® (Lamivudine), either alone or in combination with other therapies under conditions suitable for the treatment.

In another embodiment, nucleic acid molecules of the invention are used to treat HBV infected cells or a HBV infected patient wherein the HBV is resistant or the patient does not respond to treatment with Interferon, for example Infergeng®, either alone or in combination with other therapies under conditions suitable for the treatment.

In yet another preferred embodiment, the invention features the use of an enzymatic nucleic acid molecule, preferably in the hammerhead, NCH (Inozyme), G-cleaver, amberzyme, zinzyme, and/or DNAzyme motif, to inhibit the expression of HBV RNA.

By “inhibit” it is meant that the activity of HBV or level of RNAs or equivalent RNAs encoding one or more protein subunits of HBV is reduced below that observed in the absence of the nucleic acid. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence, of for example, an oligonucleotide with scrambled sequence or with mismatches. In another embodiment, inhibition of HBV RNA with the nucleic acid molecule of the instant invention is greater than in the presence of the nucleic acid molecule than in its absence.

These enzymatic nucleic acid molecules exhibit a high degree of specificity for only the viral mRNA in infected cells. Nucleic acid molecules of the instant invention targeted to highly conserved sequence regions allow the treatment of many strains of human HBV with a single compound. No treatment presently exists which specifically attacks expression of the viral gene(s) that are responsible for transformation of hepatocytes by HBV.

The methods of this invention can be used to treat human hepatitis B virus infections, which include productive virus infection, latent or persistent virus infection, and HBV-induced hepatocyte transformation. The utility can be extended to other species of HBV which infect non-human animals where such infections are of veterinary importance.

Preferred target sites are genes required for viral replication, a non-limiting example includes genes for protein synthesis, such as the 5′ most 1500 nucleotides of the HBV pregenomic mRNAs. For sequence references, see Renbao et al., 1987, Sci. Sin., 30, 507. This region controls the translational expression of the core protein (C), X protein (X) and DNA polymerase (P) genes and plays a role in the replication of the viral DNA by serving as a template for reverse transcriptase. Disruption of this region in the RNA results in deficient protein synthesis as well as incomplete DNA synthesis (and inhibition of transcription from the defective genomes). Target sequences 5′ of the encapsidation site can result in the inclusion of the disrupted 3′ RNA within the core virion structure and targeting sequences 3′ of the encapsidation site can result in the reduction in protein expression from both the 3′ and 5′ fragments.

Alternative regions outside of the 5′ most 1500 nucleotides of the pregenomic mRNA also make suitable targets of enzymatic nucleic acid mediated inhibition of HBV replication. Such targets include the mRNA regions that encode the viral S gene. Selection of particular target regions will depend upon the secondary structure of the pregenomic mRNA. Targets in the minor mRNAs can also be used, especially when folding or accessibility assays in these other RNAs reveal additional target sequences that are unavailable in the pregenomic mRNA species.

A desirable target in the pregenomic RNA is a proposed bipartite stem-loop structure in the 3′-end of the pregenomic RNA which is believed to be critical for viral replication (Kidd and Kidd-Ljunggren, 1996. Nuc. Acid Res. 24:3295-3302). The 5′ end of the HBV pregenomic RNA carries a cis-acting encapsidation signal, which has inverted repeat sequences that are thought to form a bipartite stem-loop structure. Due to a terminal redundancy in the pregenomic RNA, the putative stem-loop also occurs at the 3′-end. While it is the 5′ copy which functions in polymerase binding and encapsidation, reverse transcription actually begins from the 3′ stem-loop. To start reverse transcription, a 4 nt primer which is covalently attached to the polymerase is made, using a bulge in the 5′ encapsidation signal as template. This primer is then shifted, by an unknown mechanism, to the DR1 primer binding site in the 3′ stem-loop structure, and reverse transcription proceeds from that point. The 3′ stem-loop, and especially the DR1 primer binding site, appear to be highly effective targets for ribozyme intervention.

Sequences of the pregenomic RNA are shared by the mRNAs for surface, core, polymerase, and X proteins. Due to the overlapping nature of the HBV transcripts, all share a common 3′-end. Ribozyme targeting o this common 3′-end will thus cleave the pregenomic RNA as well as all of the mRNAs for surface, core, polymerase and X proteins.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA 260:20 3030-4).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example see FIGS. 1-5).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired (see for example Werner and Uhlenbeck, 1995, Nucleic Acids Research, 23, 2092-2096; Hatnmann et al., 1999, Antisense and Nucleic Acid Drug Dev., 9, 25-31). Such arms are shown generally in FIGS. 1-5. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long (see for example Werner and Uhlenbeck, supra; Hamman et al., supra; Hampel et al., EP0360257; Berzal-Herrance et al, 1993, EMBO J., 12, 2567-73). If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “NCH” or “Inozyme” motif is meant, an enzymatic nucleic acid molecule comprising a motif as described in Ludwig et al., U.S. Ser. No. 09/406,643, filed Sep. 27, 1999, entitled “COMPOSITIONS HAVING RNA CLEAVING ACTIVITY”, and International PCT publication Nos. WO 98/58058 and WO 98/58057, all incorporated by reference herein in their entirety, including the drawings.

By “G-cleaver” motif is meant, an enzymatic nucleic acid molecule comprising a motif as described in Eckstein et al., International PCT publication No. WO 99/16871, incorporated by reference herein in its entirety, including the drawings.

By “zinzyme” motif is meant, a class II enzymatic nucleic acid molecule comprising a motif as described in Beigelman et al., International PCT publication No. WO 99/55857, incorporated by reference herein in its entirety, including the drawings.

By “amberzyme” motif is meant, a class I enzymatic nucleic acid molecule comprising a motif as described in Beigelman et al., International PCT publication No. WO 99/55857, incorporated by reference herein in its entirety, including the drawings.

By ‘DNAzyme’ is meant, an enzymatic nucleic acid molecule lacking a ribonucleotide (2′-OH) group. In particular embodiments, the enzymatic nucleic acid molecule may have an attached linker(s) or other attached or associated groups, moieties, or chains containing one or more nucleotides with 2′-OH groups. A DNAzyme can be synthesized chemically or can be expressed by means of a single stranded DNA vector or equivalent thereof.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant, interaction of the oligonucleotides with target nucleic acid (e.g., by forming hydrogen bonds with complementary nucleotides in the target under physiological conditions).

By “equivalent” RNA to HBV is meant to include those naturally occurring RNA molecules having homology (partial or complete) to HBV proteins or encoding for proteins with similar function as HBV in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both. For a review of current antisense strategies, see Schmajuk et al., 1999, J. Biol. Chem., 274, 21783-21789, Delihas et al., 1997, Nature, 15, 751-753, Stein et al, 1997, Antisense N. A. Drug Dev., 7, 151, Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” it is meant, an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992, Proc. Natl. Acad. Sci. USA, 89, 504).

By “gene” it is meant a nucleic acid that encodes an RNA.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. -Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in HBV-specific RNAs represent a novel therapeutic approach to treat a variety of pathologic indications, including, HBV infection, hepatitis, hepatocellular carcinoma, tumorigenesis, cirrhosis, liver failure and others.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; and Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; and Li and Altman, 1996, Nucleic Acids Res. 24, 835. The Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; and Guo and Collins, 1995, EMBO. J. 14, 363). Group II introns are described by Griffin et al., 1995, Chem. Biol. 2, 761; Michels and Pyle, 1995, Biochemistry 34, 2965; and Pyle et al, International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; and Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs include the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention. and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

Exemplary enzymatic nucleic acid molecules of the invention are shown in Tables V-XI. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding HBV proteins (specifically HBV RNA) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g., bacterial cell) or eukaryotic (e.g., mammalian or plant cell).

By “HBV proteins” is meant, a protein or a mutant protein derivative thereof, comprising sequence expressed and/or encoded by the HBV genome.

By “highly conserved sequence region” is meant a nucleotide sequence of one or more regions in a target gene does not vary significantly from one generation to the other or from one biological system to the other.

The enzymatic nucleic acid-based inhibitors of HBV expression are useful for the prevention of the diseases and conditions including HBV infection, hepatitis, cancer, cirrhosis, liver failure, and any other diseases or conditions that are related to the levels of HBV in a cell or tissue.

By “related” is meant that the reduction of HBV expression (specifically HBV gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables IV to XI. Examples of such enzymatic nucleic acid molecules also are shown in Tables V to XI. Examples of such enzymatic nucleic acid molecules consist essentially of sequences defined in these tables.

In yet another embodiment, the invention features antisense nucleic acid molecules including sequences complementary to the substrate sequences shown in Tables IV to XI. Such nucleic acid molecules can include sequences as shown for the binding arms of the enzymatic nucleic acid molecules in Tables V to XI. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

In another aspect, the invention provides mammalian cells containing one or more nucleic acid molecules and/or expression vectors of this invention. The one or more nucleic acid molecules may independently be targeted to the same or different sites.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables V and VI can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 6705), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

In another aspect of the invention, ribozymes or antisense molecules that interact with target RNA molecules and inhibit HBV (specifically HBV RNA) activity are expressed from transcription units inserted into DNA or RNA vectors. The recombinant vectors are preferably DNA plasmids or viral vectors. Ribozyme or antisense expressing viral vectors could be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors might be repeatedly administered as necessary. Once expressed, the ribozymes or antisense bind to the target RNA and inhibit its function or expression. Delivery of ribozyme or antisense expressing vectors could be systemic, such as by intravenous or intramuscular administration, by administration to target cells ex-planted from the patient followed by reintroduction into the -patient, or by any other means that would allow for introduction into the desired target cell. Antisense DNA can be expressed via the use of a single stranded DNA intracellular expression vector.

By RNA is meant a molecule comprising at least one ribonucleotide residue. By “ribonucleotide” is meant a nucleotide with a hydroxyl group at the 2′ position of a β-D-ribo-furanose moiety.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs, can be used to treat diseases or conditions discussed above. For example, to treat a disease or condition associated with HBV, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat HBV infection, hepatitis, hepatocellular carcinoma, cancer, cirrhosis, and liver failure. Such therapeutic agents may include, but are not limited to nucleoside analogs selected from the group comprising Lamivudine (3TC®, L-FMAU, and/or adefovir dipivoxil (for a review of applicable nucleoside analogs, see Colacino and Staschke, 1998, Progress in Drug Research, 50, 259-322). Immunomodulators selected from the group comprising Type 1 Interferon, Therapeutic vaccines, steriods, and 2′-5′ oligoadenylates (for a review of 2′-5′ Oligoadenylates, see Charubala and Pfleiderer, 1994, Progress in Molecular and Subcellular Biology, 14, 113-138).

In another preferred embodiment, the invention features nucleic acid-based inhibitors (e.g., enzymatic nucleic acid molecules (ribozymes), antisense nucleic acids, triplex DNA, antisense nucleic acids containing RNA cleaving chemical groups) and methods for their use to down regulate or inhibit the expression of RNA (e.g., HBV) capable of progression and/or maintenance of liver disease and failure.

In another preferred embodiment, the invention features nucleic acid-based techniques (e.g., enzymatic nucleic acid molecules (ribozymes), antisense nucleic acids, triplex DNA, antisense nucleic acids containing RNA cleaving chemical groups) and methods for their use to down regulate or inhibit the expression of HBV RNA expression.

In preferred embodiments, the invention features a method for the analysis of HBV proteins. This method is useful in determining the efficacy of HBV inhibitors. Specifically, the instant invention features an assay for the analysis of HBsAg proteins and secreted alkaline phosphatase (SEAP) control proteins to determine the efficacy of agents used to modulate HBV expression.

The method consists of coating a micro-titer plate with an antibody such as anti-HBsAg Mab (for example, Biostride B88-95-31ad,ay) at 0.1 to 10 μg/ml in a buffer (for example, carbonate buffer, such as Na₂CO₃ 15 mM, NaHCO₃ 35 mM, pH 9.5) at 4° C. overnight. The microtiter wells are then washed with PBST or the equivalent thereof, (for example, PBS, 0.05% Tween 20) and blocked for 0.1-24 hr at 37° C. with PBST, 1% BSA or the equivalent thereof. Following washing as above, the wells are dried (for example, at 37° C. for 30 min). Biotinylated goat anti-HBsAg or an equivalent antibody (for example, Accurate YVS1807) is diluted (for example at 1:1000) in PBST and incubated in the wells (for example, 1 hr. at 37° C.). The wells are washed with PBST (for example, 4×). A conjugate, (for example, Streptavidin/Alkaline Phosphatase Conjugate, Pierce 21324) is diluted to 10-10,000 ng/ml in PBST, and incubated in the wells (for example, 1 hr. at 37° C.). After washing as above, a substrate (for example, p-nitrophenyl phosphate substrate, Pierce 37620) is added to the wells, which are then incubated (for example, 1 hr. at 37° C.). The optical density is then determined (for example, at 405 nm). SEAP levels are then assayed, for example, using the Great EscAPe® Detection Kit (Clontech K2041-1), as per the manufacturers instructions. In the above example, incubation times and reagent concentrations may be varied to achieve optimum results, a non-limiting example is described in Example 6.

Comparison of this HBsAg ELISA method to a commercially available assay from World Diagnostics, Inc. 15271 NW ₆₀^{th} Ave, #201, Miami Lakes, Fla. 33014 (305) 827-3304 (Cat. No. EL10018) demonstrates an increase in sensitivity (signal:noise) of 3-20 fold.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage. - - - indicate the target sequence. Lines interspersed with dots are meant to indicate tertiary interactions.—is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al, 1990, J. Biol. Chem., 265, 3587). Group II Intron: 5′ SS means 5′ splice site; 3′ SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: I-IV are meant to indicate four stem-loop structures (Been et al., U.S. Pat. No. 5,625,047). Hammerhead Ribozyme: I-III are meant to indicate three stem-loop structures; stems I-III can be of any length and may be symmetrical or asymmetrical (Usman et al, 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” ≧is 2 bases. The connecting loop can also be replaced with a non-nucleotide linker molecule. H refers to bases A, U, or C. Y refers to pyrimidine bases. “ ” refers to a covalent bond. (Burke et al., 1996, Nucleic Acids & Mol. Biol., 10, 129; Chowrira et al., U.S. Pat. No. 5,631,359).

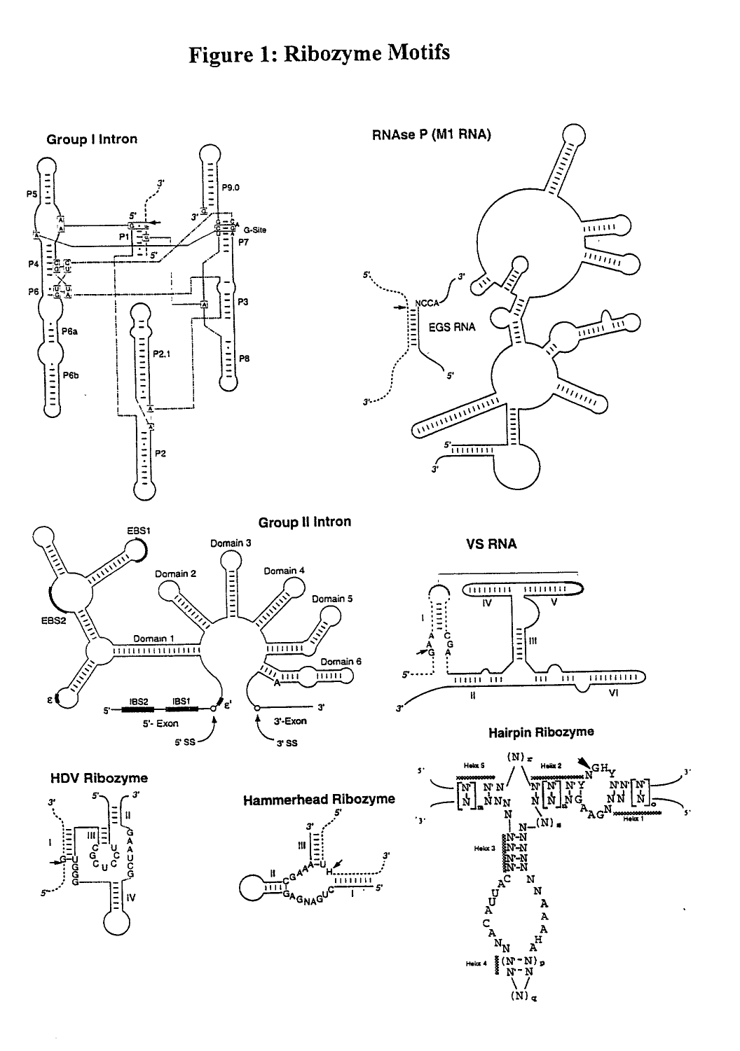

FIG. 2 shows examples of chemically stabilized ribozyme motifs. HH Rz, represents hammerhead ribozyme motif (Usman et al, 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research, 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

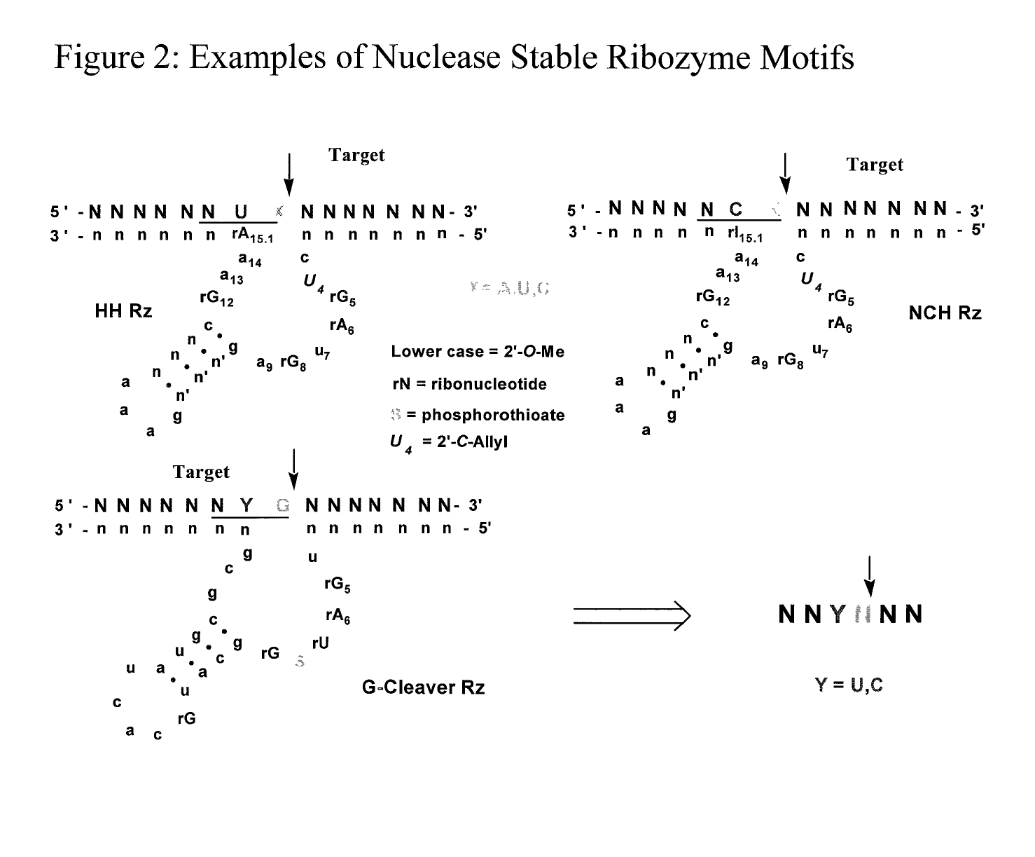

FIG. 3 shows an example of the Amberzyme ribozyme motif that is chemically stabilized (see, for example, Beigelman et al., International PCT publication No. WO 99/55857; also referred to as Class I Motif). The Amberzyme motif is a class of enzymatic nucleic acid molecules that do not require the presence of a ribonucleotide (2′-OH) group for activity.

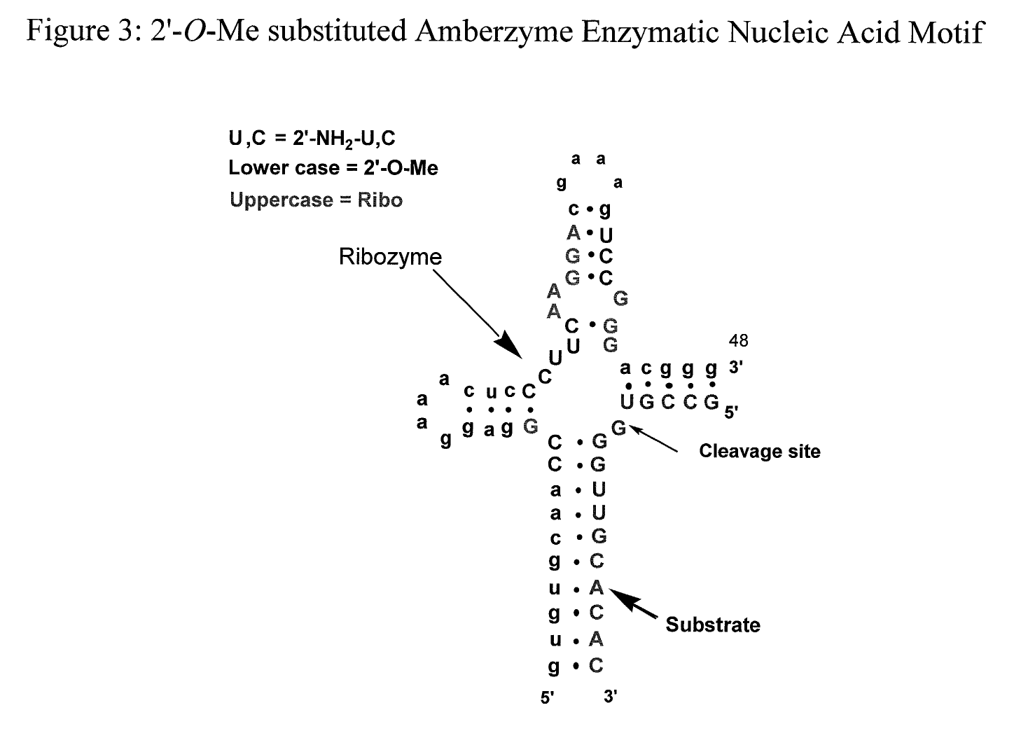

FIG. 4 shows an example of the Zinzyme A ribozyme motif that is chemically stabilized (see, for example, International PCT publication No. WO 99/55857; also referred to as Class A Motif). The Zinzyme motif is a class of enzymatic nucleic acid molecules that do not require the presence of a ribonucleotide (2′-OH) group for activity.

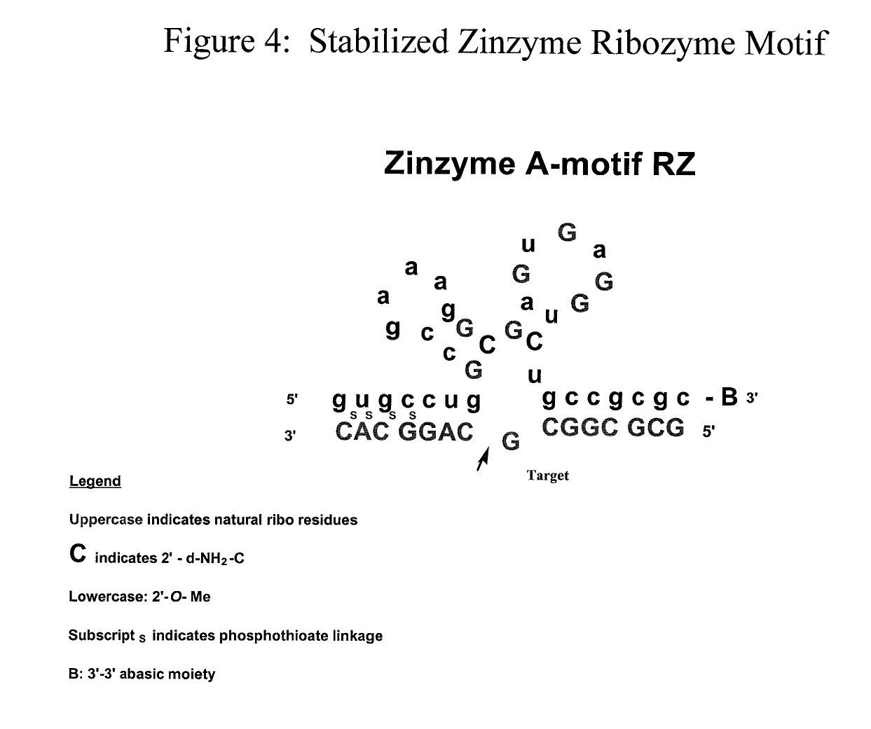

FIG. 5 shows an example of a DNAzyme motif described by Santoro et al., 1997, PNAS, 94, 4262.

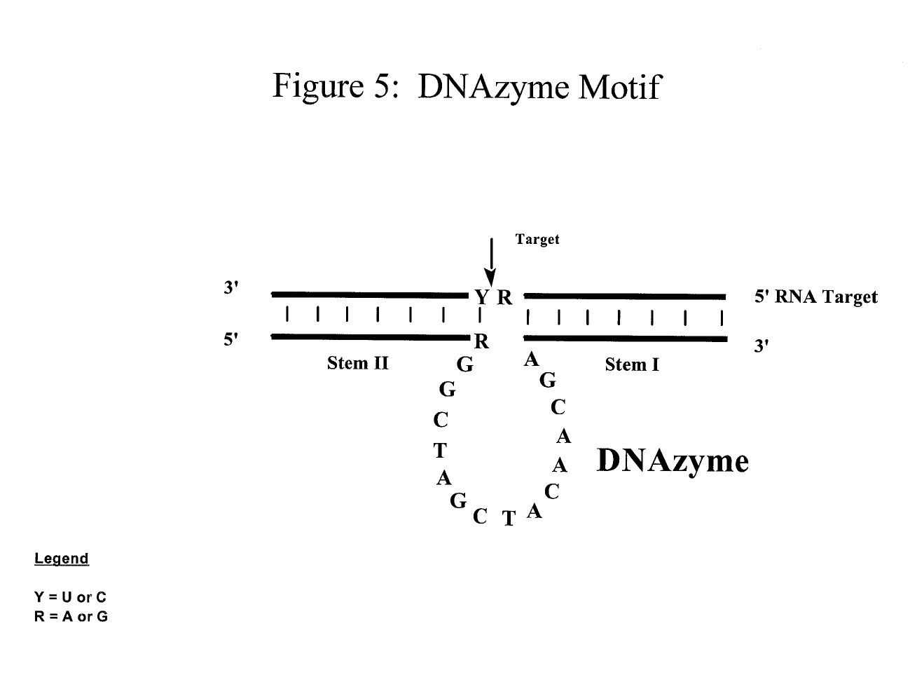

FIG. 6 is a bar graph showing the percent change in serum HBV DNA levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 7 is a bar graph showing the mean serum HBV DNA levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 8 is a bar graph showing the decrease in serum HBV DNA (log) levels following fourteen days of ribozyme treatment in HBV transgenic mice. Ribozymes targeting sites 273 (RPI.18341) and 1833 (RPI.18371) of HBV RNA administerd via continuous s.c. infusion at 10, 30, and 100 mg/kg/day are compared to continuous s.c. infusion administration of scrambled attenuated core ribozyme and saline controls, and orally administered 3TC® (300 mg/kg/day) and saline controls.

FIG. 9 is a bar graph showing the decrease in HBV DNA in HepG2.2.15 cells after treatment with ribozymes targeting sites 273 (RPI.18341), 1833 (RPI.18371), 1874 (RPI.18372), and 1873 (RPI.18418) of HBV RNA as compared to a scrambled attenuated core ribozyme (RPI.20995).

FIG. 10 is a bar graph showing reduction in HBsAg levels following treatment of HepG2 cells with anti-HBV arm, stem, and loop-variant ribozymes (RPI.18341, RPI.22644, RPI.22645, RPI.22646, RPI.22647, RPI.22648, RPI.22649, and RPI.22650) targeting site 273 of the HBV pregenomic RNA as compared to a scrambled attenuated core ribozyme (RPI.20599).

FIG. 11 is a bar graph showing reduction in HBsAg levels following treatment of HepG2 cells with RPI 18341 alone or in combination with Infergen®. At either 500 or 1000 units of Infergeng®, the addition of 200 nM of RPI.18341 results in a 75-77% increase in anti-HBV activity as judged by the level of HBsAg secreted from the treated Hep G2 cells. Conversely, the anti-HBV activity of RPI.18341(at 200 nM) is increased 31-39% when used in combination of 500 or 1000 units of Infergen®.

FIG. 12 is a bar graph showing reduction in HBsAg levels following treatment of HepG2 cells with RPI 18341 alone or in combination with Lamivudine. At 25 nM Lamivudine (3TC®, the addition of 100 nM of RPI.18341 results in a 48% increase in anti-HBV activity as judged by the level of HBsAg secreted from treated Hep G2 cells. Conversely, the anti-HBV activity of RPI.18341 (at 100 nM) is increased 31% when used in combination with 25 nM Lamivudine.

### MECHANISM OF ACTION OF NUCLEIC ACID MOLECULES OF THE INVENTION

Antisense: Antisense molecules may be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently, it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. Ser. No. 60/082,404 which was filed on Apr. 20, 1998; Hartmann et al., U.S. Ser. No. 60/101,174 which was filed on Sep. 21, 1998) all of these are incorporated by reference herein in their entirety.

Antisense DNA can be used to target RNA by means of DNA-RNA interactions, thereby activating RNase H, which digests the target RNA in the duplex. Antisense DNA can be chemically synthesized or can be expressed via the use of a single stranded DNA intracellular expression vector or the equivalent thereof.

Triplex Forming Oligonucleotides (TFO): Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra)

2′-5′ Oligoadenylates: The 2-5 A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5∝oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme. The covalent attachment of 2′-5′ oligoadenylate structures is not limited to antisense applications, and can be further elaborated to include attachment to nucleic acid molecules of the instant invention.

Enzymatic Nucleic Acid: Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent HBV protein expression and can be used to treat disease or diagnose disease associated with the levels of HBV.

The enzymatic nature of a ribozyme has significant advantages, such as the concentration of ribozyme necessary to affect a therapeutic treatment is low. This advantage reflects the ability of the ribozyme to act enzymatically, Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al, 324, Nature, 429 1986 ; Uhlenbeck, 1987 Nature, 328, 596; Kim et al., 84 Proc. Natl. Acad. Sci. USA, 8788, 1987; Dreyfus, 1988, Einstein Quart. J. Bio. Med., 6, 92; Haseloff and Gerlach, 334 Nature, 585, 1988; Cech, 260 JAMA, 3030, 1988;

Jefferies et al., 17 Nucleic Acids Research, 1371, 1989; and Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250.

The nucleic acid molecules of the instant invention are also referred to as GeneBloc™ reagents, which are essentially nucleic acid molecules (e.g.; ribozymes, antisense) capable of down-regulating gene expression.

### Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al, U.S. Pat. No. 5,525,468, and all hereby incorporated in their entirites by reference herein. Other examples include the following PCT applications, which concern inactivation of expression of disease-related genes: WO 95/23225, WO 95/13380, WO 94/02595, all incorporated by reference herein. Rather than repeat the guidance provided in those documents here, below are provided specific examples of such methods, not limiting to those in the art. Ribozymes and antisense to such targets are designed as described in those applications and synthesized to be tested in vitro and in vivo, as also described. The sequence of human HBV RNAs (for example, accession AF100308.1; HBV strain 2-18; additionally, other HBV strains can be screened by one skilled in the art, see Table III for other possible strains) were screened for optimal enzymatic nucleic acid and antisense target sites using a computer-folding algorithm. Antisense, hammerhead, DNAzyme, NCH (Inozyme), amberzyme, zinzyme or G-Cleaver ribozyme binding/cleavage sites were identified. These sites are shown in Tables V to XI (all sequences are 5′ to 3′ in the tables; X can be any base-paired sequence, the actual sequence is not relevant here). The nucleotide base position is noted in the Tables as that site to be cleaved by the designated type of enzymatic nucleic acid molecule. Table IV shows substrate positions selected from Renbo et al., 1987, Sci. Sin., 30, 507, used in Draper, U.S. Ser. No. (07/882,712), filed May 14, 1992, entitled “METHOD AND REAGENT FOR INHIBITING HEPATITIS B VIRUS REPLICATION” and Draper et al., International PCT publication No. WO 93/23569, filed Apr. 29, 1993, entitled “METHOD AND REAGENT FOR INHIBITING VIRAL REPLICATION”. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH (Inozyme), amberzyme, zinzyme or G-Cleaver ribozyme binding/cleavage sites were identified, as discussed above. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al, 1990 Nucleic Acids Res., 18, 5433; Wincott et al., 1995 Nucleic Acids Res. 23, 2677-2684; and Caruthers et al., 1992, Methods in Enzymology 211,3-19.

### Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (e.g.; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′ end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 ,μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al, 1995, Nucleic Acids Res. 23, 2677-2684 Wincott et al, 1997, Methods Mol. Bio., 74, 59, and makes use of common nucleic acid protecting and coupling groups, such as dimethoxytrityl at the 5′ end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1: 1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA 3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al, 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; see Wincott et al., supra, the totality of which is hereby incorporated herein by reference) and are re-suspended in water.

The sequences of the ribozymes and antisense constructs that are chemically synthesized, useful in this study, are shown in Tables IV to IX. Those in the art will recognize that these sequences are representative only of many more such sequences where the enzymatic portion of the ribozyme (all but the binding arms) is altered to affect activity. The ribozyme and antisense construct sequences listed in Tables IV to IX may be formed of ribonucleotides or other nucleotides or non-nucleotides. Such ribozymes with enzymatic activity are equivalent to the ribozymes described specifically in the Tables.

### Optimizing Activity of the nucleic acid molecule of the invention

Chemically synthesizing nucleic acid molecules with modifications (base, sugar and/or phosphate) that prevent their degradation by serum ribonucleases may increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein and are all hereby incorporated by reference herein). Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., US Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earmshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore, when designing nucleic acid molecules, the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid molecules are also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (are incorporated by reference herein) have expanded the ability to modify nucleic acid molecules by introducing nucleotide modifications to enhance their nuclease stability as described above.

Use of these the nucleic acid-based molecules of the invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

Therapeutic nucleic acid molecules (e.g., enzymatic nucleic acid molecules and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and ribozyme stability. In this invention, the product of these properties is increased or not significantly (less than 10-fold) decreased in vivo compared to an all RNA ribozyme or all DNA enzyme.

In yet another preferred embodiment, nucleic acid catalysts having chemical modifications which maintain or enhance enzymatic activity is provided. Such nucleic acid catalysts are also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples: the 5′-cap is selected from the group comprising inverted abasic residue (moiety); 4′, 5′-methylene nucleotide; l-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′, 4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details, see Wincott et al., International PCT publication No. WO 97/26270, incorporated by reference herein).

In yet another preferred embodiment, the 3′-cap is selected from a group comprising, 4′, 5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′, 4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO₂ or N(CH₃)_{2,} amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, N₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH-R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′—O—NH_{2,} which may be modified or unmodified. Such modified groups are described, for example, in Eckstein et al., U.S. Pat. No. 5,672,695 and Matulic-Adamic et al., WO 98/28317, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

### Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569; Beigelman et al., PCT WO99/05094, and Klimuk et al., PCT WO99/04819 all of which are incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example, oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalites by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totality by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect, the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J, 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, Hammerhead, DNAzyme, NCH, Amberzyme, Zinzyme or G-Cleaver ribozyme molecules and binding/cleavage sites within HBV RNA.

### EXAMPLE 1

### Identification of Potential Target Sites in Human HBV RNA

The sequence of human HBV was screened for accessible sites using a computer-folding algorithm. Regions of the RNA that did not form secondary folding structures and contained potential ribozyme and/or antisense binding/cleavage sites were identified. The sequences of these cleavage sites are shown in Tables IV-XI.

### EXAMPLE 2

### Selection of Enzymatic Nucleic Acid Cleavage Sites in Human HBV RNA

Ribozyme target sites were chosen by analyzing sequences of Human HBV (accession number: AF100308.1) and prioritizing the sites on the basis of folding. Ribozymes were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted herein, varying binding arm lengths can be chosen to optimize activity. Generally, at least 5 bases on each arm are able to bind to, or otherwise interact with, the target RNA.

### EXAMPLE 3

### Chemical Synthesis and Purification of Ribozymes and Antisense for Efficient Cleavage and/or blocking of HBV RNA

Ribozymes and antisense constructs were designed to anneal to various sites in the RNA message. The binding arms of the ribozymes are complementary to the target site sequences described above, while the antisense constructs are fully complementary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs were also synthesized from DNA templates using bacteriophage T7 RNA polymerase (Milligan and Uhlenbeck, 1989, Methods Enzymol. 180, 51). Ribozymes and antisense constructs were purified by gel electrophoresis using general methods or were purified by high pressure liquid chromatography (HPLC; see Wincott et al., supra; the totality of which is hereby incorporated herein by reference) and were resuspended in water. The sequences of the chemically synthesized ribozymes used in this study are shown below in Table

### EXAMPLE 4

### Ribozyme Cleavage of HBV RNA Target in vitro

Ribozymes targeted to the human HBV RNA are designed and synthesized as described above. These ribozymes can be tested for cleavage activity in vitro, for example using the following procedure. The target sequences and the nucleotide location within the HBV RNA are given in Tables IV-XI.

Cleavage Reactions: Full-length or partially full-length, internally-labeled target RNA for ribozyme cleavage assay is prepared by in vitro transcription in the presence of [α-³²p] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2×concentration of purified ribozyme in ribozyme cleavage buffer (50 mM Tris-HCl, pH 7.5 at 37° C., 10 mM MgCl₂) and the cleavage reaction was initiated by adding the 2×ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imagers® quantitation of bands representing the intact substrate and the cleavage products.

### EXAMPLE 5

### Transfection of HepG2 Cells with psHBV-I and Ribozymes

The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-1 vector (Those skilled in the art understand that other methods may be used to generate a replication competent cDNA). This was done with an EcoRI and Hind III restriction digest. Following completion of the digest, a ligation was performed under dilute conditions (20 1μg/ml) to favor intermolecular ligation. The total ligation mixture was then concentrated using Qiagen spin columns.

Secreted alkaline phosphatase (SEAP) was used to normalize the HBsAg levels to control for transfection variability. The pSEAP2-TK control vector was constructed by ligating a Bgl II-Hind III fragment of the pRL-TK vector (Promega), containing the herpes simplex virus thymidine kinase promoter region, into Bgl II/Hind III digested pSEAP2-Basic (Clontech). Hep G2 cells were plated (3×10⁴ cells/well) in 96-well microtiter plates and incubated overnight. A lipid/DNA/ribozyme complex was formed containing (at final concentrations) cationic lipid (15 μg/ml), prepared psHBV-1 (4.5 μg/ml), pSEAP2-TK (0.5 μg/ml), and ribozyme (100 μM). Following a 15 min. incubation at 37° C., the complexes were added to the plated Hep G2 cells. Media was removed from the cells 96 hr. post-transfection for HBsAg and SEAP analysis.

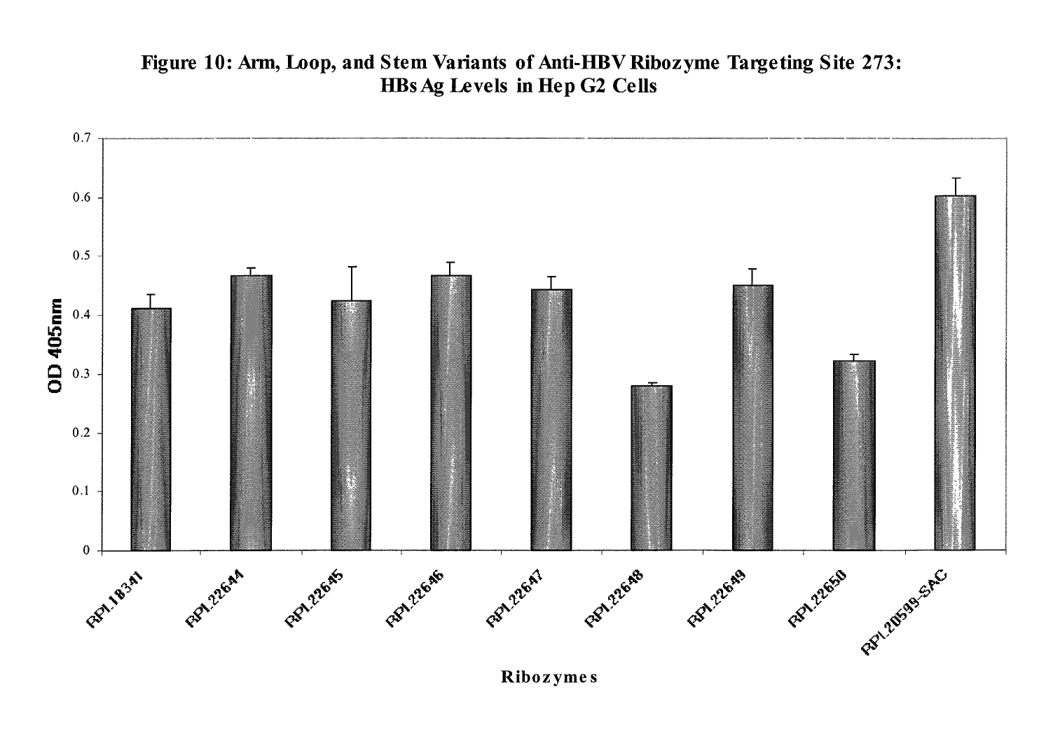

Transfection of the human hepatocellular carcinoma cell line, Hep G2, with replication competent HBV DNA results in the expression of HBV proteins and the production of virions. To investigate the potential use of ribozymes for the treatment of chronic HBV infection, a series of ribozymes that target the 3′ terminus of the HBV genome have been synthesized. Ribozymes targeting this region have the potential to cleave all four major HBV RNA transcripts as well as the potential to block the production of HBV DNA by cleavage of the pregenomic RNA. To test the efficacy of these HBV ribozymes, they were co-transfected with HBV genomic DNA into Hep G2 cells, and the subsequent levels of secreted HBV surface antigen (HBsAg) were analyzed by ELISA. To control for variability in transfection efficiency, a control vector which expresses secreted alkaline phosphatase (SEAP), was also co-transfected. The efficacy of the HBV ribozymes was determined by comparing the ratio of HBsAg:SEAP and/or HBeAg:SEAP to that of a scrambled attenuated control (SAC) ribozyme. Twenty-five ribozymes (RPI18341, RPI18356, RPI18363, RPI18364, RPI18365, RPI18366, RPI18367, RPI18368, RPI18369, RPI18370, RPI18371, RPI18372, RPI18373, RPI18374, RPI18303, RPI18405, RPI18406, RPI18407, RPI18408, RPI18409, RPI18410, RPI18411, RPI18418, RPJ18419, and RPI18422) have been identified which cause a reduction in the levels of HBsAg and/or HBeAg as compared to the corresponding SAC ribozyme. In addition, loop variant anti-HBV ribozymes targeting site 273 were tested using this system, the results of this study are summarized in FIG. 10. As indicated in the figure, the ribozymes tested demonstrate significant reduction in HepG2 HBsAg levels as compared to a scrambled attenuated core ribozyme control, with RPI 22650 and RPI 22649 showing the greatest decrease in HBsAg levels.

### EXAMPLE 6

### Analysis of HBsAg and SEAP Levels Following Ribozyme Treatment

Immulon 4 (Dynax) microtiter wells were coated overnight at 40° C. with anti-HBsAg Mab (Biostride B88-95-31ad,ay) at 1 μg/ml in Carbonate Buffer (Na2CO3 15 mM, NaHCO3 35 mM, pH 9.5). The wells were then washed 4× with PBST (PBS, 0.05% Tween® 20) and blocked for 1 hr at 37° C. with PBST, 1% BSA. Following washing as above, the wells were dried at 37° C. for 30 min. Biotinylated goat ant-HBsAg (Accurate YVS1807) was diluted 1:1000 in PBST and incubated in the wells for 1 hr. at 37° C. The wells were washed 4× with PBST. Streptavidin/Alkaline Phosphatase Conjugate (Pierce 21324) was diluted to 250 ng/ml in PBST, and incubated in the wells for 1 hr. at 370° C. After washing as above, p-nitrophenyl phosphate substrate (Pierce 37620) was added to the wells, which were then incubated for 1 hr. at 37° C. The optical density at 405 nm was then determined. SEAP levels were assayed using the Great EscAPe® Detection Kit (Clontech K2041-1), as per the manufacturers instructions.

### EXAMPLE 7

### X-gene Reporter Assay

The effect of ribozyme treatment on the level of transactivation of a SV40 promoter driven firefly luciferase gene by the HBV X-protein was analyzed in transfected Hep G2 cells. As a control for variability in transfection efficiency, a Renilla luciferase reporter driven by the TK promoter, which is not transactivated by the X protein, was used. Hep G2 cells were plated (3×10⁴ cells/well) in 96-well microtiter plates and incubated overnight. A lipid/DNA/ribozyme complex was formed containing (at final concentrations) cationic lipid (2.4 μg/ml), the X-gene vector pSBDR(2.5 μg/ml), the firefly reporter pSV40HCVluc (0.5 1g/ml), the Renilla luciferase control vector pRL-TK (0.5 μg/ml), and ribozyme (100 μM). Following a 15 min. incubation at 37° C., the complexes were added to the plated Hep G2 cells. Levels of firefly and Renilla luciferase were analyzed 48 hr. post transfection, using Promega's Dual-Luciferase Assay System.

The HBV X protein is a transactivator of a number of viral and cellular genes. Ribozymes which target the X region were tested for their ability to cause a reduction in X protein transactivation of a firefly luciferase gene driven by the SV40 promoter in transfected Hep G2 cells. As a control for transfection variability, a vector containing the Renilla luciferase gene driven by the TK promotor, which is not activated by the X protein, was included in the co-transfections. The efficacy of the HBV ribozymes was determined by comparing the ratio of firefly luciferase: Renilla luciferase to that of a scrambled attenuated control (SAC) ribozyme. Eleven ribozymes (RPI18365, RPI18367, RPI18368, RPI18371, RPI18372, RPI18373, RPI18405, RPI18406, RPI18411, RPI18418, RPI18423) were identified which cause a reduction in the level of transactivation of a reporter gene by the X protein, as compared to the corresponding SAC ribozyme.

### EXAMPLE 8

### HBV transgenic mouse study A

A transgenic mouse strain (founder strain 1.3.32 with a C57B1/6 background) that expresses HBV RNA and forms HBV viremia (Morrey et al., 1999, Antiviral Res., 42, 97-108; Guidotti et al., 1995, J. Virology, 69, 10, 6158-6169) was utilized to study the in vivo activity of ribozymes (RPI.18341, RPI.18371, RPI.18372, and RPI.18418) of the instant invention. This model is predictive in screening for anti-HBV agents. Ribozyme or the equivalent volume of saline was administered via a continuous s.c. infusion using Alzet® mini-osmotic pumps for 14 days. Alzet® pumps were filled with test material(s) in a sterile fashion according to the manufacturer's instructions. Prior to in vivo implantation, pumps were incubated at 37° C. overnight (≧18 hours) to prime the flow modulators. On the day of surgery, animals were lightly anesthetized with a ketamine/xylazine cocktail (94 mg/kg and 6 mg/kg, respectively; 0.3 ml, IP). Baseline blood samples (200 ,μl) were obtained from each animal via a retro-orbital bleed. For animals in groups 1-5 (Table XII), a 2 cm area near the base of the tail was shaved and cleansed with betadine surgical scrub and sequentially with 70% alcohol. A 1 cm incision in the skin was made with a #15 scalpel blade or a blunt pair of scissors near the base of the tail. Forceps were used to open a pocket rostrally (ie., towards the head) by spreading apart the subcutaneous connective tissue. The pump was inserted with the delivery portal pointing away from the incision. Wounds were closed with sterile 9-mm stainless steel clips or with sterile 4-0 suture. Animals were then allowed to recover from anesthesia on a warm heating pad before being returned to their cage. Wounds were checked daily. Clips or sutures were replaced as needed. Incisions typically healed completely within 7 days post-op. Animals were then deeply anesthetized with the ketamine/xylazine cocktail (150 mg/kg and 10 mg/kg, respectively; 0.5 ml, IP) on day 14 post pump implantation. A midline thoracotomy/laparatomy was performed to expose the abdominal cavity and the thoracic cavity. The left ventricle was cannulated at the base and animals exsanguinated using a 23G needle and 1 ml syringe. Serum was separated, frozen and analyzed for HBV DNA and antigen levels. Experimental groups were compared to the saline control group in respect to percent change from day 0 to day 14. HBV DNA was assayed by quantitative PCR.

### Results

Table XII is a summary of the group designation and dosage levels used in this HBV transgenic mouse study. Baseline blood samples were obtained via a retroorbital bleed and animals (N=10/group) received anti-HBV ribozymes (100 mg/kg/day) as a continuous SC infusion. After 14 days, animals treated with a ribozyme targeting site 273 (RPI.18341) of the HBV RNA showed a significant reduction in serum HBV DNA concentration, compared to the saline treated animals as measured by a quantitative PCR assay. More specifically, the saline treated animals had a 69% increase in serum HBV DNA concentrations over this 2-week period while treatment with the 273 ribozyme (RPI.18341) resulted in a 60% decrease in serum HBV DNA concentrations. Ribozymes directed against sites 1833 (RPI.18371), 1873 (RPI.18418), and 1874 (RPI.18372) decreased serum HBV DNA concentrations by 49%, 15% and 16%, respectively.

### EXAMPLE 9

### HBV transgenic mouse study B

A transgenic mouse strain (founder strain 1.3.32 with a C57B 1/6 background) that expresses HBV RNA and forms HBV viremia (Morrey et al., 1999, Antiviral Res., 42, 97-108; Guidotti et al., 1995, J. Virology, 69, 10, 6158-6169) was utilized to study the in vivo activity of ribozymes (RPI.18341 and RPI.18371) of the instant invention. This model is predictive in screening for anti-HBV agents. Ribozyme or the equivalent volume of saline was administered via a continuous s.c. infusion using Alzet® mini-osmotic pumps for 14 days. Alzet® pumps were filled with test material(s) in a sterile fashion according to the manufacturer's instructions. Prior to in vivo implantation, pumps were incubated at 37° C. overnight e>18 hours) to prime the flow modulators. On the day of surgery, animals were lightly anesthetized with a ketamine/xylazine cocktail (94 mg/kg and 6 mg/kg, respectively; 0.3 ml, IP) Baseline blood samples (200 μl) were obtained from each animal via a retro-orbital bleed. For animals in groups 1-10 (Table XIII), a 2 cm area near the base of the tail was shaved and cleansed with betadine surgical scrub and sequentially with 70% alcohol. A 1 cm incision in the skin was made with a #15 scalpel blade or a blunt pair of scissors near the base of the tail. Forceps were used to open a pocket rostrally (ie., towards the head) by spreading apart the subcutaneous connective tissue. The pump was inserted with the delivery portal pointing away from the incision. Wounds were closed with sterile 9-mm stainless steel clips or with sterile 4-0 suture. Animals were then allowed to recover from anesthesia on a warm heating pad before being returned to their cage. Wounds were checked daily. Clips or sutures were replaced as needed. Incisions typically healed completely within 7 days post-op. Animals were then deeply anesthetized with the ketamine/xylazine cocktail (150 mg/kg and 10 mg/kg, respectively; 0.5 ml, IP) on day 14 post pump implantation. A midline thoracotomy/ laparatomy was performed to expose the abdominal cavity and the thoracic cavity. The left ventricle was cannulated at the base and animals exsanguinated using a 23G needle and 1 ml syringe. Serum was separated, frozen and analyzed for HBV DNA and antigen levels. Experimental groups were compared to the saline control group in respect to percent change from day 0 to day 14. HBV DNA was assayed by quantitative PCR. Additionally, mice treated with 3TC® by oral gavage at a dose of 300 mg/kg/day for 14 days (group 11, Table XIII) were used as a positive control.

### Results

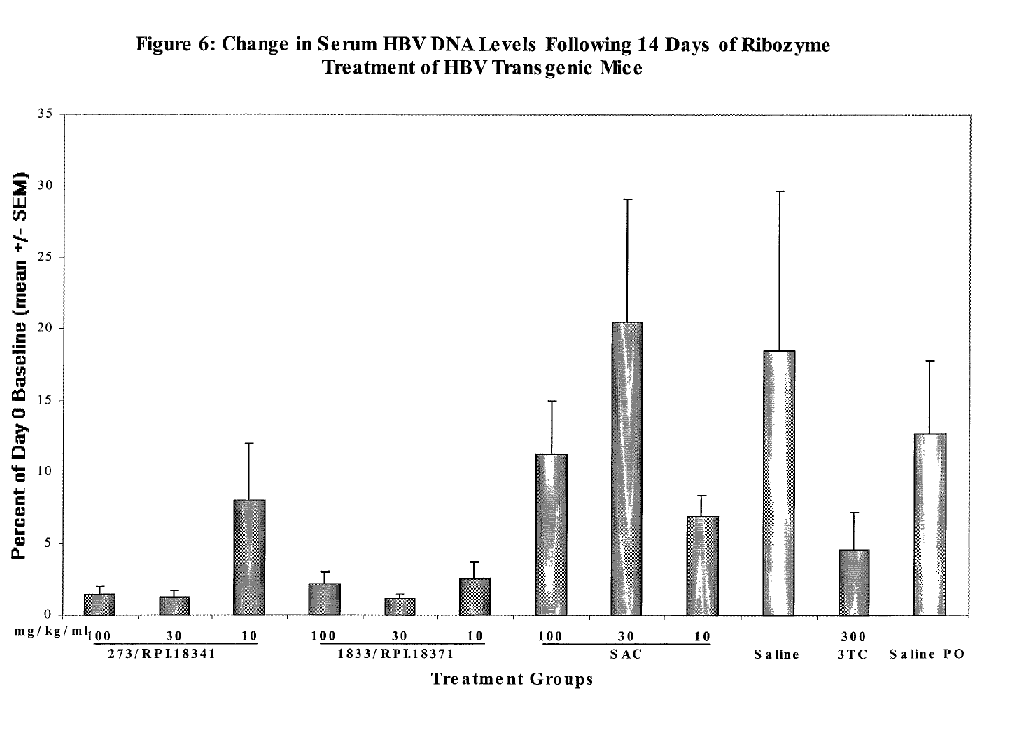

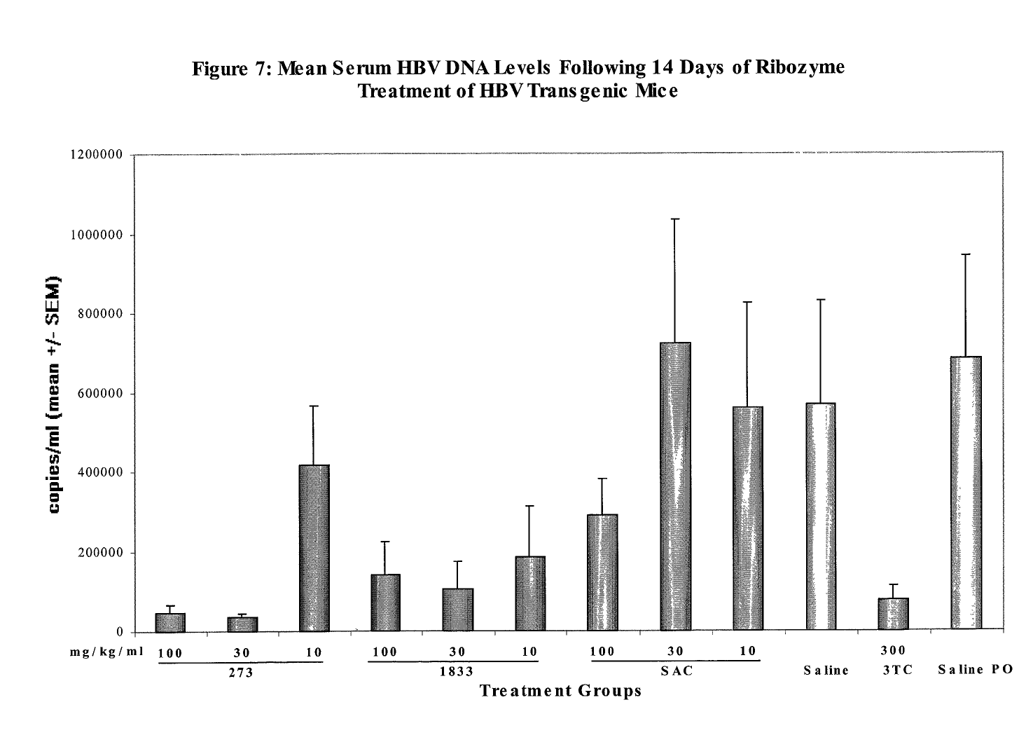

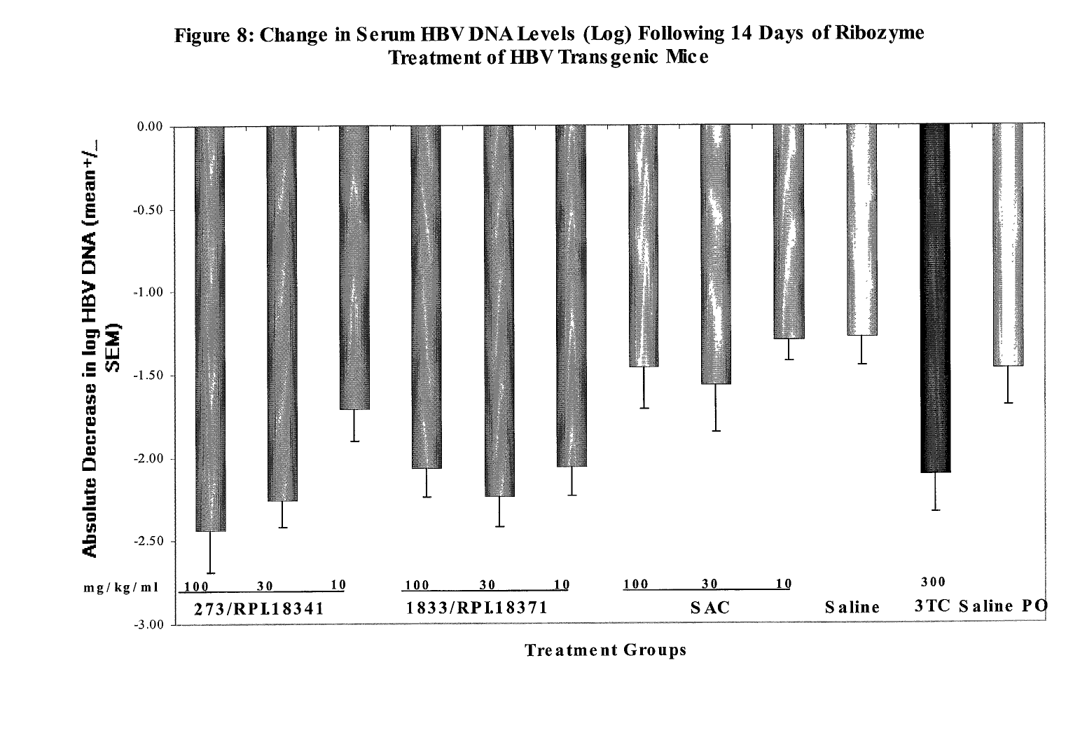

Table XIII is a summary of the group designation and dosage levels used in this HBV transgenic mouse study. Baseline blood samples were obtained via a retroorbital bleed and animals (N=15/group) received anti-HBV ribozymes (100 mg/kg/day, 30 mg/kg/day, 10 mg/kg/day) as a continuous SC infusion. The results of this study are summarized in FIGS. 6, 7, and 8. As FIGS. 6, 7, and 8 demonstrate, Ribozymes directed against sites 273 (RPI.18341) and 1833 (RPI.18371) demonstrate reduction in the serum HBV DNA levels following 14 days of ribozyme treatment in HBV transgenic mice, as compared to scrambled attenuated core (SAC) ribozyme and saline controls. Furthermore, these ribozymes provide similar, and in some cases, greater reduction of serum HBV DNA levels, as compared to the 3TCOR positive control, at lower doses than the 3TC® positive control.

### EXAMPLE 10

### HBV DNA reduction in HepG2.2.15 cells

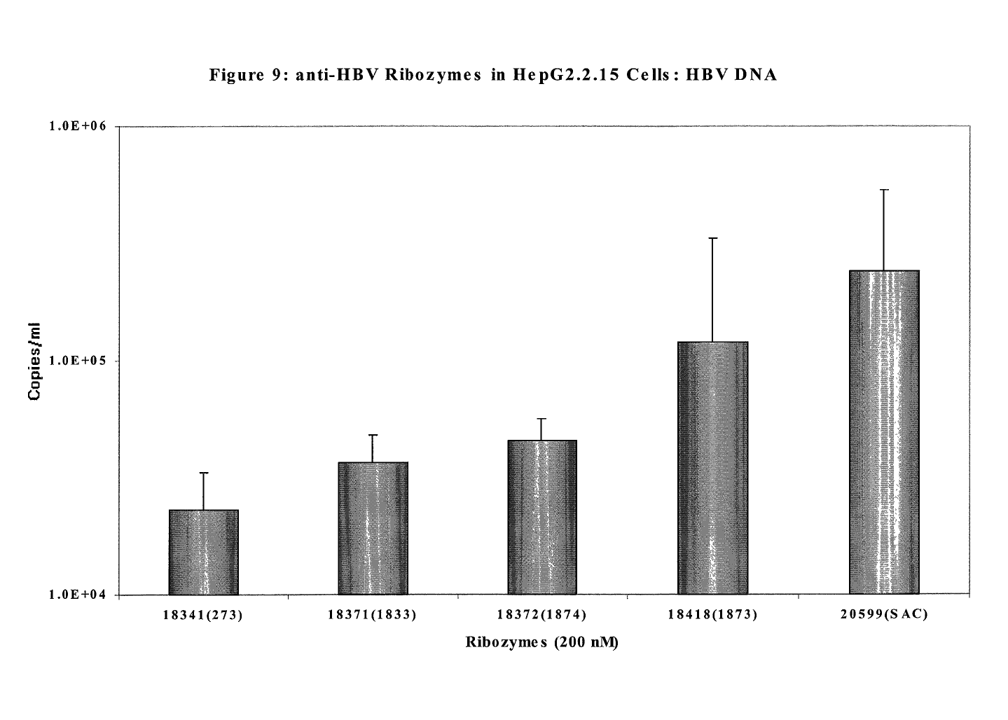

Ribozyme treatment of HepG2.2.15 cells was performed in a 96-well plate format, with 12 wells for each different ribozyme tested (RPI.18341, RPI.18371, RPI.18372, RPI.18418, RPI.20599SAC). HBV DNA levels in the media collected between 120 and 144 hours following transfection was determined using the Roche Amplicor HBV Assay. Treatment with RPI.18341 targeting site 273 resulted in a significant (P<0.05) decrease in HBV DNA levels of 62% compared to the SAC (RPI.20599). Treatment with RPI.18371 (site 1833) or RPI.18372 (site 1874) resulted in reductions in HBV DNA levels of 55% and 58% respectively, as compared to treatment with the SAC RPJ.20599 (see FIG. 9).

### EXAMPLE 11

### RPI 18341 Combination Treatment with Lamivudine/Infergeng

The therapeutic use of nucleic acid molecules of the invention either alone or in combination with current therapies, for example lamivudine or type 1 IFN, can lead to improved HBV treatment modalities. To assess the potential of combination therapy, HepG2 cells transfected with a replication competent HBV cDNA, were treated with RPI 18341(HepBzyme™), Infergen®b (Amngen, Thousand Oaks Calif.), and/or Lamivudine (Epivirg®: GlaxoSmithKline, Research Triangle Park N.C.) either alone or in combination. Results indicated that combination treatment with either RPI 18341 plus Infergeng® or combination of RPI 18341 plus lamivudine results in additive down regulation of HBsAg expression (P<0.001). These studies can be applied to the treatment of lamivudine resistant cells to further assses the potential for combination therapy of RPI 18341 plus currently available therapies for the treatment of chronic Hepatitis B.

Hep G2 cells were plated (2×104 cells/well) in 96-well microtiter plates and incubated overnight. A cationic lipid/DNA/ribozyme complex was formed containing (at final concentrations) lipid (11-15 μg/mL), re-ligated psHBV-1 (4.5 μg/mL) and ribozyme (100-200 nM) in growth media. Following a 15 min incubation at 37° C., 20 μL of the complex was added to the plated Hep G2 cells in 80 μL of growth media minus antibiotics. For combination treatment with interferon, interferon (Infergen®, Amgen, Thousand Oaks Calif.) was added at 24 hr post-transfection and then incubated for an additional 96 hr. In the case of co-treatment with Lamivudine (3TC®), the ribozyme-containing cell culture media was removed at 120 hr post-transfection, fresh media containing Lamivudine (Epivir®: GlaxoSmithKline, Research Triangle Park N.C.) was added, and then incubated for an additional 48 hours. Treatment with Lamivudine or interferon individually was done on Hep G2 cells transfected with the pSHBV-1 vector alone and then treated identically to the co-treated cells. All transfections were performed in triplicate. Analysis of HBsAg levels was performed using the Diasorin HBsAg ELISA kit.

### Results

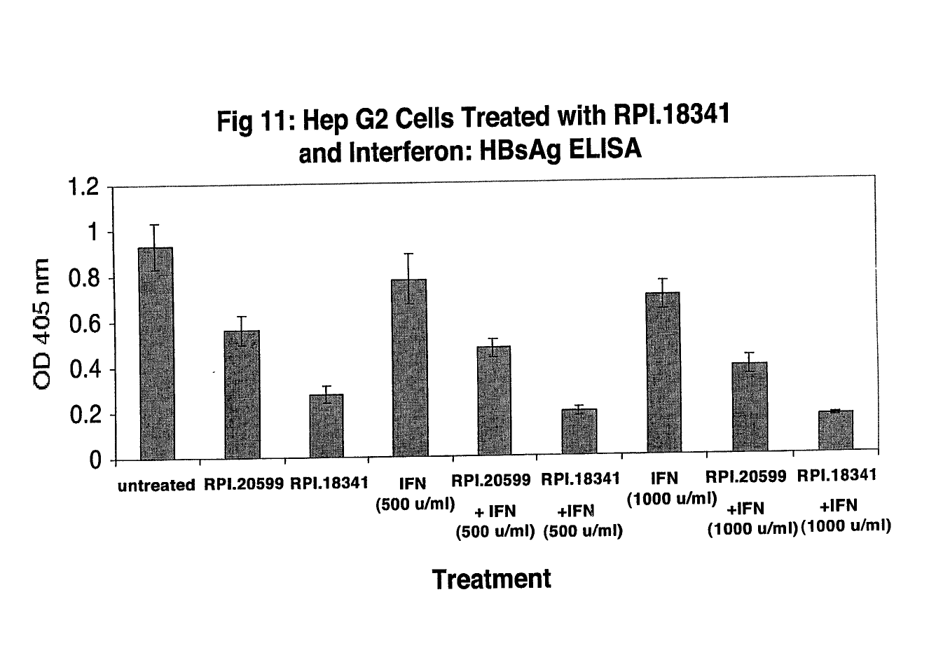

At either 500 or 1000 units of Infergen®g, the addition of 200 nM of RPI.18341 results in a 75-77% increase in anti-HBV activity as judged by the level of HBsAg secreted from the treated Hep G2 cells. Conversely, the anti-HBV activity of RPI.18341(at 200 nM) is increased 31-39% when used in combination of 500 or 1000 units of Infergen® (FIG. 11).

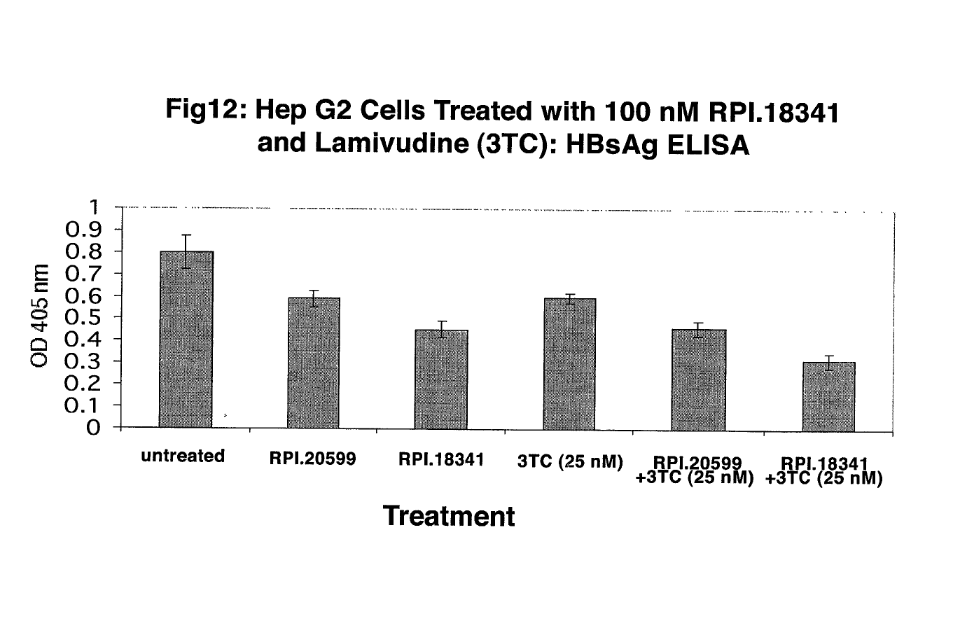

At 25 nM Lamivudine (3TC®), the addition of 100 nM of RPI.18341 results in a 48% increase in anti-HBV activity as judged by the level of HBsAg secreted from treated Hep G2 cells. Conversely, the anti-HBV activity of RPI.18341 (at 100 nM) is increased 31% when used in combination with 25 nM Lamivudine (FIG. 12).

### Cell Culture Models

As previously mentioned, HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA would be co-transfected with ribozymes in cell culture. Such an approach has been used to report intracellular ribozyme activity against HBV (zu Putlitz, et al., 1999, J Virol., 73, 5381-5387, and Kim et al., 1999, Biochem. Biophys. Res. Commun., 257, 759-765). In addition, stable hepatocyte cell lines have been generated that express HBV. In these cells only ribozyme would need to be delivered; however, a delivery screen would need to be performed. Intracellular HBV gene expression can be assayed by a Taqman® assay for HBV RNA or by ELISA for HBV protein. Extracellular virus can be assayed by PCR for DNA or ELISA for protein. Antibodies are commercially available for HBV surface antigen and core protein. A secreted alkaline phosphatase expression plasmid can be used to normalize for differences in transfection efficiency and sample recovery.

### Animal Models

There are several small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Guidotti et al., 1995, J. Virology, 69, 10, 6158-6169; Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J Viral Hepat., 3, 19-22).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

### Indications

Particular degenerative and disease states that can be associated with HBV expression modulation include but are not limited to, BBV infection, hepatitis, cancer, tumorigenesis, cirrhosis, liver failure and others.

The present body of knowledge in HBV research indicates the need for methods to assay HBV activity and for compounds that can regulate HBV expression for research, diagnostic, and therapeutic use.

Lamivudine (3TC®), L-FMAU, adefovir dipivoxil, type 1 Interferon, therapeutic vaccines, steriods, and 2′-5′ Oligoadenylates are non-limiting examples of pharmaceutical agents that can be combined with or used in conjunction with the nucleic acid molecules (e.g. ribozymes and antisense molecules) of the instant invention. Those skilled in the art will recognize that other drugs or other therapies can similarly and readily be combined with the nucleic acid molecules of the instant invention (e.g. ribozymes and antisense molecules) and are, therefore, within the scope of the instant invention.

### Diagnostic Uses

The nucleic acid molecules of this invention (e.g., ribozymes) may be used as diagnostic tools to examine genetic drift and mutations within diseased cells or to detect the presence of HBV RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with HBV-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA can be cleaved by both ribozymes to demonstrate the relative ribozyme efficiencies in the reactions and the absence of cleavage of the “non-targeted” RNA species. The cleavage products from the synthetic substrates can also serve to generate size markers for the analysis of wild-type and mutant RNAs in the sample population. Thus each analysis canl involve two ribozymes, two substrates and one unknown sample which will be combined into six reactions. The presence of cleavage products will be determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HBV) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

### Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage products with
3′-OH and 5′-guanosine.
Additional protein cofactors required in some cases to help folding and maintainance of
the active structure.
Over 300 known members of this class. Found as an intervening sequence in Tetrahymena
thermophila rRNA, fungal mitochondria, chloroplasts, phage T4, blue-green algae, and
others.
Major structural features largely established through phylogenetic comparisons,
mutagenesis, and biochemical studies ⁱ,ⁱⁱ.
Complete kinetic framework established for one ribozyme ⁱⁱⁱ,^{iv},^{v},^{vi}.
Studies of ribozyme folding and substrate docking underway ^{vii},^{viii},^{ix}.
Chemical modification investigation of important residues well established ^{x},^{xi}.
The small (4-6 nt) binding site may make this ribozyme too non-specific for targeted RNA
cleavage, however, the Tetrahymena group I intron has been used to repair a “defective”
β-galactosidase message by the ligation of new β-galactosidase sequences onto the
defective message ^{xii}.
RNAse P RNA (M1 RNA)
Size: ˜290 to 400 nucleotides.
RNA portion of a ubiquitous ribonucleoprotein enzyme.
Cleaves tRNA precursors to form mature tRNA ^{xiii}.
Reaction mechanism: possible attack by M²⁺-OH to generate cleavage products with 3′-
OH and 5′-phosphate.
RNAse P is found throughout the prokaryotes and eukaryotes. The RNA subunit has
been sequenced from bacteria, yeast, rodents, and primates.
Recruitment of endogenous RNAse P for therapeutic applications is possible through
hybridization of an External Guide Sequence (EGS) to the target RNA ^{xiv},^{xv}
Important phosphate and 2′ OH contacts recently identified ^{xvi},^{xvii}
Group II Introns
Size: >1000 nucleotides.
Trans cleavage of target RNAs recently demonstrated ^{xviii},^{xix}.
Sequence requirements not fully determined.
Reaction mechanism: 2′-OH of an internal adenosine generates cleavage products with 3′-
OH and a “lariat” RNA containing a 3′-5′ and a 2′-5′ branch point.
Only natural ribozyme with demonstrated participation in DNA cleavage ^{xx},^{xxi} in
addition to RNA cleavage and ligation.
Major structural features largely established through phylogenetic comparisons ^{xxii}.
Important 2′ OH contacts beginning to be identified ^{xxiii}
Kinetic framework under development ^{xxiv}
Neurospora VS RNA
Size: ˜144 nucleotides.
Trans cleavage of hairpin target RNAs recently demonstrated ^{xxv}.
Sequence requirements not fully determined.
Reaction mechanism: attack by 2′-OH 5′ to the scissile bond to generate cleavage products
with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage products
with 2′,3′-cyclic phosphate and 5′-OH ends.
14 known members of this class. Found in a number of plant pathogens (virusoids) that
use RNA as the infectious agent.
Essential structural features largely defined, including 2 crystal structures ^{xxvi},^{xxvii}
Minimal ligation activity demonstrated (for engineering through in vitro selection) ^{xxviii}
Complete kinetic framework established for two or more ribozymes ^{xxix}.
Chemical modification investigation of important residues well established ^{xxx}.
Hairpin Ribozyme
Size: ˜50 nucleotides.
Requires the target sequence GUC immediately 3′ of the cleavage site.
Binds 4-6 nucleotides at the 5′-side of the cleavage site and a variable number to the
3′-side of the cleavage site.
Reaction mechanism: attack by 2′-OH 5′ to the scissile bond to generate cleavage products
with 2′,3′-cyclic phosphate and 5′-OH ends.
3 known members of this class. Found in three plant pathogen (satellite RNAs of the
tobacco ringspot virus, arabis mosaic virus and chicory yellow mottle virus) which uses
RNA as the infectious agent.
Essential structural features largely defined ^{xxxi},^{xxxii},^{xxxiii},^{xxiv}
Ligation activity (in addition to cleavage activity) makes ribozyme amenable to
engineering through in vitro selection ^{xxxv}
Complete kinetic framework established for one ribozyme ^{xxxvi}.
Chemical modification investigation of important residues begun ^{xxxvii},^{xxxviii}.
Hepatitis Delta Virus (HDV) Ribozyme
Size: ˜60 nucleotides.
Trans cleavage of target RNAs demonstrated ^{xxxix}.
Binding sites and structural requirements not fully determined, although no sequences 5′
of cleavage site are required. Folded ribozyme contains a pseudoknot structure ^{xl}.
Reaction mechanism: attack by 2′-OH 5′ to the scissile bond to generate cleavage products
with 2′,3′-cyclic phosphate and 5′-OH ends.
Only 2 known members of this class. Found in human HDV.
^{xli}Circular form of HDV is active and shows increased nuclease stability ^{xlii}
ⁱ Michel, Francois; Westhof, Eric. Slippery substrates. Nat. Struct. Biol. (1994), 1(1), 5-7.
ⁱⁱ Lisacek, Frederique; Diaz, Yolande; Michel, Francois. Automatic identification of group I intron cores in genomic DNA sequences. J. Mol. Biol. (1994), 235(4), 1206-17.
ⁱⁱⁱ Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv} Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v} Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi} Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii} Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii} Banerjee, Aloke Raj; Turner, Douglas H.. The time dependence of chemical modification reveals slow steps in the folding of a group I ribozyme. Biochemistry (1995), 34(19), 6504-12.
^{ix} Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x} Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi} Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii} Sullenger, Bruce A.; Cech, Thomas R.. Ribozyme-mediated repair of defective mRNA by targeted trans-splicing. Nature (London) (1994), 371(6498), 619-22.
^{xiii} Robertson, H. D.; Altman, S.; Smith, J. D. J. Biol. Chem., 247, 5243-5251 (1972).
^{xiv} Forster, Anthony C.; Altman, Sidney. External guide sequences for an RNA enzyme. Science (Washington, D.C., 1883-) (1990), 249(4970), 783-6.
^{xv} Yuan, Y.; Hwang, E. S.; Altman, S. Targeted cleavage of mRNA by human RNase P. Proc. Natl. Acad. Sci. USA (1992) 89, 8006-10.
^{xvi} Harris, Michael E.; Pace, Norman R.. Identification of phosphates involved in catalysis by the ribozyme RNase P RNA. RNA (1995), 1(2), 210-18.
^{xvii} Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii} Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix} Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx} Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi} Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii} Michel, Francois; Ferat, Jean Luc. Structure and activities of group II introns. Annu. Rev. Biochem. (1995), 64, 435-61.
^{xxiii} Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv} Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv} Guo, Hans C. T.; Collins, Richard A.. Efficient trans-cleavage of a stem-loop RNA substrate by a ribozyme derived from Neurospora VS RNA. EMBO J. (1995), 14(2), 368-76.
^{xxvi} Scott, W. G., Finch, J. T., Aaron, K. The crystal structure of an all RNA hammerhead ribozyme: A proposed mechanism for RNA catalytic cleavage. Cell, (1995), 81, 991-1002.
^{xxvii} McKay, Structure and function of the hammerhead ribozyme: an unfinished story. RNA, (1996), 2, 395-403.
^{xxviii} Long, D., Uhlenbeck, O., Hertel, K. Ligation with hammerhead ribozymes. U.S. Pat. No. 5,633,133.
^{xxix} Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx} Beigelman, L., et al., Chemical modifications of hammerhead ribozymes. J. Biol. Chem., (1995) 270, 25702-25708.
^{xxxi} Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
^{xxxii} Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii} Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv} Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv} Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi} Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii} Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii} Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix} Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl} Perrotta, Anne T.; Been, Michael D.. A pseudoknot-like structure required for efficient self-cleavage of hepatitis delta virus RNA. Nature (London) (1991), 350(6317), 434-6.
^{xli}
^{xlii} Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| Reagent | Equivalents | Amount | | Wait Time* DNA | | Wait Time* 2′-O-methyl | | Wait Time*RNA | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B 0.2 μmol Synthesis Cycle ABI 394 Instrument

| Reagent | Equivalents | Amount | | Wait Time* DNA | | Wait Time* 2′-O-methyl | | Wait Time*RNA | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: DNA/ | Amount: DNA/2′-O- | | Wait Time* | | Wait Time* 2′-O- | | Wait Time* | |
|---|---|---|---|---|---|---|---|---|---|
| Reagent | 2′-O-methyl/Ribo | methyl/Ribo | | DNA | | methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6 8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE III
HBV Strains and Accession numbers

| Accession | |
|---|---|
| Number | NAME |
| AF100308.1 | AF100308 Hepatitis B virus strain 2-18, complete |
| AB026815.1 | AB026815 Hepatitis B virus DNA, complete genome, |
| AB033559.1 | AB033559 Hepatitis B virus DNA, complete genome, |
| AB033558.1 | AB033558 Hepatitis B virus DNA, complete genome, |
| AB033557.1 | AB033557 Hepatitis B virus DNA, complete genome, |
| AB033596.1 | AB033556 Hepatitis B virus DNA, complete genome, |
| AB033555.1 | AB033555 Hepatitis B virus DNA, complete genome, |
| AB033554.1 | AB033554 Hepatitis B virus DNA, complete genome, |
| AB033553.1 | AB033553 Hepatitis B virus DNA, complete genome, |
| AB033552.1 | AB033552 Hepatitis B virus DNA, complete genome, |
| AB033551.1 | AB033551 Hepatitis B virus DNA, complete genome, |
| AB033550.1 | AB033550 Hepatitis B virus DNA, complete genome |
| AF143308.1 | AF143308 Hepatitis B virus clone WB1254, complete |
| AF143307.1 | AF143307 Hepatitis B virus clone RM518, complete |
| AP143306.1 | AF143306 Hepatitis B virus clone RM5l7, complete |
| AF143305.1 | AF143305 Hepatitis B virus clone RM501, complete |
| AF143304.1 | AF143304 Hepatitis B virus clone HD319, complete |
| AF143303.1 | AF143303 Hepatitis B virus clone HD1406, complete |
| AF143302.1 | AF143302 Hepatitis B virus clone HD1402, complete |
| AF143301.1 | AF143301 Hepatitis B virus clone BW1903, complete |
| AF143300.1 | AF143300 Hepatitis B virus clone 7832-G4, complete |
| AF143299.1 | AF143299 Hepatitis B virus clone 7744-G9, complete |
| AF143298.1 | AF143298 Hepatitis B virus clone 7720-G8, complete |
| AB026814.1 | AB026814 Hepatitis B virus DNA, complete genome, |
| AB026813.1 | AB026813 Hepatitis B virus DNA, complete genome, |
| AB026812.1 | AB026812 Hepatitis B virus DNA, complete genome, |
| AB026S11.1 | AB026811 Hepatitis B virus DNA, complete genome, |
| AJ131956.1 | HBV131956 Hepatitis B virus complete genome, |
| AF151735.1 | AF151735 Hepatitis B virus, complete genome |
| AF090842.1 | AF090842 Hepatitis B virus strain G5.27295, complete |
| AF090841.1 | AF090841 Hepatitis B virus strain G4.27241, complete |
| AF090840.1 | AF090840 Hepatitis B virus strain G3.27270, complete |
| AF090839.1 | AF090839 Hepatitis B virus strain G2.27246, complete |
| AF090838.1 | AF090838 Hepatitis B virus strain P1.27239, complete |
| Y18858.1 | HBV18838 Hepatitis B virus complete genome, isolate |
| Y18857.1 | HBV18857 Hepatitis B virus complete genome, isolate |
| D12980.1 | HPBCG Hepatitis B virus subtype adr(SRADR) DNA, |
| Y18856.1 | HBV18856 Hepatitis B virus complete genome, isolate |
| Y18855.1 | HBV18855 Hepatitis B virus complete genome, isolate |
| AJ131133.1 | HBV131133 Hepatitis B virus, complete genome, strain |
| X80925.1 | HBVP6PCXX Hepatitis B virus (patient 6) complete |
| X80926.1 | HBVP5PCXX Hepatitis B virus (patient 5) complete |
| X80924.1 | HBVP4PCXX Hepatitis B virus (patient 4) complete |
| AF100309.1 | Hepatitis B virus strain 56, complete qenome |
| AF068756.1 | AF068756 Hepatitis B virus, complete genome |
| AF043593.1 | AF043593 Hepatitis B virus isolate 6/89, complete |
| Y07587.1 | HBVAYWGEN Hepatitis B virus, complete genome |
| D28880.1 | D28880 Hepatitis B virus DNA, complete genome, strain |
| X98076.1 | HBVDEFVP3 Hepatitis B virus complete genome with |
| X98075.1 | HBVDEFVP2 Hepatitis B virus complete genome with |
| X98074.1 | HBVDEFVP1 Hepatitis B virus complete genome with |
| X98077.1 | HBVCGWITY Hepatitis B virus complete genome, wild |
| | type |
| X98072.1 | HBVCGINSC Hepatitis B virus complete genome with |
| X98073.1 | HBVCGINCX Hepatitis B virus complete genome with |
| U95551.1 | U95551 Hepatitis B virus subtype ayw, complete genome |
| D23684.1 | HPBC6T588 Hepatitis B virus (C6-TKBSB8) complete |
| | genome |
| D23683.1 | HPBC5HKO2 Hepatitis B virus (C5-HBVKO2) complete |
| | genome |
| D23682.1 | HPBB5HKO1 Hepatitis B virus (B5-HBVKO1) complete |
| | genome |
| D23681.1 | HPBC4HST2 Hepatitis H virus (C4-HBVST2) complete |
| | genome |
| D23680.1 | HPBB4HST1 Hepatitis B virus (B4-HBVST1) complete |
| | genome |
| D00331.1 | HPBADW3 Hepatitis B virus genome, complete genome |
| D00330.1 | HPBADW2 Hepatitis B virus genome, complete genome |
| D50489.1 | HPBA11A Hepatitis B virus DNA, complete genome |
| D23679.1 | HPBA3HMS2 Hepatitis B virus (A3-HBVMS2) complete |
| | genome |
| D23678.1 | HPBA2HYS2 Hepatitis B virus (A2-HBVYS2) complete |
| | genome |
| D23677.1 | HPBA1HKK2 Hepatitis B virus (A1-HBVKK2) complete |
| | genome |
| D16665.1 | HPBADRM Hepatitis B virus DNA, complete genome |
| D00329.1 | HPBADW1 Hepatitis B virus (HBV) genome, complete |
| | genome |
| X97851.1 | HBVP6CSX Hepatitis B virus (patient 6) complete |
| | genome |
| X97850.1 | HBVP4CSX Hepatitis B virus (patient 4) complete |
| | genome |
| X97849.1 | HBVP3CSX Hepatitis B virus (patient 3) complete |
| | genome |
| X97848.1 | HBVP2CSX Hepatitis B virus (patient 2) complete |
| | genome |
| X51970.1 | HVHEPB Hepatitis B virus (HBV 991) complete genome |
| M38636.1 | HPBCGADR Hepatitis B virus, subtype adr, complete |
| | genome |
| X59795.1 | HBVAYWMCG Hepatitis B virus (ayw subtype mutant) |
| M38454.1 | HPBADR1CG Hepatitis B virus , complete genome |
| M32138.1 | HPBHBVAA Hepatitis B virus variant HBV-alpha1, |
| | complete |
| J02203.1 | HPBAYW Human hepatitis B virus (subtype ayw), |
| | complete |
| M12906.1 | HPBADPA Hepatitis B virus subtype adr, complete |
| | genome |
| M54923.1 | HPBADWZ Hepatitis B virus (subtype adw), complete |
| | genome |
| L27106.1 | HPBMUT Hepatitis B virus mutant complete |
| | genome |

TABLE IV
HBV Substrate Sequence

| NT Position* | SUBSTRATE | SEQ ID |
|---|---|---|
| 82 | CUAUCGUCCCCUUCUUCAUC | 1. |
| 101 | CUACCGUUCCGGCC | 2. |
| 159 | CUUCUCAUCU | 3. |
| 184 | CUUCCCUUCACCAC | 4. |
| 269 | GACUCUCAGAAUGUCAACGAC | 5. |
| 381 | CUGUAGGCAUAAAUGGUCUG | 6. |
| 401 | GUUCACCAGCACCAUGCAACUUUUU | 7. |
| 424 | UUUCACGUCUGCCUAAUCAUC | 8. |
| 524 | AUUUGGAGCUUC | 9. |
| 562 | CUGACUUCUUUCCUUCUAUUC | 10. |
| 649 | CUCACCAUACCGCACUCA | 11. |
| 667 | GGCAAGCUAUUCUGUG | 12. |
| 717 | GGAAGUAAUUUGGAAGAC | 13. |
| 758 | CAGCUAUGUCAAUGUUAA | 14. |
| 783 | CUAAAAUCGGCCUAAAAUCAGAC | 15. |
| 812 | CAUUUCCUGUCUCACUUUUGGAAGAG | 16. |
| 887 | UCCUGCUUACAGAC | 17. |
| 922 | CAACACUUCCGGAAACUACUGUUGUUAG | 18. |
| 989 | CUCGCCUCGCAGACGAAGGUCUC | 19. |
| 1009 | CAAUCGCCGCGUCGCAGAAG | 20. |
| 1031 | AUCUCAAUCUCGGGAAUCUCAA | 21. |
| 1052 | AUGUUAGUAUCCCUUGGACUC | 22. |
| 1072 | CAUAAGGUGGGAAACUUUACUG | 23. |
| 1109 | CUGUACCUAUUCUUUAAAUCC | 24. |
| 1127 | CUGAGUGGCAAACUCCC | 25. |
| 1271 | CCAAAUAUCUGCCCUUGGACAA | 26. |
| 1297 | AUUAAACCAUAUUAUCCUGAACA | 27. |
| 1319 | AUGCAGUUAAUCAUUACUUCAAAACUA | 28. |
| 1340 | AAACUAGGCAUUA | 29. |
| 1370 | AGGCGGGCAUUCUAUAUAAGAGAG | 30. |
| 1393 | GAAACUACGCGCAGCGCCUCAUUUUGU | 31. |
| 1412 | CAUUUUGUGGGUCACCAUA | 32. |
| 1441 | CAAGAGCUACAGCAUGGG | 33. |

LOCUS HPBADR1CG 3221 bpDNA circular VRL Mar. 06, 1995
DEFINITION Hepatitis B virus, complete genome.
ACCESSION M38454
*The nucleotide number referred to in that table is the position of the 5′ end of the oligo in this sequence.

TABLE V

HUMAN HBV HAMMERHEAD RIBOZYME AND TARGET SEQUENCE

| | | | | |
|---|---|---|---|---|
| | | Seq | | Seq |
| Pos | Substrate | ID | Hammerhead | ID |

| | | | | |
|---|---|---|---|---|
| 13 | CCACCACU U UCCACCAA | 34 | UUGGUGGA CUGAUGAG GCCGUUAGGC CGAA AGUGGUGG | 2543 |
| 14 | CACCACUU U CCACCAAA | 35 | UUUGGUGG CUGAUGAG GCCGUUAGGC CGAA AAGUGGUG | 2544 |
| 15 | ACCACUUU C CACCAAAC | 36 | GUUUGGUG CUGAUGAG GCCGUUAGGC CGAA AAAGUGGU | 2545 |
| 25 | ACCAAACU C UUCAAGAU | 37 | AUCUUGAA CUGAUGAG GCCGUUAGGC CGAA AGUUUGGU | 2546 |
| 27 | CAAACUCU U CAAGAUCC | 38 | GGAUCUUG CUGAUGAG GCCGUUAGGC CGAA AGAGUUUG | 2547 |
| 28 | AAACUCUU C AAGAUCCC | 39 | GGGAUCUU CUGAUGAG GCCGUUAGGC CGAA AAGAGUUU | 2548 |
| 34 | UUCAAGAU C CCAGAGUC | 40 | GACUCUGG CUGAUGAG GCCGUUAGGC CGAA AUCUUGAA | 2549 |
| 42 | CCCAGAGU C AGGGCCCU | 41 | AGGGCCCU CUGAUGAG GCCGUUAGGC CGAA ACUCUGGG | 2550 |
| 53 | GGCCCUGU A CUUUCCUG | 42 | CAGGAAAG CUGAUGAG GCCGUUAGGC CGAA ACAGGGCC | 2551 |
| 56 | CCUGUACU U UCCUGCUG | 43 | CAGCAGGA CUGAUGAG GCCGUUAGGC CGAA AGUACAGG | 2552 |
| 57 | CUGUACUU U CCUGCUGG | 44 | CCAGCAGG CUGAUGAG GCCGUUAGGC CGAA AAGUACAG | 2553 |
| 58 | UGUACUUU C CUGCUGGU | 45 | ACCAGCAG CUGAUGAG GCCGUUAGGC CGAA AAAGUACA | 2554 |
| 71 | UGGUGGCU C CAGUUCAG | 46 | CUGAACUG CUGAUGAG GCCGUUAGGC CGAA AGCCACCA | 2555 |
| 76 | GCUCCAGU U CAGGAACA | 47 | UGUUCCUG CUGAUGAG GCCGUUAGGC CGAA ACUGGAGC | 2556 |
| 77 | CUCCAGUU C AGGAACAG | 48 | CUGUUCCU CUGAUGAG GCCGUUAGGC CGAA AACUGGAG | 2557 |
| 97 | GCCCUGCU C AGAAUACU | 49 | AGUAUUCU CUGAUGAG GCCGUUAGGC CGAA AGCAGGGC | 2558 |
| 103 | CUCAGAAU A CUGUCUCU | 50 | AGAGACAG CUGAUGAG GCCGUUAGGC CGAA AUUCUGAG | 2559 |
| 108 | AAUACUGU C UCUGCCAU | 51 | AUGGCAGA CUGAUGAG GCCGUUAGGC CGAA ACAGUAUU | 2560 |
| 110 | UACUGUCU C UGCCAUAU | 52 | AUAUGGCA CUGAUGAG GCCGUUAGGC CGAA AGACAGUA | 2561 |
| 117 | UCUGCCAU A UCGUCAAU | 53 | AUUGACGA CUGAUGAG GCCGUUAGGC CGAA AUGGCAGA | 2562 |
| 119 | UGCCAUAU C GUCAAUCU | 54 | AGAUUGAC CUGAUGAG GCCGUUAGGC CGAA AUAUGGCA | 2563 |
| 122 | CAUAUCGU C AAUCUUAU | 55 | AUAAGAUU CUGAUGAG GCCGUUAGGC CGAA ACGAUAUG | 2564 |
| 126 | UCGUCAAU C UUAUCGAA | 56 | UUCGAUAA CUGAUGAG GCCGUUAGGC CGAA AUUGACCA | 2565 |
| 128 | GUCAAUCU U AUCGAAGA | 57 | UCUUCGAU CUGAUGAG GCCGUUAGGC CGAA AGAUUGAC | 2566 |
| 129 | UCAAUCUU A UCGAAGAC | 58 | GUCUUCGA CUGAUGAG GCCGUUAGGC CGAA AAGAUUGA | 2567 |
| 131 | AAUCUUAU C GAAGACUG | 59 | CAGUCUUC CUGAUGAG GCCGUUAGGC CGAA AUAAGAUU | 2568 |
| 150 | GACCCUGU A CCGAACAU | 60 | AUGUUCGG CUGAUGAG GCCGUUAGGC CGAA ACAGGGUC | 2569 |
| 168 | GAGAACAU C GCAUCAGG | 61 | CCUGAUGC CUGAUGAG GCCGUUAGGC CGAA AUGUUCUC | 2570 |
| 173 | CAUCGCAU C AGGACUCC | 62 | GGAGUCCU CUGAUGAG GCCGUUAGGC CGAA AUGCGAUG | 2571 |
| 180 | UCAGGACU C CUAGGACC | 63 | GGUCCUAG CUGAUGAG GCCGUUAGGC CGAA AGUCCUGA | 2572 |
| 183 | GGACUCCU A GGACCCCU | 64 | AGGGGUCC CUGAUGAG GCCGUUAGGC CGAA AGGAGUCC | 2573 |
| 195 | CCCCUGCU C GUGUUACA | 65 | UGUAACAC CUGAUGAG GCCGUUAGGC CGAA AGCAGGGG | 2574 |
| 200 | GCUCGUGU U ACAGGCGG | 66 | CCGCCUGU CUGAUGAG GCCGUUAGGC CGAA ACACGAGC | 2575 |
| 201 | CUCGUGUU A CAGGCGGG | 67 | CCCGCCUG CUGAUGAG GCCGUUAGGC CGAA AACACGAG | 2576 |
| 212 | GGCGGGGU U UUUCUUGU | 68 | ACAAGAAA CUGAUGAG GCCGUUAGGC CGAA ACCCCGCC | 2577 |
| 213 | GCGGGGUU U UUCUUGUU | 69 | AACAAGAA CUGAUGAG GCCGUUAGGC CGAA AACCCCGC | 2578 |
| 214 | CGGGGUUU U UCUUGUUG | 70 | CAACAAGA CUGAUGAG GCCGUUAGGC CGAA AAACCCCG | 2579 |
| 215 | GGGGUUUU U CUUGUUGA | 71 | UCAACAAG CUGAUGAG GCCGUUAGGC CGAA AAAACCCC | 2580 |
| 216 | GGGUUUUU C UUGUUGAC | 72 | GUCAACAA CUGAUGAG GCCGUUAGGC CGAA AAAAACCC | 2581 |
| 218 | GUUUUUCU U GUUGACAA | 73 | UUGUCAAC CUGAUGAG GCCGUUAGGC CGAA AGAAAAAC | 2582 |
| 221 | UUUCUUGU U GACAAAAA | 74 | UUUUUGUC CUGAUGAG GCCGUUAGGC CGAA ACAAGAAA | 2583 |
| 231 | ACAAAAAU C CUCACAAU | 75 | AUUGUGAG CUGAUGAG GCCGUUAGGC CGAA AUUUUUGU | 2584 |
| 234 | AAAAUCCU C ACAAUACC | 76 | GGUAUUGU CUGAUGAG GCCGUUAGGC CGAA AGGAUUUU | 2585 |
| 240 | CUCACAAU A CCACAGAG | 77 | CUCUGUGG CUGAUGAG GCCGUUAGGC CGAA AUUGUGAG | 2586 |
| 250 | CACAGAGU C UAGACUCG | 78 | CGAGUCUA CUGAUGAG GCCGUUAGGC CGAA ACUCUGUG | 2587 |
| 252 | CAGAGUCU A GACUCGUG | 79 | CACGAGUC CUGAUGAG GCCGUUAGGC CGAA AGACUCUG | 2588 |
| 257 | UCUAGACU C GUGGUGGA | 80 | UCCACCAC CUGAUGAG GCCGUUAGGC CGAA AGUCUAGA | 2589 |
| 268 | GGUGGACU U CUCUCAAU | 81 | AUUGAGAG CUGAUGAG GCCGUUAGGC CGAA AGUCCACC | 2590 |
| 269 | GUGGACUU C UCUCAAUU | 82 | AAUUGAGA CUGAUGAG GCCGUUAGGC CGAA AAGUCCAC | 2591 |
| 271 | GGACUUCU C UCAAUUUU | 83 | AAAAUUGA CUGAUGAG GCCGUUAGGC CGAA AGAAGUCC | 2592 |
| 273 | ACUUCUCU C AAUUUUCU | 84 | AGAAAAUU CUGAUGAG GCCGUUAGGC CGAA AGAGAAGU | 2593 |
| 277 | CUCUCAAU U UUCUAGGG | 85 | CCCUAGAA CUGAUGAG GCCGUUAGGC CGAA AUUGAGAG | 2594 |
| 278 | UCUCAAUU U UCUAGGGG | 86 | CCCCUAGA CUGAUGAG GCCGUUAGGC CGAA AAUUGAGA | 2595 |
| 279 | CUCAAUUU U CUAGGGGG | 87 | CCCCCUAG CUGAUGAG GCCGUUAGGC CGAA AAAUUGAG | 2596 |
| 280 | UCAAUUUU C UAGGGGGA | 88 | UCCCCCUA CUGAUGAG GCCGUUAGGC CGAA AAAAUUGA | 2597 |
| 282 | AAUUUUCU A GGGGGAAC | 89 | GUUCCCCC CUGAUGAG GCCGUUAGGC CGAA AGAAAAUU | 2598 |
| 301 | CCGUGUGU C UUGGCCAA | 90 | UUGGCCAA CUGAUGAG GCCGUUAGGC CGAA ACACACGG | 2599 |
| 303 | GUGUGUCU U GGCCAAAA | 91 | UUUUGGCC CUGAUGAG GCCGUUAGGC CGAA AGACACAC | 2600 |
| 313 | GCCAAAAU U CGCAGUCC | 92 | GGACUGCG CUGAUGAG GCCGUUAGGC CGAA AUUUUGGC | 2601 |
| 314 | CCAAAAUU C GCAGUCCC | 93 | GGGACUGC CUGAUGAG GCCGUUAGGC CGAA AAUUUUGG | 2602 |
| 320 | UUCGCAGU C CCAAAUCU | 94 | AGAUUUGG CUGAUGAG GCCGUUAGGC CGAA ACUGCGAA | 2603 |
| 327 | UCCCAAAU C UCCAGUCA | 95 | UGACUGGA CUGAUGAG GCCGUUAGGC CGAA AUUUGGGA | 2604 |
| 329 | CCAAAUCU C CAGUCACU | 96 | AGUGACUG CUGAUGAG GCCGUUAGGC CGAA AGAUUUGG | 2605 |
| 334 | UCUCCAGU C ACUCACCA | 97 | UGGUGAGU CUGAUGAG GCCGUUAGGC CGAA ACUGGAGA | 2606 |
| 338 | CAGUCACU C ACCAACCU | 98 | AGGUUGGU CUGAUGAG GCCGUUAGGC CGAA AGUGACUG | 2607 |
| 349 | CAACCUGU U GUCCUCCA | 99 | UGGAGGAC CUGAUGAG GCCGUUAGGC CGAA ACAGGUUG | 2608 |
| 352 | CCUGUUGU C CUCCAAUU | 100 | AAUUGGAG CUGAUGAG GCCGUUAGGC CGAA ACAACAGG | 2609 |
| 355 | GUUGUCCU C CAAUUUGU | 101 | ACAAAUUG CUGAUGAG GCCGUUAGGC CGAA AGGACAAC | 2610 |
| 360 | CCUCCAAU U UGUCCUGG | 102 | CCAGGACA CUGAUGAG GCCGUUAGGC CGAA AUUGGAGG | 2611 |
| 361 | CUCCAAUU U GUCCUGGU | 103 | ACCAGGAC CUGAUGAG GCCGUUAGGC CGAA AAUUGGAG | 2612 |
| 364 | CAAUUUGU C CUGGUUAU | 104 | AUAACCAG CUGAUGAG GCCGUUAGGC CGAA ACAAAUUG | 2613 |
| 370 | GUCCUGGU U AUCGCUGG | 105 | CCAGCGAU CUGAUGAG GCCGUUAGGC CGAA ACCAGGAC | 2614 |
| 371 | UCCUGGUU A UCGCUGGA | 106 | UCCAGCGA CUGAUGAG GCCGUUAGGC CGAA AACCAGGA | 2615 |
| 373 | CUGGUUAU C GCUGGAUG | 107 | CAUCCAGC CUGAUGAG GCCGUUAGGC CGAA AUAACCAG | 2616 |
| 385 | GGAUGUGU C UGCGGCGU | 108 | ACGCCGCA CUGAUGAG GCCGUUAGGC CGAA ACACAUCC | 2617 |
| 394 | UGCGGCGU U UUAUCAUC | 109 | GAUGAUAA CUGAUGAG GCCGUUAGGC CGAA ACGCCGCA | 2618 |
| 395 | GCGGCGUU U UAUCAUCU | 110 | AGAUGAUA CUGAUGAG GCCGUUAGGC CGAA AACGCCGC | 2619 |
| 396 | CGGCGUUU U AUCAUCUU | 111 | AAGAUGAU CUGAUGAG GCCGUUAGGC CGAA AAACGCCG | 2620 |
| 397 | GGCGUUUU A UCAUCUUC | 112 | GAAGAUGA CUGAUGAG GCCGUUAGGC CGAA AAAACGCC | 2621 |
| 399 | CGUUUUAU C AUCUUCCU | 113 | AGGAAGAU CUGAUGAG GCCGUUAGGC CGAA AUAAAACG | 2622 |
| 402 | UUUAUCAU C UUCCUCUG | 114 | CAGAGGAA CUGAUGAG GCCGUUAGGC CGAA AUGAUAAA | 2623 |
| 404 | UAUCAUCU U CCUCUGCA | 115 | UGCAGAGG CUGAUGAG GCCGUUAGGC CGAA AGAUGAUA | 2624 |
| 405 | AUCAUCUU C CUCUGCAU | 116 | AUGCAGAG CUGAUGAG GCCGUUAGGC CGAA AAGAUGAU | 2625 |
| 408 | AUCUUCCU C UGCAUCCU | 117 | AGGAUGCA CUGAUGAG GCCGUUAGGC CGAA AGGAAGAU | 2626 |
| 414 | CUCUGCAU C CUGCUGCU | 118 | AGCAGCAG CUGAUGAG GCCGUUAGGC CGAA AUGCAGAG | 2627 |
| 423 | CUGCUGCU A UGCCUCAU | 119 | AUGAGGCA CUGAUGAG GCCGUUAGGC CGAA AGCAGCAG | 2628 |
| 429 | CUAUGCCU C AUCUUCUU | 120 | AAGAAGAU CUGAUGAG GCCGUUAGGC CGAA AGGCAUAG | 2629 |
| 432 | UGCCUCAU C UUCUUGUU | 121 | AACAAGAA CUGAUGAG GCCGUUAGGC CGAA AUGAGGCA | 2630 |
| 434 | CCUCAUCU U CUUGUUGG | 122 | CCAACAAG CUGAUGAG GCCGUUAGGC CGAA AGAUGAGG | 2631 |
| 435 | CUCAUCUU C UUGUUGGU | 123 | ACCAACAA CUGAUGAG GCCGUUAGGC CGAA AAGAUGAG | 2632 |
| 437 | CAUCUUCU U GUUGGUUC | 124 | GAACCAAC CUGAUGAG GCCGUUAGGC CGAA AGAAGAUG | 2633 |
| 440 | CUUCUUGU U GGUUCUUC | 125 | GAAGAACC CUGAUGAG GCCGUUAGGC CGAA ACAAGAAG | 2634 |
| 444 | UUGUUGGU U CUUCUGGA | 126 | UCCAGAAG CUGAUGAG GCCGUUAGGC CGAA ACCAACAA | 2635 |
| 445 | UGUUGGUU C UUCUGGAC | 127 | GUCCAGAA CUGAUGAG GCCGUUAGGC CGAA AACCAACA | 2636 |
| 447 | UUGGUUCU U CUGGACUA | 128 | UAGUCCAG CUGAUGAG GCCGUUAGGC CGAA AGAACCAA | 2637 |
| 448 | UGGUUCUU C UGGACUAU | 129 | AUAGUCCA CUGAUGAG GCCGUUAGGC CGAA AAGAACCA | 2638 |
| 455 | UCUGGACU A UCAAGGUA | 130 | UACCUUGA CUGAUGAG GCCGUUAGGC CGAA AGUCCAGA | 2639 |
| 457 | UGGACUAU C AAGGUAUG | 131 | CAUACCUU CUGAUGAG GCCGUUAGGC CGAA AUAGUCCA | 2640 |
| 463 | AUCAAGGU A UGUUGCCC | 132 | GGGCAACA CUGAUGAG GCCGUUAGGC CGAA ACCUUGAU | 2641 |
| 467 | AGGUAUGU U GCCCGUUU | 133 | AAACGGGC CUGAUGAG GCCGUUAGGC CGAA ACAUACCU | 2642 |
| 474 | UUGCCCGU U UGUCCUCU | 134 | AGAGGACA CUGAUGAG GCCGUUAGGC CGAA ACGGGCAA | 2643 |
| 475 | UGCCCGUU U GUCCUCCA | 135 | UAGAGGAC CUGAUGAG GCCGUUAGGC CGAA AACGGGCA | 2644 |
| 478 | CCGUUUGU C CUCUAAUU | 136 | AAUUAGAG CUGAUGAG GCCGUUAGGC CGAA ACAAACGG | 2645 |
| 481 | UUUGUCCU C UAAUUCCA | 137 | UGGAAUUA CUGAUGAG GCCGUUAGGC CGAA AGGACAAA | 2646 |
| 483 | UGUCCUCU A AUUCCAGG | 138 | CCUGGAAU CUGAUGAG GCCGUUAGGC CGAA AGAGGACA | 2647 |
| 486 | CCUCUAAU U CCAGGAUC | 139 | GAUCCUGG CUGAUGAG GCCGUUAGGC CGAA AUUAGAGG | 2648 |
| 487 | CUCUAAUU C CAGGAUCA | 140 | UGAUCCUG CUGAUGAG GCCGUUAGGC CGAA AAUUAGAG | 2649 |
| 494 | UCCAGGAU C AUCAACAA | 141 | UUGUUGAU CUGAUGAG GCCGUUAGGC CGAA AUCCUGGA | 2650 |
| 497 | AGGAUCAU C AACAACCA | 142 | UGGUUGUU CUGAUGAG GCCGUUAGGC CGAA AUGAUCCU | 2651 |
| 535 | GCACAACU C CUGCUCAA | 143 | UUGAGCAG CUGAUGAG GCCGUUAGGC CGAA AGUUGUGC | 2652 |
| 541 | CUCCUGCU C AAGGAUCC | 144 | GGUUCCUU CUGAUGAG GCCGUUAGGC CGAA AGCAGGAG | 2653 |
| 551 | AGGAACCU C UAUGUUUC | 145 | GAAACAUA CUGAUGAG GCCGUUAGGC CGAA AGGUUCCU | 2654 |
| 553 | GAACCUCU A UGUUUCCC | 146 | GGGAAACA CUGAUGAG GCCGUUAGGC CGAA AGAGGUUC | 2655 |
| 557 | CUCUAUGU U UCCCUCAU | 147 | AUGAGGGA CUGAUGAG GCCGUUAGGC CGAA ACAUAGAG | 2656 |
| 558 | UCUAUGUU U CCCUCAUG | 148 | CAUGAGGG CUGAUGAG GCCGUUAGGC CGAA AACAUAGA | 2657 |
| 559 | CUAUGUUU C CCUCAUGU | 149 | ACAUGAGG CUGAUGAG GCCGUUAGGC CGAA AAACAUAG | 2658 |
| 563 | GUUUCCCU C AUGUUGCU | 150 | AGCAACAU CUGAUGAG GCCGUUAGGC CGAA AGGGAAAC | 2659 |
| 568 | CCUCAUGU U GCUGUACA | 151 | UGUACAGC CUGAUGAG GCCGUUAGGC CGAA ACAUGAGG | 2660 |
| 574 | GUUGCUGU A CAAAACCU | 152 | AGGUUUUG CUGAUGAG GCCGUUAGGC CGAA ACAGCAAC | 2661 |
| 583 | CAAAACCU A CGGACGGA | 153 | UCCGUCCG CUGAUGAG GCCGUUAGGC CGAA AGGUUUUG | 2662 |
| 604 | GCACCUGU A UUCCCAUC | 154 | GAUGGGAA CUGAUGAG GCCGUUAGGC CGAA ACAGGUGC | 2663 |
| 606 | ACCUGUAU U CCCAUCCC | 155 | GGGAUGGG CUGAUGAG GCCGUUAGGC CGAA AUACAGGU | 2664 |
| 607 | CCUGUAUU C CCAUCCCA | 156 | UGGGAUGG CUGAUGAG GCCGUUAGGC CGAA AAUACAGG | 2665 |
| 612 | AUUCCCAU C CCAUCAUC | 157 | GAUGAUGG CUGAUGAG GCCGUUAGGC CGAA AUGGGAAU | 2666 |
| 617 | CAUCCCAU C AUCUUGGG | 158 | CCCAAGAU CUGAUGAG GCCGUUAGGC CGAA AUGGGAUG | 2667 |
| 620 | CCCAUCAU C UUGGGCUU | 159 | AAGCCCAA CUGAUGAG GCCGUUAGGC CGAA AUGAUGGG | 2668 |
| 622 | CAUCAUCU U GGGCUUUC | 160 | GAAAGCCC CUGAUGAG GCCGUUAGGC CGAA AGAUGAUG | 2669 |
| 628 | CUUGGGCU U UCGCAAAA | 161 | UUUUGCGA CUGAUGAG GCCGUUAGGC CGAA AGCCCAAG | 2670 |
| 629 | UUGGGCUU U CGCAAAAU | 162 | AUUUUGCG CUGAUGAG GCCGUUAGGC CGAA AAGCCCAA | 2671 |
| 630 | UGGGCUUU C GCAAAAUA | 163 | UAUUUUGC CUGAUGAG GCCGUUAGGC CGAA AAAGCCCA | 2672 |
| 638 | CGCAAAAU A CCUAUGGG | 164 | CCCAUAGG CUGAUGAG GCCGUUAGGC CGAA AUUUUGCG | 2673 |
| 642 | AAAUACCU A UGGGAGUG | 165 | CACUCCCA CUGAUGAG GCCGUUAGGC CGAA AGGUAUUU | 2674 |
| 656 | GUGGGCCU C AGUCCGUU | 166 | AACGGACU CUGAUGAG GCCGUUAGGC CGAA AGGCCCAC | 2675 |
| 660 | GCCUCAGU C CGUUUCUC | 167 | GAGAAACG CUGAUGAG GCCGUUAGGC CGAA ACUGAGGC | 2676 |
| 664 | CAGUCCGU U UCUCUUGG | 168 | CCAAGAGA CUGAUGAG GCCGUUAGGC CGAA ACGGACUG | 2677 |
| 665 | AGUCCGUU U CUCUUGGC | 169 | GCCAAGAG CUGAUGAG GCCGUUAGGC CGAA AACGGACU | 2678 |
| 666 | GUCCGUUU C UCUUGGCU | 170 | AGCCAAGA CUGAUGAG GCCGUUAGGC CGAA AAACGGAC | 2679 |
| 668 | CCGUUUCU C UUGGCUCA | 171 | UGAGCCAA CUGAUGAG GCCGUUAGGC CGAA AGAAACGG | 2680 |
| 670 | GUUUCUCU U GGCUCAGU | 172 | ACUGAGCC CUGAUGAG GCCGUUAGGC CGAA AGAGAAAC | 2681 |
| 675 | UCUUGGCU C AGUUUACU | 173 | AGUAAACU CUGAUGAG GCCGUUAGGC CGAA AGCCAAGA | 2682 |
| 679 | GGCUCAGU U UACUAGUG | 174 | CACUAGUA CUGAUGAG GCCGUUAGGC CGAA ACUGAGCC | 2683 |
| 680 | GCUCAGUU U ACUAGUGC | 175 | GCACUAGU CUGAUGAG GCCGUUAGGC CGAA AACUGAGC | 2684 |
| 682 | CUCAGUUU A CUAGUGCC | 176 | GGCACUAG CUGAUGAG GCCGUUAGGC CGAA AAACUGAG | 2685 |
| 684 | AGUUUACU A GUGCCAUU | 177 | AAUGGCAC CUGAUGAG GCCGUUAGGC CGAA AGUAAACU | 2686 |
| 692 | AGUGCCAU U UGUUCAGU | 178 | ACUGAACA CUGAUGAG GCCGUUAGGC CGAA AUGGCACU | 2687 |
| 693 | GUGCCAUU U GUUCAGUG | 179 | CACUGAAC CUGAUGAG GCCGUUAGGC CGAA AAUGGCAC | 2688 |
| 696 | CCAUUUGU U CAGUGGUU | 180 | AACCACUG CUGAUGAG GCCGUUAGGC CGAA ACAAAUGG | 2689 |
| 697 | CAUUUGUU C AGUGGUUC | 181 | GAACCACU CUGAUGAG GCCGUUAGGC CGAA AACAAAUG | 2690 |
| 704 | UCAGUGGU U CGUAGGGC | 182 | GCCCUACG CUGAUGAG GCCGUUAGGC CGAA ACCACUGA | 2691 |
| 705 | CAGUGGUU C GUAGGGCU | 183 | AGCCCUAC CUGAUGAG GCCGUUAGGC CGAA AACCACUG | 2692 |
| 708 | UGGUUCGU A GGGCUUUC | 184 | GAAAGCCC CUGAUGAG GCCGUUAGGC CGAA ACGAACCA | 2693 |
| 714 | GUAGGGCU U UCCCCCAC | 185 | GUGGGGGA CUGAUGAG GCCGUUAGGC CGAA AGCCCUAC | 2694 |
| 715 | UAGGGCUU U CCCCCACU | 186 | AGUGGGGG CUGAUGAG GCCGUUAGGC CGAA AAGCCCUA | 2695 |
| 716 | AGGGCUUU C CCCCACUG | 187 | CAGUGGGG CUGAUGAG GCCGUUAGGC CGAA AAAGCCCU | 2696 |
| 726 | CCCACUGU C UGGCUUUC | 188 | GAAAGCCA CUGAUGAG GCCGUUAGGC CGAA ACAGUGGG | 2697 |
| 732 | GUCUGGCU U UCAGUUAU | 189 | AUAACUGA CUGAUGAG GCCGUUAGGC CGAA AGCCAGAC | 2698 |
| 733 | UCUGGCUU U CAGUUAUA | 190 | UAUAACUG CUGAUGAG GCCGUUAGGC CGAA AAGCCAGA | 2699 |
| 734 | CUGGCUUU C AGUUAUAU | 191 | AUAUAACU CUGAUGAG GCCGUUAGGC CGAA AAAGCCAG | 2700 |
| 738 | CUUUCAGU U AUAUGGAU | 192 | AUCCAUAU CUGAUGAG GCCGUUAGGC CGAA ACUGAAAG | 2701 |
| 739 | UUUCAGUU A UAUGGAUG | 193 | CAUCCAUA CUGAUGAG GCCGUUAGGC CGAA AACUGAAA | 2702 |
| 742 | UCAGUUAU A UGGAUGAU | 194 | AUCAUCCA CUGAUGAG GCCGUUAGGC CGAA AUAACUGA | 2703 |
| 755 | GAUGUGGU U UUGGGGGC | 195 | GCCCCCAA CUGAUGAG GCCGUUAGGC CGAA ACCACAUC | 2704 |
| 756 | AUGUGGUU U UGGGGGCC | 196 | GGCCCCCA CUGAUGAG GCCGUUAGGC CGAA AACCACAU | 2705 |
| 757 | UGUGGUUU U GGGGGCCA | 197 | UGGCCCCC CUGAUGAG GCCGUUAGGC CGAA AAACCACA | 2706 |
| 769 | GGCCAAGU C UGUACAAC | 198 | GUUGUACA CUGAUGAG GCCGUUAGGC CGAA ACUUGGCC | 2707 |
| 773 | AAGUCUGU A CAACAUCU | 199 | AGAUGUUG CUGAUGAG GCCGUUAGGC CGAA ACAGACUU | 2708 |
| 780 | UACAACAU C UUGAGUCC | 200 | GGACUCAA CUGAUGAG GCCGUUAGGC CGAA AUGUUGUA | 2709 |
| 782 | CAACAUCU U GAGUCCCU | 201 | AGGGACUC CUGAUGAG GCCGUUAGGC CGAA AGAUGUUG | 2710 |
| 787 | UCUUGAGU C CCUUUAUG | 202 | CAUAAAGG CUGAUGAG GCCGUUAGGC CGAA ACUCAAGA | 2711 |
| 791 | GAGUCCCU U UAUGCCGC | 203 | GCGGCAUA CUGAUGAG GCCGUUAGGC CGAA AGGGACUC | 2712 |
| 792 | AGUCCCUU U AUGCCGCU | 204 | AGCGGCAU CUGAUGAG GCCGUUAGGC CGAA AAGGGACU | 2713 |
| 793 | GUCCCUUU A UGCCGCUG | 205 | CAGCGGCA CUGAUGAG GCCGUUAGGC CGAA AAAGGGAC | 2714 |
| 803 | GCCGCUGU U ACCAAUUU | 206 | AAAUUGGU CUGAUGAG GCCGUUAGGC CGAA ACAGCGGC | 2715 |
| 804 | CCGCUGUU A CCAAUUUU | 207 | AAAAUUGG CUGAUGAG GCCGUUAGGC CGAA AACAGCGG | 2716 |
| 810 | UUACCAAU U UUCUUUUG | 208 | CAAAAGAA CUGAUGAG GCCGUUAGGC CGAA AUUGGUAA | 2717 |
| 811 | UACCAAUU U UCUUUUGU | 209 | ACAAAAGA CUGAUGAG GCCGUUAGGC CGAA AAUUGGUA | 2718 |
| 812 | ACCAAUUU U CUUUUGUC | 210 | GACAAAAG CUGAUGAG GCCGUUAGGC CGAA AAAUUGGU | 2719 |
| 813 | CCAAUUUU C UUUUGUCU | 211 | AGACAAAA CUGAUGAG GCCGUUAGGC CGAA AAAAUUGG | 2720 |
| 815 | AAUUUUCU U UUGUCUUU | 212 | AAAGACAA CUGAUGAG GCCGUUAGGC CGAA AGAAAAUU | 2721 |
| 816 | AUUUUCUU U UGUCUUUG | 213 | CAAAGACA CUGAUGAG GCCGUUAGGC CGAA AAGAAAAU | 2722 |
| 817 | UUUUCUUU U GUCUUUGG | 214 | CCAAAGAC CUGAUGAG GCCGUUAGGC CGAA AAAGAAAA | 2723 |
| 820 | UCUUUUGU C UUUGGGUA | 215 | UACCCAAA CUGAUGAG GCCGUUAGGC CGAA ACAAAAGA | 2724 |
| 822 | UUUUGUCU U UGGGUAUA | 216 | UAUACCCA CUGAUGAG GCCGUUAGGC CGAA AGACAAAA | 2725 |
| 823 | UUUGUCUU U GGGUAUAC | 217 | GUAUACCC CUGAUGAG GCCGUUAGGC CGAA AAGACAAA | 2726 |
| 828 | CUUUGGGU A UACAUUUA | 218 | UAAAUGUA CUGAUGAG GCCGUUAGGC CGAA ACCCAAAG | 2727 |
| 830 | UUGGGUAU A CAUUUAAA | 219 | UUUAAAUG CUGAUGAG GCCGUUAGGC CGAA AUACCCAA | 2728 |
| 834 | GUAUACAU U UAAACCCU | 220 | AGGGUUUA CUGAUGAG GCCGUUAGGC CGAA AUGUAUAC | 2729 |
| 835 | UAUACAUU U AAACCCUC | 221 | GAGGGUUU CUGAUGAG GCCGUUAGGC CGAA AAUGUAUA | 2730 |
| 836 | AUACAUUU A AACCCUCA | 222 | UGAGGGUU CUGAUGAG GCCGUUAGGC CGAA AAAUGUAU | 2731 |
| 843 | UAAACCCU C ACAAAACA | 223 | UGUUUUGU CUGAUGAG GCCGUUAGGC CGAA AGGGUUUA | 2732 |
| 865 | AUGGGGAU A UUCCCUUA | 224 | UAAGGGAA CUGAUGAG GCCGUUAGGC CGAA AUCCCCAU | 2733 |
| 867 | GGGGAUAU U CCCUUAAC | 225 | GUUAAGGG CUGAUGAG GCCGUUAGGC CGAA AUAUCCCC | 2734 |
| 868 | GGGAUAUU C CCUUAACU | 226 | AGUUAAGG CUGAUGAG GCCGUUAGGC CGAA AAUAUCCC | 2735 |
| 872 | UAUUCCCU U AACUUCAU | 227 | AUGAAGUU CUGAUGAG GCCGUUAGGC CGAA AGGGAAUA | 2736 |
| 873 | AUUCCCUU A ACUUCAUG | 228 | CAUGAAGU CUGAUGAG GCCGUUAGGC CGAA AAGGGAAU | 2737 |
| 877 | CCUUAACU U CAUGGGAU | 229 | AUCCCAUG CUGAUGAG GCCGUUAGGC CGAA AGUUAAGG | 2738 |
| 878 | CUUAACUU C AUGGGAUA | 230 | UAUCCCAU CUGAUGAG GCCGUUAGGC CGAA AAGUUAAG | 2739 |
| 886 | CAUGGGAU A UGUAAUUG | 231 | CAAUUACA CUGAUGAG GCCGUUAGGC CGAA AUCCCAUG | 2740 |
| 890 | GGAUAUGU A AUUGGGAG | 232 | CUCCCAAU CUGAUGAG GCCGUUAGGC CGAA ACAUAUCC | 2741 |
| 893 | UAUGUAAU U GGGAGUUG | 233 | CAACUCCC CUGAUGAG GCCGUUAGGC CGAA AUUACAUA | 2742 |
| 900 | UUGGGAGU U GGGGCACA | 234 | UGUGCCCC CUGAUGAG GCCGUUAGGC CGAA ACUCCCAA | 2743 |
| 910 | GGGCACAU U GCCACAGG | 235 | CCUGUGGC CUGAUGAG GCCGUUAGGC CGAA AUGUGCCC | 2744 |
| 924 | AGGAACAU A UUGUACAA | 236 | UUGUACAA CUGAUGAG GCCGUUAGGC CGAA AUGUUCCU | 2745 |
| 926 | GAACAUAU U GUACAAAA | 237 | UUUUGUAC CUGAUGAG GCCGUUAGGC CGAA AUAUGUUC | 2746 |
| 929 | CAUAUUGU A CAAAAAAU | 238 | AUUUUUUG CUGAUGAG GCCGUUAGGC CGAA ACAAUAUG | 2747 |
| 938 | CAAAAAAU C AAAAUGUG | 239 | CACAUUUU CUGAUGAG GCCGUUAGGC CGAA AUUUUUUG | 2748 |
| 948 | AAAUGUGU U UUAGGAAA | 240 | UUUCCUAA CUGAUGAG GCCGUUAGGC CGAA ACACAUUU | 2749 |
| 949 | AAUGUGUU U UAGGAAAC | 241 | GUUUCCUA CUGAUGAG GCCGUUAGGC CGAA AACACAUU | 2750 |
| 950 | AUGUGUUU U AGGAAACU | 242 | AGUUUCCU CUGAUGAG GCCGUUAGGC CGAA AAACACAU | 2751 |
| 951 | UGUGUUUU A GGAAACUU | 243 | AAGUUUCC CUGAUGAG GCCGUUAGGC CGAA AAAACACA | 2752 |
| 959 | AGGAAACU U CCUGUAAA | 244 | UUUACAGG CUGAUGAG GCCGUUAGGC CGAA AGUUUCCU | 2753 |
| 960 | GGAAACUU C CUGUAAAC | 245 | GUUUACAG CUGAUGAG GCCGUUAGGC CGAA AAGUUUCC | 2754 |
| 965 | CUUCCUGU A AACAGGCC | 246 | GGCCUGUU CUGAUGAG GCCGUUAGGC CGAA ACAGGAAG | 2755 |
| 975 | ACAGGCCU A UUGAUUGG | 247 | CCAAUCAA CUGAUGAG GCCGUUAGGC CGAA AGGCCUGU | 2756 |
| 977 | AGGCCUAU U GAUUGGAA | 248 | UUCCAAUC CUGAUGAG GCCGUUAGGC CGAA AUAGGCCU | 2757 |
| 981 | CUAUUGAU U GGAAAGUA | 249 | UACUUUCC CUGAUGAG GCCGUUAGGC CGAA AUCAAUAG | 2758 |
| 989 | UGGAAAGU A UGUCAACG | 250 | CGUUGACA CUGAUGAG GCCGUUAGGC CGAA ACUUUCCA | 2759 |
| 993 | AAGUAUGU C AACGAAUU | 251 | AAUUCGUU CUGAUGAG GCCGUUAGGC CGAA ACAUACUU | 2760 |
| 1001 | CAACGAAU U GUGGGUCU | 252 | AGACCCAC CUGAUGAG GCCGUUAGGC CGAA AUUCGUUG | 2761 |
| 1008 | UUGUGGGU C UUUUGGGG | 253 | CCCCAAAA CUGAUGAG GCCGUUAGGC CGAA ACCCACAA | 2762 |
| 1010 | GUGGGUCU U UUGGGGUU | 254 | AACCCCAA CUGAUGAG GCCGUUAGGC CGAA AGACCCAC | 2763 |
| 1011 | UGGGUCUU U UGGGGUUU | 255 | AAACCCCA CUGAUGAG GCCGUUAGGC CGAA AAGACCCA | 2764 |
| 1012 | GGGUCUUU U GGGGUUUG | 256 | CAAACCCC CUGAUGAG GCCGUUAGGC CGAA AAAGACCC | 2765 |
| 1018 | UUUGGGGU U UGCCGCCC | 257 | GGGCGGCA CUGAUGAG GCCGUUAGGC CGAA ACCCCAAA | 2766 |
| 1019 | UUGGGGUU U GCCGCCCC | 258 | GGGGCGGC CUGAUGAG GCCGUUAGGC CGAA AACCCCAA | 2767 |
| 1029 | CCGCCCCU U UCACGCAA | 259 | UUGCGUGA CUGAUGAG GCCGUUAGGC CGAA AGGGGCGG | 2768 |
| 1030 | CGCCCCUU U CACGCAAU | 260 | AUUGCGUG CUGAUGAG GCCGUUAGGC CGAA AAGGGGCG | 2769 |
| 1031 | GCCCCUUU C ACGCAAUG | 261 | CAUUGCGU CUGAUGAG GCCGUUAGGC CGAA AAAGGGGC | 2770 |
| 1045 | AUGUGGAU A UUCUGCUU | 262 | AAGCAGAA CUGAUGAG GCCGUUAGGC CGAA AUCCACAU | 2771 |
| 1047 | GUGGAUAU U CUGCUUUA | 263 | UAAAGCAG CUGAUGAG GCCGUUAGGC CGAA AUAUCCAC | 2772 |
| 1048 | UGGAUAUU C UGCUUUAA | 264 | UUAAAGCA CUGAUGAG GCCGUUAGGC CGAA AAUAUCCA | 2773 |
| 1053 | AUUCUGCU U UAAUGCCU | 265 | AGGCAUUA CUGAUGAG GCCGUUAGGC CGAA AGCAGAAU | 2774 |
| 1054 | UUCUGCUU U AAUGCCUU | 266 | AAGGCAUU CUGAUGAG GCCGUUAGGC CGAA AAGCAGAA | 2775 |
| 1055 | UCUGCUUU A AUGCCUUU | 267 | AAAGGCAU CUGAUGAG GCCGUUAGGC CGAA AAAGCAGA | 2776 |
| 1062 | UAAUGCCU U UAUAUGCA | 268 | UGCAUAUA CUGAUGAG GCCGUUAGGC CGAA AGGCAUUA | 2777 |
| 1063 | AAUGCCUU U AUAUGCAU | 269 | AUGCAUAU CUGAUGAG GCCGUUAGGC CGAA AAGGCAUU | 2778 |
| 1064 | AUGCCUUU A UAUGCAUG | 270 | CAUGCAUA CUGAUGAG GCCGUUAGGC CGAA AAAGGCAU | 2779 |
| 1066 | GCCUUUAU A UGCAUGCA | 271 | UGCAUGCA CUGAUGAG GCCGUUAGGC CGAA AUAAAGGC | 2780 |
| 1076 | GCAUGCAU A CAAGCAAA | 272 | UUUGCUUG CUGAUGAG GCCGUUAGGC CGAA AUGCAUGC | 2781 |
| 1092 | AACAGGCU U UUACUUUC | 273 | GAAAGUAA CUGAUGAG GCCGUUAGGC CGAA AGCCUGUU | 2782 |
| 1093 | ACAGGCUU U UACUUUCU | 274 | AGAAAGUA CUGAUGAG GCCGUUAGGC CGAA AAGCCUGU | 2783 |
| 1094 | CAGGCUUU U ACUUUCUC | 275 | GAGAAAGU CUGAUGAG GCCGUUAGGC CGAA AAAGCCUG | 2784 |
| 1095 | AGGCUUUU A CUUUCUCG | 276 | CGAGAAAG CUGAUGAG GCCGUUAGGC CGAA AAAAGCCU | 2785 |
| 1098 | CUUUUACU U UCUCGCCA | 277 | UGGCGAGA CUGAUGAG GCCGUUAGGC CGAA AGUAAAAG | 2786 |
| 1099 | UUUUACUU U CUCGCCAA | 278 | UUGGCGAG CUGAUGAG GCCGUUAGGC CGAA AAGUAAAA | 2787 |
| 1100 | UUUACUUU C UCGCCAAC | 279 | GUUGGCGA CUGAUGAG GCCGUUAGGC CGAA AAAGUAAA | 2788 |
| 1102 | UACUUUCU C GCCAACUU | 280 | AAGUUGGC CUGAUGAG GCCGUUAGGC CGAA AGAAAGUA | 2789 |
| 1110 | CGCCAACU U ACAAGGCC | 281 | GGCCUUGU CUGAUGAG GCCGUUAGGC CGAA AGUUGGCG | 2790 |
| 1111 | GCCAACUU A CAAGGCCU | 282 | AGGCCUUG CUGAUGAG GCCGUUAGGC CGAA AAGUUGGC | 2791 |
| 1120 | CAAGGCCU U UCUAAGUA | 283 | UACUUAGA CUGAUGAG GCCGUUAGGC CGAA AGGCCUUG | 2792 |
| 1121 | AAGGCCUU U CUAAGUAA | 284 | UUACUUAG CUGAUGAG GCCGUUAGGC CGAA AAGGCCUU | 2793 |
| 1122 | AGGCCUUU C UAAGUAAA | 285 | UUUACUUA CUGAUGAG GCCGUUAGGC CGAA AAAGGCCU | 2794 |
| 1124 | GCCUUUCU A AGUAAACA | 286 | UGUUUACU CUGAUGAG GCCGUUAGGC CGAA AGAAAGGC | 2795 |
| 1128 | UUCUAAGU A AACAGUAU | 287 | AUACUGUU CUGAUGAG GCCGUUAGGC CGAA ACUUAGAA | 2796 |
| 1135 | UAAACAGU A UGUGAACC | 288 | GGUUCACA CUGAUGAG GCCGUUAGGC CGAA ACUGUUUA | 2797 |
| 1145 | GUGAACCU U UACCCCGU | 289 | ACGGGGUA CUGAUGAG GCCGUUAGGC CGAA AGGUUCAC | 2798 |
| 1146 | UGAACCUU U ACCCCGUU | 290 | AACGGGGU CUGAUGAG GCCGUUAGGC CGAA AAGGUUCA | 2799 |
| 1147 | GAACCUUU A CCCCGUUG | 291 | CAACGGGG CUGAUGAG GCCGUUAGGC CGAA AAAGGUUC | 2800 |
| 1154 | UACCCCGU U GCUCGGCA | 292 | UGCCGAGC CUGAUGAG GCCGUUAGGC CGAA ACGGGGUA | 2801 |
| 1158 | CCGUUGCU C GGCAACGG | 293 | CCGUUGCC CUGAUGAG GCCGUUAGGC CGAA AGCAACGG | 2802 |
| 1173 | GGCCUGGU C UAUGCCAA | 294 | UUGGCAUA CUGAUGAG GCCGUUAGGC CGAA ACCAGGCC | 2803 |
| 1175 | CCUGGUCU A UGCCAAGU | 295 | ACUUGGCA CUGAUGAG GCCGUUAGGC CGAA AGACCAGG | 2804 |
| 1186 | CCAAGUGU U UGCUGACG | 296 | CGUCAGCA CUGAUGAG GCCGUUAGGC CGAA ACACUUGG | 2805 |
| 1187 | CAAGUGUU U GCUGACGC | 297 | GCGUCAGC CUGAUGAG GCCGUUAGGC CGAA AACACUUG | 2806 |
| 1209 | CCACUGGU U GGGGCUUG | 298 | CAAGCCCC CUGAUGAG GCCGUUAGGC CGAA ACCAGUGG | 2807 |
| 1216 | UUGGGGCU U GGCCAUAG | 299 | CUAUGGCC CUGAUGAG GCCGUUAGGC CGAA AGCCCCAA | 2808 |
| 1223 | UUGGCCAU A GGCCAUCA | 300 | UGAUGGCC CUGAUGAG GCCGUUAGGC CGAA AUGGCCAA | 2809 |
| 1230 | UAGGCCAU C AGCGCAUG | 301 | CAUGCGCU CUGAUGAG GCCGUUAGGC CGAA AUGGCCUA | 2810 |
| 1249 | UGGAACCU U UGUGUCUC | 302 | GAGACACA CUGAUGAG GCCGUUAGGC CGAA AGGUUCCA | 2811 |
| 1250 | GGAACCUU U GUGUCUCC | 303 | GGAGACAC CUGAUGAG GCCGUUAGGC CGAA AAGGUUCC | 2812 |
| 1255 | CUUUGUGU C UCCUCUGC | 304 | GCAGAGGA CUGAUGAG GCCGUUAGGC CGAA ACACAAAG | 2813 |
| 1257 | UUGUGUCU C CUCUGCCG | 305 | CGGCAGAG CUGAUGAG GCCGUUAGGC CGAA AGACACAA | 2814 |
| 1260 | UGUCUCCU C UGCCGAUC | 306 | GAUCGGCA CUGAUGAG GCCGUUAGGC CGAA AGGAGACA | 2815 |
| 1268 | CUGCCGAU C CAUACCGC | 307 | GCGGUAUG CUGAUGAG GCCGUUAGGC CGAA AUCGGCAG | 2816 |
| 1272 | CGAUCCAU A CCGCGGAA | 308 | UUCCGCGG CUGAUGAG GCCGUUAGGC CGAA AUGGAUCG | 2817 |
| 1283 | GCGGAACU C CUAGCCGC | 309 | GCGGCUAG CUGAUGAG GCCGUUAGGC CGAA AGUUCCGC | 2818 |
| 1286 | GAACUCCU A GCCGCUUG | 310 | CAAGCGGC CUGAUGAG GCCGUUAGGC CGAA AGGAGUUC | 2819 |
| 1293 | UAGCCGCU U GUUUUGCU | 311 | AGCAAAAC CUGAUGAG GCCGUUAGGC CGAA AGCGGCUA | 2820 |
| 1296 | CCGCUUGU U UUGCUCGC | 312 | GCGAGCAA CUGAUGAG GCCGUUAGGC CGAA ACAAGCGG | 2821 |
| 1297 | CGCUUGUU U UGCUCGCA | 313 | UGCGAGCA CUGAUGAG GCCGUUAGGC CGAA AACAAGCG | 2822 |
| 1298 | GCUUGUUU U GCUCGCAG | 314 | CUGCGAGC CUGAUGAG GCCGUUAGGC CGAA AAACAAGC | 2823 |
| 1302 | GUUUUGCU C GCAGCAGG | 315 | CCUGCUGC CUGAUGAG GCCGUUAGGC CGAA AGCAAAAC | 2824 |
| 1312 | CAGCAGGU C UGGGGCAA | 316 | UUGCCCCA CUGAUGAG GCCGUUAGGC CGAA ACCUGCUG | 2825 |
| 1325 | GCAAAACU C AUCGGGAC | 317 | GUCCCGAU CUGAUGAG GCCGUUAGGC CGAA AGUUUUGC | 2826 |
| 1328 | AAACUCAU C GGGACUGA | 318 | UCAGUCCC CUGAUGAG GCCGUUAGGC CGAA AUGAGUUU | 2827 |
| 1341 | CUGACAAU U CUGUCGUG | 319 | CACGACAG CUGAUGAG GCCGUUAGGC CGAA AUUGUCAG | 2828 |
| 1342 | UGACAAUU C UGUCGUGC | 320 | GCACGACA CUGAUGAG GCCGUUAGGC CGAA AAUUGUCA | 2829 |
| 1346 | AAUUCUGU C GUGCUCUC | 321 | GAGAGCAC CUGAUGAG GCCGUUAGGC CGAA ACAGAAUU | 2830 |
| 1352 | GUCGUGCU C UCCCGCAA | 322 | UUGCGGGA CUGAUGAG GCCGUUAGGC CGAA AGCACGAC | 2831 |
| 1354 | CGUGCUCU C CCGCAAAU | 323 | AUUUGCGG CUGAUGAG GCCGUUAGGC CGAA AGAGCACG | 2832 |
| 1363 | CCGCAAAU A UACAUCAU | 324 | AUGAUGUA CUGAUGAG GCCGUUAGGC CGAA AUUUGCGG | 2833 |
| 1365 | GCAAAUAU A CAUCAUUU | 325 | AAAUGAUG CUGAUGAG GCCGUUAGGC CGAA AUAUUUGC | 2834 |
| 1369 | AUAUACAU C AUUUCCAU | 326 | AUGGAAAU CUGAUGAG GCCGUUAGGC CGAA AUGUAUAU | 2835 |
| 1372 | UACAUCAU U UCCAUGGC | 327 | GCCAUGGA CUGAUGAG GCCGUUAGGC CGAA AUGAUGUA | 2836 |
| 1373 | ACAUCAUU U CCAUGGCU | 328 | AGCCAUGG CUGAUGAG GCCGUUAGGC CGAA AAUGAUGU | 2837 |
| 1374 | CAUCAUUU C CAUGGCUG | 329 | CAGCCAUG CUGAUGAG GCCGUUAGGC CGAA AAAUGAUG | 2838 |
| 1385 | UGGCUGCU A GGCUGUGC | 330 | GCACAGCC CUGAUGAG GCCGUUAGGC CGAA AGCAGCCA | 2839 |
| 1406 | AACUGGAU C CUACGCGG | 331 | CCGCGUAG CUGAUGAG GCCGUUAGGC CGAA AUCCAGUU | 2840 |
| 1409 | UGGAUCCU A CGCGGGAC | 332 | GUCCCGCG CUGAUGAG GCCGUUAGGC CGAA AGGAUCCA | 2841 |
| 1420 | CGGGACGU C CUUUGUUU | 333 | AAACAAAG CUGAUGAG GCCGUUAGGC CGAA ACGUCCCG | 2842 |
| 1423 | GACGUCCU U UGUUUACG | 334 | CGUAAACA CUGAUGAG GCCGUUAGGC CGAA AGGACGUC | 2843 |
| 1424 | ACGUCCUU U GUUUACGU | 335 | ACGUAAAC CUGAUGAG GCCGUUAGGC CGAA AAGGACGU | 2844 |
| 1427 | UCCUUUGU U UACGUCCC | 336 | GGGACGUA CUGAUGAG GCCGUUAGGC CGAA ACAAAGGA | 2845 |
| 1428 | CCUUUGUU U ACGUCCCG | 337 | CGGGACGU CUGAUGAG GCCGUUAGGC CGAA AACAAAGG | 2846 |
| 1429 | CUUUGUUU A CGUCCCGU | 338 | ACGGGACG CUGAUGAG GCCGUUAGGC CGAA AAACAAAG | 2847 |
| 1433 | GUUUACGU C CCGUCGGC | 339 | GCCGACGG CUGAUGAG GCCGUUAGGC CGAA ACGUAAAC | 2848 |
| 1438 | CGUCCCGU C GGCGCUGA | 340 | UCAGCGCC CUGAUGAG GCCGUUAGGC CGAA ACGGGACG | 2849 |
| 1449 | CGCUGAAU C CCGCGGAC | 341 | GUCCGCGG CUGAUGAG GCCGUUAGGC CGAA AUUCAGCG | 2850 |
| 1465 | CGACCCCU C CCGGGGCC | 342 | GGCCCCGG CUGAUGAG GCCGUUAGGC CGAA AGGGGUCG | 2851 |
| 1477 | GGGCCGCU U GGGGCUCU | 343 | AGAGCCCC CUGAUGAG GCCGUUAGGC CGAA AGCGGCCC | 2852 |
| 1484 | UUGGGGCU C UACCGCCC | 344 | GGGCGGUA CUGAUGAG GCCGUUAGGC CGAA AGCCCCAA | 2853 |
| 1486 | GGGGCUCU A CCGCCCGC | 345 | GCGGGCGG CUGAUGAG GCCGUUAGGC CGAA AGAGCCCC | 2854 |
| 1496 | CGCCCGCU U CUCCGCCU | 346 | AGGCGGAG CUGAUGAG GCCGUUAGGC CGAA AGCGGGCG | 2855 |
| 1497 | GCCCGCUU C UCCGCCUA | 347 | UAGGCGGA CUGAUGAG GCCGUUAGGC CGAA AAGCGGGC | 2856 |
| 1499 | CCGCUUCU C CGCCUAUU | 348 | AAUAGGCG CUGAUGAG GCCGUUAGGC CGAA AGAAGCGG | 2857 |
| 1505 | CUCCGCCU A UUGUACCG | 349 | CGGUACAA CUGAUGAG GCCGUUAGGC CGAA AGGCGGAG | 2858 |
| 1507 | CCGCCUAU U GUACCGAC | 350 | GUCGGUAC CUGAUGAG GCCGUUAGGC CGAA AUAGGCGG | 2859 |
| 1510 | CCUAUUGU A CCGACCGU | 351 | ACGGUCGG CUGAUGAG GCCGUUAGGC CGAA ACAAUAGG | 2860 |
| 1519 | CCGACCGU C CACGGGGC | 352 | GCCCCGUG CUGAUGAG GCCGUUAGGC CGAA ACGGUCGG | 2861 |
| 1534 | GCGCACCU C UCUUUACG | 353 | CGUAAAGA CUGAUGAG GCCGUUAGGC CGAA AGGUGCGC | 2862 |
| 1536 | GCACCUCU C UUUACGCG | 354 | CGCGUAAA CUGAUGAG GCCGUUAGGC CGAA AGAGGUGC | 2863 |
| 1538 | ACCUCUCU U UACGCGGA | 355 | UCCGCGUA CUGAUGAG GCCGUUAGGC CGAA AGAGAGGU | 2864 |
| 1539 | CCUCUCUU U ACGCGGAC | 356 | GUCCGCGU CUGAUGAG GCCGUUAGGC CGAA AAGAGAGG | 2865 |
| 1540 | CUCUCUUU A CGCGGACU | 357 | AGUCCGCG CUGAUGAG GCCGUUAGGC CGAA AAAGAGAG | 2866 |
| 1549 | CGCGGACU C CCCGUCUG | 358 | CAGACGGG CUGAUGAG GCCGUUAGGC CGAA AGUCCGCG | 2867 |
| 1555 | CUCCCCGU C UGUGCCUU | 359 | AAGGCACA CUGAUGAG GCCGUUAGGC CGAA ACGGGGAG | 2868 |
| 1563 | CUGUGCCU U CUCAUCUG | 360 | CAGAUGAG CUGAUGAG GCCGUUAGGC CGAA AGGCACAG | 2869 |
| 1564 | UGUGCCUU C UCAUCUGC | 361 | GCAGAUGA CUGAUGAG GCCGUUAGGC CGAA AAGGCACA | 2870 |
| 1566 | UGCCUUCU C AUCUGCCG | 362 | CGGCAGAU CUGAUGAG GCCGUUAGGC CGAA AGAAGGCA | 2871 |
| 1569 | CUUCUCAU C UGCCGGAC | 363 | GUCCGGCA CUGAUGAG GCCGUUAGGC CGAA AUGAGAAG | 2872 |
| 1588 | UGUGCACU U CGCUUCAC | 364 | GUGAAGCG CUGAUGAG GCCGUUAGGC CGAA AGUGCACA | 2873 |
| 1589 | GUGCACUU C GCUUCACC | 365 | GGUGAAGC CUGAUGAG GCCGUUAGGC CGAA AAGUGCAC | 2874 |
| 1593 | ACUUCGCU U CACCUCUG | 366 | CAGAGGUG CUGAUGAG GCCGUUAGGC CGAA AGCGAAGU | 2875 |
| 1594 | CUUCGCUU C ACCUCUGC | 367 | GCAGAGGU CUGAUGAG GCCGUUAGGC CGAA AAGCGAAG | 2876 |
| 1599 | CUUCACCU C UGCACGUC | 368 | GACGUGCA CUGAUGAG GCCGUUAGGC CGAA AGGUGAAG | 2877 |
| 1607 | CUGCACGU C GCAUGGAG | 369 | CUCCAUGC CUGAUGAG GCCGUUAGGC CGAA ACGUGCAG | 2878 |
| 1651 | CCCAAGGU C UUGCAUAA | 370 | UUAUGCAA CUGAUGAG GCCGUUAGGC CGAA ACCUUGGG | 2879 |
| 1653 | CAAGGUCU U GCAUAAGA | 371 | UCUUAUGC CUGAUGAG GCCGUUAGGC CGAA AGACCUUG | 2880 |
| 1658 | UCUUGCAU A AGAGGACU | 372 | AGUCCUCU CUGAUGAG GCCGUUAGGC CGAA AUGCAAGA | 2881 |
| 1667 | AGAGGACU C UUGGACUU | 373 | AAGUCCAA CUGAUGAG GCCGUUAGGC CGAA AGUCCUCU | 2882 |
| 1669 | AGGACUCU U GGACUUUC | 374 | GAAAGUCC CUGAUGAG GCCGUUAGGC CGAA AGAGUCCU | 2883 |
| 1675 | CUUGGACU U UCAGCAAU | 375 | AUUGCUGA CUGAUGAG GCCGUUAGGC CGAA AGUCCAAG | 2884 |
| 1676 | UUGGACUU U CAGCAAUG | 376 | CAUUGCUG CUGAUGAG GCCGUUAGGC CGAA AAGUCCAA | 2885 |
| 1677 | UGGACUUU C AGCAAUGU | 377 | ACAUUGCU CUGAUGAG GCCGUUAGGC CGAA AAAGUCCA | 2886 |
| 1686 | AGCAAUGU C AACGACCG | 378 | CGGUCGUU CUGAUGAG GCCGUUAGGC CGAA ACAUUGCU | 2887 |
| 1699 | ACCGACCU U GAGGCAUA | 379 | UAUGCCUC CUGAUGAG GCCGUUAGGC CGAA AGGUCGGU | 2888 |
| 1707 | UGAGGCAU A CUUCAAAG | 380 | CUUUGAAG CUGAUGAG GCCGUUAGGC CGAA AUGCCUCA | 2889 |
| 1710 | GGCAUACU U CAAAGACU | 381 | AGUCUUUG CUGAUGAG GCCGUUAGGC CGAA AGUAUGCC | 2890 |
| 1711 | GCAUACUU C AAAGACUG | 382 | CAGUCUUU CUGAUGAG GCCGUUAGGC CGAA AAGUAUGC | 2891 |
| 1725 | CUGUGUGU U UAUUGAGU | 383 | ACUCAUUA CUGAUGAG GCCGUUAGGC CGAA ACACACAG | 2892 |
| 1726 | UGUGUGUU U AAUGAGUG | 384 | CACUCAUU CUGAUGAG GCCGUUAGGC CGAA AACACACA | 2893 |
| 1727 | GUGUGUUU A AUGAGUGG | 385 | CCACUCAU CUGAUGAG GCCGUUAGGC CGAA AAACACAC | 2894 |
| 1743 | GGAGGAGU U GGGGGAGG | 386 | CCUCCCCC CUGAUGAG GCCGUUAGGC CGAA ACUCCUCC | 2895 |
| 1756 | GAGGAGGU U AGGUUAAA | 387 | UUUAACCU CUGAUGAG GCCGUUAGGC CGAA ACCUCCUC | 2896 |
| 1757 | AGGAGGUU A GGUUAAAG | 388 | CUUUAACC CUGAUGAG GCCGUUAGGC CGAA AACCUCCU | 2897 |
| 1761 | GGUUAGGU U AAAGGUCU | 389 | AGACCUUU CUGAUGAG GCCGUUAGGC CGAA ACCUAACC | 2898 |
| 1762 | GUUAGGUU A AAGGUCUU | 390 | AAGACCUU CUGAUGAG GCCGUUAGGC CGAA AACCUAAC | 2899 |
| 1768 | UUAAAGGU C UUUGUACU | 391 | AGUACAAA CUGAUGAG GCCGUUAGGC CGAA ACCUUUAA | 2900 |
| 1770 | AAAGGUCU U UGUACUAG | 392 | CUAGUACA CUGAUGAG GCCGUUAGGC CGAA AGACCUUU | 2901 |
| 1771 | AAGGUCUU U GUACUAGG | 393 | CCUAGUAC CUGAUGAG GCCGUUAGGC CGAA AAGACCUU | 2902 |
| 1774 | GUCUUUGU A CUAGGAGG | 394 | CCUCCUAG CUGAUGAG GCCGUUAGGC CGAA ACAAAGAC | 2903 |
| 1777 | UUUGUACU A GGAGGCUG | 395 | CAGCCUCC CUGAUGAG GCCGUUAGGC CGAA AGUACAAA | 2904 |
| 1787 | GAGGCUGU A GGCAUAAA | 396 | UUUAUGCC CUGAUGAG GCCGUUAGGC CGAA ACAGCCUC | 2905 |
| 1793 | GUAGGCAU A AAUUGGUG | 397 | CACCAAUU CUGAUGAG GCCGUUAGGC CGAA AUGCCUAC | 2906 |
| 1797 | GCAUAAAU U GGUGUGUU | 398 | AACACACC CUGAUGAG GCCGUUAGGC CGAA AUUUAUGC | 2907 |
| 1805 | UGGUGUGU U CACCAGCA | 399 | UGCUGGUG CUGAUGAG GCCGUUAGGC CGAA ACACACCA | 2908 |
| 1806 | GGUGUGUU C ACCAGCAC | 400 | GUGCUGGU CUGAUGAG GCCGUUAGGC CGAA AACACACC | 2909 |
| 1824 | AUGCAACU U UUUCACCU | 401 | AGGUGAAA CUGAUGAG GCCGUUAGGC CGAA AGUUGCAU | 2910 |
| 1825 | UGCAACUU U UUCACCUC | 402 | GAGGUGAA CUGAUGAG GCCGUUAGGC CGAA AAGUUGCA | 2911 |
| 1826 | GCAACUUU U UCACCUCU | 403 | AGAGGUGA CUGAUGAG GCCGUUAGGC CGAA AAAGUUGC | 2912 |
| 1827 | CAACUUUU U CACCUCUG | 404 | CAGAGGUG CUGAUGAG GCCGUUAGGC CGAA AAAAGUUG | 2913 |
| 1828 | AACUUUUU C ACCUCUGC | 405 | GCAGAGGU CUGAUGAG GCCGUUAGGC CGAA AAAAAGUU | 2914 |
| 1833 | UUUCACCU C UGCCUAAU | 406 | AUUAGGCA CUGAUGAG GCCGUUAGGC CGAA AGGUGAAA | 2915 |
| 1839 | CUCUGCCU A AUCAUCUC | 407 | GAGAUGAU CUGAUGAG GCCGUUAGGC CGAA AGGCAGAG | 2916 |
| 1842 | UGCCUAAU C AUCUCAUG | 408 | CAUGAGAU CUGAUGAG GCCGUUAGGC CGAA AUUAGGCA | 2917 |
| 1845 | CUAAUCAU C UCAUGUUC | 409 | GAACAUGA CUGAUGAG GCCGUUAGGC CGAA AUGAUUAG | 2918 |
| 1847 | AAUCAUCU C AUGUUCAU | 410 | AUGAACAU CUGAUGAG GCCGUUAGGC CGAA AGAUGAUU | 2919 |
| 1852 | UCUCAUGU U CAUGUCCU | 411 | AGGACAUG CUGAUGAG GCCGUUAGGC CGAA ACAUGAGA | 2920 |
| 1853 | CUCAUGUU C AUGUCCUA | 412 | UAGGACAU CUGAUGAG GCCGUUAGGC CGAA AACAUGAG | 2921 |
| 1858 | GUUCAUGU C CUACUGUU | 413 | AACAGUAG CUGAUGAG GCCGUUAGGC CGAA ACAUGAAC | 2922 |
| 1861 | CAUGUCCU A CUGUUCAA | 414 | UUGAACAG CUGAUGAG GCCGUUAGGC CGAA AGGACAUG | 2923 |
| 1866 | CCUACUGU U CAAGCCUC | 415 | GAGGCUUG CUGAUGAG GCCGUUAGGC CGAA ACAGUAGG | 2924 |
| 1867 | CUACUGUU C AAGCCUCC | 416 | GGAGGCUU CUGAUGAG GCCGUUAGGC CGAA AACAGUAG | 2925 |
| 1874 | UCAAGCCU C CAAGCUGU | 417 | ACAGCUUG CUGAUGAG GCCGUUAGGC CGAA AGGCUUGA | 2926 |
| 1887 | CUGUGCCU U GGGUGGCU | 418 | AGCCACCC CUGAUGAG GCCGUUAGGC CGAA AGGCACAG | 2927 |
| 1896 | GGGUGGCU U UGGGGCAU | 419 | AUGCCCCA CUGAUGAG GCCGUUAGGC CGAA AGCCACCC | 2928 |
| 1897 | GGUGGCUU U GGGGCAUG | 420 | CAUGCCCC CUGAUGAG GCCGUUAGGC CGAA AAGCCACC | 2929 |
| 1911 | AUGGACAU U GACCCGUA | 421 | UACGGGUC CUGAUGAG GCCGUUAGGC CGAA AUGUCCAU | 2930 |
| 1919 | UGACCCGU A UAAAGAAU | 422 | AUUCUUUA CUGAUGAG GCCGUUAGGC CGAA ACGGGUCA | 2931 |
| 1921 | ACCCGUAU A AAGAAUUU | 423 | AAAUUCUU CUGAUGAG GCCGUUAGGC CGAA AUACGGGU | 2932 |
| 1928 | UAAAGAAU U UGGAGCUU | 424 | AAGCUCCA CUGAUGAG GCCGUUAGGC CGAA AUUCUUUA | 2933 |
| 1929 | AAAGAAUU U GGAGCUUC | 425 | GAAGCUCC CUGAUGAG GCCGUUAGGC CGAA AAUUCUUU | 2934 |
| 1936 | UUGGAGCU U CUGUGGAG | 426 | CUCCACAG CUGAUGAG GCCGUUAGGC CGAA AGCUCCAA | 2935 |
| 1937 | UGGAGCUU C UGUGGAGU | 427 | ACUCCACA CUGAUGAG GCCGUUAGGC CGAA AAGCUCCA | 2936 |
| 1946 | UGUGGAGU U ACUCUCUU | 428 | AAGAGAGU CUGAUGAG GCCGUUAGGC CGAA ACUCCACA | 2937 |
| 1947 | GUGGAGUU A CUCUCUUU | 429 | AAAGAGAG CUGAUGAG GCCGUUAGGC CGAA AACUCCAC | 2938 |
| 1950 | GAGUUACU C UCUUUUUU | 430 | AAAAAAGA CUGAUGAG GCCGUUAGGC CGAA AGUAACUC | 2939 |
| 1952 | GUUACUCU C UUUUUUGC | 431 | GCAAAAAA CUGAUGAG GCCGUUAGGC CGAA AGAGUAAC | 2940 |
| 1954 | UACUCUCU U UUUUGCCU | 432 | AGGCAAAA CUGAUGAG GCCGUUAGGC CGAA AGAGAGUA | 2941 |
| 1955 | ACUCUCUU U UUUGCCUU | 433 | AAGGCAAA CUGAUGAG GCCGUUAGGC CGAA AAGAGAGU | 2942 |
| 1956 | CUCUCUUU U UUGCCUUC | 434 | GAAGGCAA CUGAUGAG GCCGUUAGGC CGAA AAAGAGAG | 2943 |
| 1957 | UCUCUUUU U UGCCUUCU | 435 | AGAAGGCA CUGAUGAG GCCGUUAGGC CGAA AAAAGAGA | 2944 |
| 1958 | CUCUUUUU U GCCUUCUG | 436 | CAGAAGGC CUGAUGAG GCCGUUAGGC CGAA AAAAAGAG | 2945 |
| 1963 | UUUUGCCU U CUGACUUC | 437 | GAAGUCAG CUGAUGAG GCCGUUAGGC CGAA AGGCAAAA | 2946 |
| 1964 | UUUGCCUU C UGACUUCU | 438 | AGAAGUCA CUGAUGAG GCCGUUAGGC CGAA AAGGCAAA | 2947 |
| 1970 | UUCUGACU U CUUUCCUU | 439 | AAGGAAAG CUGAUGAG GCCGUUAGGC CGAA AGUCAGAA | 2948 |
| 1971 | UCUGACUU C UUUCCUUC | 440 | GAAGGAAA CUGAUGAG GCCGUUAGGC CGAA AAGUCAGA | 2949 |
| 1973 | UGACUUCU U UCCUUCUA | 441 | UAGAAGGA CUGAUGAG GCCGUUAGGC CGAA AGAAGUCA | 2950 |
| 1974 | GACUUCUU U CCUUCUAU | 442 | AUAGAAGG CUGAUGAG GCCGUUAGGC CGAA AAGAAGUC | 2951 |
| 1975 | ACUUCUUU C CUUCUAUC | 443 | AAUAGAAG CUGAUGAG GCCGUUAGGC CGAA AAAGAAGU | 2952 |
| 1978 | UCUUUCCU U CUAUUCGA | 444 | UCGAAUAG CUGAUGAG GCCGUUAGGC CGAA AGGAAAGA | 2953 |
| 1979 | CUUUCCUU C UAUUCGAG | 445 | CUCGAAUA CUGAUGAG GCCGUUAGGC CGAA AAGGAAAG | 2954 |
| 1981 | UUCCUUCU A UUCGAGAU | 446 | AUCUCGAA CUGAUGAG GCCGUUAGGC CGAA AGAAGGAA | 2955 |
| 1983 | CCUUCUAU U CGAGAUCU | 447 | AGAUCUCG CUGAUGAG GCCGUUAGGC CGAA AUAGAAGG | 2956 |
| 1984 | CUUCUAUU C GAGAUCUC | 448 | GAGAUCUC CUGAUGAG GCCGUUAGGC CGAA AAUAGAAG | 2957 |
| 1990 | UUCGAGAU C UCCUCGAC | 449 | GUCGAGGA CUGAUGAG GCCGUUAGGC CGAA AUCUCGAA | 2958 |
| 1992 | CGAGAUCU C CUCGACAC | 450 | GUGUCGAG CUGAUGAG GCCGUUAGGC CGAA AGAUCUCG | 2959 |
| 1995 | GAUCUCCU C GACACCGC | 451 | GCGGUGUC CUGAUGAG GCCGUUAGGC CGAA AGGAGAUC | 2960 |
| 2006 | CACCGCCU C UGCUCUGU | 452 | ACAGAGCA CUGAUGAG GCCGUUAGGC CGAA AGGCGGUG | 2961 |
| 2011 | CCUCUGCU C UGUAUCGG | 453 | CCGAUACA CUGAUGAG GCCGUUAGGC CGAA AGCAGAGG | 2962 |
| 2015 | UGCUCUGU A UCGGGGGG | 454 | CCCCCCGA CUGAUGAG GCCGUUAGGC CGAA ACAGACCA | 2963 |
| 2017 | CUCUGUAU C GGGGGGCC | 455 | GGCCCCCC CUGAUGAG GCCGUUAGGC CGAA AUACAGAG | 2964 |
| 2027 | GGGGGCCU U AGAGUCUC | 456 | GAGACUCU CUGAUGAG GCCGUUAGGC CGAA AGGCCCCC | 2965 |
| 2028 | GGGGCCUU A GAGUCUCC | 457 | GGAGACUC CUGAUGAG GCCGUUAGGC CGAA AAGGCCCC | 2966 |
| 2033 | CUUAGAGU C UCCGGAAC | 458 | GUUCCGGA CUGAUGAG GCCGUUAGGC CGAA ACUCUAAG | 2967 |
| 2035 | UAGAGUCU C CGGAACAU | 459 | AUGUUCCG CUGAUGAG GCCGUUAGGC CGAA AGACUCUA | 2968 |
| 2044 | CGGAACAU U GUUCACCU | 460 | AGGUGAAC CUGAUGAG GCCGUUAGGC CGAA AUGUUCCG | 2969 |
| 2047 | AACAUUGU U CACCUCAC | 461 | GUGAGGUG CUGAUGAG GCCGUUAGGC CGAA ACAAUGUU | 2970 |
| 2048 | ACAUUGUU C ACCUCACC | 462 | GGUGAGGU CUGAUGAG GCCGUUAGGC CGAA AACAAUGU | 2971 |
| 2053 | GUUCACCU C ACCAUACG | 463 | CGUAUGGU CUGAUGAG GCCGUUAGGC CGAA AGGUGAAC | 2972 |
| 2059 | CUCACCAU A CGGCACUC | 464 | GAGUGCCG CUGAUGAG GCCGUUAGGC CGAA AUGGUGAG | 2973 |
| 2067 | ACGGCACU C AGGCAAGC | 465 | GCUUGCCU CUGAUGAG GCCGUUAGGC CGAA AGUGCCGU | 2974 |
| 2077 | GGCAAGCU A UUCUGUGU | 466 | ACACAGAA CUGAUGAG GCCGUUAGGC CGAA AGCUUGCC | 2975 |
| 2079 | CAAGCUAU U CUGUGUUG | 467 | CAACACAG CUGAUGAG GCCGUUAGGC CGAA AUAGCUUG | 2976 |
| 2080 | AAGCUAUU C UGUGUUGG | 468 | CCAACACA CUGAUGAG GCCGUUAGGC CGAA AAUAGCUU | 2977 |
| 2086 | UUCUGUGU U GGGGUGAG | 469 | CUCACCCC CUGAUGAG GCCGUUAGGC CGAA ACACAGAA | 2978 |
| 2096 | GGGUGAGU U GAUGAAUC | 470 | GAUUCAUC CUGAUGAG GCCGUUAGGC CGAA ACUCACCC | 2979 |
| 2104 | UGAUGAAU C UAGCCACC | 471 | GGUGGCUA CUGAUGAG GCCGUUAGGC CGAA AUUCAUCA | 2980 |
| 2106 | AUGAAUCU A GCCACCUG | 472 | CAGGUGGC CUGAUGAG GCCGUUAGGC CGAA AGAUUCAU | 2981 |
| 2125 | UGGGAAGU A AUUUGGAA | 473 | UUCCAAAU CUGAUGAG GCCGUUAGGC CGAA ACUUCCCA | 2982 |
| 2128 | GAAGUAAU U UGGAAGAU | 474 | AUCUUCCA CUGAUGAG GCCGUUAGGC CGAA AUUACUUC | 2983 |
| 2129 | AAGUAAUU U GGAAGAUC | 475 | GAUCUUCC CUGAUGAG GCCGUUAGGC CGAA AAUUACUU | 2984 |
| 2137 | UGGAAGAU C CAGCAUCC | 476 | GGAUGCUG CUGAUGAG GCCGUUAGGC CGAA AUCUUCCA | 2985 |
| 2144 | UCCACCAU C CAGGGAAU | 477 | AUUCCCUG CUGAUGAG GCCGUUAGGC CGAA AUGCUGGA | 2986 |
| 2153 | CAGGGAAU U AGUAGUCA | 478 | UGACUACU CUGAUGAG GCCGUUAGGC CGAA AUUCCCUG | 2987 |
| 2154 | AGGGAAUU A GUAGUCAG | 479 | CUGACUAC CUGAUGAG GCCGUUAGGC CGAA AAUUCCCU | 2988 |
| 2157 | GAAUUAGU A GUCAGCUA | 480 | UAGCUGAC CUGAUGAG GCCGUUAGGC CGAA ACUAAUUC | 2989 |
| 2160 | UUAGUAGU C AGCUAUGU | 481 | ACAUAGCU CUGAUGAG GCCGUUAGGC CGAA ACUACUAA | 2990 |
| 2165 | AGUCAGCU A UGUCAACG | 482 | CGUUGACA CUGAUGAG GCCGUUAGGC CGAA AGCUGACU | 2991 |
| 2169 | AGCUAUGU C AACGUUAA | 483 | UUAACGUU CUGAUGAG GCCGUUAGGC CGAA ACAUAGCU | 2992 |
| 2175 | GUCAACGU U AAUAUGGG | 484 | CCCAUAUU CUGAUGAG GCCGUUAGGC CGAA ACGUUGAC | 2993 |
| 2176 | UCAACGUU A AUAUGGGC | 485 | GCCCAUAU CUGAUGAG GCCGUUAGGC CGAA AACGUUGA | 2994 |
| 2179 | ACGUUAAU A UGGGCCUA | 486 | UAGGCCCA CUGAUGAG GCCGUUAGGC CGAA AUUAACGU | 2995 |
| 2187 | AUGGGCCU A AAAAUCAG | 487 | CUGAUUUU CUGAUGAG GCCGUUAGGC CGAA AGGCCCAU | 2996 |
| 2193 | CUAAAAAU C AGACAACU | 488 | AGUUGUCU CUGAUGAG GCCGUUAGGC CGAA AUUUUUAG | 2997 |
| 2202 | AGACAACU A UUGUGGUU | 489 | AACCACAA CUGAUGAG GCCGUUAGGC CGAA AGUUGUCU | 2998 |
| 2204 | ACAACUAU U GUGGUUUC | 490 | GAAACCAC CUGAUGAG GCCGUUAGGC CGAA AUAGUUGU | 2999 |
| 2210 | AUUGUGGU U UCACAUUU | 491 | AAAUGUGA CUGAUGAG GCCGUUAGGC CGAA ACCACAAU | 3000 |
| 2211 | UUGUGGUU U CACAUUUC | 492 | GAAAUGUG CUGAUGAG GCCGUUAGGC CGAA AACCACAA | 3001 |
| 2212 | UGUGGUUU C ACAUUUCC | 493 | GGAAAUGU CUGAUGAG GCCGUUAGGC CGAA AAACCACA | 3002 |
| 2217 | UUUCACAU U UCCUGUCU | 494 | AGACAGGA CUGAUGAG GCCGUUAGGC CGAA AUGUGAAA | 3003 |
| 2218 | UUCACAUU U CCUGUCUU | 495 | AAGACAGG CUGAUGAG GCCGUUAGGC CGAA AAUGUGAA | 3004 |
| 2219 | UCACAUUU C CUGUCUUA | 496 | UAAGACAG CUGAUGAG GCCGUUAGGC CGAA AAAUGUGA | 3005 |
| 2224 | UUUCCUGU C UUACUUUU | 497 | AAAAGUAA CUGAUGAG GCCGUUAGGC CGAA ACAGGAAA | 3006 |
| 2226 | UCCUGUCU U ACUUUUGG | 498 | CCAAAAGU CUGAUGAG GCCGUUAGGC CGAA AGACAGGA | 3007 |
| 2227 | CCUGUCUU A CUUUUGGG | 499 | CCCAAAAG CUGAUGAG GCCGUUAGGC CGAA AAGACAGG | 3008 |
| 2230 | GUCUUACU U UUGGGCGA | 500 | UCGCCCAA CUGAUGAG GCCGUUAGGC CGAA AGUAAGAC | 3009 |
| 2231 | UCUUACUU U UGGGCGAG | 501 | CUCGCCCA CUGAUGAG GCCGUUAGGC CGAA AAGUAAGA | 3010 |
| 2232 | CUUACUUU U GGGCGAGA | 502 | UCUCGCCC CUGAUGAG GCCGUUAGGC CGAA AAAGUAAG | 3011 |
| 2247 | GAAACUGU U CUUGAAUA | 503 | UAUUCAAG CUGAUGAG GCCGUUAGGC CGAA ACAGUUUC | 3012 |
| 2248 | AAACUGUU C UUGAAUAU | 504 | AUAUUCAA CUGAUGAG GCCGUUAGGC CGAA AACAGUUU | 3013 |
| 2250 | ACUGUUCU U GAAUAUUU | 505 | AAAUAUUC CUGAUGAG GCCGUUAGGC CGAA AGAACAGU | 3014 |
| 2255 | UCUUGAAU A UUUGGUGU | 506 | ACACCAAA CUGAUGAG GCCGUUAGGC CGAA AUUCAAGA | 3015 |
| 2257 | UUGAAUAU U UGGUGUCU | 507 | AGACACCA CUGAUGAG GCCGUUAGGC CGAA AUAUUCAA | 3016 |
| 2258 | UGAAUAUU U GGUGUCUU | 508 | AAGACACC CUGAUGAG GCCGUUAGGC CGAA AAUAUUCA | 3017 |
| 2264 | UUUGGUGU C UUUUGGAG | 509 | CUCCAAAA CUGAUGAG GCCGUUAGGC CGAA ACACCAAA | 3018 |
| 2266 | UGGUGUCU U UUGGAGUG | 510 | CACUCCAA CUGAUGAG GCCGUUAGGC CGAA AGACACCA | 3019 |
| 2267 | GGUGUCUU U UGGAGUGU | 511 | ACACUCCA CUGAUGAG GCCGUUAGGC CGAA AAGACACC | 3020 |
| 2268 | GUGUCUUU U GGAGUGUG | 512 | CACACUCC CUGAUGAG GCCGUUAGGC CGAA AAAGACAC | 3021 |
| 2280 | GUGUGGAU U CGCACUCC | 513 | GGAGUGCG CUGAUGAG GCCGUUAGGC CGAA AUCCACAC | 3022 |
| 2281 | UGUGGAUU C GCACUCCU | 514 | AGGAGUGC CUGAUGAG GCCGUUAGGC CGAA AAUCCACA | 3023 |
| 2287 | UUCGCACU C CUCCUGCA | 515 | UGCAGGAG CUGAUGAG GCCGUUAGGC CGAA AGUGCGAA | 3024 |
| 2290 | GCACUCCU C CUGCAUAU | 516 | AUAUGCAG CUGAUGAG GCCGUUAGGC CGAA AGGAGUGC | 3025 |
| 2297 | UCCUGCAU A UAGACCAC | 517 | GUGGUCUA CUGAUGAG GCCGUUAGGC CGAA AUGCAGGA | 3026 |
| 2299 | CUGCAUAU A GACCACCA | 518 | UGGUGGUC CUGAUGAG GCCGUUAGGC CGAA AUAUGCAG | 3027 |
| 2317 | AUGCCCCU A UCUUAUCA | 519 | UGAUAAGA CUGAUGAG GCCGUUAGGC CGAA AGGGGCAU | 3028 |
| 2319 | GCCCCUAU C UUAUCAAC | 520 | GUUGAUAA CUGAUGAG GCCGUUAGGC CGAA AUAGGGGC | 3029 |
| 2321 | CCCUAUCU U AUCAACAC | 521 | GUGUUGAU CUGAUGAG GCCGUUAGGC CGAA AGAUAGGG | 3030 |
| 2322 | CCUAUCUU A UCAACACU | 522 | AGUGUUGA CUGAUGAG GCCGUUAGGC CGAA AAGAUAGG | 3031 |
| 2324 | UAUCUUAU C AACACUUC | 523 | GAAGUGUU CUGAUGAG GCCGUUAGGC CGAA AUAAGAUA | 3032 |
| 2331 | UCAACACU U CCGGAAAC | 524 | GUUUCCGG CUGAUGAG GCCGUUAGGC CGAA AGUGUUGA | 3033 |
| 2332 | CAACACUU C CGGAAACU | 525 | AGUUUCCG CUGAUGAG GCCGUUAGGC CGAA AAGUGUUG | 3034 |
| 2341 | CGGAAACU A CUGUUGUU | 526 | AACAACAG CUGAUGAG GCCGUUAGGC CGAA AGUUUCCG | 3035 |
| 2346 | ACUACUGU U GUUAGACG | 527 | CGUCUAAC CUGAUGAG GCCGUUAGGC CGAA ACAGUAGU | 3036 |
| 2349 | ACUGUUGU U AGACGAAG | 528 | CUUCGUCU CUGAUGAG GCCGUUAGGC CGAA ACAACAGU | 3037 |
| 2350 | CUGUUGUU A GACGAAGA | 529 | UCUUCGUC CUGAUGAG GCCGUUAGGC CGAA AACAACAG | 3038 |
| 2366 | AGGCAGGU C CCCUAGAA | 530 | UUCUAGGG CUGAUGAG GCCGUUAGGC CGAA ACCUGCCU | 3039 |
| 2371 | GGUCCCCU A GAAGAAGA | 531 | UCUUCUUC CUGAUGAG GCCGUUAGGC CGAA AGGGGACC | 3040 |
| 2383 | GAAGAACU C CCUCGCCU | 532 | AGGCGAGG CUGAUGAG GCCGUUAGGC CGAA AGUUCUUC | 3041 |
| 2387 | AACUCCCU C GCCUCGCA | 533 | UGCGAGGC CUGAUGAG GCCGUUAGGC CGAA AGGGAGUU | 3042 |
| 2392 | CCUCGCCU C GCAGACGA | 534 | UCGUCUGC CUGAUGAG GCCGUUAGGC CGAA AGGCGAGG | 3043 |
| 2405 | ACGAAGGU C UCAAUCGC | 535 | GCGAUUGA CUGAUGAG GCCGUUAGGC CGAA ACCUUCGU | 3044 |
| 2407 | GAAGGUCU C AAUCGCCG | 536 | CGGCGAUU CUGAUGAG GCCGUUAGGC CGAA AGACCUUC | 3045 |
| 2411 | GUCUCAAU C GCCGCGUC | 537 | GACGCGGC CUGAUGAG GCCGUUAGGC CGAA AUUGAGAC | 3046 |
| 2419 | CGCCGCGU C GCAGAAGA | 538 | UCUUCUGC CUGAUGAG GCCGUUAGGC CGAA ACGCGGCG | 3047 |
| 2429 | CAGAAGAU C UCAAUCUC | 539 | GAGAUUGA CUGAUGAG GCCGUUAGGC CGAA AUCUUCUG | 3048 |
| 2431 | GAAGAUCU C AAUCUCGG | 540 | CCGAGAUU CUGAUGAG GCCGUUAGGC CGAA AGAUCUUC | 3049 |
| 2435 | AUCUCAAU C UCGGGAAU | 541 | AUUCCCGA CUGAUGAG GCCGUUAGGC CGAA AUUGAGAU | 3050 |
| 2437 | CUCAAUCU C GGGAAUCU | 542 | AGAUUCCC CUGAUGAG GCCGUUAGGC CGAA AGAUUGAG | 3051 |
| 2444 | UCGGGAAU C UCAAUGUU | 543 | AACAUUGA CUGAUGAG GCCGUUAGGC CGAA AUUCCCGA | 3052 |
| 2446 | GGGAAUCU C AAUGUUAG | 544 | CUAACAUU CUGAUGAG GCCGUUAGGC CGAA AGAUUCCC | 3053 |
| 2452 | CUCAAUGU U AGUAUUCC | 545 | GGAAUACU CUGAUGAG GCCGUUAGGC CGAA ACAUUGAG | 3054 |
| 2453 | UCAAUGUU A GUAUUCCU | 546 | AGGAAUAC CUGAUGAG GCCGUUAGGC CGAA AACAUUGA | 3055 |
| 2456 | AUGUUAGU A UUCCUUGG | 547 | CCAAGGAA CUGAUGAG GCCGUUAGGC CGAA ACUAACAU | 3056 |
| 2458 | GUUAGUAU U CCUUGGAC | 548 | GUCCAAGG CUGAUGAG GCCGUUAGGC CGAA AUACUAAC | 3057 |
| 2459 | UUAGUAUU C CUUGGACA | 549 | UGUCCAAG CUGAUGAG GCCGUUAGGC CGAA AAUACUAA | 3058 |
| 2462 | GUAUUCCU U GGACACAU | 550 | AUGUGUCC CUGAUGAG GCCGUUAGGC CGAA AGGAAUAC | 3059 |
| 2471 | GGACACAU A AGGUGGGA | 551 | UCCCACCU CUGAUGAG GCCGUUAGGC CGAA AUGUGUCC | 3060 |
| 2484 | GGGAAACU U UACGGGGC | 552 | GCCCCGUA CUGAUGAG GCCGUUAGGC CGAA AGUUUCCC | 3061 |
| 2485 | GGAAACUU U ACGGGGCU | 553 | AGCCCCGU CUGAUGAG GCCGUUAGGC CGAA AAGUUUCC | 3062 |
| 2486 | GAAACUUU A CGGGGCUU | 554 | AAGCCCCG CUGAUGAG GCCGUUAGGC CGAA AAAGUUUC | 3063 |
| 2494 | ACGGGGCU U UAUUCUUC | 555 | GAAGAAUA CUGAUGAG GCCGUUAGGC CGAA AGCCCCGU | 3064 |
| 2495 | CGGGGCUU U AUUCUUCU | 556 | AGAAGAAU CUGAUGAG GCCGUUAGGC CGAA AAGCCCCG | 3065 |
| 2496 | GGGGCUUU A UUCUUCUA | 557 | UAGAAGAA CUGAUGAG GCCGUUAGGC CGAA AAAGCCCC | 3066 |
| 2498 | GGCUUUAU U CUUCUACG | 558 | CGUAGAAG CUGAUGAG GCCGUUAGGC CGAA AUAAAGCC | 3067 |
| 2499 | GCUUUAUU C UUCUACGG | 559 | CCGUAGAA CUGAUGAG GCCGUUAGGC CGAA AAUAAAGC | 3068 |
| 2501 | UUUAUUCU U CUACGGUA | 560 | UACCGUAG CUGAUGAG GCCGUUAGGC CGAA AGAAUAAA | 3069 |
| 2502 | UUAUUCUU C UACGGUAC | 561 | GUACCGUA CUGAUGAG GCCGUUAGGC CGAA AAGAAUAA | 3070 |
| 2504 | AUUCUUCU A CGGUACCU | 562 | AGGUACCG CUGAUGAG GCCGUUAGGC CGAA AGAAGAAU | 3071 |
| 2509 | UCUACGGU A CCUUGCUU | 563 | AAGCAAGG CUGAUGAG GCCGUUAGGC CGAA ACCGUAGA | 3072 |
| 2513 | CGGUACCU U GCUUUAAU | 564 | AUUAAAGC CUGAUGAG GCCGUUAGGC CGAA AGGUACCG | 3073 |
| 2517 | ACCUUGCU U UAAUCCUA | 565 | UAGGAUUA CUGAUGAG GCCGUUAGGC CGAA AGCAAGGU | 3074 |
| 2518 | CCUUGCUU U AAUCCUAA | 566 | UUAGGAUU CUGAUGAG GCCGUUAGGC CGAA AAGCAAGG | 3075 |
| 2519 | CUUGCUUU A AUCCUAAA | 567 | UUUAGGAU CUGAUGAG GCCGUUAGGC CGAA AAAGCAAG | 3076 |
| 2522 | GCUUUAAU C CUAAAUGG | 568 | CCAUUUAG CUGAUGAG GCCGUUAGGC CGAA AUUAAAGC | 3077 |
| 2525 | UUAAUCCU A AAUGGCAA | 569 | UUGCCAUU CUGAUGAG GCCGUUAGGC CGAA AGGAUUAA | 3078 |
| 2537 | GGCAAACU C CUUCUUUU | 570 | AAAAGAAG CUGAUGAG GCCGUUAGGC CGAA AGUUUGCC | 3079 |
| 2540 | AAACUCCU U CUUUUCCU | 571 | AGGAAAAG CUGAUGAG GCCGUUAGGC CGAA AGGAGUUU | 3080 |
| 2541 | AACUCCUU C UUUUCCUG | 572 | CAGGAAAA CUGAUGAG GCCGUUAGGC CGAA AAGGAGUU | 3081 |
| 2543 | CUCCUUCU U UUCCUGAC | 573 | GUCAGGAA CUGAUGAG GCCGUUAGGC CGAA AGAAGGAG | 3082 |
| 2544 | UCCUUCUU U UCCUGACA | 574 | UGUCAGGA CUGAUGAG GCCGUUAGGC CGAA AAGAAGGA | 3083 |
| 2545 | CCUUCUUU U CCUGACAU | 575 | AUGUCAGG CUGAUGAG GCCGUUAGGC CGAA AAAGAAGG | 3084 |
| 2546 | CUUCUUUU C CUGACAUU | 576 | AAUGUCAG CUGAUGAG GCCGUUAGGC CGAA AAAAGAAG | 3085 |
| 2554 | CCUGACAU U CAUUUGCA | 577 | UGCAAAUG CUGAUGAG GCCGUUAGGC CGAA AUGUCAGG | 3086 |
| 2555 | CUGACAUU C AUUUGCAG | 578 | CUGCAAAU CUGAUGAG GCCGUUAGGC CGAA AAUGUCAG | 3087 |
| 2558 | ACAUUCAU U UGCAGGAG | 579 | CUCCUGCA CUGAUGAG GCCGUUAGGC CGAA AUGAAUGU | 3088 |
| 2559 | CAUUCAUU U GCAGGAGG | 580 | CCUCCUGC CUGAUGAG GCCGUUAGGC CGAA AAUGAAUG | 3089 |
| 2572 | GAGGACAU U GUUGAUAG | 581 | CUAUCAAC CUGAUGAG GCCGUUAGGC CGAA AUGUCCUC | 3090 |
| 2575 | GACAUUGU U GAUAGAUG | 582 | CAUCUAUC CUGAUGAG GCCGUUAGGC CGAA ACAAUGUC | 3091 |
| 2579 | UUGUUGAU A GAUGUAAG | 583 | CUUACAUC CUGAUGAG GCCGUUAGGC CGAA AUCAACAA | 3092 |
| 2585 | AUAGAUGU A AGCAAUUU | 584 | AAAUUGCU CUGAUGAG GCCGUUAGGC CGAA ACAUCUAU | 3093 |
| 2592 | UAAGCAAU U UGUGGGGC | 585 | GCCCCACA CUGAUGAG GCCGUUAGGC CGAA AUUGCUUA | 3094 |
| 2593 | AAGCAAUU U GUGGGGCC | 586 | GGCCCCAC CUGAUGAG GCCGUUAGGC CGAA AAUUGCUU | 3095 |
| 2605 | GGGCCCCU U ACAGUAAA | 587 | UUUACUGU CUGAUGAG GCCGUUAGGC CGAA AGGGGCCC | 3096 |
| 2606 | GGCCCCUU A CAGUAAAU | 588 | AUUUACUG CUGAUGAG GCCGUUAGGC CGAA AAGGGGCC | 3097 |
| 2611 | CUUACAGU A AAUGAAAA | 589 | UUUUCAUU CUGAUGAG GCCGUUAGGC CGAA ACUGUAAG | 3098 |
| 2629 | AGGAGACU U AAAUUAAC | 590 | GUUAAUUU CUGAUGAG GCCGUUAGGC CGAA AGUCUCCU | 3099 |
| 2630 | GGAGACUU A AAUUAACU | 591 | AGUUAAUU CUGAUGAG GCCGUUAGGC CGAA AAGUCUCC | 3100 |
| 2634 | ACUUAAAU U AACUAUGC | 592 | GCAUAGUU CUGAUGAG GCCGUUAGGC CGAA AUUUAAGU | 3101 |
| 2635 | CUUAAAUU A ACUAUGCC | 593 | GGCAUAGU CUGAUGAG GCCGUUAGGC CGAA AAUUUAAG | 3102 |
| 2639 | AAUUAACU A UGCCUGCU | 594 | AGCAGGCA CUGAUGAG GCCGUUAGGC CGAA AGUUAAUU | 3103 |
| 2648 | UGCCUGCU A GGUUUUAU | 595 | AUAAAACC CUGAUGAG GCCGUUAGGC CGAA AGCAGGCA | 3104 |
| 2652 | UGCUAGGU U UUAUCCCA | 596 | UGGGAUAA CUGAUGAG GCCGUUAGGC CGAA ACCUAGCA | 3105 |
| 2653 | GCUAGGUU U UAUCCCAA | 597 | UUGGGAUA CUGAUGAG GCCGUUAGGC CGAA AACCUAGC | 3106 |
| 2654 | CUAGGUUU U AUCCCAAU | 598 | AUUGGGAU CUGAUGAG GCCGUUAGGC CGAA AAACCUAG | 3107 |
| 2655 | UAGGUUUU A UCCCAAUG | 599 | CAUUGGGA CUGAUGAG GCCGUUAGGC CGAA AAAACCUA | 3108 |
| 2657 | GGUUUUAU C CCAAUGUU | 600 | AACAUUGG CUGAUGAG GCCGUUAGGC CGAA AUAAAACC | 3109 |
| 2665 | CCCAAUGU U ACUAAAUA | 601 | UAUUUAGU CUGAUGAG GCCGUUAGGC CGAA ACAUUGGG | 3110 |
| 2666 | CCAAUGUU A CUAAAUAU | 602 | AUAUUUAG CUGAUGAG GCCGUUAGGC CGAA AACAUUGG | 3111 |
| 2669 | AUGUUACU A AAUAUUUG | 603 | CAAAUAUU CUGAUGAG GCCGUUAGGC CGAA AGUAACAU | 3112 |
| 2673 | UACUAAAU A UUUGCCCU | 604 | AGGGCAAA CUGAUGAG GCCGUUAGGC CGAA AUUUAGUA | 3113 |
| 2675 | CUAAAUAU U UGCCCUUA | 605 | UAAGGGCA CUGAUGAG GCCGUUAGGC CGAA AUAUUUAG | 3114 |
| 2676 | UAAAUAUU U GCCCUUAG | 606 | CUAAGGGC CUGAUGAG GCCGUUAGGC CGAA AAUAUUUA | 3115 |
| 2682 | UUUGCCCU U AGAUAAAG | 607 | CUUUAUCU CUGAUGAG GCCGUUAGGC CGAA AGGGCAAA | 3116 |
| 2683 | UUGCCCUU A GAUAAAGG | 608 | CCUUUAUC CUGAUGAG GCCGUUAGGC CGAA AAGGGCAA | 3117 |
| 2687 | CCUUAGAU A AAGGGAUC | 609 | GAUCCCUU CUGAUGAG GCCGUUAGGC CGAA AUCUAAGG | 3118 |
| 2695 | AAAGGGAU C AAACCGUA | 610 | UACGGUUU CUGAUGAG GCCGUUAGGC CGAA AUCCCUUU | 3119 |
| 2703 | CAAACCGU A UUAUCCAG | 611 | CUGGAUAA CUGAUGAG GCCGUUAGGC CGAA ACGGUUUG | 3120 |
| 2705 | AACCGUAU U AUCCAGAG | 612 | CUCUGGAU CUGAUGAG GCCGUUAGGC CGAA AUACGGUU | 3121 |
| 2706 | ACCGUAUU A UCCAGAGU | 613 | ACUCUGGA CUGAUGAG GCCGUUAGGC CGAA AAUACGGU | 3122 |
| 2708 | CGUAUUAU C CAGAGUAU | 614 | AUACUCUG CUGAUGAG GCCGUUAGGC CGAA AUAAUACG | 3123 |
| 2715 | UCCAGAGU A UGUAGUUA | 615 | UAACUACA CUGAUGAG GCCGUUAGGC CGAA ACUCUGGA | 3124 |
| 2719 | GAGUAUGU A GUUAAUCA | 616 | UGAUUAAC CUGAUGAG GCCGUUAGGC CGAA ACAUACUC | 3125 |
| 2722 | UAUGUAGU U AAUCAUUA | 617 | UAAUGAUU CUGAUGAG GCCGUUAGGC CGAA ACUACAUA | 3126 |
| 2723 | AUGUAGUG A AUCAUUAU | 618 | GUAAUGAU CUGAUGAG GCCGUUAGGC CGAA AACUACAU | 3127 |
| 2726 | UAGUUAAU C AUUACUUC | 619 | GAAGUAAU CUGAUGAG GCCGUUAGGC CGAA AUUAACUA | 3128 |
| 2729 | UUAAUCAU U ACUUCCAG | 620 | CUGGAAGU CUGAUGAG GCCGUUAGGC CGAA AUGAUUAA | 3129 |
| 2730 | UAAUCAUU A CUUCCAGA | 621 | UCUGGAAG CUGAUGAG GCCGUUAGGC CGAA AAUGAUUA | 3130 |
| 2733 | UCAUUACU U CCAGACGC | 622 | GCGUCUGG CUGAUGAG GCCGUUAGGC CGAA AGUAAUGA | 3131 |
| 2734 | CAUUACUU C CAGACGCG | 623 | CGCGUCUG CUGAUGAG GCCGUUAGGC CGAA AAGUAAUG | 3132 |
| 2747 | CGCGACAU U AUUUACAC | 624 | GUGUAAAU CUGAUGAG GCCGUUAGGC CGAA AUGUCGCG | 3133 |
| 2748 | GCGACAUU A UUUACACA | 625 | UGUGUAAA CUGAUGAG GCCGUUAGGC CGAA AAUGUCGC | 3134 |
| 2750 | GACAUUAU U UACACACU | 626 | AGUGUGUA CUGAUGAG GCCGUUAGGC CGAA AUAAUGUC | 3135 |
| 2751 | ACAUUAUU U ACACACUC | 627 | GAGUGUGU CUGAUGAG GCCGUUAGGC CGAA AAUAAUGU | 3136 |
| 2752 | CAUUAUUU A CACACUCU | 628 | AGAGUGUG CUGAUGAG GCCGUUAGGC CGAA AAAUAAUG | 3137 |
| 2759 | UACACACU C UUUGGAAG | 629 | CUUCCAAA CUGAUGAG GCCGUUAGGC CGAA AGUGUGUA | 3138 |
| 2761 | CACACUCU U UGGAAGGC | 630 | GCCUUCCA CUGAUGAG GCCGUUAGGC CGAA AGAGUGUG | 3139 |
| 2762 | ACACUCUU U GGAAGGCG | 631 | CGCCUUCC CUGAUGAG GCCGUUAGGC CGAA AAGAGUGU | 3140 |
| 2776 | GCGGGGAU C UUAUAUAA | 632 | UUAUAUAA CUGAUGAG GCCGUUAGGC CGAA AUCCCCGC | 3141 |
| 2778 | GGGGAUCU U AUAUAAAA | 633 | UUUUAUAU CUGAUGAG GCCGUUAGGC CGAA AGAUCCCC | 3142 |
| 2779 | GGGAUCUU A UAUAAAAG | 634 | CUUUUAUA CUGAUGAG GCCGUUAGGC CGAA AAGAUCCC | 3143 |
| 2781 | GAUCUUAU A UAAAAGAG | 635 | CUCUUUUA CUGAUGAG GCCGUUAGGC CGAA AUAAGAUC | 3144 |
| 2783 | UCUUAUAU A AAAGAGAG | 636 | CUCUCUUU CUGAUGAG GCCGUUAGGC CGAA AUAUAAGA | 3145 |
| 2793 | AAGAGAGU C CACACGUA | 637 | UACGUGUG CUGAUGAG GCCGUUAGGC CGAA ACUCUCUU | 3146 |
| 2801 | CCACACGU A GCGCCUCA | 638 | UGAGGCGC CUGAUGAG GCCGUUAGGC CGAA ACGUGUGG | 3147 |
| 2808 | UAGCGCCU C AUUUUGCG | 639 | CGCAAAAU CUGAUGAG GCCGUUAGGC CGAA AGGCGCUA | 3148 |
| 2811 | CGCCUCAU U UUGCGGGU | 640 | ACCCGCAA CUGAUGAG GCCGUUAGGC CGAA AUGAGGCG | 3149 |
| 2812 | GCCUCAUU U UGCGGGUC | 641 | GACCCGCA CUGAUGAG GCCGUUAGGC CGAA AAUGAGGC | 3150 |
| 2813 | CCUCAUUU U GCGGGUCA | 642 | UGACCCGC CUGAUGAG GCCGUUAGGC CGAA AAAUGAGG | 3151 |
| 2820 | UUGCGGGU C ACCAUAUU | 643 | AAUAUGGU CUGAUGAG GCCGUUAGGC CGAA ACCCGCAA | 3152 |
| 2826 | GUCACCAU A UUCUUGGG | 644 | CCCAAGAA CUGAUGAG GCCGUUAGGC CGAA AUGGUGAC | 3153 |
| 2828 | CACCAUAU U CUUGGGAA | 645 | UUCCCAAG CUGAUGAG GCCGUUAGGC CGAA AUAUGGUG | 3154 |
| 2829 | ACCAUAUU C UUGGGAAC | 646 | GUUCCCAA CUGAUGAG GCCGUUAGGC CGAA AAUAUGGU | 3155 |
| 2831 | CAUAUUCU U GGGAACAA | 647 | UUGUUCCC CUGAUGAG GCCGUUAGGC CGAA AGAAUAUG | 3156 |
| 2843 | AACAAGAU C UACAGCAU | 648 | AUGCUGUA CUGAUGAG GCCGUUAGGC CGAA AUCUUGUU | 3157 |
| 2845 | CAAGAUCU A CAGCAUGG | 649 | CCAUGCUG CUGAUGAG GCCGUUAGGC CGAA AGAUCCUG | 3158 |
| 2859 | UGGGAGGU U GGUCUUCC | 650 | GGAAGACC CUGAUGAG GCCGUUAGGC CGAA ACCUCCCA | 3159 |
| 2863 | AGGUUGGU C UUCCAAAC | 651 | GUUUGGAA CUGAUGAG GCCGUUAGGC CGAA ACCAACCU | 3160 |
| 2865 | GUUGGUCU U CCAAACCU | 652 | AGGUUUGG CUGAUGAG GCCGUUAGGC CGAA AGACCAAC | 3161 |
| 2866 | UUGGUCUU C CAAACCUC | 653 | GAGGUUUG CUGAUGAG GCCGUUAGGC CGAA AAGACCAA | 3162 |
| 2874 | CCAAACCU C GAAAAGGC | 654 | GCCUUUUC CUGAUGAG GCCGUUAGGC CGAA AGGUUUGG | 3163 |
| 2895 | GGACAAAU C UUUCUGUC | 655 | GACAGAAA CUGAUGAG GCCGUUAGGC CGAA AUUUGUCC | 3164 |
| 2897 | ACAAAUCU U UCUGUCCC | 656 | GGGACAGA CUGAUGAG GCCGUUAGGC CGAA AGAUUUGU | 3165 |
| 2898 | CAAAUCUU U CUGUCCCC | 657 | GGGGACAG CUGAUGAG GCCGUUAGGC CGAA AAGAUUUG | 3166 |
| 2899 | AAAUCUUU C UGUCCCCA | 658 | UGGGGACA CUGAUGAG GCCGUUAGGC CGAA AAAGAUUU | 3167 |
| 2903 | CUUUCUGU C CCCAAUCC | 659 | GGAUUGGG CUGAUGAG GCCGUUAGGC CGAA ACAGAAAG | 3168 |
| 2910 | UCCCCAAU C CCCUGGGA | 660 | UCCCAGGG CUGAUGAG GCCGUUAGGC CGAA AUUGGGGA | 3169 |
| 2920 | CCUGGGAU U CUUCCCCG | 661 | CGGGGAAG CUGAUGAG GCCGUUAGGC CGAA AUCCCAGG | 3170 |
| 2921 | CUGGGAUU C UUCCCCGA | 662 | UCGGGGAA CUGAUGAG GCCGUUAGGC CGAA AAUCCCAG | 3171 |
| 2923 | GGGAUUCU U CCCCGAUC | 663 | GAUCGGGG CUGAUGAG GCCGUUAGGC CGAA AGAAUCCC | 3172 |
| 2924 | GGAUUCUU C CCCGAUCA | 664 | UGAUCGGG CUGAUGAG GCCGUUAGGC CGAA AAGAAUCC | 3173 |
| 2931 | UCCCCGAU C AUCAGUUG | 665 | CAACUGAU CUGAUGAG GCCGUUAGGC CGAA AUCGGGGA | 3174 |
| 2934 | CCGAUCAU C AGUUGGAC | 666 | GUCCAACU CUGAUGAG GCCGUUAGGC CGAA AUGAUCGG | 3175 |
| 2938 | UCAUCAGU U GGACCCUG | 667 | CAGGGUCC CUGAUGAG GCCGUUAGGC CGAA ACUGAUGA | 3176 |
| 2950 | CCCUGCAU U CAAAGCCA | 668 | UGGCUUUG CUGAUGAG GCCGUUAGGC CGAA AUGCAGGG | 3177 |
| 2951 | CCUGCAUU C AAAGCCAA | 669 | UUGGCUUU CUGAUGAG GCCGUUAGGC CGAA AAUGCAGG | 3178 |
| 2962 | AGCCAACU C AGUAAAUC | 670 | GAUUUACU CUGAUGAG GCCGUUAGGC CGAA AGUUGGCU | 3179 |
| 2966 | AACUCAGU A AAUCCAGA | 671 | UCUGGAUU CUGAUGAG GCCGUUAGGC CGAA ACUGAGUU | 3180 |
| 2970 | CAGUAAAU C CAGAUUGG | 672 | CCAAUCUG CUGAUGAG GCCGUUAGGC CGAA AUCUACUG | 3181 |
| 2976 | AUCCAGAU U GGGACCUC | 673 | GAGGUCCC CUGAUGAG GCCGUUAGGC CGAA AUCUGGAU | 3182 |
| 2984 | UGGGACCU C AACCCGCA | 674 | UGCGGGUU CUGAUGAG GCCGUUAGGC CGAA AGGUCCCA | 3183 |
| 3037 | GGGAGCAU U CGGGCCAG | 675 | CUGGCCCG CUGAUGAG GCCGUUAGGC CGAA AUGCUCCC | 3184 |
| 3038 | GGAGCAUU C GGGCCAGG | 676 | CCUGGCCC CUGAUGAG GCCGUUAGGC CGAA AAUGCUCC | 3185 |
| 3049 | GCCAGGGU U CACCCCUC | 677 | GAGGGGUG CUGAUGAG GCCGUUAGGC CGAA ACCCUGGC | 3186 |
| 3050 | CCAGGGUU C ACCCCUCC | 678 | GGAGGGGU CUGAUGAG GCCGUUAGGC CGAA AACCCUGG | 3187 |
| 3057 | UCACCCCU C CCCAUGGG | 679 | CCCAUGGG CUGAUGAG GCCGUUAGGC CGAA AGGGGUGA | 3188 |
| 3073 | GGGACUGU U GGGGUGGA | 680 | UCCACCCC CUGAUGAG GCCGUUAGGC CGAA ACAGUCCC | 3189 |
| 3087 | GGAGCCCU C ACGCUCAG | 681 | CUGAGCGU CUGAUGAG GCCGUUAGGC CGAA AGGGCUCC | 3190 |
| 3093 | CUCACGCU C AGGGCCUA | 682 | UAGGCCCU CUGAUGAG GCCGUUAGGC CGAA AGCGUGAG | 3191 |
| 3101 | CAGGGCCU A CUCACAAC | 683 | GUUGUGAG CUGAUGAG GCCGUUAGGC CGAA AGGCCCUG | 3192 |
| 3104 | GGCCUACU C ACAACUGU | 684 | ACAGUUGU CUGAUGAG GCCGUUAGGC CGAA AGUAGGCC | 3193 |
| 3123 | CAGCAGCU C CUCCUCCU | 685 | AGGAGGAG CUGAUGAG GCCGUUAGGC CGAA AGCUGCUG | 3194 |
| 3126 | CAGCUCCU C CUCCUGCC | 686 | GGCAGGAG CUGAUGAG GCCGUUAGGC CGAA AGGAGCUG | 3195 |
| 3129 | CUCCUCCU C CUGCCUCC | 687 | GGAGGCAG CUGAUGAG GCCGUUAGGC CGAA AGGAGGAG | 3196 |
| 3136 | UCCUGCCU C CACCAAUC | 688 | GAUUGGUG CUGAUGAG GCCGUUAGGC CGAA AGGCAGGA | 3197 |
| 3144 | CCACCAAU C GGCAGUCA | 689 | UGACUGCC CUGAUGAG GCCGUUAGGC CGAA AUUGGUGG | 3198 |
| 3151 | UCGGCAGU C AGGAAGGC | 690 | GCCUUCCU CUGAUGAG GCCGUUAGGC CGAA ACUGCCGA | 3199 |
| 3165 | GGCAGCCU A CUCCCUUA | 691 | UAAGGGAG CUGAUGAG GCCGUUAGGC CGAA AGGCUGCC | 3200 |
| 3168 | AGCCUACU C CCUUAUCU | 692 | AGAUAAGG CUGAUGAG GCCGUUAGGC CGAA AGUAGGCU | 3201 |
| 3172 | UACUCCCU U AUCUCCAC | 693 | GUGGAGAU CUGAUGAG GCCGUUAGGC CGAA AGGGAGUA | 3202 |
| 3173 | ACUCCCUU A UCUCCACC | 694 | GGUGGAGA CUGAUGAG GCCGUUAGGC CGAA AAGGGAGU | 3203 |
| 3175 | UCCCUUAU C UCCACCUC | 695 | GAGGUGGA CUGAUGAG GCCGUUAGGC CGAA AUAAGGGA | 3204 |
| 3177 | CCUUAUCU C CACCUCUA | 696 | UAGAGGUG CUGAUGAG GCCGUUAGGC CGAA AGAUAAGG | 3205 |
| 3183 | CUCCACCU C UAAGGGAC | 697 | GUCCCUUA CUGAUGAG GCCGUUAGGC CGAA AGGUGGAG | 3206 |
| 3185 | CCACCUCU A AGGGACAC | 698 | GUGUCCCU CUGAUGAG GCCGUUAGGC CGAA AGAGGUGG | 3207 |
| 3195 | GGGACACU C AUCCUCAG | 699 | CUGAGGAU CUGAUGAG GCCGUUAGGC CGAA AGUGUCCC | 3208 |
| 3198 | ACACUCAU C CUCAGGCC | 700 | GGCCUGAG CUGAUGAG GCCGUUAGGC CGAA AUGAGUGU | 3209 |
| 3201 | CUCAUCCU C AGGCCAUG | 701 | CAUGGCCU CUGAUGAG GCCGUUAGGC CGAA AGGAUGAG | 3210 |

Input Sequence = AF100308. Cut Site = UH/.
Stem Length = 8 . Core Sequence = CUGAUGAG GCCGUUAGGC CGAA
AF100308 (Hepatitis B virus strain 2-18, 3215 bp)
Underlined region can be any X sequence or linker, as described herein.

TABLE VI

HUMAN HBV INOZYME AND SUBSTRATE SEQUENCE

| | | | | |
|---|---|---|---|---|
| | | | | Seq |
| Pos | Substrate | Seq ID | Inozyme | ID |

| | | | | |
|---|---|---|---|---|
| 9 | AACUCCAC C ACUUUCCA | 702 | UGGAAAGU CUGAUGAG GCCGUUAGGC CGAA IUGGAGUU | 3211 |
| 10 | ACUCCACC A CUUUCCAC | 703 | GUGGAAAG CUGAUGAG GCCGUUAGGC CGAA IGUGGAGU | 3212 |
| 12 | UCCACCAC U UUCCACCA | 704 | UGGUGGAA CUGAUGAG GCCGUUAGGC CGAA IUGGUGGA | 3213 |
| 16 | CCACUUUC C ACCAAACU | 705 | AGUUUGGU CUGAUGAG GCCGUUAGGC CGAA IAAAGUGG | 3214 |
| 17 | CACUUUCC A CCAAACUC | 706 | GAGUUUGG CUGAUGAG GCCGUUAGGC CGAA IGAAAGUG | 3215 |
| 19 | CUUUCCAC C AAACUCUU | 707 | AAGAGUUU CUGAUGAG GCCGUUAGGC CGAA IUGGAAAG | 3216 |
| 20 | UUUCCACC A AACUCUUC | 708 | GAAGAGUU CUGAUGAG GCCGUUAGGC CGAA IGUGGAAA | 3217 |
| 24 | CACCAAAC U CUUCAAGA | 709 | UCUUGAAG CUGAUGAG GCCGUUAGGC CGAA IUUUGGUG | 3218 |
| 26 | CCAAACUC U UCAAGAUC | 710 | GAUCUUGA CUGAUGAG GCCGUUAGGC CGAA IAGUUUGG | 3219 |
| 29 | AACUCUUC A AGAUCCCA | 711 | UGGGAUCU CUGAUGAG GCCGUUAGGC CGAA IAAGAGUU | 3220 |
| 35 | UCAAGAUC C CAGAGUCA | 712 | UGACUCUG CUGAUGAG GCCGUUAGGC CGAA IAUCUUGA | 3221 |
| 36 | CAAGAUCC C AGAGUCAG | 713 | CUGACUCU CUGAUGAG GCCGUUAGGC CGAA IGAUCUUG | 3222 |
| 37 | AAGAUCCC A GAGUCAGG | 714 | CCUGACUC CUGAUGAG GCCGUUAGGC CGAA IGGAUCUU | 3223 |
| 43 | CCAGAGUC A GGGCCCUG | 715 | CAGGGCCC CUGAUGAG GCCGUUAGGC CGAA IACUCUGG | 3224 |
| 48 | GUCAGGGC C CUGUACUU | 716 | AAGUACAG CUGAUGAG GCCGUUAGGC CGAA ICCCUGAC | 3225 |
| 49 | UCAGGGCC C UGUACUUU | 717 | AAAGUACA CUGAUGAG GCCGUUAGGC CGAA IGCCCUGA | 3226 |
| 50 | CAGGGCCC U GUACUUUC | 718 | GAAAGUAC CUGAUGAG GCCGUUAGGC CGAA IGGCCCUG | 3227 |
| 55 | CCCUGUAC U UUCCUGCU | 719 | AGCAGGAA CUGAUGAG GCCGUUAGGC CGAA IUACAGGG | 3228 |
| 59 | GUACUCUC C UGCUGGUG | 720 | CACCAGCA CUGAUGAG GCCGUUAGGC CGAA IAAAGUAC | 3229 |
| 60 | UACUUUCC U GCUGGUGG | 721 | CCACCAGC CUGAUGAG GCCGUUAGGC CGAA IGAAAGUA | 3230 |
| 63 | UUUCCUGC U GGUGGCUC | 722 | GAGCCACC CUGAUGAG GCCGUUAGGC CGAA ICAGGAAA | 3231 |
| 70 | CUGGUGGC U CCAGUUCA | 723 | UGAACUGG CUGAUGAG GCCGUUAGGC CGAA ICCACCAG | 3232 |
| 72 | GGUGGCUC C AGUUCAGG | 724 | CCUGAACU CUGAUGAG GCCGUUAGGC CGAA IAGCCACC | 3233 |
| 73 | GUGGCUCC A GUUCAGGA | 725 | UCCUGAAC CUGAUGAG GCCGUUAGGC CGAA IGAGCCAC | 3234 |
| 78 | UCCAGUUC A GGAACAGU | 726 | ACUGUUCC CUGAUGAG GCCGUUAGGC CGAA IAACUGGA | 3235 |
| 84 | UCAGGAAC A GUGAGCCC | 727 | GGGCUCAC CUGAUGAG GCCGUUAGGC CGAA IUUCCUGA | 3236 |
| 91 | CAGUGAGC C CUGCUCAG | 728 | CUGAGCAG CUGAUGAG GCCGUUAGGC CGAA ICUCACUG | 3237 |
| 92 | AGUGAGCC C UGCUCAGA | 729 | UCUGAGCA CUGAUGAG GCCGUUAGGC CGAA IGCUCACU | 3238 |
| 93 | GUGAGCCC U GCUCAGAA | 730 | UUCUGAGC CUGAUGAG GCCGUUAGGC CGAA IGGCUCAC | 3239 |
| 96 | AGCCCUGC U CAGAAUAC | 731 | GUAUUCUG CUGAUGAG GCCGUUAGGC CGAA ICAGGGCU | 3240 |
| 98 | CCCUGCUC A GAAUACUG | 732 | CAGUAUUC CUGAUGAG GCCGUUAGGC CGAA IAGCAGGG | 3241 |
| 105 | CAGAAUAC U GUCUCUGC | 733 | GCAGAGAC CUGAUGAG GCCGUUAGGC CGAA IUAUUCUG | 3242 |
| 109 | AUACUGUC U CUGCCAUA | 734 | UAUGGCAG CUGAUGAG GCCGUUAGGC CGAA IACAGUAU | 3243 |
| 111 | ACUGUCUC U GCCAUAUC | 735 | GAUAUGGC CUGAUGAG GCCGUUAGGC CGAA IAGACAGU | 3244 |
| 114 | GUCUCUGC C AUAUCGUC | 736 | GACGAUAU CUGAUGAG GCCGUUAGGC CGAA ICAGAGAC | 3245 |
| 115 | UCUCUGCC A UAUCGUCA | 737 | UGACGAUA CUGAUGAG GCCGUUAGGC CGAA ICCAGAGA | 3246 |
| 123 | AUAUCGUC A AUCUUAUC | 738 | GAUAAGAU CUGAUGAG GCCGUUAGGC CGAA IACGAUAU | 3247 |
| 127 | CGUCAAUC U UAUCGAAG | 739 | CUUCGAUA CUGAUGAG GCCGUUAGGC CGAA IAUUGACG | 3248 |
| 138 | UCGAAGAC U GGGGACCC | 740 | GGGUCCCC CUGAUGAG GCCGUUAGGC CGAA IUCUUCGA | 3249 |
| 145 | CUGGGGAC C CUGUACCG | 741 | CGGUACAG CUGAUGAG GCCGUUAGGC CGAA IUCCCCAG | 3250 |
| 146 | UGGGGACC C UGUACCGA | 742 | UCGGUACA CUGAUGAG GCCGUUAGGC CGAA IGUCCCCA | 3251 |
| 147 | GGGGACCC U GUACCGAA | 743 | UUCGGUAC CUGAUGAG GCCGUUAGGC CGAA IGGUCCCC | 3252 |
| 152 | CCCUGUAC C GAACAUGG | 744 | CCAUGUUC CUGAUGAG GCCGUUAGGC CGAA IUACAGGG | 3253 |
| 157 | UACCGAAC A UGGAGAAC | 745 | GUUCUCCA CUGAUGAG GCCGUUAGGC CGAA IUUCGGUA | 3254 |
| 166 | UGGAGAAC A UCGCAUCA | 746 | UGAUGCGA CUGAUGAG GCCGUUAGGC CGAA IUUCUCCA | 3255 |
| 171 | AACAUCGC A UCAGGACU | 747 | AGUCCUGA CUGAUGAG GCCGUUAGGC CGAA ICGAUGUU | 3256 |
| 174 | AUCGCAUC A GGACUCCU | 748 | AGGAGUCC CUGAUGAG GCCGUUAGGC CGAA IAUGCGAU | 3257 |
| 179 | AUCAGGAC U CCUAGGAC | 749 | GUCCUAGG CUGAUGAG GCCGUUAGGC CGAA IUCCUGAU | 3258 |
| 181 | CAGGACUC C UAGGACCC | 750 | GGGUCCUA CUGAUGAG GCCGUUAGGC CGAA IAGUCCUG | 3259 |
| 182 | AGGACUCC U AGGACCCC | 751 | GGGGUCCU CUGAUGAG GCCGUUAGGC CGAA IGAGUCCU | 3260 |
| 188 | CCUAGGAC C CCUGCUCG | 752 | CGAGCAGG CUGAUGAG GCCGUUAGGC CGAA IUCCUAGG | 3261 |
| 189 | CUAGGACC C CUGCUCGU | 753 | ACGAGCAG CUGAUGAG GCCGUUAGGC CGAA IGUCCUAG | 3262 |
| 190 | UAGGACCC C UGCUCGUG | 754 | CACGAGCA CUGAUGAG GCCGUUAGGC CGAA IGGUCCUA | 3263 |
| 191 | AGGACCCC U GCUCGUGU | 755 | ACACGAGC CUGAUGAG GCCGUUAGGC CGAA IGGGUCCU | 3264 |
| 194 | ACCCCUGC U CGUGUUAC | 756 | GUAACACG CUGAUGAG GCCGUUAGGC CGAA ICAGGGGU | 3265 |
| 203 | CGUGUUAC A GGCGGGGU | 757 | ACCCCGCC CUGAUGAG GCCGUUAGGC CGAA IUAACACG | 3266 |
| 217 | GGUUUUUC U UGUUGACA | 758 | UGUCAACA CUGAUGAG GCCGUUAGGC CGAA IAAAAACC | 3267 |
| 225 | UUGUUGAC A AAAAUCCU | 759 | AGGAUUUU CUGAUGAG GCCGUUAGGC CGAA IUCAACAA | 3268 |
| 232 | CAAAAAUC C UCACAAUA | 760 | UAUUGUGA CUGAUGAG GCCGUUAGGC CGAA IAUUUUUG | 3269 |
| 233 | AAAAAUCC U CACAAUAC | 761 | GUAUUGUG CUGAUGAG GCCGUUAGGC CGAA IGAUUUUU | 3270 |
| 235 | AAAUCCUC A CAAUACCA | 762 | UGGUAUUG CUGAUGAG GCCGUUAGGC CGAA IAGGAUUU | 3271 |
| 237 | AUCCUCAC A AUACCACA | 763 | UGUGGUAU CUGAUGAG GCCGUUAGGC CGAA IUGAGGAU | 3272 |
| 242 | CACAAUAC C ACAGAGUC | 764 | GACUCUGU CUGAUGAG GCCGUUAGGC CGAA IUAUUGUG | 3273 |
| 243 | ACAAUACC A CAGAGUCU | 765 | AGACUCUG CUGAUGAG GCCGUUAGGC CGAA IGUAUUGU | 3274 |
| 245 | AAUACCAC A GAGUCUAG | 766 | CUAGACUC CUGAUGAG GCCGUUAGGC CGAA IUGGUAUU | 3275 |
| 251 | ACAGAGUC U AGACUCGU | 767 | ACGAGUCU CUGAUGAG GCCGUUAGGC CGAA IACUCUGU | 3276 |
| 256 | GUCUAGAC U CGUGGUGG | 768 | CCACCACG CUGAUGAG GCCGUUAGGC CGAA IUCUAGAC | 3277 |
| 267 | UGGUGGAC U UCUCUCAA | 769 | UUGAGAGA CUGAUGAG GCCGUUAGGC CGAA IUCCACCA | 3278 |
| 270 | UGGACUUC U CUCAAUUU | 770 | AAAUUGAG CUGAUGAG GCCGUUAGGC CGAA IAAGUCCA | 3279 |
| 272 | GACUUCUC U CAAUUUUC | 771 | GAAAAUUG CUGAUGAG GCCGUUAGGC CGAA IAGAAGUC | 3280 |
| 274 | CUUCUCUC A AUUUUCUA | 772 | UAGAAAAU CUGAUGAG GCCGUUAGGC CGAA IAGAGAAG | 3281 |
| 281 | CAAUUUUC U AGGGGGAA | 773 | UUCCCCCU CUGAUGAG GCCGUUAGGC CGAA IAAAAUUG | 3282 |
| 291 | GGGGGAAC A CCCGUGUG | 774 | CACACGGG CUGAUGAG GCCGUUAGGC CGAA IUUCCCCC | 3283 |
| 293 | GGGAACAC C CGUGUGUC | 775 | GACACACG CUGAUGAG GCCGUUAGGC CGAA IUGUUCCC | 3284 |
| 294 | GGAACACC C GUGUGUCU | 776 | AGACACAC CUGAUGAG GCCGUUAGGC CGAA IGUGUUCC | 3285 |
| 302 | CGUGUGUC U UGGCCAAA | 777 | UUUGGCCA CUGAUGAG GCCGUUAGGC CGAA IACACACG | 3286 |
| 307 | GUCUUGGC C AAAAUUCG | 778 | CGAAUUUU CUGAUGAG GCCGUUAGGC CGAA ICCAAGAC | 3287 |
| 308 | UCUUGGCC A AAAUUCGC | 779 | GCGAAUUU CUGAUGAG GCCGUUAGGC CGAA IGCCAAGA | 3288 |
| 317 | AAAUUCGC A GUCCCAAA | 780 | UUUGGGAC CUGAUGAG GCCGUUAGGC CGAA ICGAAUUU | 3289 |
| 321 | UCGCAGUC C CAAAUCUC | 781 | GAGAUUUG CUGAUGAG GCCGUUAGGC CGAA IACUGCGA | 3290 |
| 322 | CGCAGUCC C AAAUCUCC | 782 | GGAGAUUU CUGAUGAG GCCGUUAGGC CGAA IGACUGCG | 3291 |
| 323 | GCAGUCCC A AAUCUCCA | 783 | UGGAGAUU CUGAUGAG GCCGUUAGGC CGAA IGGACUGC | 3292 |
| 328 | CCCAAAUC U CCAGUCAC | 784 | GUGACUGG CUGAUGAG GCCGUUAGGC CGAA IAUUUGGG | 3293 |
| 330 | CAAAUCUC C AGUCACUC | 785 | GAGUGACU CUGAUGAG GCCGUUAGGC CGAA IAGAUUUG | 3294 |
| 331 | AAAUCUCC A GUCACUCA | 786 | UGAGUGAC CUGAUGAG GCCGUUAGGC CGAA IGAGAUUU | 3295 |
| 335 | CUCCAGUC A CUCACCAA | 787 | UUGGUGAG CUGAUGAG GCCGUUAGGC CGAA IACUGGAG | 3296 |
| 337 | CCAGUCAC U CACCAACC | 788 | GGUUGGUG CUGAUGAG GCCGUUAGGC CGAA IUGACUGG | 3297 |
| 339 | AGUCACUC A CCAACCUG | 789 | CAGGUUGG CUGAUGAG GCCGUUAGGC CGAA IAGUGACU | 3298 |
| 341 | UCACUCAC C AACCUGUU | 790 | AACAGGUU CUGAUGAG GCCGUUAGGC CGAA IUGAGUGA | 3299 |
| 342 | CACUCACC A ACCUGUUG | 791 | CAACAGGU CUGAUGAG GCCGUUAGGC CGAA IGUGAGUG | 3300 |
| 345 | UCACCAAC C UGUUGUCC | 792 | GGACAACA CUGAUGAG GCCGUUAGGC CGAA IUUGGUGA | 3301 |
| 346 | CACCAACC U GUUGUCCU | 793 | AGGACAAC CUGAUGAG GCCGUUAGGC CGAA IGUUGGUG | 3302 |
| 353 | CUGUUGUC C UCCAAUUU | 794 | AAAUUGGA CUGAUGAG GCCGUUAGGC CGAA IACAACAG | 3303 |
| 354 | UGUUGUCC U CCAAUUUG | 795 | CAAAUUGG CUGAUGAG GCCGUUAGGC CGAA IGACAACA | 3304 |
| 356 | UUGUCCUC C AAUUUGUC | 796 | GACAAAUU CUGAUGAG GCCGUUAGGC CGAA IAGGACAA | 3305 |
| 357 | UGUCCUCC A AUUUGUCC | 797 | GGACAAAU CUGAUGAG GCCGUUAGGC CGAA IGAGGACA | 3306 |
| 365 | AAUUUGUC C UGGUUAUC | 798 | GAUAACCA CUGAUGAG GCCGUUAGGC CGAA IACAAAUU | 3307 |
| 366 | AUUUGUCC U GGUUAUCG | 799 | CGAUAACC CUGAUGAG GCCGUUAGGC CGAA IGACAAAU | 3308 |
| 376 | GUUAUCGC U GGAUGUGU | 800 | ACACAUCC CUGAUGAG GCCGUUAGGC CGAA ICGAUAAC | 3309 |
| 386 | GAUGUGUC U GCGGCGUU | 801 | AACGCCGC CUGAUGAG GCCGUUAGGC CGAA IACACAUC | 3310 |
| 400 | GUUUUAUC A UCUUCCUC | 802 | GAGGAAGA CUGAUGAG GCCGUUAGGC CGAA IAUAAAAC | 3311 |
| 403 | UUAUCAUC U UCCUCUGC | 803 | GCAGAGGA CUGAUGAG GCCGUUAGGC CGAA IAUGAUAA | 3312 |
| 406 | UCAUCUUC C UCUGCAUC | 804 | GAUGCAGA CUGAUGAG GCCGUUAGGC CGAA IAAGAUGA | 3313 |
| 407 | CAUCUUCC U CUGCAUCC | 805 | GGAUGCAG CUGAUGAG GCCGUUAGGC CGAA IGAAGAUG | 3314 |
| 409 | UCUUCCUC U GCAUCCUG | 806 | CAGGAUGC CUGAUGAG GCCGUUAGGC CGAA IAGGAAGA | 3315 |
| 412 | UCCUCUGC A UCCUGCUG | 807 | CAGCAGGA CUGAUGAG GCCGUUAGGC CGAA ICAGAGGA | 3316 |
| 415 | UCUGCAUC C UGCUGCUA | 808 | UAGCAGCA CUGAUGAG GCCGUUAGGC CGAA IAUGCAGA | 3317 |
| 416 | CUGCAUCC U GCUGCUAU | 809 | AUAGCAGC CUGAUGAG GCCGUUAGGC CGAA IGAUGCAG | 3318 |
| 419 | CAUCCUGC U GCUAUGCC | 810 | GGCAUAGC CUGAUGAG GCCGUUAGGC CGAA ICAGGAUG | 3319 |
| 422 | CCUGCUGC U AUGCCUCA | 811 | UGAGGCAU CUGAUGAG GCCGUUAGGC CGAA ICAGCAGG | 3320 |
| 427 | UGCUAUGC C UCAUCUUC | 812 | GAAGAUGA CUGAUGAG GCCGUUAGGC CGAA ICAUAGCA | 3321 |
| 428 | GCUAUGCC U CAUCUUCU | 813 | AGAAGAUG CUGAUGAG GCCGUUAGGC CGAA IGCAUAGC | 3322 |
| 430 | UAUGCCUC A UCUUCUUG | 814 | CAAGAAGA CUGAUGAG GCCGUUAGGC CGAA IAGGCAUA | 3323 |
| 433 | GCCUCAUC U UCUUGUUG | 815 | CAACAAGA CUGAUGAG GCCGUUAGGC CGAA IAUGAGGC | 3324 |
| 436 | UCAUCUUC U UGUUGGUU | 816 | AACCAACA CUGAUGAG GCCGUUAGGC CGAA IAAGAUGA | 3325 |
| 446 | GUUGGUUC U UCUGGACU | 817 | AGUCCAGA CUGAUGAG GCCGUUAGGC CGAA IAACCAAC | 3326 |
| 449 | GGUUCUUC U GGACUAUC | 818 | GAUAGUCC CUGAUGAG GCCGUUAGGC CGAA IAAGAACC | 3327 |
| 454 | UUCUGGAC U AUCAAGGU | 819 | ACCUUGAU CUGAUGAG GCCGUUAGGC CGAA IUCCAGAA | 3328 |
| 458 | GGACUAUC A AGGUAUGU | 820 | ACAUACCU CUGAUGAG GCCGUUAGGC CGAA IAUAGUCC | 3329 |
| 470 | UAUGUUGC C CGUUUGUC | 821 | GACAAACG CUGAUGAG GCCGUUAGGC CGAA ICAACAUA | 3330 |
| 471 | AUGUUGCC C GUUUGUCC | 822 | GGACAAAC CUGAUGAG GCCGUUAGGC CGAA IGCAACAU | 3331 |
| 479 | CGUUUGUC C UCUAAUUC | 823 | GAAUUAGA CUGAUGAG GCCGUUAGGC CGAA IACAAACG | 3332 |
| 480 | GUUUGUCC U CUAAUUCC | 824 | GGAAUUAG CUGAUGAG GCCGUUAGGC CGAA IGACAAAC | 3333 |
| 482 | UUGUCCUC U AAUUCCAG | 825 | CUGGAAUU CUGAUGAG GCCGUUAGGC CGAA IAGGACAA | 3334 |
| 488 | UCUAAUUC C AGGAUCAU | 826 | AUGAUCCU CUGAUGAG GCCGUUAGGC CGAA IAAUUAGA | 3335 |
| 489 | CUAAUUCC A GGAUCAUC | 827 | GAUGAUCC CUGAUGAG GCCGUUAGGC CGAA IGAAUUAG | 3336 |
| 495 | CCAGGAUC A UCAACAAC | 828 | GUUGUUGA CUGAUGAG GCCGUUAGGC CGAA IAUCCUGG | 3337 |
| 498 | GGAUCAUC A ACAACCAG | 829 | CUGGUUGU CUGAUGAG GCCGUUAGGC CGAA IAUGAUCC | 3338 |
| 501 | UCAUCAAC A ACCAGCAC | 830 | GUGCUGGU CUGAUGAG GCCGUUAGGC CGAA IUUGAUGA | 3339 |
| 504 | UCAACAAC C AGCACCGG | 831 | CCGGUGCU CUGAUGAG GCCGUUAGGC CGAA IUUGUUGA | 3340 |
| 505 | CAACAACC A GCACCGGA | 832 | UCCGGUGC CUGAUGAG GCCGUUAGGC CGAA IGUUGUUG | 3341 |
| 508 | CAACCAGC A CCGGACCA | 833 | UGGUCCGG CUGAUGAG GCCGUUAGGC CGAA ICUGGUUG | 3342 |
| 510 | ACCAGCAC C GGACCAUG | 834 | CAUGGUCC CUGAUGAG GCCGUUAGGC CGAA IUGCUGGU | 3343 |
| 515 | CACCGGAC C AUGCAAAA | 835 | UUUUGCAU CUGAUGAG GCCGUUAGGC CGAA IUCCGGUG | 3344 |
| 516 | ACCGGACC A UGCAAAAC | 836 | GUUUUGCA CUGAUGAG GCCGUUAGGC CGAA IGUCCGGU | 3345 |
| 520 | GACCAUGC A AAACCUGC | 837 | GCAGGUUU CUGAUGAG GCCGUUAGGC CGAA ICAUGGUC | 3346 |
| 525 | UGCAAAAC C UGCACAAC | 838 | GUUGUGCA CUGAUGAG GCCGUUAGGC CGAA IUUUUGCA | 3347 |
| 526 | GCAAAACC U GCACAACU | 839 | AGUUGUGC CUGAUGAG GCCGUUAGGC CGAA IGUUUUGC | 3348 |
| 529 | AAACCUGC A CAACUCCU | 840 | AGGAGUUG CUGAUGAG GCCGUUAGGC CGAA ICAGGUUU | 3349 |
| 531 | ACCUGCAC A ACUCCUGC | 841 | GCAGGAGU CUGAUGAG GCCGUUAGGC CGAA IUGCAGGU | 3350 |
| 534 | UGCACAAC U CCUGCUCA | 842 | UGAGCAGG CUGAUGAG GCCGUUAGGC CGAA IUUGUGCA | 3351 |
| 536 | CACAACUC C UGCUCAAG | 843 | CUUGAGCA CUGAUGAG GCCGUUAGGC CGAA IAGUUGUG | 3352 |
| 537 | ACAACUCC U GCUCAAGG | 844 | CCUUGAGC CUGAUGAG GCCGUUAGGC CGAA IGAGUUGU | 3353 |
| 540 | ACUCCUGC U CAAGGAAC | 845 | GUUCCUUG CUGAUGAG GCCGUUAGGC CGAA ICAGGAGU | 3354 |
| 542 | UCCUGCUC A AGGAACCU | 846 | AGGUUCCU CUGAUGAG GCCGUUAGGC CGAA IAGCAGGA | 3355 |
| 549 | CAAGGAAC C UCUAUGUU | 847 | AACAUAGA CUGAUGAG GCCGUUAGGC CGAA IUUCCUUG | 3356 |
| 550 | AAGGAACC U CUAUGUUU | 848 | AAACAUAG CUGAUGAG GCCGUUAGGC CGAA IGUUCCUU | 3357 |
| 552 | GGAACCUC U AUGUUUCC | 849 | GGAAACAU CUGAUGAG GCCGUUAGGC CGAA IAGGUUCC | 3358 |
| 560 | UAUGUUUC C CUCAUGUU | 850 | AACAUGAG CUGAUGAG GCCGUUAGGC CGAA IAAACAUA | 3359 |
| 561 | AUGUUUCC C UCAUGUUG | 851 | CAACAUGA CUGAUGAG GCCGUUAGGC CGAA IGAAACAU | 3360 |
| 562 | UGUUUCCC U CAUGUUGC | 852 | GCAACAUG CUGAUGAG GCCGUUAGGC CGAA IGGAAACA | 3361 |
| 564 | UUUCCCUC A UGUUGCUG | 853 | CAGCAACA CUGAUGAG GCCGUUAGGC CGAA IAGGGAAA | 3362 |
| 571 | CAUGUUGC U GUACAAAA | 854 | UUUUGUAC CUGAUGAG GCCGUUAGGC CGAA ICAACAUG | 3363 |
| 576 | UGCUGUAC A AAACCUAC | 855 | GUAGGUUU CUGAUGAG GCCGUUAGGC CGAA IUACAGCA | 3364 |
| 581 | UACAAAAC C UACGGACG | 856 | CGUCCGUA CUGAUGAG GCCGUUAGGC CGAA IUUUUGUA | 3365 |
| 582 | ACAAAACC U ACGGACGG | 857 | CCGUCCGU CUGAUGAG GCCGUUAGGC CGAA IGUUUUGU | 3366 |
| 595 | ACGGAAAC U GCACCUGU | 858 | ACAGGUGC CUGAUGAG GCCGUUAGGC CGAA IUUUCCGU | 3367 |
| 598 | GAAACUGC A CCUGUAUU | 859 | AAUACAGG CUGAUGAG GCCGUUAGGC CGAA ICAGUUUC | 3368 |
| 600 | AACUGCAC C UGUAUUCC | 860 | GGAAUACA CUGAUGAG GCCGUUAGGC CGAA IUGCAGUU | 3369 |
| 601 | ACUGCACC U GUAUUCCC | 861 | GGGAAUAC CUGAUGAG GCCGUUAGGC CGAA IGUGCAGU | 3370 |
| 608 | CUGUAUUC C CAUCCCAU | 862 | AUGGGAUG CUGAUGAG GCCGUUAGGC CGAA IAAUACAG | 3371 |
| 609 | UGUAUUCC C AUCCCAUC | 863 | GAUGGGAU CUGAUGAG GCCGUUAGGC CGAA IGAAUACA | 3372 |
| 610 | GUAUUCCC A UCCCAUCA | 864 | UGAUGGGA CUGAUGAG GCCGUUAGGC CGAA IGGAAUAC | 3373 |
| 613 | UUCCCAUC C CAUCAUCU | 865 | AGAUGAUG CUGAUGAG GCCGUUAGGC CGAA IAUGGGAA | 3374 |
| 614 | UCCCAUCC C AUCAUCUU | 866 | AAGAUGAU CUGAUGAG GCCGUUAGGC CGAA IGAUGGGA | 3375 |
| 615 | CCCAUCCC A UCAUCUUG | 867 | CAAGAUGA CUGAUGAG GCCGUUAGGC CGAA IGGAUGGG | 3376 |
| 618 | AUCCCAUC A UCUUGGGC | 868 | GCCCAAGA CUGAUGAG GCCGUUAGGC CGAA IAUGGGAU | 3377 |
| 621 | CCAUCAUC U UGGGCUUU | 869 | AAAGCCCA CUGAUGAG GCCGUUAGGC CGAA IAUGAUGG | 3378 |
| 627 | UCUUGGGC U UUCGCAAA | 870 | UUUGCGAA CUGAUGAG GCCGUUAGGC CGAA ICCCAAGA | 3379 |
| 633 | GCUUUCGC A AAAUACCU | 871 | AGGUAUUU CUGAUGAG GCCGUUAGGC CGAA ICGAAAGC | 3380 |
| 640 | CAAAAUAC C UAUGGGAG | 872 | CUCCCAUA CUGAUGAG GCCGUUAGGC CGAA IUAUUUUG | 3381 |
| 641 | AAAAUACC U AUGGGAGU | 873 | ACUCCCAU CUGAUGAG GCCGUUAGGC CGAA IGUAUUUU | 3382 |
| 654 | GAGUGGGC C UCAGUCCG | 874 | CGGACUGA CUGAUGA3 GCCGUUAGGC CGAA ICCCACUC | 3383 |
| 655 | AGUGGGCC U CAGUCCGU | 875 | ACGGACUG CUGAUGAG GCCGUUAGGC CGAA IGCCCACU | 3384 |
| 657 | UGGGCCUC A GUCCGUUU | 876 | AAACGGAC CUGAUGAG GCCGUUAGGC CGAA IAGGCCCA | 3385 |
| 661 | CCUCAGUC C GUUUCUCU | 877 | AGAGAAAC CUGAUGAG GCCGUUAGGC CGAA IACUGAGG | 3386 |
| 667 | UCCGUUUC U CUUGGCUC | 878 | GAGCCAAG CUGAUGAG GCCGUUAGGC CGAA IAAACGGA | 3387 |
| 669 | CGUUUCUC U UGGCUCAG | 879 | CUGAGCCA CUGAUGAG GCCGUUAGGC CGAA IAGAAACG | 3388 |
| 674 | CUCUUGGC U CAGUUUAC | 880 | GUAAACUG CUGAUGAG GCCGUUAGGC CGAA ICCAAGAG | 3389 |
| 676 | CUUGGCUC A GUUUACUA | 881 | UAGUAAAC CUGAUGAG GCCGUUAGGC CGAA IAGCCAAG | 3390 |
| 683 | CAGUUUAC U AGUGCCAU | 882 | AUGGCACU CUGAUGAG GCCGUUAGGC CGAA IUAAACUG | 3391 |
| 689 | ACUAGUGC C AUUUGUUC | 883 | GAACAAAU CUGAUGAG GCCGUUAGGC CGAA ICACUAGU | 3392 |
| 690 | CUAGUGCC A UUUGUUCA | 884 | UGAACAAA CUGAUGAG GCCGUUAGGC CGAA IGCACUAG | 3393 |
| 698 | AUUUGUUC A GUGGUUCG | 885 | CGAACCAC CUGAUGAG GCCGUUAGGC CGAA IAACAAAU | 3394 |
| 713 | CGUAGGGC U UUCCCCCA | 886 | UGGGGGAA CUGAUGAG GCCGUUAGGC CGAA ICCCUACG | 3395 |
| 717 | GGGCUUUC C CCCACUGU | 887 | ACAGUGGG CUGAUGAG GCCGUUAGGC CGAA IAAAGCCC | 3396 |
| 718 | GGCUUUCC C CCACUGUC | 888 | GACAGUGG CUGAUGAG GCCGUUAGGC CGAA IGAAAGCC | 3397 |
| 719 | GCUUUCCC C CACUGUCU | 889 | AGACAGUG CUGAUGAG GCCGUUAGGC CGAA IGGAAAGC | 3398 |
| 720 | CUUUCCCC C ACUGUCUG | 890 | CAGACAGU CUGAUGAG GCCGUUAGGC CGAA IGGGAAAG | 3399 |
| 721 | UUUCCCCC A CUGUCUGG | 891 | CCAGACAG CUGAUGAG GCCGUUAGGC CGAA IGGGGAAA | 3400 |
| 723 | UCCCCCAC U GUCUGGCU | 892 | AGCCAGAC CUGAUGAG GCCGUUAGGC CGAA IUGGGGGA | 3401 |
| 727 | CCACUGUC U GGCUUUCA | 893 | UGAAAGCC CUGAUGAG GCCGUUAGGC CGAA IACAGUGG | 3402 |
| 731 | UGUCUGGC U UUCAGUUA | 894 | UAACUGAA CUGAUGAG GCCGUUAGGC CGAA ICCAGACA | 3403 |
| 735 | UGGCUUUC A GUUAUAUG | 895 | CAUAUAAC CUGAUGAG GCCGUUAGGC CGAA IAAAGCCA | 3404 |
| 764 | UUGGGGGC C AAGUCUGU | 896 | ACAGACUU CUGAUGAG GCCGUUAGGC CGAA ICCCCCAA | 3405 |
| 765 | UGGGGGCC A AGUCUGUA | 897 | UACAGACU CUGAUGAG GCCGUUAGGC CGAA IGCCCCCA | 3406 |
| 770 | GCCAAGUC U GUACAACA | 898 | UGUUGUAC CUGAUGAG GCCGUUAGGC CGAA IACUUGGC | 3407 |
| 775 | GUCUGUAC A ACAUCUUG | 899 | CAAGAUGU CUGAUGAG GCCGUUAGGC CGAA IUACAGAC | 3408 |
| 778 | UGUACAAC A UCUUGAGU | 900 | ACUCAAGA CUGAUGAG GCCGUUAGGC CGAA IUUGUACA | 3409 |
| 781 | ACAACAUC U UCAGUCCC | 901 | GGGACUCA CUGAUGAG GCCGUUAGGC CGAA IAUGUUGU | 3410 |
| 788 | CUUGAGUC C CUUUAUGC | 902 | GCAUAAAG CUGAUGAG GCCGUUAGGC CGAA IACUCAAG | 3411 |
| 789 | UUGAGUCC C UUUAUGCC | 903 | GGCAUAAA CUCAUGAG GCCGUUAGGC CGAA IGACUCAA | 3412 |
| 790 | UGAGUCCC U UUAUGCCG | 904 | CGGCAUAA CUGAUGAG GCCGUUAGGC CGAA IGGACUCA | 3413 |
| 797 | CUUUAUGC C GCUGUUAC | 905 | GUAACAGC CUGAUGAG GCCGUUAGGC CGAA ICAUAAAG | 3414 |
| 800 | UAUGCCGC U GUUACCAA | 906 | UUGGUAAC CUGAUGAG GCCGUUAGGC CGAA ICGGCAUA | 3415 |
| 806 | GCUGUUAC C AAUUUUCU | 907 | AGAAAAUU CUGAUGAG GCCGUUAGGC CGAA IUAACAGC | 3416 |
| 807 | CUGUUACC A AUUUUCUU | 908 | AAGAAAAU CUGAUGAG GCCGUUAGGC CGAA IGUAACAG | 3417 |
| 814 | CAAUUUUC U UUUGUCUU | 909 | AAGACAAA CUGAUGAG GCCGUUAGGC CGAA IAAAAUUG | 3418 |
| 821 | CUUUUGUC U UUGGGUAU | 910 | AUACCCAA CUGAUGAG GCCGUUAGGC CGAA IACAAAAG | 3419 |
| 832 | GGGUAUAC A UUUAAACC | 911 | GGUUUAAA CUGAUGAG GCCGUUAGGC CGAA IUAUACCC | 3420 |
| 840 | AUUUAAAC C CUCACAAA | 912 | UUUGUGAG CUGAUGAG GCCGUUAGGC CGAA IUUUAAAU | 3421 |
| 841 | UUUAAACC C UCACAAAA | 913 | UUUUGUGA CUGAUGAG GCCGUUAGGC CGAA IGUUUAAA | 3422 |
| 842 | UUAAACCC U CACAAAAC | 914 | GUUUUGUG CUGAUGAG GCCGUUAGGC CGAA IGGUUUAA | 3423 |
| 844 | AAACCCUC A CAAAACAA | 915 | UUGUUUUG CUGAUGAG GCCGUUAGGC CGAA IAGGGUUU | 3424 |
| 846 | ACCCUCAC A AAACAAAA | 916 | UUUUGUUU CUGAUGAG GCCGUUAGGC CGAA IUGAGGGU | 3425 |
| 851 | CACAAAAC A AAAAGAUG | 917 | CAUCUUUU CUGAUGAG GCCGUUAGGC CGAA IUUUUGUG | 3426 |
| 869 | GGAUAUUC C CUUAACUU | 918 | AAGUUAAG CUGAUGAG GCCGUUAGGC CGAA IAAUAUCC | 3427 |
| 870 | GAUAUUCC C UUAACUUC | 919 | GAAGUUAA CUGAUGAG GCCGUUAGGC CGAA IGAAUAUC | 3428 |
| 871 | AUAUUCCC U UAACUUCA | 920 | UGAAGUUA CUGAUGAG GCCGUUAGGC CGAA IGGAAUAU | 3429 |
| 876 | CCCUUAAC U UCAUGGGA | 921 | UCCCAUGA CUGAUGAG GCCGUUAGGC CGAA IUUAAGGG | 3430 |
| 879 | UUAACUUC A UGGGAUAU | 922 | AUAUCCCA CUGAUGAG GCCGUUAGGC CGAA IAAGUUAA | 3431 |
| 906 | GUUGGGGC A CAUUGCCA | 923 | UGGCAAUG CUGAUGAG GCCGUUAGGC CGAA ICCCCAAC | 3432 |
| 908 | UGGGGCAC A UUGCCACA | 924 | UGUGGCAA CUGAUGAG GCCGUUAGGC CGAA IUGCCCCA | 3433 |
| 913 | CACAUUGC C ACAGGAAC | 925 | GUUCCUGU CUGAUGAG GCCGUUAGGC CGAA ICAAUGUG | 3434 |
| 914 | ACAUUGCC A CAGGAACA | 926 | UGUUCCUG CUGAUGAG GCCGUUAGGC CGAA IGCAAUGU | 3435 |
| 916 | AUUGCCAC A GGAACAUA | 927 | UAUGUUCC CUGAUGAG GCCGUUAGGC CGAA IUGGCAAU | 3436 |
| 922 | ACAGGAAC A UAUUGUAC | 928 | GUACAAUA CUGAUGAG GCCGUUAGGC CGAA IUUCCUGU | 3437 |
| 931 | UAUUGUAC A AAAAAUCA | 929 | UGAUUUUU CUGAUGAG GCCGUUAGGC CGAA IUACAAUA | 3438 |
| 939 | AAAAAAUC A AAAUGUGU | 930 | ACACAUUU CUGAUGAG GCCGUUAGGC CGAA IAUUUUUU | 3439 |
| 958 | UAGGAAAC U UCCUGUAA | 931 | UUACAGGA CUGAUGAG GCCGUUAGGC CGAA IUUUCCUA | 3440 |
| 961 | GAAACUUC C UGUAAACA | 932 | UGUUUACA CUGAUGAG GCCGUUAGGC CGAA IAAGUUUC | 3441 |
| 962 | AAACUUCC U GUAAACAG | 933 | CUGUUUAC CUGAUGAG GCCGUUAGGC CGAA IGAAGUUU | 3442 |
| 969 | CUGUAAAC A GGCCUAUU | 934 | AAUAGGCC CUGAUGAG GCCGUUAGGC CGAA IUUUACAG | 3443 |
| 973 | AAACAGGC C UAUUGAUU | 935 | AAUCAAUA CUGAUGAG GCCGUUAGGC CGAA ICCUGUUU | 3444 |
| 974 | AACAGGCC U AUUGAUUG | 936 | CAAUCAAU CUGAUGAG GCCGUUAGGC CGAA IGCCUGUU | 3445 |
| 994 | AGUAUGUC A ACGAAUUG | 937 | CAAUUCGU CUGAUGAG GCCGUUAGGC CGAA IACAUACU | 3446 |
| 1009 | UGUGGGUC U UUUGGGGU | 938 | ACCCCAAA CUGAUGAG GCCGUUAGGC CGAA IACCCACA | 3447 |
| 1022 | GGGUUUGC C GCCCCUUU | 939 | AAAGGGGC CUGAUGAG GCCGUUAGGC CGAA ICAAACCC | 3448 |
| 1025 | UUUGCCGC C CCUUUCAC | 940 | GUGAAAGG CUGAUGAG GCCGUUAGGC CGAA ICGGCAAA | 3449 |
| 1026 | UUGCCGCC C CUUUCACG | 941 | CGUGAAAG CUGAUGAG GCCGUUAGGC CGAA IGCGGCAA | 3450 |
| 1027 | UGCCGCCC C UUUCACGC | 942 | GCGUGAAA CUGAUGAG GCCGUUAGGC CGAA IGGCGGCA | 3451 |
| 1028 | GCCGCCCC U UUCACGCA | 943 | UGCGUGAA CUGAUGAG GCCGUUAGGC CGAA IGGGCGGC | 3452 |
| 1032 | CCCCUUUC A CGCAAUGU | 944 | ACAUUGCG CUGAUGAG GCCGUUAGGC CGAA IAAAGGGG | 3453 |
| 1036 | UUUCACGC A AUGUGGAU | 945 | AUCCACAU CUGAUGAG GCCGUUAGGC CGAA ICGUGAAA | 3454 |
| 1049 | GGAUAUUC U GCUUUAAU | 946 | AUUAAAGC CUGAUGAG GCCGUUAGGC CGAA IAAUAUCC | 3455 |
| 1052 | UAUUCUGC U UUAAUGCC | 947 | GGCAUUAA CUGAUGAG GCCGUUAGGC CGAA ICAGAAUA | 3456 |
| 1060 | UUUAAUGC C UUUAUAUG | 948 | CAUAUAAA CUGAUGAG GCCGUUAGGC CGAA ICAUUAAA | 3457 |
| 1061 | UUAAUGCC U UUAUAUGC | 949 | GCAUAUAA CUGAUGAG GCCGUUAGGC CGAA IGCAUUAA | 3458 |
| 1070 | UUAUAUGC A UGCAUACA | 950 | UGUAUGCA CUGAUGAG GCCGUUAGGC CGAA ICAUAUAA | 3459 |
| 1074 | AUGCAUGC A UACAAGCA | 951 | UGCUUGUA CUGAUGAG GCCGUUAGGC CGAA ICAUGCAU | 3460 |
| 1078 | AUGCAUAC A AGCAAAAC | 952 | GUUUUGCU CUGAUGAG GCCGUUAGGC CGAA IUAUGCAU | 3461 |
| 1082 | AUACAAGC A AAACAGGC | 953 | GCCUGUUU CUGAUGAG GCCGUUAGGC CGAA ICUUGUAU | 3462 |
| 1087 | AGCAAAAC A GGCUUUUA | 954 | UAAAAGCC CUGAUGAG GCCGUUAGGC CGAA IUUUUGCU | 3463 |
| 1091 | AAACAGGC U UUUACUUU | 955 | AAAGUAAA CUGAUGAG GCCGUUAGGC CGAA ICCUGUUU | 3464 |
| 1097 | GCUUUUAC U UUCUCGCC | 956 | GGCGAGAA CUGAUGAG GCCGUUAGGC CGAA IUAAAAGC | 3465 |
| 1101 | UUACUUUC U CGCCAACU | 957 | AGUUGGCG CUGAUGAG GCCGUUAGGC CGAA IAAAGUAA | 3466 |
| 1105 | UUUCUCGC C AACUUACA | 958 | UGUAAGUU CUGAUGAG GCCGUUAGGC CGAA ICGAGAAA | 3467 |
| 1106 | UUCUCGCC A ACUUACAA | 959 | UUGUAAGU CUGAUGAG GCCGUUAGGC CGAA IGCGAGAA | 3468 |
| 1109 | UCGCCAAC U UACAAGGC | 960 | GCCUUGUA CUGAUGAG GCCGUUAGGC CGAA IUUGGCGA | 3469 |
| 1113 | CAACUUAC A AGGCCUUU | 961 | AAAGGCCU CUGAUGAG GCCGUUAGGC CGAA IUAAGUUG | 3470 |
| 1118 | UACAAGGC C UUUCUAAG | 962 | CUUAGAAA CUGAUGAG GCCGUUAGGC CGAA ICCUUGUA | 3471 |
| 1119 | ACAAGGCC U UUCUAAGU | 963 | ACUUAGAA CUGAUGAG GCCGUUAGGC CGAA IGCCUUGU | 3472 |
| 1123 | GGCCUUUC U AAGUAAAC | 964 | GUUUACUU CUGAUGAG GCCGUUAGGC CGAA IAAAGGCC | 3473 |
| 1132 | AAGUAAAC A GUAUGUGA | 965 | UCACAUAC CUGAUGAG GCCGUUAGGC CGAA IUUUACUU | 3474 |
| 1143 | AUGUGAAC C UUUACCCC | 966 | GGGGUAAA CUGAUGAG GCCGUUAGGC CGAA IUUCACAU | 3475 |
| 1144 | UGUGAACC U UUACCCCG | 967 | CGGGGUAA CUGAUGAG GCCGUUAGGC CGAA IGUUCACA | 3476 |
| 1149 | ACCUUUAC C CCGUUGCU | 968 | AGCAACGG CUGAUGAG GCCGUUAGGC CGAA IUAAAGGU | 3477 |
| 1150 | CCUUUACC C CGUUGCUC | 969 | GAGCAACG CUGAUGAG GCCGUUAGGC CGAA IGUAAAGG | 3478 |
| 1151 | CUUUACCC C GUUGCUCG | 970 | CGAGCAAC CUGAUGAG GCCGUUAGGC CGAA IGGUAAAG | 3479 |
| 1157 | CCCGUUGC U CGGCAACG | 971 | CGUUGCCG CUGAUGAG GCCGUUAGGC CGAA ICAACGGG | 3480 |
| 1162 | UGCUCGGC A ACGGCCUG | 972 | CAGGCCGU CUGAUGAG GCCGUUAGGC CGAA ICCGAGCA | 3481 |
| 1168 | GCAACGGC C UGGUCUAU | 973 | AUAGACCA CUGAUGAG GCCGUUAGGC CGAA ICCGUUGC | 3482 |
| 1169 | CAACGGCC U GGUCUAUG | 974 | CAUAGACC CUGAUGAG GCCGUUAGGC CGAA IGCCGUUG | 3483 |
| 1174 | GCCUGGUC U AUGCCAAG | 975 | CUUGGCAU CUGAUGAG GCCGUUAGGC CGAA IACCAGGC | 3484 |
| 1179 | GUCUAUGC C AAGUGUUU | 976 | AAACACUU CUGAUGAG GCCGUUAGGC CGAA ICAUAGAC | 3485 |
| 1180 | UCUAUGCC A AGUGUUUG | 977 | CAAACACU CUGAUGAG GCCGUUAGGC CGAA IGCAUAGA | 3486 |
| 1190 | GUGUUUGC U GACGCAAC | 978 | GUUGCGUC CUGAUGAG GCCGUUAGGC CGAA ICAAACAC | 3487 |
| 1196 | GCUGACGC A ACCCCCAC | 979 | GUGGGGGU CUGAUGAG GCCGUUAGGC CGAA ICGUCAGC | 3488 |
| 1199 | GACGCAAC C CCCACUGG | 980 | CCAGUGGG CUGAUGAG GCCGUUAGGC CGAA IUUGCGUC | 3489 |
| 1200 | ACGCAACC C CCACUGGU | 981 | ACCAGUGG CUGAUGAG GCCGUUAGGC CGAA IGUUGCGU | 3490 |
| 1201 | CGCAACCC C CACUGGUU | 982 | AACCAGUG CUGAUGAG GCCGUUAGGC CGAA IGGUUGCG | 3491 |
| 1202 | GCAACCCC C ACUGGUUG | 983 | CAACCAGU CUGAUGAG GCCGUUAGGC CGAA IGGGUUGC | 3492 |
| 1203 | CAACCCCC A CUGGUUGG | 984 | CCAACCAG CUGAUGAG GCCGUUAGGC CGAA IGGGGUUG | 3493 |
| 1205 | ACCCCCAC U GGUUGGGG | 985 | CCCCAACC CUGAUGAG GCCGUUAGGC CGAA IUGGGGGU | 3494 |
| 1215 | GUUGGGGC U UGGCCAUA | 986 | UAUGGCCA CUGAUGAG GCCGUUAGGC CGAA ICCCCAAC | 3495 |
| 1220 | GGCUUGGC C AUAGGCCA | 987 | UGGCCUAU CUGAUGAG GCCGUUAGGC CGAA ICCAAGCC | 3496 |
| 1221 | GCUUGGCC A UAGGCCAU | 988 | AUGGCCUA CUGAUGAG GCCGUUAGGC CGAA IGCCAAGC | 3497 |
| 1227 | CCAUAGGC C AUCAGCGC | 989 | GCGCUGAU CUGAUGAG GCCGUUAGGC CGAA ICCUAUGG | 3498 |
| 1228 | CAUAGGCC A UCAGCGCA | 990 | UGCGCUGA CUGAUGAG GCCGUUAGGC CGAA IGCCUAUG | 3499 |
| 1231 | AGGCCAUC A GCGCAUGC | 991 | GCAUGCGC CUGAUGAG GCCGUUAGGC CGAA IAUGGCCU | 3500 |
| 1236 | AUCAGCGC A UGCGUGGA | 992 | UCCACGCA CUGAUGAG GCCGUUAGGC CGAA ICGCUGAU | 3501 |
| 1247 | CGUGGAAC C UUUGUGUC | 993 | GACACAAA CUGAUGAG GCCGUUAGGC CGAA IUUCCACG | 3502 |
| 1248 | GUGGAACC U UUGUGUCU | 994 | AGACACAA CUGAUGAG GCCGUUAGGC CGAA IGUUCCAC | 3503 |
| 1256 | UUUGUGUC U CCUCUGCC | 995 | GGCAGAGG CUGAUGAG GCCGUUAGGC CGAA IACACAAA | 3504 |
| 1258 | UGUGUCUC C UCUGCCGA | 996 | UCGGCAGA CUGAUGAG GCCGUUAGGC CGAA IAGACACA | 3505 |
| 1259 | GUGUCUCC U CUGCCGAU | 997 | AUCGGCAG CUGAUGAG GCCGUUAGGC CGAA IGAGACAC | 3506 |
| 1261 | GUCUCCUC U GCCGAUCC | 998 | GGAUCGGC CUGAUGAG GCCGUUAGGC CGAA IAGGAGAC | 3507 |
| 1264 | UCCUCUGC C GAUCCAUA | 999 | UAUGGAUC CUGAUGAG GCCGUUAGGC CGAA ICAGAGGA | 3508 |
| 1269 | UGCCGAUC C AUACCGCG | 1000 | CGCGGUAU CUGAUGAG GCCGUUAGGC CGAA IAUCGGCA | 3509 |
| 1270 | GCCGAUCC A UACCGCGG | 1001 | CCGCGGUA CUGAUGAG GCCGUUAGGC CGAA IGAUCGGC | 3510 |
| 1274 | AUCCAUAC C GCGGAACU | 1002 | AGUUCCGC CUGAUGAG GCCGUUAGGC CGAA IUAUGGAU | 3511 |
| 1282 | CGCGGAAC U CCUAGCCG | 1003 | CGGCUAGG CUGAUGAG GCCGUUAGGC CGAA IUUCCGCG | 3512 |
| 1284 | CGGAACUC C UAGCCGCU | 1004 | AGCGGCUA CUGAUGAG GCCGUUAGGC CGAA IAGUUCCG | 3513 |
| 1285 | GGAACUCC U AGCCGCUU | 1005 | AAGCGGCU CUGAUGAG GCCGUUAGGC CGAA IGAGUUCC | 3514 |
| 1289 | CUCCUAGC C GCUUGUUU | 1006 | AAACAAGC CUGAUGAG GCCGUUAGGC CGAA ICUAGGAG | 3515 |
| 1292 | CUAGCCGC U UGUUUUGC | 1007 | GCAAAACA CUGAUGAG GCCGUUAGGC CGAA ICGGCUAG | 3516 |
| 1301 | UGUUUUGC U CGCAGCAG | 1008 | CUGCUGCG CUGAUGAG GCCGUUAGGC CGAA ICAAAACA | 3517 |
| 1305 | UUGCUCGC A GCAGGUCU | 1009 | AGACCUGC CUGAUGAG GCCGUUAGGC CGAA ICGAGCAA | 3518 |
| 1308 | CUCGCAGC A GGUCUGGG | 1010 | CCCAGACC CUGAUGAG GCCGUUAGGC CGAA ICUGCGAG | 3519 |
| 1313 | AGCAGGUC U GGGGCAAA | 1011 | UUUGCCCC CUGAUGAG GCCGUUAGGC CGAA IACCUGCU | 3520 |
| 1319 | UCUGGGGC A AAACUCAU | 1012 | AUGAGUUU CUGAUGAG GCCGUUAGGC CGAA ICCCCAGA | 3521 |
| 1324 | GGCAAAAC U CAUCGGGA | 1013 | UCCCGAUG CUGAUGAG GCCGUUAGGC CGAA IUUUUGCC | 3522 |
| 1326 | CAAAACUC A UCGGGACU | 1014 | AGUCCCGA CUGAUGAG GCCGUUAGGC CGAA IAGUUUUG | 3523 |
| 1334 | AUCGGGAC U GACAAUUC | 1015 | GAAUUGUC CUGAUGAG GCCGUUAGGC CGAA IUCCCGAU | 3524 |
| 1338 | GGACUGAC A AUUCUGUC | 1016 | GACAGAAU CUGAUGAG GCCGUUAGGC CGAA IUCAGUCC | 3525 |
| 1343 | GACAAUUC U GUCGUGCU | 1017 | AGCACGAC CUGAUGAG GCCGUUAGGC CGAA IAAUUGUC | 3526 |
| 1351 | UGUCGUGC U CUCCCGCA | 1018 | UGCGGGAG CUGAUGAG GCCGUUAGGC CGAA ICACCACA | 3527 |
| 1353 | UCGUGCUC U CCCGCAAA | 1019 | UUUGCGGG CUGAUGAG GCCGUUAGGC CGAA IAGCACGA | 3528 |
| 1355 | GUGCUCUC C CGCAAAUA | 1020 | UAUUUGCG CUGAUGAG GCCGUUAGGC CGAA IAGAGCAC | 3529 |
| 1356 | UGCUCUCC C GCAAAUAU | 1021 | AUAUUUGC CUGAUGAG GCCGUUAGGC CGAA IGAGAGCA | 3530 |
| 1359 | UCUCCCGC A AAUAUACA | 1022 | UGUAUAUU CUGAUGAG GCCGUUAGGC CGAA ICGGGAGA | 3531 |
| 1367 | AAAUAUAC A UCAUUUCC | 1023 | GGAAAUGA CUGAUGAG GCCGUUAGGC CGAA IUAUAUUU | 3532 |
| 1370 | UAUACAUC A UUUCCAUG | 1024 | CAUGGAAA CUGAUGAG GCCGUUAGGC CGAA IAUGUAUA | 3533 |
| 1375 | AUCAUUUC C AUGGCUGC | 1025 | GCAGCCAU CUGAUGAG GCCGUUAGGC CGAA IAAAUGAU | 3534 |
| 1376 | UCAUUUCC A UGGCUGCU | 1026 | AGCAGCCA CUGAUGAG GCCGUUAGGC CGAA IGAAAUGA | 3535 |
| 1381 | UCCAUGGC U GCUAGGCU | 1027 | AGCCUAGC CUGAUGAG GCCGUUAGGC CGAA ICCAUGGA | 3536 |
| 1384 | AUGGCUGC U AGGCUGUG | 1028 | CACAGCCU CUGAUGAG GCCGUUAGGC CGAA ICAGCCAU | 3537 |
| 1389 | UGCUAGGC U GUGCUGCC | 1029 | GGCAGCAC CUGAUGAG GCCGUUAGGC CGAA ICCUAGCA | 3538 |
| 1394 | GGCUGUGC U GCCAACUG | 1030 | CAGUUGGC CUGAUGAG GCCGUUAGGC CGAA ICACAGCC | 3539 |
| 1397 | UGUGCUGC C AACUGGAU | 1031 | AUCCAGUU CUGAUGAG GCCGUUAGGC CGAA ICAGCACA | 3540 |
| 1398 | GUGCUGCC A ACUGGAUC | 1032 | GAUCCAGU CUGAUGAG GCCGUUAGGC CGAA IGCAGCAC | 3541 |
| 1401 | CUGCCAAC U GGAUCCUA | 1033 | UAGGAUCC CUGAUGAG GCCGUUAGGC CGAA IUUGGCAG | 3542 |
| 1407 | ACUGGAUC C UACGCGGG | 1034 | CCCGCGUA CUGAUGAG GCCGUUAGGC CGAA IAUCCAGU | 3543 |
| 1408 | CUGGAUCC U ACGCGGGA | 1035 | UCCCGCGU CUGAUGAG GCCGUUAGGC CGAA IGAUCCAG | 3544 |
| 1421 | GGGACGUC C UUUGUUUA | 1036 | UAAACAAA CUGAUGAG GCCGUUAGGC CGAA IACGUCCC | 3545 |
| 1422 | GGACGUCC U UUGUUUAC | 1037 | GUAAACAA CUGAUGAG GCCGUUAGGC CGAA IGACGUCC | 3546 |
| 1434 | UUUACGUC C CGUCGGCG | 1038 | CGCCGACG CUGAUGAG GCCGUUAGGC CGAA IACGUAAA | 3547 |
| 1435 | UUACGUCC C GUCGGCGC | 1039 | GCGCCGAC CUGAUGAG GCCGUUAGGC CGAA IGACGUAA | 3548 |
| 1444 | GUCGGCGC U GAAUCCCG | 1040 | CGGGAUUC CUGAUGAG GCCGUUAGGC CGAA ICGCCGAC | 3549 |
| 1450 | GCUGAAUC C CGCGGACG | 1041 | CGUCCGCG CUGAUGAG GCCGUUAGGC CGAA IAUUCAGC | 3550 |
| 1451 | CUGAAUCC C GCGGACGA | 1042 | UCGUCCGC CUGAUGAG GCCGUUAGGC CGAA IGAGUCAG | 3551 |
| 1461 | CGGACGAC C CCUCCCGG | 1043 | CCGGGAGG CUGAUGAG GCCGUUAGGC CGAA IUCGUCCG | 3552 |
| 1462 | GGACGACC C CUCCCGGG | 1044 | CCCGGGAG CUGAUGAG GCCGUUAGGC CGAA IGUCGUCC | 3553 |
| 1463 | GACGACCC C UCCCGGGG | 1045 | CCCCGGGA CUGAUGAG GCCGUUAGGC CGAA IGGUCGUC | 3554 |
| 1464 | ACGACCCC U CCCGGGGC | 1046 | GCCCCGGG CUGAUGAG GCCGUUAGGC CGAA IGGGUCGU | 3555 |
| 1466 | GACCCCUC C CGGGGCCG | 1047 | CGGCCCCG CUGAUGAG GCCGUUAGGC CGAA IAGGGGUC | 3556 |
| 1467 | ACCCCUCC C GGGGCCGC | 1048 | GCGGCCCC CUGAUGAG GCCGUUAGGC CGAA IGAGGGGU | 3557 |
| 1473 | CCCGGGGC C GCUUGGGG | 1049 | CCCCAAGC CUGAUGAG GCCGUUAGGC CGAA ICCCCGGG | 3558 |
| 1476 | GGGGCCGC U UGGGGCUC | 1050 | GAGCCCCA CUGAUGAG GCCGUUAGGC CGAA ICGGCCCC | 3559 |
| 1483 | CUUGGGGC U CUACCGCC | 1051 | GGCGGUAG CUGAUGAG GCCGUUAGGC CGAA ICCCCAAG | 3560 |
| 1485 | UGGGGCUC U ACCGCCCG | 1052 | CGGGCGGU CUGAUGAG GCCGUUAGGC CGAA IAGCCCCA | 3561 |
| 1488 | GGCUCUAC C GCCCGCUU | 1053 | AAGCGGGC CUGAUGAG GCCGUUAGGC CGAA IUAGAGCC | 3562 |
| 1491 | UCUACCGC C CGCUUCUC | 1054 | GAGAAGCG CUGAUGAG GCCGUUAGGC CGAA ICGGUAGA | 3563 |
| 1492 | CUACCGCC C GCUUCUCC | 1055 | GGAGAAGC CUGAUGAG GCCGUUAGGC CGAA IGCGGUAG | 3564 |
| 1495 | CCGCCCGC U UCUCCGCC | 1056 | GGCGGAGA CUGAUGAG GCCGUUAGGC CGAA ICGGGCGG | 3565 |
| 1493 | CCCGCUUC U CCGCCUAU | 1057 | AUAGGCGG CUGAUGAG GCCGUUAGGC CGAA IAAGCGGG | 3566 |
| 1500 | CGCUUCUC C GCCUAUUG | 1058 | CAAUAGGC CUGAUGAG GCCGUUAGGC CGAA IAGAAGCG | 3567 |
| 1503 | UUCUCCGC C UAUUGUAC | 1059 | GUACAAUA CUGAUGAG GCCGUUAGGC CGAA ICGGAGAA | 3568 |
| 1504 | UCUCCGCC U AUUGUACC | 1060 | GGUACAAU CUGAUGAG GCCGUUAGGC CGAA IGCGGAGA | 3569 |
| 1512 | UAUUGUAC C GACCGUCC | 1061 | GGACGGUC CUGAUGAG GCCGUUAGGC CGAA IUACAAUA | 3570 |
| 1516 | GUACCGAC C GUCCACGG | 1062 | CCGUGGAC CUGAUGAG GCCGUUAGGC CGAA IUCGGUAC | 3571 |
| 1520 | CGACCGUC C ACGGGGCG | 1063 | CGCCCCGU CUGAUGAG GCCGUUAGGC CGAA IACGGUCG | 3572 |
| 1521 | GACCGUCC A CGGGGCGC | 1064 | GCGCCCCG CUGAUGAG GCCGUUAGGC CGAA IGACGGUC | 3573 |
| 1530 | CGGGGCGC A CCUCUCUU | 1065 | AAGAGAGG CUGAUGAG GCCGUUAGGC CGAA ICGCCCCG | 3574 |
| 1532 | GGGCGCAC C UCUCUUUA | 1066 | UAAAGAGA CUGAUGAG GCCGUUAGGC CGAA IUGCGCCC | 3575 |
| 1533 | GGCGCACC U CUCUUUAC | 1067 | GUAAAGAG CUGAUGAG GCCGUUAGGC CGAA IGUGCGCC | 3576 |
| 1535 | CGCACCUC U CUUUACGC | 1068 | GCGUAAAG CUGAUGAG GCCGUUAGGC CGAA IAGGUGCG | 3577 |
| 1537 | CACCUCUC U UUACGCGG | 1069 | CCGCGUAA CUGAUGAG GCCGUUAGGC CGAA IAGAGGUG | 3578 |
| 1548 | ACGCGGAC U CCCCGUCU | 1070 | AGACGGGG CUGAUGAG GCCGUUAGGC CGAA IUCCGCGU | 3579 |
| 1550 | GCGGACUC C CCGUCUGU | 1071 | ACAGACGG CUGAUGAG GCCGUUAGGC CGAA IAGUCCGC | 3580 |
| 1551 | CGGACUCC C CGUCUGUG | 1072 | CACAGACG CUGAUGAG GCCGUUAGGC CGAA IGAGUCCG | 3581 |
| 1552 | GGACUCCC C GUCUGUGC | 1073 | GCACAGAC CUGAUGAG GCCGUUAGGC CGAA IGGAGUCC | 3582 |
| 1556 | UCCCCGUC U GUGCCUUC | 1074 | GAAGGCAC CUGAUGAG GCCGUUAGGC CGAA IACGGGGA | 3583 |
| 1561 | GUCUGUGC C UUCUCAUC | 1075 | GAUGAGAA CUGAUGAG GCCGUUAGGC CGAA ICACAGAC | 3584 |
| 1562 | UCUGUGCC U UCUCAUCU | 1076 | AGAUGAGA CUGAUGAG GCCGUUAGGC CGAA IGCACAGA | 3585 |
| 1565 | GUGCCUUC U CAUCUGCC | 1077 | GGCAGAUG CUGAUGAG GCCGUUAGGC CGAA IAAGGCAC | 3586 |
| 1567 | GCCUUCUC A UCUGCCGG | 1078 | CCGGCAGA CUGAUGAG GCCGUUAGGC CGAA IAGAAGGC | 3587 |
| 1570 | UUCUCAUC U GCCGGACC | 1079 | GGUCCGGC CUGAUGAG GCCGUUAGGC CGAA IAUGAGAA | 3588 |
| 1573 | UCAUCUGC C GGACCGUG | 1080 | CACGGUCC CUGAUGAG GCCGUUAGGC CGAA ICAGAUGA | 3589 |
| 1578 | UGCCGGAC C GUGUGCAC | 1081 | GUGCACAC CUGAUGAG GCCGUUAGGC CGAA IUCCGGCA | 3590 |
| 1585 | CCGUGUGC A CUUCGCUU | 1082 | AAGCGAAG CUGAUGAG GCCGUUAGGC CGAA ICACACGG | 3591 |
| 1587 | GUGUGCAC U UCGCUUCA | 1083 | UGAAGCGA CUGAUGAG GCCGUUAGGC CGAA IUGCACAC | 3592 |
| 1592 | CACUUCGC U UCACCUCU | 1084 | AGAGGUGA CUGAUGAG GCCGUUAGGC CGAA ICGAAGUG | 3593 |
| 1595 | UUCGCUUC A CCUCUGCA | 1085 | UGCAGAGG CUGAUGAG GCCGUUAGGC CGAA IAAGCGAA | 3594 |
| 1597 | CGCUUCAC C UCUGCACG | 1086 | CGUGCAGA CUGAUGAG GCCGUUAGGC CGAA IUGAAGCG | 3595 |
| 1598 | GCUUCACC U CUGCACGU | 1087 | ACGUGCAG CUGAUGAG GCCGUUAGGC CGAA IGUGAAGC | 3596 |
| 1600 | UUCACCUC U GCACGUCG | 1088 | CGACGUGC CUGAUGAG GCCGUUAGGC CGAA IAGGUGAA | 3597 |
| 1603 | ACCUCUGC A CGUCGCAU | 1089 | AUGCGACG CUGAUGAG GCCGUUAGGC CGAA ICAGAGGU | 3598 |
| 1610 | CACGUCGC A UGGAGACC | 1090 | GGUCUCCA CUGAUGAG GCCGUUAGGC CGAA ICGACGUG | 3599 |
| 1618 | AUGGAGAC C ACCGUGAA | 1091 | UUCACGGU CUGAUGAG GCCGUUAGGC CGAA IUCUCCAU | 3600 |
| 1619 | UGGAGACC A CCGUGAAC | 1092 | GUUCACGG CUGAUGAG GCCGUUAGGC CGAA IGUCUCCA | 3601 |
| 1621 | GAGACCAC C GUGAACGC | 1093 | GCGUUCAC CUGAUGAG GCCGUUAGGC CGAA IUGGUCUC | 3602 |
| 1630 | GUGAACGC C CACAGGAA | 1094 | UUCCUGUG CUGAUGAG GCCGUUAGGC CGAA ICGUUCAC | 3603 |
| 1631 | UGAACGCC C ACAGGAAC | 1095 | GUUCCUGU CUGAUGAG GCCGUUAGGC CGAA IGCGUUCA | 3604 |
| 1632 | GAACGCCC A CAGGAACC | 1096 | GGUUCCUG CUGAUGAG GCCGUUAGGC CGAA IGGCGUUC | 3605 |
| 1634 | ACGCCCAC A GGAACCUG | 1097 | CAGGUUCC CUGAUGAG GCCGUUAGGC CGAA IUGGGCGU | 3606 |
| 1640 | ACAGGAAC C UGCCCAAG | 1098 | CUUGGGCA CUGAUGAG GCCGUUAGGC CGAA IUUCCUGU | 3607 |
| 1641 | CAGGAACC U GCCCAAGG | 1099 | CCUUGGGC CUGAUGAG GCCGUUAGGC CGAA IGUUCCUG | 3608 |
| 1644 | GAACCUGC C CAAGGUCU | 1100 | AGACCUUG CUGAUGAG GCCGUUAGGC CGAA ICAGGUUC | 3609 |
| 1645 | AACCUGCC C AAGGUCUU | 1101 | AAGACCUU CUGAUGAG GCCGUUAGGC CGAA IGCAGGUU | 3610 |
| 1646 | ACCUGCCC A AGGUCUUG | 1102 | CAAGACCU CUGAUGAG GCCGUUAGGC CGAA IGGCAGGU | 3611 |
| 1652 | CCAAGGUC U UGCAUAAG | 1103 | CUUAUGCA CUGAUGAG GCCGUUAGGC CGAA IACCUUGG | 3612 |
| 1656 | GGUCUUGC A UAAGAGGA | 1104 | UCCUCUUA CUGAUGAG GCCGUUAGGC CGAA ICAAGACC | 3613 |
| 1666 | AAGAGGAC U CUUGGACU | 1105 | AGUCCAAG CUGAUGAG GCCGUUAGGC CGAA IUCCUCUU | 3614 |
| 1668 | GAGGACUC U UGGACUUU | 1106 | AAAGUCCA CUGAUGAG GCCGUUAGGC CGAA IAGUCCUC | 3615 |
| 1674 | UCUUGGAC U UUCAGCAA | 1107 | UUGCUGAA CUGAUGAG GCCGUUAGGC CGAA IUCCAAGA | 3616 |
| 1678 | GGACUUUC A GCAAUGUC | 1108 | GACAUUGC CUGAUGAG GCCGUUAGGC CGAA IAAAGUCC | 3617 |
| 1681 | CUUUCAGC A AUGUCAAC | 1109 | GUUGACAU CUGAUGAG GCCGUUAGGC CGAA ICUGAAAG | 3618 |
| 1687 | GCAAUGUC A ACGACCGA | 1110 | UCGGUCGU CUGAUGAG GCCGUUAGGC CGAA IACAUUGC | 3619 |
| 1693 | UCAACGAC C GACCUUGA | 1111 | UCAAGGUC CUGAUGAG GCCGUUAGGC CGAA IUCGUUGA | 3620 |
| 1697 | CGACCGAC C UUGAGGCA | 1112 | UGCCUCAA CUGAUGAG GCCGUUAGGC CGAA IUCGGUCG | 3621 |
| 1698 | GACCGACC U UGAGGCAU | 1113 | AUGCCUCA CUGAUGAG GCCGUUAGGC CGAA IGUCGGUC | 3622 |
| 1705 | CUUGAGGC A UACUUCAA | 1114 | UUGAAGUA CUGAUGAG GCCGUUAGGC CGAA ICCUCAAG | 3623 |
| 1709 | AGGCAUAC U UCAAAGAC | 1115 | GUCUUUGA CUGAUGAG GCCGUUAGGC CGAA IUAUGCCU | 3624 |
| 1712 | CAUACUUC A AAGACUGU | 1116 | ACAGUCUU CUGAUGAG GCCGUUAGGC CGAA IAAGUAUG | 3625 |
| 1718 | UCAAAGAC U GUGUGUUU | 1117 | AAACACAC CUGAUGAG GCCGUUAGGC CGAA IUCUUUGA | 3626 |
| 1769 | UAAAGGUC U UUGUACUA | 1118 | UAGUACAA CUGAUGAG GCCGUUAGGC CGAA IACCUUUA | 3627 |
| 1776 | CUUUGUAC U AGGAGGCU | 1119 | AGCCUCCU CUGAUGAG GCCGUUAGGC CGAA IUACAAAG | 3628 |
| 1784 | UAGGAGGC U GUAGGCAU | 1120 | AUGCCUAC CUGAUGAG GCCGUUAGGC CGAA ICCUCCUA | 3629 |
| 1791 | CUGUAGGC A UAAAUUGG | 1121 | CCAAUUUA CUGAUGAG GCCGUUAGGC CGAA ICCUACAG | 3630 |
| 1807 | GUGUGUUC A CCAGCACC | 1122 | GGUGCUGG CUGAUGAG GCCGUUAGGC CGAA IAACACAC | 3631 |
| 1809 | GUGUUCAC C AGCACCAU | 1123 | AUGGUGCU CUGAUGAG GCCGUUAGGC CGAA IUGAACAC | 3632 |
| 1810 | UGUUCACC A GCACCAUG | 1124 | CAUGGUGC CUGAUGAG GCCGUUAGGC CGAA IGUGAACA | 3633 |
| 1813 | UCACCAGC A CCAUGCAA | 1125 | UUGCAUGG CUGAUGAG GCCGUUAGGC CGAA ICUGGUGA | 3634 |
| 1815 | ACCAGCAC C AUGCAACU | 1126 | AGUUGCAU CUGAUGAG GCCGUUAGGC CGAA IUGCUGGU | 3635 |
| 1816 | CCAGCACC A UGCAACUU | 1127 | AAGUUGCA CUGAUGAG GCCGUUAGGC CGAA IGUGCUGG | 3636 |
| 1820 | CACCAUGC A ACUUUUUC | 1128 | GAAAAAGU CUGAUGAG GCCGUUAGGC CGAA ICAUGGUG | 3637 |
| 1823 | CAUGCAAC U UUUUCACC | 1129 | GGUGAAAA CUGAUGAG GCCGUUAGGC CGAA IUUGCAUG | 3638 |
| 1829 | ACUUUUUC A CCUCUGCC | 1130 | GGCAGAGG CUGAUGAG GCCGUUAGGC CGAA IAAAAAGU | 3639 |
| 1831 | UUUUUCAC C UCUGCCUA | 1131 | UAGGCAGA CUGAUGAG GCCGUUAGGC CGAA IUGAAAAA | 3640 |
| 1832 | UUUUCACC U CUGCCUAA | 1132 | UUAGGCAG CUGAUGAG GCCGUUAGGC CGAA IGUGAAAA | 3641 |
| 1834 | UUCACCUC U GCCUAAUC | 1133 | GAUUAGGC CUGAUGAG GCCGUUAGGC CGAA IAGGUGAA | 3642 |
| 1837 | ACCUCUGC C UAAUCAUC | 1134 | GAUGAUUA CUGAUGAG GCCGUUAGGC CGAA ICAGAGGU | 3643 |
| 1838 | CCUCUGCC U AAUCAUCU | 1135 | AGAUGAUU CUGAUGAG GCCGUUAGGC CGAA IGCAGAGG | 3644 |
| 1843 | GCCUAAUC A UCUCAUGU | 1136 | ACAUGAGA CUGAUGAG GCCGUUAGGC CGAA IAUUAGGC | 3645 |
| 1846 | UAAUCAUC U CAUGUUCA | 1137 | UGAACAUG CUGAUGAG GCCGUUAGGC CGAA IAUGAUUA | 3646 |
| 1848 | AUCAUCUC A UGUUCAUG | 1138 | CAUGAACA CUGAUGAG GCCGUUAGGC CGAA IAGAUGAU | 3647 |
| 1854 | UCAUGUUC A UGUCCUAC | 1139 | GUAGGACA CUGAUGAG GCCGUUAGGC CGAA IAACAUGA | 3648 |
| 1859 | UUCAUGUC C UACUGUUC | 1140 | GAACAGUA CUGAUGAG GCCGUUAGGC CGAA IACAUGAA | 3649 |
| 1860 | UCAUGUCC U ACUGUUCA | 1141 | UGAACAGU CUGAUGAG GCCGUUAGGC CGAA IGACAUGA | 3650 |
| 1863 | UGUCCUAC U GUUCAAGC | 1142 | GCUUGAAC CUGAUGAG GCCGUUAGGC CGAA IUAGGACA | 3651 |
| 1868 | UACUGUUC A AGCCUCCA | 1143 | UGGAGGCU CUGAUGAG GCCGUUAGGC CGAA IAACAGUA | 3652 |
| 1872 | GUUCAAGC C UCCAAGCU | 1144 | AGCUUGGA CUGAUGAG GCCGUUAGGC CGAA ICUUGAAC | 3653 |
| 1873 | UUCAAGCC U CCAAGCUG | 1145 | CAGCUUGG CUGAUGAG GCCGUUAGGC CGAA IGCUUGAA | 3654 |
| 1875 | CAAGCCUC C AAGCUGUG | 1146 | CACAGCUU CUGAUGAG GCCGUUAGGC CGAA IAGGCUUG | 3655 |
| 1876 | AAGCCUCC A AGCUGUGC | 1147 | GCACAGCU CUGAUGAG GCCGUUAGGC CGAA IGAGGCUU | 3656 |
| 1880 | CUCCAAGC U GUGCCUUG | 1148 | CAAGGCAC CUGAUGAG GCCGUUAGGC CGAA ICUUGGAG | 3657 |
| 1885 | AGCUGUGC C UUGGGUGG | 1149 | CCACCCAA CUGAUGAG GCCGUUAGGC CGAA ICACAGCU | 3658 |
| 1886 | GCUGUGCC U UGGGUGGC | 1150 | GCCACCCA CUGAUGAG GCCGUUAGGC CGAA IGCACAGC | 3659 |
| 1895 | UGGGUGGC U UUGGGGCA | 1151 | UGCCCCAA CUGAUGAG GCCGUUAGGC CGAA ICCACCCA | 3660 |
| 1903 | UUUGGGGC A UGGACAUU | 1152 | AAUGUCCA CUGAUGAG GCCGUUAGGC CGAA ICCCCAAA | 3661 |
| 1909 | GCAUGGAC A UUGACCCG | 1153 | CGGGUCAA CUGAUGAG GCCGUUAGGC CGAA IUCCAUGC | 3662 |
| 1915 | ACAUUGAC C CGUAUAAA | 1154 | UUUAUACG CUGAUGAG GCCGUUAGGC CGAA IUCAAUGU | 3663 |
| 1916 | CAUUGACC C GUAUAAAG | 1155 | CUUUAUAC CUGAUGAG GCCGUUAGGC CGAA IGUCAAUG | 3664 |
| 1935 | UUUGGAGC U UCUGUGGA | 1156 | UCCACAGA CUGAUGAG GCCGUUAGGC CGAA ICUCCAAA | 3665 |
| 1938 | GGAGCUUC U GUGGAGUU | 1157 | AACUCCAC CUGAUGAG GCCGUUAGGC CGAA IAAGCUCC | 3666 |
| 1949 | GGAGUUAC U CUCUUUUU | 1158 | AAAAAGAG CUGAUGAG GCCGUUAGGC CGAA IUAACUCC | 3667 |
| 1951 | AGUUACUC U CUUUUUUG | 1159 | CAAAAAAG CUGAUGAG GCCGUUAGGC CGAA IAGUAACU | 3668 |
| 1953 | UUACUCUC U UUUUUGCC | 1160 | GGCAAAAA CUGAUGAG GCCGUUAGGC CGAA IAGAGUAA | 3669 |
| 1961 | UUUUUUGC C UUCUGACU | 1161 | AGUCAGAA CUGAUGAG GCCGUUAGGC CGAA ICAAAAAA | 3670 |
| 1962 | UUUUUGCC U UCUGACUU | 1162 | AAGUCAGA CUGAUGAG GCCGUUAGGC CGAA IGCAAAAA | 3671 |
| 1965 | UUGCCUUC U GACUUCUU | 1163 | AAGAAGUC CUGAUGAG GCCGUUAGGC CGAA IAAGGCAA | 3672 |
| 1969 | CUUCUGAC U UCUUUCCU | 1164 | AGGAAAGA CUGAUGAG GCCGUUAGGC CGAA IUCAGAAG | 3673 |
| 1972 | CUGACUUC U UUCCUUCU | 1165 | AGAAGGAA CUGAUGAG GCCGUUAGGC CGAA IAAGUCAG | 3674 |
| 1976 | CUUCUUUC C UUCUAUUC | 1166 | GAAUAGAA CUGAUGAG GCCGUUAGGC CGAA IAAAGAAG | 3675 |
| 1977 | UUCUUUCC U UCUAUUCG | 1167 | CGAAUAGA CUGAUGAG GCCGUUAGGC CGAA IGAAAGAA | 3676 |
| 1980 | UUUCCUUC U AUUCGAGA | 1168 | UCUCGAAU CUGAUGAG GCCGUUAGGC CGAA IAAGGAAA | 3677 |
| 1991 | UCGAGAUC U CCUCGACA | 1169 | UGUCGAGG CUGAUGAG GCCGUUAGGC CGAA IAUCUCGA | 3678 |
| 1993 | GAGAUCUC C UCGACACC | 1170 | GGUGUCGA CUGAUGAG GCCGUUAGGC CGAA IAGAUCUC | 3679 |
| 1994 | AGAUCUCC U CGACACCG | 1171 | CGGUGUCG CUGAUGAG GCCGUUAGGC CGAA IGAGAUCU | 3680 |
| 1999 | UCCUCGAC A CCGCCUCU | 1172 | AGAGGCGG CUGAUGAG GCCGUUAGGC CGAA IUCGAGGA | 3681 |
| 2001 | CUCGACAC C GCCUCUGC | 1173 | GCAGAGGC CUGAUGAG GCCGUUAGGC CGAA IUGUGGAG | 3682 |
| 2004 | GACACCGC C UCUGCUCU | 1174 | AGAGCAGA CUGAUGAG GCCGUUAGGC CGAA ICGGUGUC | 3683 |
| 2005 | ACACCGCC U CUGCUCUG | 1175 | CAGAGCAG CUGAUGAG GCCGUUAGGC CGAA IGCGGUGU | 3684 |
| 2007 | ACCGCCUC U GCUCUGUA | 1176 | UACAGAGC CUGAUGAG GCCGUUAGGC CGAA IAGGCGGU | 3685 |
| 2010 | GCCUCUGC U CUGUAUCG | 1177 | CGAUACAG CUGAUGAG GCCGUUAGGC CGAA ICAGAGGC | 3686 |
| 2012 | CUCUGCUC U GUAUCGGG | 1178 | CCCGAUAC CUGAUGAG GCCGUUAGGC CGAA IAGCAGAG | 3687 |
| 2025 | CGGGGGGC C UUAGAGUC | 1179 | GACUCUAA CUGAUGAG GCCGUUAGGC CGAA ICCCCCCG | 3688 |
| 2026 | GGGGGGCC U UAGAGUCU | 1180 | AGACUCUA CUGAUGAG GCCGUUAGGC CGAA IGCCCCCC | 3689 |
| 2034 | UUAGAGUC U CCGGAACA | 1181 | UGUUCCGG CUGAUGAG GCCGUUAGGC CGAA IACUCUAA | 3690 |
| 2036 | AGAGUCUC C GGAACAUU | 1182 | AAUGUUCC CUGAUGAG GCCGUUAGGC CGAA IAGACUCU | 3691 |
| 2042 | UCCGGAAC A UUGUUCAC | 1183 | GUGAACAA CUGAUGAG GCCGUUAGGC CGAA IUUCCGGA | 3692 |
| 2049 | CAUUGUUC A CCUCACCA | 1184 | UGGUGAGG CUGAUGAG GCCGUUAGGC CGAA IAACAAUG | 3693 |
| 2051 | UUGUUCAC C UCACCAUA | 1185 | UAUGGUGA CUGAUGAG GCCGUUAGGC CGAA IUGAACAA | 3694 |
| 2052 | UGUUCACC U CACCAUAC | 1186 | GUAUGGUG CUGAUGAG GCCGUUAGGC CGAA IGUGAACA | 3695 |
| 2054 | UUCACCUC A CCAUACGG | 1187 | CCGUAUGG CUGAUGAG GCCGUUAGGC CGAA IAGGUGAA | 3696 |
| 2056 | CACCUCAC C AUACGGCA | 1188 | UGCCGUAU CUGAUGAG GCCGUUAGGC CGAA IUGAGGUG | 3697 |
| 2057 | ACCUCACC A UACGGCAC | 1189 | GUGCCGUA CUGAUGAG GCCGUUAGGC CGAA IGUGAGGU | 3698 |
| 2064 | CAUACGGC A CUCAGGCA | 1190 | UGCCUGAG CUGAUGAG GCCGUUAGGC CGAA ICCGUAUG | 3699 |
| 2066 | UACGGCAC U CAGGCAAG | 1191 | CUUGCCUG CUGAUGAG GCCGUUAGGC CGAA IUGCCGUA | 3700 |
| 2068 | CGGCACUC A GGCAAGCU | 1192 | AGCUUGCC CUGAUGAG GCCGUUAGGC CGAA IAGUGCCG | 3701 |
| 2072 | ACUCAGGC A AGCUAUUC | 1193 | GAAUAGCU CUGAUGAG GCCGUUAGGC CGAA ICCUGAGU | 3702 |
| 2076 | AGGCAAGC U ACUCUGUG | 1194 | CACAGAAU CUGAUGAG GCCGUUAGGC CGAA ICUUGCCU | 3703 |
| 2081 | AGCUAUUC U GUGUUGGG | 1195 | CCCAACAC CUGAUGAG GCCGUUAGGC CGAA IAAUAGCU | 3704 |
| 2105 | GAUGAAUC U AGCCACCU | 1196 | AGGUGGCU CUGAUGAG GCCGUUAGGC CGAA IAUUCAUC | 3705 |
| 2109 | AAUCUAGC C ACCUGGGU | 1197 | ACCCAGGU CUGAUGAG GCCGUUAGGC CGAA ICUAGAUU | 3706 |
| 2110 | AUCUAGCC A CCUGGGUG | 1198 | CACCCAGG CUGAUGAG GCCGUUAGGC CGAA IGCUAGAU | 3707 |
| 2112 | CUAGCCAC C UGGGUGGG | 1199 | CCCACCCA CUGAUGAG GCCGUUAGGC CGAA IUGGCUAG | 3708 |
| 2113 | UAGCCACC U GGGUGGGA | 1200 | UCCCACCC CUGAUGAG GCCGUUAGGC CGAA IGUGGCUA | 3709 |
| 2138 | GGAAGAUC C AGCAUCCA | 1201 | UGGAUGCU CUGAUGAG GCCGUUAGGC CGAA IAUCUUCC | 3710 |
| 2139 | GAAGAUCC A GCAUCCAG | 1202 | CUGGAUGC CUGAUGAG GCCGUUAGGC CGAA IGAUCUUC | 3711 |
| 2142 | GAUCCAGC A UCCAGGGA | 1203 | UCCCUGGA CUGAUGAG GCCGUUAGGC CGAA ICUGGAUC | 3712 |
| 2145 | CCAGCAUC C AGGGAAUU | 1204 | AAUUCCCU CUGAUGAG GCCGUUAGGC CGAA IAUGCUGG | 3713 |
| 2146 | CAGCAUCC A GGGAAUUA | 1205 | UAAUUCCC CUGAUGAG GCCGUUAGGC CGAA IGAUCCUG | 3714 |
| 2161 | UAGUAGUC A GCUAUGUC | 1206 | GACAUAGC CUGAUGAG GCCGUUAGGC CGAA IACUACUA | 3715 |
| 2164 | UAGUCAGC U AUGUCAAC | 1207 | GUUGACAU CUGAUGAG GCCGUUAGGC CGAA ICUGACUA | 3716 |
| 2170 | GCUAUGUC A ACGUUAAU | 1208 | AUUAACGU CUGAUGAG GCCGUUAGGC CGAA IACAUAGC | 3717 |
| 2185 | AUAUGGGC C UAAAAAUC | 1209 | GAUUUUUA CUGAUGAG GCCGUUAGGC CGAA ICCCAUAU | 3718 |
| 2186 | UAUGGGCC U AAAAAUCA | 1210 | UGAUUUUU CUGAUGAG GCCGUUAGGC CGAA IGCCCAUA | 3719 |
| 2194 | UAAAAAUC A GACAACUA | 1211 | UAGUUGUC CUGAUGAG GCCGUUAGGC CGAA IAUUUUUA | 3720 |
| 2198 | AAUCAGAC A ACUAUUGU | 1212 | ACAAUAGU CUGAUGAG GCCGUUAGGC CGAA IUCUGAUU | 3721 |
| 2201 | CAGACAAC U AUUGUGGU | 1213 | ACCACAAU CUGAUGAG GCCGUUAGGC CGAA IUUGUCUG | 3722 |
| 2213 | GUGGUUUC A CAUUUCCU | 1214 | AGGAAAUG CUGAUGAG GCCGUUAGGC CGAA IAAACCAC | 3723 |
| 2215 | GGUUUCAC A UUUCCUGU | 1215 | ACAGGAAA CUGAUGAG GCCGUUAGGC CGAA IUGAAACC | 3724 |
| 2220 | CACAUUUC C UGUCUUAC | 1216 | GUAAGACA CUGAUGAG GCCGUUAGGC CGAA IAAAUGUG | 3725 |
| 2221 | ACAUUUCC U GUCUUACU | 1217 | AGUAAGAC CUGAUGAG GCCGUUAGGC CGAA IGAAAUGU | 3726 |
| 2225 | UUCCUGUC U UACUUUUG | 1218 | CAAAAGUA CUGAUGAG GCCGUUAGGC CGAA IACAGGAA | 3727 |
| 2229 | UGUCUUAC U UUUGGGCG | 1219 | CGCCCAAA CUGAUGAG GCCGUUAGGC CGAA IUAAGACA | 3728 |
| 2244 | CGAGAAAC U GUUCCUGA | 1220 | UCAAGAAC CUGAUGAG GCCGUUAGGC CGAA IUUUCUCG | 3729 |
| 2249 | AACUGUUC U UGAAUAUU | 1221 | AAUAUUCA CUGAUGAG GCCGUUAGGC CGAA IAACAGUU | 3730 |
| 2265 | UUGGUGUC U UUUGGAGU | 1222 | ACUCCAAA CUGAUGAG GCCGUUAGGC CGAA IACACCAA | 3731 |
| 2284 | GGAUUCGC A CUCCUCCU | 1223 | AGGAGGAG CUGAUGAG GCCGUUAGGC CGAA ICGAAUCC | 3732 |
| 2286 | AUUCGCAC U CCUCCUGC | 1224 | GCAGGAGG CUGAUGAG GCCGUUAGGC CGAA IUGCGAAU | 3733 |
| 2288 | UCGCACUC C UCCUGCAU | 1225 | AUGCAGGA CUGAUGAG GCCGUUAGGC CGAA IAGUGCGA | 3734 |
| 2289 | CGCACUCC U CCUGCAUA | 1226 | UAUGCAGG CUGAUGAG GCCGUUAGGC CGAA IGAGUGCG | 3735 |
| 2291 | CACUCCUC C UGCAUAUA | 1227 | UAUAUGCA CUGAUGAG GCCGUUAGGC CGAA IAGGAGUG | 3736 |
| 2292 | ACUCCUCC U GCAUAUAG | 1228 | CUAUAUGC CUGAUGAG GCCGUUAGGC CGAA IGAGGAGU | 3737 |
| 2295 | CCUCCUGC A UAUAGACC | 1229 | GGUCUAUA CUGAUGAG GCCGUUAGGC CGAA ICAGGAGG | 3738 |
| 2303 | AUAUAGAC C ACCAAAUG | 1230 | CAUUUGGU CUGAUGAG GCCGUUAGGC CGAA IUCUAUAU | 3739 |
| 2304 | UAUAGACC A CCAAAUGC | 1231 | GCAUUUGG CUGAUGAG GCCGUUAGGC CGAA IGUCUAUA | 3740 |
| 2306 | UAGACCAC C AAAUGCCC | 1232 | GGGCAUUU CUGAUGAG GCCGUUAGGC CGAA IUGGUCUA | 3741 |
| 2307 | AGACCACC A AAUGCCCC | 1233 | GGGGCAUU CUGAUGAG GCCGUUAGGC CGAA IGUGGUCU | 3742 |
| 2313 | CCAAAUGC C CCUAUCUU | 1234 | AAGAUAGG CUGAUGAG GCCGUUAGGC CGAA ICAUUUGG | 3743 |
| 2314 | CAAAUGCC C CUAUCUUA | 1235 | UAAGAUAG CUGAUGAG GCCGUUAGGC CGAA IGCAUUUG | 3744 |
| 2315 | AAAUGCCC C UAUCUUAU | 1236 | AUAAGAUA CUGAUGAG GCCGUUAGGC CGAA IGGCAUUU | 3745 |
| 2316 | AAUGCCCC U AUCUUAUC | 1237 | GAUAAGAU CUGAUGAG GCCGUUAGGC CGAA IGGGCAUU | 3746 |
| 2320 | CCCCUAUC U UAUCAACA | 1238 | UGUUGAUA CUGAUGAG GCCGUUAGGC CGAA IAUAGGGG | 3747 |
| 2325 | AUCUUAUC A ACACUUCC | 1239 | GGAAGUGU CUGAUGAG GCCGUUAGGC CGAA IAUAAGAU | 3748 |
| 2328 | UUAUCAAC A CUUCCGGA | 1240 | UCCGGAAG CUGAUGAG GCCGUUAGGC CGAA IUUGAUAA | 3749 |
| 2330 | AUCAACAC U UCCGGAAA | 1241 | UUUCCGGA CUGAUGAG GCCGUUAGGC CGAA IUGUUGAU | 3750 |
| 2333 | AACACUUC C GGAAACUA | 1242 | UAGUUUCC CUGAUGAG GCCGUUAGGC CGAA IAAGUGUU | 3751 |
| 2340 | CCGGAAAC U ACUGUUGU | 1243 | ACAACAGU CUGAUGAG GCCGUUAGGC CGAA IUUUCCGG | 3752 |
| 2343 | GAAACUAC U GUUGUUAG | 1244 | CUAACAAC CUGAUGAG GCCGUUAGGC CGAA IUAGUUUC | 3753 |
| 2362 | GAAGAGGC A GGUCCCCU | 1245 | AGGGGACC CUGAUGAG GCCGUUAGGC CGAA ICCUCUUC | 3754 |
| 2367 | GGCAGGUC C CCUAGAAG | 1246 | CUUCUAGG CUGAUGAG GCCGUUAGGC CGAA IACCUGCC | 3755 |
| 2368 | GCAGGUCC C CUAGAAGA | 1247 | UCUUCUAG CUGAUGAG GCCGUUAGGC CGAA IGACCUGC | 3756 |
| 2369 | CAGGUCCC C UAGAAGAA | 1248 | UUCUUCUA CUGAUGAG GCCGUUAGGC CGAA IGGACCUG | 3757 |
| 2370 | AGGUCCCC U AGAAGAAG | 1249 | CUUCUUCU CUGAUGAG GCCGUUAGGC CGAA IGGGACCU | 3758 |
| 2382 | AGAAGAAC U CCCUCGCC | 1250 | GGCGAGGG CUGAUGAG GCCGUUAGGC CGAA IUUCUUCU | 3759 |
| 2384 | AAGAACUC C CUCGCCUC | 1251 | GAGGCGAG CUGAUGAG GCCGUUAGGC CGAA IAGUUCUU | 3760 |
| 2385 | AGAACUCC C UCGCCUCG | 1252 | CGAGGCGA CUGAUGAG GCCGUUAGGC CGAA IGAGUUCU | 3761 |
| 2386 | GAACUCCC U CGCCUCGC | 1253 | GCGAGGCG CUGAUGAG GCCGUUAGGC CGAA IGGAGUUC | 3762 |
| 2390 | UCCCUCGC C UCGCAGAC | 1254 | GUCUGCGA CUGAUGAG GCCGUUAGGC CGAA ICGAGGGA | 3763 |
| 2391 | CCCUCGCC U CGCAGACG | 1255 | CGUCUGCG CUGAUGAG GCCGUUAGGC CGAA IGCGAGGG | 3764 |
| 2395 | CGCCUCGC A GACGAAGG | 1256 | CCUUCGUC CUGAUGAG GCCGUUAGGC CGAA ICGAGGCG | 3765 |
| 2406 | CGAAGGUC U CAAUCGCC | 1257 | GGCGAUUG CUGAUGAG GCCGUUAGGC CGAA IACCUUCG | 3766 |
| 2408 | AAGGUCUC A AUCGCCGC | 1258 | GCGGCGAU CUGAUGAG GCCGUUAGGC CGAA IAGACCUU | 3767 |
| 2414 | UCAAUCGC C GCGUCGCA | 1259 | UGCGACGC CUGAUGAG GCCGUUAGGC CGAA ICGAUUGA | 3768 |
| 2422 | CGCGUCGC A GAAGAUCU | 1260 | AGAUCUUC CUGAUGAG GCCGUUAGGC CGAA ICGACGCG | 3769 |
| 2430 | AGAAGAUC U CAAUCUCG | 1261 | CGAGAUUG CUGAUGAG GCCGUUAGGC CGAA IAUCUUCU | 3770 |
| 2432 | AAGAUCUC A AUCUCGGG | 1262 | CCCGAGAU CUGAUGAG GCCGUUAGGC CGAA IAGAUCUU | 3771 |
| 2436 | UCUCAAUC U CGGGAAUC | 1263 | GAUUCCCG CUGAUGAG GCCGUUAGGC CGAA IAUGGAGA | 3772 |
| 2445 | CGGGAAUC U CAAUGUUA | 1264 | UAACAUUG CUGAUGAG GCCGUUAGGC CGAA IAUUCCCG | 3773 |
| 2447 | GGAAUCUC A AUGUUAGU | 1265 | ACUAACAU CUGAUGAG GCCGUUAGGC CGAA IAGAUUCC | 3774 |
| 2460 | UAGUAUUC C UUGGACAC | 1266 | GUGUCCAA CUGAUGAG GCCGUUAGGC CGAA IAAUACUA | 3775 |
| 2461 | AGUAUUCC U UGGACACA | 1267 | UGUGUCCA CUGAUGAG GCCGUUAGGC CGAA IGAAUACU | 3776 |
| 2467 | CCUUGGAC A CAUAAGGU | 1268 | ACCUUAUG CUGAUGAG GCCGUUAGGC CGAA IUCCAAGG | 3777 |
| 2469 | UUGGACAC A UAAGGUGG | 1269 | CCACCUUA CUGAUGAG GCCGUUAGGC CGAA IUGUCCAA | 3778 |
| 2483 | UGGGAAAC U UUACGGGG | 1270 | CCCCGUAA CUGAUGAG GCCGUUAGGC CGAA IUUUCCCA | 3779 |
| 2493 | UACGGGGC U UUAUUCUU | 1271 | AAGAAUAA CUGAUGAG GCCGUUAGGC CGAA ICCCCGUA | 3780 |
| 2500 | CUUUAUUC U UCUACGGU | 1272 | ACCGUAGA CUGAUGAG GCCGUUAGGC CGAA IAAUAAAG | 3781 |
| 2503 | UAUUCUUC U ACGGUACC | 1273 | GGUACCGU CUGAUGAG GCCGUUAGGC CGAA IAAGAAUA | 3782 |
| 2511 | UACGGUAC C UUGCUUUA | 1274 | UAAAGCAA CUGAUGAG GCCGUUAGGC CGAA IUACCGUA | 3783 |
| 2512 | ACGGUACC U UGCUUUAA | 1275 | UUAAAGCA CUGAUGAG GCCGUUAGGC CGAA IGUACCGU | 3784 |
| 2516 | UACCUUGC U UUAAUCCU | 1276 | AGGAUUAA CUGAUGAG GCCGUUAGGC CGAA ICAAGGUA | 3785 |
| 2523 | CUUUAAUC C UAAAUGGC | 1277 | GCCAUUUA CUGAUGAG GCCGUUAGGC CGAA IAUUAAAG | 3786 |
| 2524 | UUUAAUCC U AAAUGGCA | 1278 | UGCCAUUU CUGAUGAG GCCGUUAGGC CGAA IGAUUAAA | 3787 |
| 2532 | UAAAUGGC A AACUCCUU | 1279 | AAGGAGUU CUGAUGAG GCCGUUAGGC CGAA ICCAUUUA | 3788 |
| 2536 | UGGCAAAC U CCUUCUUU | 1280 | AAAGAAGG CUGAUGAG GCCGUUAGGC CGAA IUUUGCCA | 3789 |
| 2538 | GCAAACUC C UUCUUUUC | 1281 | GAAAAGAA CUGAUGAG GCCGUUAGGC CGAA IAGUUUGC | 3790 |
| 2539 | CAAACUCC U UCUUUUCC | 1282 | GGAAAAGA CUGAUGAG GCCGUUAGGC CGAA IGAGUUUG | 3791 |
| 2542 | ACUCCUUC U UUUCCUGA | 1283 | UCAGGAAA CUGAUGAG GCCGUUAGGC CGAA IAAGGAGU | 3792 |
| 2547 | UUCUUUUC C UGACAGUC | 1284 | GAAUGUCA CUGAUGAG GCCGUUAGGC CGAA IAAAAGAA | 3793 |
| 2548 | UCUUUUCC U GACAUUCA | 1285 | UGAAUGUC CUGAUGAG GCCGUUAGGC CGAA IGAAAAGA | 3794 |
| 2552 | UUCCUGAC A UUCAUUUG | 1286 | CAAAUGAA CUGAUGAG GCCGUUAGGC CGAA IUCAGGAA | 3795 |
| 2556 | UGACAUUC A UUUGCAGG | 1287 | CCUGCAAA CUGAUGAG GCCGUUAGGC CGAA IAAUGUCA | 3796 |
| 2562 | UCAUUUGC A GGAGGACA | 1288 | UGUCCUCC CUGAUGAG GCCGUUAGGC CGAA ICAAAUGA | 3797 |
| 2570 | AGGAGGAC A UUGUUGAU | 1289 | AUCAACAA CUGAUGAG GCCGUUAGGC CGAA IUCCUCCU | 3798 |
| 2589 | AUGUAAGC A AUUUGUGG | 1290 | CCACAAAU CUGAUGAG GCCGUUAGGC CGAA ICUUACAU | 3799 |
| 2601 | UGUGGGGC C CCUUACAG | 1291 | CUGUAAGG CUGAUGAG GCCGUUAGGC CGAA ICCCCACA | 3800 |
| 2602 | GUGGGGCC C CUUACAGU | 1292 | ACUGUAAG CUGAUGAG GCCGUUAGGC CGAA IGCCCCAC | 3801 |
| 2603 | UGGGGCCC C UUACAGUA | 1293 | UACUGUAA CUGAUGAG GCCGUUAGGC CGAA IGGCCCCA | 3802 |
| 2604 | GGGGCCCC U UACAGUAA | 1294 | UUACUGUA CUGAUGAG GCCGUUAGGC CGAA IGGGCCCC | 3803 |
| 2608 | CCCCUUAC A GUAAAUGA | 1295 | UCAUUUAC CUGAUGAG GCCGUUAGGC CGAA IUAAGGGG | 3804 |
| 2621 | AUGAAAAC A GGAGACUU | 1296 | AAGUCUCC CUGAUGAG GCCGUUAGGC CGAA IUUUUCAU | 3805 |
| 2628 | CAGGAGAC U UAAAUUAA | 1297 | UUAAUUUA CUGAUGAG GCCGUUAGGC CGAA IUCUCCUG | 3806 |
| 2638 | AAAUUAAC U AUGCCUGC | 1298 | GCAGGCAU CUGAUGAG GCCGUUAGGC CGAA IUUAAUUU | 3807 |
| 2643 | AACUAUGC C UGCUAGGU | 1299 | ACCUAGCA CUGAUGAG GCCGUUAGGC CGAA ICAUAGUU | 3808 |
| 2644 | ACUAUGCC U GCUAGGUU | 1300 | AACCUAGC CUGAUGAG GCCGUUAGGC CGAA IGCAUAGU | 3809 |
| 2647 | AUGCCUGC U AGGUDUUA | 1301 | UAAAACCU CUGAUGAG GCCGUUAGGC CGAA ICAGGCAU | 3810 |
| 2658 | GUUUUAUC C CAAUGUUA | 1302 | UAACAUUG CUGAUGAG GCCGUUAGGC CGAA IAUAAAAC | 3811 |
| 2659 | UUUUAUCC C AAUGUUAC | 1303 | GUAACAUU CUGAUGAG GCCGUUAGGC CGAA IGAUAAAA | 3812 |
| 2660 | UUUAUCCC A AUGUUACU | 1304 | AGUAACAU CUGAUGAG GCCGUUAGGC CGAA IGGAUAAA | 3813 |
| 2668 | AAUGUUAC U AAAUAUUU | 1305 | AAAUAUUU CUGAUGAG GCCGUUAGGC CGAA IUAACAUU | 3814 |
| 2679 | AUAUUUGC C CUUAGAUA | 1306 | UAUCUAAG CUGAUGAG GCCGUUAGGC CGAA ICAAAUAU | 3815 |
| 2680 | UAUUUGCC C UUAGAUAA | 1307 | UUAUCUAA CUGAUGAG GCCGUUAGGC CGAA IGCAAAUA | 3816 |
| 2681 | AUUUGCCC U UAGAUAAA | 1308 | UUUAUCUA CUGAUGAG GCCGUUAGGC CGAA IGGCAAAU | 3817 |
| 2696 | AAGGGAUC A AACCGUAU | 1309 | AUACGGUU CUGAUGAG GCCGUUAGGC CGAA IAUCCCUU | 3818 |
| 2700 | GAUCAAAC C GUAUUAUC | 1310 | GAUAAUAC CUGAUGAG GCCGUUAGGC CGAA IUUUGAUC | 3819 |
| 2709 | GUAUUAUC C AGAGUAUG | 1311 | CAUACUCU CUGAUGAG GCCGUUAGGC CGAA IAUAAUAC | 3820 |
| 2710 | UAUUAUCC A GAGUAUGU | 1312 | ACAUACUC CUGAUGAG GCCGUUAGGC CGAA IGAUAAUA | 3821 |
| 2727 | AGUUAAUC A UUACUUCC | 1313 | GGAAGUAA CUGAUGAG GCCGUUAGGC CGAA IAUUAACU | 3822 |
| 2732 | AUCAUUAC U UCCAGACG | 1314 | CGUCUGGA CUGAUGAG GCCGUUAGGC CGAA IUAAUGAU | 3823 |
| 2735 | AUUACUUC C AGACGCGA | 1315 | UCGCGUCU CUGAUGAG GCCGUUAGGC CGAA IAAGUAAU | 3824 |
| 2736 | UUACUUCC A GACGCGAC | 1316 | GUCGCGUC CUGAUGAG GCCGUUAGGC CGAA IGAAGUAA | 3825 |
| 2745 | GACGCGAC A UUAUUUAC | 1317 | GUAAAUAA CUGAUGAG GCCGUUAGGC CGAA IUCGCGUC | 3826 |
| 2754 | UUAUUUAC A CACUCUUU | 1318 | AAAGAGUG CUGAUGAG GCCGUUAGGC CGAA IUAAAUAA | 3827 |
| 2756 | AUUUACAC A CUCUUUGG | 1319 | CCAAAGAG CUGAUGAG GCCGUUAGGC CGAA IUGUAAAU | 3828 |
| 2758 | UUACACAC U CUUUGGAA | 1320 | UUCCAAAG CUGAUGAG GCCGUUAGGC CGAA IUGUGUAA | 3829 |
| 2760 | ACACACUC U UUGGAAGG | 1321 | CCUUCCAA CUGAUGAG GCCGUUAGGC CGAA IAGUGUGU | 3830 |
| 2777 | CGGGGAUC U UAUAUAAA | 1322 | UUUAUAUA CUGAUGAG GCCGUUAGGC CGAA IAUCCCCG | 3831 |
| 2794 | AGAGAGUC C ACACGUAG | 1323 | CUACGUGU CUGAUGAG GCCGUUAGGC CGAA IACUCUCU | 3832 |
| 2795 | GAGAGUCC A CACGUAGC | 1324 | GCUACGUG CUGAUGAG GCCGUUAGGC CGAA IGACUCUC | 3833 |
| 2797 | GAGUCCAC A CGUAGCGC | 1325 | GCGCUACG CUGAUGAG GCCGUUAGGC CGAA IUGGACUC | 3834 |
| 2806 | CGUAGCGC C UCAUUUUG | 1326 | CAAAAUGA CUGAUGAG GCCGUUAGGC CGAA ICGCUACG | 3835 |
| 2807 | GUAGCGCC U CAUUUUGC | 1327 | GCAAAAUG CUGAUGAG GCCGUUAGGC CGAA IGCGCUAC | 3836 |
| 2809 | AGCGCCUC A UUUUGCGG | 1328 | CCGCAAAA CUGAUGAG GCCGUUAGGC CGAA IAGGCGCU | 3837 |
| 2821 | UGCGGGUC A CCAUAUUC | 1329 | GAAUAUGG CUGAUGAG GCCGUUAGGC CGAA IACCCGCA | 3838 |
| 2823 | CGGGUCAC C AUAUUCUU | 1330 | AAGAAUAU CUGAUGAG GCCGUUAGGC CGAA IUGACCCG | 3839 |
| 2824 | GGGUCACC A UAUUCUUG | 1331 | CAAGAAUA CUGAUGAG GCCGUUAGGC CGAA IGUGACCC | 3840 |
| 2830 | CCAUAUUC U UGGGAACA | 1332 | UGUUCCCA CUGAUGAG GCCGUUAGGC CGAA IAAUAUGG | 3841 |
| 2838 | UUGGGAAC A AGAUCUAC | 1333 | GUAGAUCU CUGAUGAG GCCGUUAGGC CGAA IUUCCCAA | 3842 |
| 2844 | ACAAGAUC U ACAGCAUG | 1334 | CAUGCUGU CUGAUGAG GCCGUUAGGC CGAA IAUCUUGU | 3843 |
| 2847 | AGAUCUAC A GCAUGGGA | 1335 | UCCCAUGC CUGAUGAG GCCGUUAGGC CGAA IUAGAUCU | 3844 |
| 2850 | UCUACAGC A UGGGAGGU | 1336 | ACCUCCCA CUGAUGAG GCCGUUAGGC CGAA ICUGUAGA | 3845 |
| 2864 | GGUUGGUC U UCCAAACC | 1337 | GGUUUGGA CUGAUGAG GCCGUUAGGC CGAA IACCAACC | 3846 |
| 2867 | UGGUCUUC C AAACCUCG | 1338 | CGAGGUUU CUGAUGAG GCCGUUAGGC CGAA IAAGACCA | 3847 |
| 2868 | GGUCUUCC A AACCUCGA | 1339 | UCGAGGUU CUGAUGAG GCCGUUAGGC CGAA IGAAGACC | 3848 |
| 2872 | UUCCAAAC C UCGAAAAG | 1340 | CUUUUCGA CUGAUGAG GCCGUUAGGC CGAA IUUUGGAA | 3849 |
| 2873 | UCCAAACC U CGAAAAGG | 1341 | CCUUUUCG CUGAUGAG GCCGUUAGGC CGAA IGUUUGGA | 3850 |
| 2883 | GAAAAGGC A UGGGGACA | 1342 | UGUCCCCA CUGAUGAG GCCGUUAGGC CGAA ICCUUUUC | 3851 |
| 2891 | AUGGGGAC A AAUCUUUC | 1343 | GAAAGAUU CUGAUGAG GCCGUUAGGC CGAA IUCCCCAU | 3852 |
| 2896 | GACAAAUC U UUCUGUCC | 1344 | GGACAGAA CUGAUGAG GCCGUUAGGC CGAA IAUUUGUC | 3853 |
| 2900 | AAUCUUUC U GUCCCCAA | 1345 | UUGGGGAC CUGAUGAG GCCGUUAGGC CGAA IAAAGAUU | 3854 |
| 2904 | UUUCUGUC C CCAAUCCC | 1346 | GGGAUUGG CUGAUGAG GCCGUUAGGC CGAA IACAGAAA | 3855 |
| 2905 | UUCUGUCC C CAAUCCCC | 1347 | GGGGAUUG CUGAUGAG GCCGUUAGGC CGAA IGACAGAA | 3856 |
| 2906 | UCUGUCCC C AAUCCCCU | 1348 | AGGGGAUU CUGAUGAG GCCGUUAGGC CGAA IGGACAGA | 3857 |
| 2907 | CUGUCCCC A AUCCCCUG | 1349 | CAGGGGAU CUGAUGAG GCCGUUAGGC CGAA IGGGACAG | 3858 |
| 2911 | CCCCAAUC C CCUGGGAU | 1350 | AUCCCAGG CUGAUGAG GCCGUUAGGC CGAA IAUUGGGG | 3859 |
| 2912 | CCCAAUCC C CUGGGAUU | 1351 | AAUCCCAG CUGAUGAG GCCGUUAGGC CGAA IGAUUGGG | 3860 |
| 2913 | CCAAUCCC C UGGGAUUC | 1352 | GAAUCCCA CUGAUGAG GCCGUUAGGC CGAA IGGAUUGG | 3861 |
| 2914 | CAAUCCCC U GGGAUUCU | 1353 | AGAAUCCC CUGAUGAG GCCGUUAGGC CGAA IGGGAUUG | 3862 |
| 2922 | UGGGAUUC U UCCCCGAU | 1354 | AUCGGGGA CUGAUGAG GCCGUUAGGC CGAA IAAUCCCA | 3863 |
| 2925 | GAUUCUUC C CCGAUCAU | 1355 | AUGAUCGG CUGAUGAG GCCGUUAGGC CGAA IAAGAAUC | 3864 |
| 2926 | AUUCUUCC C CGAUCAUC | 1356 | GAUGAUCG CUGAUGAG GCCGUUAGGC CGAA IGAAGAAU | 3865 |
| 2927 | UUCUUCCC C GAUCAUCA | 1357 | UGAUGAUC CUGAUGAG GCCGUUAGGC CGAA IGGAAGAA | 3866 |
| 2932 | CCCCGAUC A UCAGUUGG | 1358 | CCAACUGA CUGAUGAG GCCGUUAGGC CGAA IAUCGGGG | 3867 |
| 2935 | CGAUCAUC A GUUGGACC | 1359 | GGUCCAAC CUGAUGAG GCCGUUAGGC CGAA IAUGAUCG | 3868 |
| 2943 | AGUUGGAC C CUGCAUUC | 1360 | GAAUGCAG CUGAUGAG GCCGUUAGGC CGAA IUCCAACU | 3869 |
| 2944 | GUUGGACC C UGCAUUCA | 1361 | UGAAUGCA CUGAUGAG GCCGUUAGGC CGAA IGUCCAAC | 3870 |
| 2945 | UUGGACCC U GCAUUCAA | 1362 | UUGAAUGC CUGAUGAG GCCGUUAGGC CGAA IGGUCCAA | 3871 |
| 2948 | GACCCUGC A UUCAAAGC | 1363 | GCUUUGAA CUGAUGAG GCCGUUAGGC CGAA ICAGGGUC | 3872 |
| 2952 | CUGCAUUC A AAGCCAAC | 1364 | GUUGGCUU CUGAUGAG GCCGUUAGGC CGAA IAAUGCAG | 3873 |
| 2957 | UUCAAAGC C AACUCAGU | 1365 | ACUGAGUU CUGAUGAG GCCGUUAGGC CGAA ICUUUGAA | 3874 |
| 2958 | UCAAAGCC A ACUCAGUA | 1366 | UACUGAGU CUGAUGAG GCCGUUAGGC CGAA IGCUUUGA | 3875 |
| 2961 | AAGCCAAC U CAGUAAAU | 1367 | AUUUACUG CUGAUGAG GCCGUUAGGC CGAA IUUGGCUU | 3876 |
| 2963 | GCCAACUC A GUAAAUCC | 1368 | GGAUUUAC CUGAUGAG GCCGUUAGGC CGAA IAGUUGGC | 3877 |
| 2971 | AGUAAAUC C AGAUUGGG | 1369 | CCCAAUCU CUGAUGAG GCCGUUAGGC CGAA IAUUUACU | 3878 |
| 2972 | GUAAAUCC A GAUUGGGA | 1370 | UCCCAAUC CUGAUGAG GCCGUUAGGC CGAA IGAUUUAC | 3879 |
| 2982 | AUUGGGAC C UCAACCCG | 1371 | CGGGUUGA CUGAUGAG GCCGUUAGGC CGAA IUCCCAAU | 3880 |
| 2983 | UUGGGACC U CAACCCGC | 1372 | GCGGGUUG CUGAUGAG GCCGUUAGGC CGAA IGUCCCAA | 3881 |
| 2985 | GGGACCUC A ACCCGCAC | 1373 | GUGCGGGU CUGAUGAG GCCGUUAGGC CGAA IAGGUCCC | 3882 |
| 2988 | ACCUCAAC C CGCACAAG | 1374 | CUUGUGCG CUGAUGAG GCCGUUAGGC CGAA IUUGAGGU | 3883 |
| 2989 | CCUCAACC C GCACAAGG | 1375 | CCUUGUGC CUGAUGAG GCCGUUAGGC CGAA IGUUGAGG | 3884 |
| 2992 | CAACCCGC A CAAGGACA | 1376 | UGUCCUUG CUGAUGAG GCCGUUAGGC CGAA ICGGGUUG | 3885 |
| 2994 | ACCCGCAC A AGGACAAC | 1377 | GUUGUCCU CUGAUGAG GCCGUUAGGC CGAA IUGCGGGU | 3886 |
| 3000 | ACAAGGAC A ACUGGCCG | 1378 | CGGCCAGU CUGAUGAG GCCGUUAGGC CGAA IUCCUUGU | 3887 |
| 3003 | AGGACAAC U GGCCGGAC | 1379 | GUCCGGCC CUGAUGAG GCCGUUAGGC CGAA IUUGUCCU | 3888 |
| 3007 | CAACUGGC C GGACGCCA | 1380 | UGGCGUCC CUGAUGAG GCCGUUAGGC CGAA ICCAGUUG | 3889 |
| 3014 | CCGGACGC C AACAAGGU | 1381 | ACCUUGUU CUGAUGAG GCCGUUAGGC CGAA ICGUCCGG | 3890 |
| 3015 | CGGACGCC A ACAAGGUG | 1382 | CACCUUGU CUGAUGAG GCCGUUAGGC CGAA IGCGUCCG | 3891 |
| 3018 | ACGCCAAC A AGGUGGGA | 1383 | UCCCACCU CUGAUGAG GCCGUUAGGC CGAA IUUGGCGU | 3892 |
| 3035 | GUGGGAGC A UUCGGGCC | 1384 | GGCCCGAA CUGAUGAG GCCGUUAGGC CGAA ICUCCCAC | 3893 |
| 3043 | AUUCGGGC C AGGGUUCA | 1385 | UGAACCCU CUGAUGAG GCCGUUAGGC CGAA ICCCGAAU | 3894 |
| 3044 | UUCGGGCC A GGGUUCAC | 1386 | GUGAACCC CUGAUGAG GCCGUUAGGC CGAA IGCCCGAA | 3895 |
| 3051 | CAGGGUUC A CCCCUCCC | 1387 | GGGAGGGG CUGAUGAG GCCGUUAGGC CGAA IAACCCUG | 3896 |
| 3053 | GGGUUCAC C CCUCCCCA | 1388 | UGGGGAGG CUGAUGAG GCCGUUAGGC CGAA IUGAACCC | 3897 |
| 3054 | GGUUCACC C CUCCCCAU | 1389 | AUGGGGAG CUGAUGAG GCCGUUAGGC CGAA IGUGAACC | 3898 |
| 3055 | GUUCACCC C UCCCCAUG | 1390 | CAUGGGGA CUGAUGAG GCCGUUAGGC CGAA IGGUGAAC | 3899 |
| 3056 | UUCACCCC U CCCCAUGG | 1391 | CCAUGGGG CUGAUGAG GCCGUUAGGC CGAA IGGGUGAA | 3900 |
| 3058 | CACCCCUC C CCAUGGGG | 1392 | CCCCAUGG CUGAUGAG GCCGUUAGGC CGAA IAGGGGUG | 3901 |
| 3059 | ACCCCUCC C CAUGGGGG | 1393 | CCCCCAUG CUGAUGAG GCCGUUAGGC CGAA IGAGGGGU | 3902 |
| 3060 | CCCCUCCC C AUGGGGGA | 1394 | UCCCCCAU CUGAUGAG GCCGUUAGGC CGAA IGGAGGGG | 3903 |
| 3061 | CCCUCCCC A UGGGGGAC | 1395 | GUCCCCCA CUGAUGAG GCCGUUAGGC CGAA IGGGAGGG | 3904 |
| 3070 | UGGGGGAC U GUUGGGGU | 1396 | ACCCCAAC CUGAUGAG GCCGUUAGGC CGAA IUCCCCCA | 3905 |
| 3084 | GGUGGAGC C CUCACGCU | 1397 | AGCGUGAG CUGAUGAG GCCGUUAGGC CGAA ICUCCACC | 3906 |
| 3085 | GUGGAGCC C UCACGCUC | 1398 | GAGCGUGA CUGAUGAG GCCGUUAGGC CGAA IGCUCCAC | 3907 |
| 3086 | UGGAGCCC U CACGCUCA | 1399 | UGAGCGUG CUGAUGAG GCCGUUAGGC CGAA IGGCUCCA | 3908 |
| 3088 | GAGCCCUC A CGCUCAGG | 1400 | CCUGAGCG CUGAUGAG GCCGUUAGGC CGAA IAGGGCUC | 3909 |
| 3092 | CCUCACGC U CAGGGCCU | 1401 | AGGCCCUG CUGAUGAG GCCGUUAGGC CGAA ICGUGAGG | 3910 |
| 3094 | UCACGCUC A GGGCCUAC | 1402 | GUAGGCCC CUGAUGAG GCCGUUAGGC CGAA IAGCGUGA | 3911 |
| 3099 | CUCAGGGC C UACUCACA | 1403 | UGUGAGUA CUGAUGAG GCCGUUAGGC CGAA ICCCUGAG | 3912 |
| 3100 | UCAGGGCC U ACUCACAA | 1404 | UUGUGAGU CUGAUGAG GCCGUUAGGC CGAA IGCCCUGA | 3913 |
| 3103 | GGGCCUAC U CACAACUG | 1405 | CAGUUGUG CUGAUGAG GCCGUUAGGC CGAA IUAGGCCC | 3914 |
| 3105 | GCCUACUC A CAACUGUG | 1406 | CACAGUUG CUGAUGAG GCCGUUAGGC CGAA IAGUAGGC | 3915 |
| 3107 | CUACUCAC A ACUGUGCC | 1407 | GGCACAGU CUGAUGAG GCCGUUAGGC CGAA IUGAGUAG | 3916 |
| 3110 | CUCACAAC U GUGCCAGC | 1408 | GCUGGCAC CUGAUGAG GCCGUUAGGC CGAA IUUGUGAG | 3917 |
| 3115 | AACUGUGC C AGCAGCUC | 1409 | GAGCUGCU CUGAUGAG GCCGUUAGGC CGAA ICACAGUU | 3918 |
| 3116 | ACUGUGCC A GCAGCUCC | 1410 | GGAGCUGC CUGAUGAG GCCGUUAGGC CGAA IGCACAGU | 3919 |
| 3119 | GUGCCAUC A GCUCCUCC | 1411 | GGAGGAGC CUGAUGAG GCCGUUAGGC CGAA ICUGGCAC | 3920 |
| 3122 | CCAGCAGC U CCUCCUCC | 1412 | GGAGGAGG CUGAUGAG GCCGUUAGGC CGAA ICUGCUGG | 3921 |
| 3124 | AGCAGCUC C UCCUCCUG | 1413 | CAGGAGGA CUGAUGAG GCCGUUAGGC CGAA IAGCUGCU | 3922 |
| 3125 | GCAGCUCC U CCUCCUGC | 1414 | GCAGGAGG CUGAUGAG GCCGUUAGGC CGAA IGAGCUGC | 3923 |
| 3127 | AGCUCCUC C UCCUGCCU | 1415 | AGGCAGGA CUGAUGAG GCCGUUAGGC CGAA IAGGAGCU | 3924 |
| 3128 | GCUCCUCC U CCUGCCUC | 1416 | GAGGCAGG CUGAUGAG GCCGUUAGGC CGAA IGAGGAGC | 3925 |
| 3130 | UCCUCCUC C UGCCUCCA | 1417 | UGGAGGCA CUGAUGAG GCCGUUAGGC CGAA IAGGAGGA | 3926 |
| 3131 | CCUCCUCC U GCCUCCAC | 1418 | GUGGAGGC CUGAUGAG GCCGUUAGGC CGAA IGAGGAGG | 3927 |
| 3134 | CCUCCUGC C UCCACCAA | 1419 | UUGGUGGA CUGAUGAG GCCGUUAGGC CGAA ICAGGAGG | 3928 |
| 3135 | CUCCUGCC U CCACCAAU | 1420 | AUUGGUGG CUGAUGAG GCCGUUAGGC CGAA IGCAGGAG | 3929 |
| 3137 | CCUGCCUC C ACCAAUCG | 1421 | CGAUUGGU CUGAUGAG GCCGUUAGGC CGAA IAGGCAGG | 3930 |
| 3138 | CUGCCUCC A CCAAUCGG | 1422 | CCGAUUGG CUGAUGAG GCCGUUAGGC CGAA IGAGGCAG | 3931 |
| 3140 | GCCUCCAC C AAUCGGCA | 1423 | UGCCGAUU CUGAUGAG GCCGUUAGGC CGAA IUGGAGGC | 3932 |
| 3141 | CCUCCACC A AUCGGCAG | 1424 | CUGCCGAU CUGAUGAG GCCGUUAGGC CGAA IGUGGAGG | 3933 |
| 3148 | CAAUCGGC A GUCAGGAA | 1425 | UUCCUGAC CUGAUGAG GCCGUUAGGC CGAA ICCGAUUG | 3934 |
| 3152 | CGGCAGUC A GGAAGGCA | 1426 | UGCCUUCC CUGAUGAG GCCGUUAGGC CGAA IACUGCCG | 3935 |
| 3160 | AGGAAGGC A GCCUACUC | 1427 | GAGUAGGC CUGAUGAG GCCGUUAGGC CGAA ICCUUCCU | 3936 |
| 3163 | AAGGCAGC C UACUCCCU | 1428 | AGGGAGUA CUGAUGAG GCCGUUAGGC CGAA ICUGCCUU | 3937 |
| 3164 | AGGCAGCC U ACUCCCUU | 1429 | AAGGGAGU CUGAUGAG GCCGUUAGGC CGAA IGCUGCCU | 3938 |
| 3167 | CAGCCUAC U CCCUUAUC | 1430 | GAUAAGGG CUGAUGAG GCCGUUAGGC CGAA IUAGGCUG | 3939 |
| 3169 | GCCUACUC C CUUAUCUC | 1431 | GAGAUAAG CUGAUGAG GCCGUUAGGC CGAA IAGUAGGC | 3940 |
| 3170 | CCUACUCC C UUAUCUCC | 1432 | GGAGAUAA CUGAUGAG GCCGUUAGGC CGAA IGAGUAGG | 3941 |
| 3171 | CUACUCCC U UAUCUCCA | 1433 | UGGAGAUA CUGAUGAG GCCGUUAGGC CGAA IGGAGUAG | 3942 |
| 3176 | CCCUUAUC U CCACCUCU | 1434 | AGAGGUGG CUGAUGAG GCCGUUAGGC CGAA IAUAAGGG | 3943 |
| 3178 | CUUAUCUC C ACCUCUAA | 1435 | UUAGAGGU CUGAUGAG GCCGUUAGGC CGAA IAGAUAAG | 3944 |
| 3179 | UUAUCUCC A CCUCUAAG | 1436 | CUUAGAGG CUGAUGAG GCCGUUAGGC CGAA IGAGAUAA | 3945 |
| 3181 | AUCUCCAC C UCUAAGGG | 1437 | CCCUUAGA CUGAUGAG GCCGUUAGGC CGAA IUGGAGAU | 3946 |
| 3182 | UCUCCACC U CUAAGGGA | 1438 | UCCCUUAG CUGAUGAG GCCGUUAGGC CGAA IGUGGAGA | 3947 |
| 3184 | UCCACCUC U AAGGGACA | 1439 | UGUCCCUU CUGAUGAG GCCGUUAGGC CGAA IAGGUGGA | 3948 |
| 3192 | UAAGGGAC A CUCAUCCU | 1440 | AGGAUGAG CUGAUGAG GCCGUUAGGC CGAA IUCCCUUA | 3949 |
| 3194 | AGGGACAC U CAUCCUCA | 1441 | UGAGGAUG CUGAUGAG GCCGUUAGGC CGAA IUGUCCCU | 3950 |
| 3196 | GGACACUC A UCCUCAGG | 1442 | CCUGAGGA CUGAUGAG GCCGUUAGGC CGAA IAGUGUCC | 3951 |
| 3199 | CACUCAUC C UCAGGCCA | 1443 | UGGCCUGA CUGAUGAG GCCGUUAGGC CGAA IAUGAGUG | 3952 |
| 3200 | ACUCAUCC U CAGGCCAU | 1444 | AUGGCCUG CUGAUGAG GCCGUUAGGC CGAA IGAUGAGU | 3953 |
| 3202 | UCAUCCUC A GGCCAUGC | 1445 | GCAUGGCC CUGAUGAG GCCGUUAGGC CGAA IAGGAUGA | 3954 |
| 3206 | CCUCAGGC C AUGCAGUG | 1446 | CACUGCAU CUGAUGAG GCCGUUAGGC CGAA ICCUGAGG | 3955 |
| 3207 | CUCAGGCC A UGCAGUGG | 1447 | CCACUGCA CUGAUGAG GCCGUUAGGC CGAA IGCCUGAG | 3956 |

Input Sequence AF100308. Cut Site= CH/.
Stem Length= 8 . Core Sequence= CUGAUGAG X CGAA (X=GCCGUUAGGC or other stem II)
AF100308 (Hepatitis B virus strain 2-18, 3215 bp)
Underlined region can be any X sequence or linker, as described herein.
“I” stands for Inosime

TABLE VII

HUMAN HBV G-CLEAVER AND SUBSTRATE SEQUENCE

| | | | | |
|---|---|---|---|---|
| Pos | Substrate | Seq ID | G-cleaver | Seq ID |

| | | | | |
|---|---|---|---|---|
| 61 | ACUUUCCU G CUGGUGGC | 1448 | GCCACCAG UGAUG GCAUGCACUAUGC GCG AGGAAAGU | 3957 |
| 87 | GGAACAGU G AGCCCUGC | 1449 | GCAGGGCU UGAUG GCAUGCACUAUGC GCG ACUGUUCC | 3958 |
| 94 | UGAGCCCU G CUCAGAAU | 1450 | AUUCUGAG UGAUG GCAUGCACUAUGC GCG AGGGCUCA | 3959 |
| 112 | CUGUCUCU G CCAUAUCG | 1451 | CGAUAUGG UGAUG GCAUGCACUAUGC GCG AGAGACAG | 3960 |
| 132 | AUCUUAUC G AAGACUGG | 1452 | CCAGUCUU UGAUG GCAUGCACUAUGC GCG GAUAAGAU | 3961 |
| 153 | CCUGUACC G AACAUGGA | 1453 | UCCAUGUU UGAUG GCAUGCACUAUGC GCG GGUACAGG | 3962 |
| 169 | AGAACAUC G CAUCAGGA | 1454 | UCCUGAUG UGAUG GCAUGCACUAUGC GCG GAUGUUCU | 3963 |
| 192 | GGACCCCU G CUCGUGUU | 1455 | AACACGAG UGAUG GCAUGCACUAUGC GCG AGGGGUCC | 3964 |
| 222 | UUCUUGUU G ACAAAAAU | 1456 | AUUUUUGU UGAUG GCAUGCACUAUGC GCG AACAAGAA | 3965 |
| 315 | CAAAAUUC G CAGUCCCA | 1457 | UGGGACUG UGAUG GCAUGCACUAUGC GCG GAAUUUUG | 3966 |
| 374 | UGGUUAUC G CUGGAUGU | 1458 | ACAUCCAG UGAUG GCAUGCACUAUGC GCG GAUAACCA | 3967 |
| 387 | AUGUGUCU G CGGCGUUU | 1459 | AAACGCCG UGAUG GCAUGCACUAUGC GCG AGACACAU | 3968 |
| 410 | CUUCCUCU G CAUCCUGC | 1460 | GCAGGAUG UGAUG GCAUGCACUAUGC GCG AGAGGAAG | 3969 |
| 417 | UGCAUCCU G CUGCUAUG | 1461 | CAUAGCAG UGAUG GCAUGCACUAUGC GCG AGGAUGCA | 3970 |
| 420 | AUCCUGCU G CUAUGCCU | 1462 | AGGCAUAG UGAUG GCAUGCACUAUGC GCG AGCAGGAU | 3971 |
| 425 | GCUGCUAU G CCUCAUCU | 1463 | AGAUGAGG UGAUG GCAUGCACUAUGC GCG AUAGCAGC | 3972 |
| 468 | GGUAUGUU G CCCGUUUG | 1464 | CAAACGGG UGAUG GCAUGCACUAUGC GCG AACAUACC | 3973 |
| 518 | CGGACCAU G CAAAACCU | 1465 | AGGUUUUG UGAUG GCAUGCACUAUGC GCG AUGGUCCG | 3974 |
| 527 | CAAAACCU G CACAACUC | 1466 | GAGUUGUG UGAUG GCAUGCACUAUGC GCG AGGUUUUG | 3975 |
| 538 | CAACUCCU G CUCAAGGA | 1467 | UCCUUGAG UGAUG GCAUGCACUAUGC GCG AGGAGUUG | 3976 |
| 569 | CUCAUGUU G CUGUACAA | 1468 | UUGUACAG UGAUG GCAUGCACUAUGC GCG AACAUGAG | 3977 |
| 596 | CGGAAACU G CACCUGUA | 1469 | UACAGGUG UGAUG GCAUGCACUAUGC GCG AGUUUCCG | 3978 |
| 631 | GGGCUUUC G CAAAAUAC | 1470 | GUAUUUUG UGAUG GCAUGCACUAUGC GCG GAAAGCCC | 3979 |
| 687 | UUACUAGU G CCAUUUGU | 1471 | ACAAAUGG UGAUG GCAUGCACUAUGC GCG ACUAGUAA | 3980 |
| 747 | AUAUGGAU G AUGUGGUU | 1472 | AACCACAU UGAUG GCAUGCACUAUGC GCG AUCCAUAU | 3981 |
| 783 | AACAUCUU G AGUCCCUU | 1473 | AAGGGACU UGAUG GCAUGCACUAUGC GCG AAGAUGUU | 3982 |
| 795 | CCCUUUAU G CCGCUGUU | 1474 | AACAGCGG UGAUG GCAUGCACUAUGC GCG AUAAAGGG | 3983 |
| 798 | UUUAUGCC G CUGUUACC | 1475 | GGUAACAG UGAUG GCAUGCACUAUGC GCG GGCAUAAA | 3984 |
| 911 | GGCACAUU G CCACAGGA | 1476 | UCCUGUGG UGAUG GCAUGCACUAUGC GCG AAUGUGCC | 3985 |
| 978 | GGCCUAUU G AUUGGAAA | 1477 | UUUCCAAU UGAUG GCAUGCACUAUGC GCG AAUAGGCC | 3986 |
| 997 | AUGUCAAC G AAUUGUGG | 1478 | CCACAAUU UGAUG GCAUGCACUAUGC GCG GUUGACAU | 3987 |
| 1020 | UGGGGUUU G CCGCCCCU | 1479 | AGGGGCGG UGAUG GCAUGCACUAUGC GCG AAACCCCA | 3988 |
| 1023 | GGUUUGCC G CCCCUUUC | 1480 | GAAAGGGG UGAUG GCAUGCACUAUGC GCG GGCAAACC | 3989 |
| 1034 | CCUUUCAC G CAAUGUGG | 1481 | CCACAUUG UGAUG GCAUGCACUAUGC GCG GUGAAAGG | 3990 |
| 1050 | GAUAUUCU G CUUUAAUG | 1482 | CAUUAAAG UGAUG GCAUGCACUAUGC GCG AGAAUAUC | 3991 |
| 1058 | GCUUUAAU G CCUUUAUA | 1483 | UAUAAAGG UGAUG GCAUGCACUAUGC GCG AUUAAAGC | 3992 |
| 1068 | CUUUAUAU G CAUGCAUA | 1484 | UAUGCAUG UGAUG GCAUGCACUAUGC GCG AUAUAAAG | 3993 |
| 1072 | AUAUGCAU G CAUACAAG | 1485 | CUUGUAUG UGAUG GCAUGCACUAUGC GCG AUGCAUAU | 3994 |
| 1103 | ACUUUCUC G CCAACUUA | 1486 | UAAGUUGG UGAUG GCAUGCACUAUGC GCG GAGAAAGU | 3995 |
| 1139 | CAGUAUGU G AACCUUUA | 1487 | UAAAGGUU UGAUG GCAUGCACUAUGC GCG ACAUACUG | 3996 |
| 1155 | ACCCCGUU G CUCGGCAA | 1488 | UUGCCGAG UGAUG GCAUGCACUAUGC GCG AACGGGGU | 3997 |
| 1177 | UGGUCUAU G CCAAGUGU | 1489 | ACACUUGG UGAUG GCAUGCACUAUGC GCG AUAGACCA | 3998 |
| 1188 | AAGUGUUU G CUGACGCA | 1490 | UGCGUCAG UGAUG GCAUGCACUAUGC GCG AAACACUU | 3999 |
| 1191 | UGUUUGCU G ACGCAACC | 1491 | GGUUGCGU UGAUG GCAUGCACUAUGC GCG AGCAAACA | 4000 |
| 1194 | UUGCUGAC G CAACCCCC | 1492 | GGGGGUUG UGAUG GCAUGCACUAUGC GCG GUCAGCAA | 4001 |
| 1234 | CCAUCAGC G CAUGCGUG | 1493 | CACGCAUG UGAUG GCAUGCACUAUGC GCG GCUGAUGG | 4002 |
| 1238 | CAGCGCAU G CGUGGAAC | 1494 | GUUCCACG UGAUG GCAUGCACUAUGC GCG AUGCGCUG | 4003 |
| 1262 | UCUCCUCU G CCGAUCCA | 1495 | UGGAUCGG UGAUG GCAUGCACUAUGC GCG AGAGGAGA | 4004 |
| 1265 | CCUCUGCC G AUCCAUAC | 1496 | GUAUGGAU UGAUG GCAUGCACUAUGC GCG GGCAGAGG | 4005 |
| 1275 | UCCAUACC G CGGAACUC | 1497 | GAGUUCCG UGAUG GCAUGCACUAUGC GCG GGUAUGGA | 4006 |
| 1290 | UCCUAGCC G CUUGUUUU | 1498 | AAAACAAG UGAUG GCAUGCACUAUGC GCG GGCUAGGA | 4007 |
| 1299 | CUUGUUUU G CUCGCAGC | 1499 | GCUGCGAG UGAUG GCAUGCACUAUGC GCG AAAACAAG | 4008 |
| 1303 | UUUUGCUC G CAGCAGGU | 1500 | ACCUGCUG UGAUG GCAUGCACUAUGC GCG GAGCAAAA | 4009 |
| 1335 | UCGGGACU G ACAAUUCU | 1501 | AGAAUUGU UGAUG GCAUGCACUAUGC GCG AGUCCCGA | 4010 |
| 1349 | UCUGUCGU G CUCUCCCG | 1502 | CGGGAGAG UGAUG GCAUGCACUAUGC GCG ACGACAGA | 4011 |
| 1357 | GCUCUCCC G CAAAUAUA | 1503 | UAUAUUUG UGAUG GCAUGCACUAUGC GCG GGGAGAGC | 4012 |
| 1382 | CCAUGGCU G CUAGGCUG | 1504 | CAGCCUAG UGAUG GCAUGCACUAUGC GCG AGCCAUGG | 4013 |
| 1392 | UAGGCUGU G CUGCCAAC | 1505 | GUUGGCAG UGAUG GCAUGCACUAUGC GCG ACAGCCUA | 4014 |
| 1395 | GCUGUGCU G CCAACUGG | 1506 | CCAGUUGG UGAUG GCAUGCACUAUGC GCG AGCACAGC | 4015 |
| 1411 | GAUCCUAC G CGGGACGU | 1507 | ACGUCCCG UGAUG GCAUGCACUAUGC GCG GUAGGAUC | 4016 |
| 1442 | CCGUCGGC G CUGAAUCC | 1508 | GGAUUCAG UGAUG GCAUGCACUAUGC GCG GCCGACGG | 4017 |
| 1445 | UCGGCGCU G AAUCCCGC | 1509 | GCGGGAUU UGAUG GCAUGCACUAUGC GCG AGCGCCGA | 4018 |
| 1452 | UGAAUCCC G CGGACGAC | 1510 | GUCGUCCG UGAUG GCAUGCACUAUGC GCG GGGAUUCA | 4019 |
| 1458 | CCGCGGAC G ACCCCUCC | 1511 | GGAGGGGU UGAUG GCAUGCACUAUGC GCG GUCCGCGG | 4020 |
| 1474 | CCGGGGCC G CUUGGGGC | 1512 | GCCCCAAG UGAUG GCAUGCACUAUGC GCG GGCCCCGG | 4021 |
| 1489 | GCUCUACC G CCCGCUUC | 1513 | GAAGCGGG UGAUG GCAUGCACUAUGC GCG GGUAGAGC | 4022 |
| 1493 | UACCGCCC G CUUCUCCG | 1514 | CGGAGAAG UGAUG GCAUGCACUAUGC GCG GGGCGGUA | 4023 |
| 1501 | GCUUCUCC G CCUAUUGU | 1515 | ACAAUAGG UGAUG GCAUGCACUAUGC GCG GGAGAAGC | 4024 |
| 1513 | AUUGUACC G ACCGUCCA | 1516 | UGGACGGU UGAUG GCAUGCACUAUGC GCG GGUACAAU | 4025 |
| 1528 | CACGGGGC G CACCUCUC | 1517 | GAGAGGUG UGAUG GCAUGCACUAUGC GCG GCCCCGUG | 4026 |
| 1542 | CUCUUUAC G CGGACUCC | 1518 | GGAGUCCG UGAUG GCAUGCACUAUGC GCG GUAAAGAG | 4027 |
| 1559 | CCGUCUGU G CCUUCUCA | 1519 | UGAGAAGG UGAUG GCAUGCACUAUGC GCG ACAGACGG | 4028 |
| 1571 | UCUCAUCU G CCGGACCG | 1520 | CGGUCCGG UGAUG GCAUGCACUAUGC GCG AGAUGAGA | 4029 |
| 1583 | GACCGUGU G CACUUCGC | 1521 | GCGAAGUG UGAUG GCAUGCACUAUGC GCG ACACGGUC | 4030 |
| 1590 | UGCACUUC G CUUCACCU | 1522 | AGGUGAAG UGAUG GCAUGCACUAUGC GCG GAAGUGCA | 4031 |
| 1601 | UCACCUCU G CACGUCGC | 1523 | GCGACGUG UGAUG GCAUGCACUAUGC GCG AGAGGUGA | 4032 |
| 1608 | UGCACGUC G CAUGGAGA | 1524 | UCUCCAUG UGAUG GCAUGCACUAUGC GCG GACGUGCA | 4033 |
| 1624 | ACCACCGU G AACGCCCA | 1525 | UGGGCGUU UGAUG GCAUGCACUAUGC GCG ACGGUGGU | 4034 |
| 1628 | CCGUGAAC G CCCACAGG | 1526 | CCUGUGGG UGAUG GCAUGCACUAUGC GCG GUUCACGG | 4035 |
| 1642 | AGGAACCU G CCCAAGGU | 1527 | ACCUUGGG UGAUG GCAUGCACUAUGC GCG AGGUUCCU | 4036 |
| 1654 | AAGGUCUU G CAUAAGAG | 1528 | CUCUUAUG UGAUG GCAUGCACUAUGC GCG AAGACCUU | 4037 |
| 1690 | AUGUCAAC G ACCGACCU | 1529 | AGGUCGGU UGAUG GCAUGCACUAUGC GCG GUUGACAU | 4038 |
| 1694 | CAACGACC G ACCUUGAG | 1530 | CUCAAGGU UGAUG GCAUGCACUAUGC GCG GGUCGUUG | 4039 |
| 1700 | CCGACCUU G AGGCAUAC | 1531 | GUAUGCCU UGAUG GCAUGCACUAUGC GCG AAGGUCGG | 4040 |
| 1730 | UGUUUAAU G AGUGGGAG | 1532 | CUCCCACU UGAUG GCAUGCACUAUGC GCG AUUAAACA | 4041 |
| 1818 | AGCACCAU G CAACUUUU | 1533 | AAAAGUUG UGAUG GCAUGCACUAUGC GCG AUGGUGCU | 4042 |
| 1835 | UCACCUCU G CCUAAUCA | 1534 | UGAUUAGG UGAUG GCAUGCACUAUGC GCG AGAGGUGA | 4043 |
| 1883 | CAAGCUGU G CCUUGGGU | 1535 | ACCCAAGG UGAUG GCAUGCACUAUGC GCG ACAGCUUG | 4044 |
| 1912 | UGGACAUU G ACCCGUAU | 1536 | AUACGGGU UGAUG GCAUGCACUAUGC GCG AAUGUCCA | 4045 |
| 1959 | UCUUUUUU G CCUUCUGA | 1537 | UCAGAAGG UGAUG GCAUGCACUAUGC GCG AAAAAAGA | 4046 |
| 1966 | UGCCUUCU G ACUUCUUU | 1538 | AAAGAAGU UGAUG GCAUGCACUAUGC GCG AGAAGGCA | 4047 |
| 1985 | UUCUAUUC G AGAUCUCC | 1539 | GGAGAUCU UGAUG GCAUGCACUAUGC GCG GAAUAGAA | 4048 |
| 1996 | AUCUCCUC G ACACCGCC | 1540 | GGCGGUGU UGAUG GCAUGCACUAUGC GCG GAGGAGAU | 4049 |
| 2002 | UCGACACC G CCUCUGCU | 1541 | AGCAGAGG UGAUG GCAUGCACUAUGC GCG GGUGUCGA | 4050 |
| 2008 | CCGCCUCU G CUCUGUAU | 1542 | AUACAGAG UGAUG GCAUGCACUAUGC GCG AGAGGCGG | 4051 |
| 2092 | GUUGGGGU G AGUUGAUG | 1543 | CAUCAACU UGAUG GCAUGCACUAUGC GCG ACCCCAAC | 4052 |
| 2097 | GGUGAGUU G AUGAAUCU | 1544 | AGAUUCAU UGAUG GCAUGCACUAUGC GCG AACUCACC | 4053 |
| 2100 | GAGUUGAU G AAUCUAGC | 1545 | GCUAGAUU UGAUG GCAUGCACUAUGC GCG AUCAACUC | 4054 |
| 2237 | UUUUGGGC G AGAAACUG | 1546 | CAGUUUCU UGAUG GCAUGCACUAUGC GCG GCCCAAAA | 4055 |
| 2251 | CUGUUCUU G AAUAUUUG | 1547 | CAAAUAUU UGAUG GCAUGCACUAUGC GCG AAGAACAG | 4056 |
| 2282 | GUGGAUUC G CACUCCUC | 1548 | GAGGAGUG UGAUG GCAUGCACUAUGC GCG GAAUCCAC | 4057 |
| 2293 | CUCCUCCU G CAUAUAGA | 1549 | UCUAUAUG UGAUG GCAUGCACUAUGC GCG AGGAGGAG | 4058 |
| 2311 | CACCAAAU G CCCCUAUC | 1550 | GAUAGGGG UGAUG GCAUGCACUAUGC GCG AUUUGGUG | 4059 |
| 2354 | UGUUAGAC G AAGAGGCA | 1551 | UGCCUCUU UGAUG GCAUGCACUAUGC GCG GUCUAACA | 4060 |
| 2388 | ACUCCCUC G CCUCGCAG | 1552 | CUGCGAGG UGAUG GCAUGCACUAUGC GCG GAGGGAGU | 4061 |
| 2393 | CUCGCCUC G CAGACGAA | 1553 | UUCGUCUG UGAUG GCAUGCACUAUGC GCG GAGGCGAG | 4062 |
| 2399 | UCGCAGAC G AAGGUCUC | 1554 | GAGACCUU UGAUG GCAUGCACUAUGC GCG GUCUGCGA | 4063 |
| 2412 | UCUCAAUC G CCGCGUCG | 1555 | CGACGCGG UGAUG GCAUGCACUAUGC GCG GAUUGAGA | 4064 |
| 2415 | CAAUCGCC G CGUCGCAG | 1556 | CUGCGACG UGAUG GCAUGCACUAUGC GCG GGCGAUUG | 4065 |
| 2420 | GCCGCGUC G CAGAAGAU | 1557 | AUCUUCUG UGAUG GCAUGCACUAUGC GCG GACGCGGC | 4066 |
| 2514 | GGUACCUU G CUUUAAUC | 1558 | GAUUAAAG UGAUG GCAUGCACUAUGC GCG AAGGUACC | 4067 |
| 2549 | CUUUUCCU G ACAUUCAU | 1559 | AUGAAUGU UGAUG GCAUGCACUAUGC GCG AGGAAAAG | 4068 |
| 2560 | AUUCAUUU G CAGGAGGA | 1560 | UCCUCCUG UGAUG GCAUGCACUAUGC GCG AAAUGAAU | 4069 |
| 2576 | ACAUUGUU G AUAGAUGU | 1561 | ACAUCUAU UGAUG GCAUGCACUAUGC GCG AACAAUGU | 4070 |
| 2615 | CAGUAAAU G AAAACAGG | 1562 | CCUGUUUU UGAUG GCAUGCACUAUGC GCG AUUUACUG | 4071 |
| 2641 | UUAACUAU G CCUGCUAG | 1563 | CUAGCAGG UGAUG GCAUGCACUAUGC GCG AUAGUUAA | 4072 |
| 2645 | CUAUGCCU G CUAGGUUU | 1564 | AAACCUAG UGAUG GCAUGCACUAUGC GCG AGGCAUAG | 4073 |
| 2677 | AAAUAUUU G CCCUUAGA | 1565 | UCUAAGGG UGAUG GCAUGCACUAUGC GCG AAAUAUUU | 4074 |
| 2740 | UUCCAGAC G CGACAUUA | 1566 | UAAUGUCG UGAUG GCAUGCACUAUGC GCG GUCUGGAA | 4075 |
| 2742 | CCAGACGC G ACAUUAUU | 1567 | AAUAAUGU UGAUG GCAUGCACUAUGC GCG GCGUCUGG | 4076 |
| 2804 | CACGUAGC G CCUCAUUU | 1568 | AAAUGAGG UGAUG GCAUGCACUAUGC GCG GCUACGUG | 4077 |
| 2814 | CUCAUUUU G CGGGUCAC | 1569 | GUGACCCG UGAUG GCAUGCACUAUGC GCG AAAAUGAG | 4078 |
| 2875 | CAAACCUC G AAAAGGCA | 1570 | UGCCUUUU UGAUG GCAUGCACUAUGC GCG GAGGUUUG | 4079 |
| 2928 | UCUUCCCC G AUCAUCAG | 1571 | CUGAUGAU UGAUG GCAUGCACUAUGC GCG GGGGAAGA | 4080 |
| 2946 | UGGACCCU G CAUUCAAA | 1572 | UUUGAAUG UGAUG GCAUGCACUAUGC GCG AGGGUCCA | 4081 |
| 2990 | CUCAACCC G CACAAGGA | 1573 | UCCUUGUG UGAUG GCAUGCACUAUGC GCG GGGUUGAG | 4082 |
| 3012 | GGCCGGAC G CCAACAAG | 1574 | CUUGUUGG UGAUG GCAUGCACUAUGC GCG GUCCGGCC | 4083 |
| 3090 | GCCCUCAC G CUCAGGGC | 1575 | GCCCUGAG UGAUG GCAUGCACUAUGC GCG GUGAGGGC | 4084 |
| 3113 | ACAACUGU G CCAGCAGC | 1576 | GCUGCUGG UGAUG GCAUGCACUAUGC GCG ACAGUUGU | 4085 |
| 3132 | CUCCUCCU G CCUCCACC | 1577 | GGUGGAGG UGAUG GCAUGCACUAUGC GCG AGGAGGAG | 4086 |
| 51 | AGGGCCCU G UACUUUCC | 1578 | GGAAAGUA UGAUG GCAUGCACUAUGC GCG AGGGCCCU | 4087 |
| 106 | AGAAUACU G UCUCUGCC | 1579 | GGCAGAGA UGAUG GCAUGCACUAUGC GCG AGUAUUCU | 4088 |
| 148 | GGGACCCU G UACCGAAC | 1580 | GUUCGGUA UGAUG GCAUGCACUAUGC GCG AGGGUCCC | 4089 |
| 198 | CUGCUCGU G UUACAGGC | 1581 | GCCUGUAA UGAUG GCAUGCACUAUGC GCG ACGAGCAG | 4090 |
| 219 | UUUUUCUU G UUGACAAA | 1582 | UUUGUCAA UGAUG GCAUGCACUAUGC GCG AAGAAAAA | 4091 |
| 297 | ACACCCGU G UGUCUUGG | 1583 | CCAAGACA UGAUG GCAUGCACUAUGC GCG ACGGGUGU | 4092 |
| 299 | ACCCGUGU G UCUUGGCC | 1584 | GGCCAAGA UGAUG GCAUGCACUAUGC GCG ACACGGGU | 4093 |
| 347 | ACCAACCU G UUGUCCUC | 1585 | GAGGACAA UGAUG GCAUGCACUAUGC GCG AGGUUGGU | 4094 |
| 350 | AACCUGUU G UCCUCCAA | 1586 | UUGGAGGA UGAUG GCAUGCACUAUGC GCG AACAGGUU | 4095 |
| 362 | UCCAAUUU G UCCUGGUU | 1587 | AACCAGGA UGAUG GCAUGCACUAUGC GCG AAAUUGGA | 4096 |
| 381 | CGCUGGAU G UGUCUGCG | 1588 | CGCAGACA UGAUG GCAUGCACUAUGC GCG AUCCAGCG | 4097 |
| 383 | CUGGAUGU G UCUGCGGC | 1589 | GCCGCAGA UGAUG GCAUGCACUAUGC GCG ACAUCCAG | 4098 |
| 438 | AUCUUCUU G UUGGUUCU | 1590 | AGAACCAA UGAUG GCAUGCACUAUGC GCG AAGAAGAU | 4099 |
| 465 | CAAGGUAU G UUGCCCGU | 1591 | ACGGGCAA UGAUG GCAUGCACUAUGC GCG AUACCUUG | 4100 |
| 476 | GCCCGUUU G UCCUCUAA | 1592 | UUAGAGGA UGAUG GCAUGCACUAUGC GCG AAACGGGC | 4101 |
| 555 | ACCUCUAU G UUUCCCUC | 1593 | GAGGGAAA UGAUG GCAUGCACUAUGC GCG AUAGAGGU | 4102 |
| 566 | UCCCUCAU G UUGCUGUA | 1594 | UACAGCAA UGAUG GCAUGCACUAUGC GCG AUGAGGGA | 4103 |
| 572 | AUGUUGCU G UACAAAAC | 1595 | GUUUUGUA UGAUG GCAUGCACUAUGC GCG AGCAACAU | 4104 |
| 602 | CUGCACCU G UAUUCCCA | 1596 | UGGGAAUA UGAUG GCAUGCACUAUGC GCG AGGUGCAG | 4105 |
| 694 | UGCCAUUU G UUCAGUGG | 1597 | CCACUGAA UGAUG GCAUGCACUAUGC GCG AAAUGGCA | 4106 |
| 724 | CCCCCACU G UCUGGCUU | 1598 | AAGCCAGA UGAUG GCAUGCACUAUGC GCG AGUGGGGG | 4107 |
| 750 | UGGAUGAU G UGGUUUUG | 1599 | CAAAACCA UGAUG GCAUGCACUAUGC GCG AUCAUCCA | 4108 |
| 771 | CCAAGUCU G UACAACAU | 1600 | AUGUUGUA UGAUG GCAUGCACUAUGC GCG AGACUUGG | 4109 |
| 801 | AUGCCGCU G UUACCAAU | 1601 | AUUGGUAA UGAUG GCAUGCACUAUGC GCG AGCGGCAU | 4110 |
| 818 | UUUCUUUU G UCUUUGGG | 1602 | CCCAAAGA UGAUG GCAUGCACUAUGC GCG AAAAGAAA | 4111 |
| 888 | UGGGAUAU G UAAUUGGG | 1603 | CCCAAUUA UGAUG GCAUGCACUAUGC GCG AUAUCCCA | 4112 |
| 927 | AACAUAUU G UACAAAAA | 1604 | UUUUUGUA UGAUG GCAUGCACUAUGC GCG AAUAUGUU | 4113 |
| 944 | AUCAAAAU G UGUUUUAG | 1605 | CUAAAACA UGAUG GCAUGCACUAUGC GCG AUUUUGAU | 4114 |
| 946 | CAAAAUGU G UUUUAGGA | 1606 | UCCUAAAA UGAUG GCAUGCACUAUGC GCG ACAUUUUG | 4115 |
| 963 | AACUUCCU G UAAACAGG | 1607 | CCUGUUUA UGAUG GCAUGCACUAUGC GCG AGGAAGUU | 4116 |
| 991 | GAAAGUAU G UCAACGAA | 1608 | UUCGUUGA UGAUG GCAUGCACUAUGC GCG AUACUUUC | 4117 |
| 1002 | AACGAAUU G UGGGUCUU | 1609 | AAGACCCA UGAUG GCAUGCACUAUGC GCG AAUUCGUU | 4118 |
| 1039 | CACGCAAU G UGGAUAUU | 1610 | AAUAUCCA UGAUG GCAUGCACUAUGC GCG AUUGCGUG | 4119 |
| 1137 | AACAGUAU G UGAACCUU | 1611 | AAGGUUCA UGAUG GCAUGCACUAUGC GCG AUACUGUU | 4120 |
| 1184 | UGCCAAGU G UUUGCUGA | 1612 | UCAGCAAA UGAUG GCAUGCACUAUGC GCG ACUUGGCA | 4121 |
| 1251 | GAACCUUU G UGUCUCCU | 1613 | AGGAGACA UGAUG GCAUGCACUAUGC GCG AAAGGUUC | 4122 |
| 1253 | ACCUUUGU G UCUCCUCU | 1614 | AGAGGAGA UGAUG GCAUGCACUAUGC GCG ACAAAGGU | 4123 |
| 1294 | AGCCGCUU G UUUUGCUC | 1615 | GAGCAAAA UGAUG GCAUGCACUAUGC GCG AAGCGGCU | 4124 |
| 1344 | ACAAUUCU G UCGUGCUC | 1616 | GAGCACGA UGAUG GCAUGCACUAUGC GCG AGAAUUGU | 4125 |
| 1390 | GCUAGGCU G UGCUGCCA | 1617 | UGGCAGCA UGAUG GCAUGCACUAUGC GCG AGCCUAGC | 4126 |
| 1425 | CGUCCUUU G UUUACGUC | 1618 | GACGUAAA UGAUG GCAUGCACUAUGC GCG AAAGGACG | 4127 |
| 1508 | CGCCUAUU G UACCGACC | 1619 | GGUCGGUA UGAUG GCAUGCACUAUGC GCG AAUAGGCG | 4128 |
| 1557 | CCCCGUCU G UGCCUUCU | 1620 | AGAAGGCA UGAUG GCAUGCACUAUGC GCG AGACGGGG | 4129 |
| 1581 | CGGACCGU G UGCACUUC | 1621 | CAAGUGCA UGAUG GCAUGCACUAUGC GCG ACGGUCCG | 4130 |
| 1684 | UCAGCAAU G UCAACGAC | 1622 | GUCGUUGA UGAUG GCAUGCACUAUGC GCG AUUGCUGA | 4131 |
| 1719 | CAAAGACU G UGUGUUUA | 1623 | UAAACACA UGAUG GCAUGCACUAUGC GCG AGUCUUUG | 4132 |
| 1721 | AAGACUGU G UGUUUAAU | 1624 | AUUAAACA UGAUG GCAUGCACUAUGC GCG ACAGUCUU | 4133 |
| 1723 | GACUGUGU G UUUAAUGA | 1625 | UCAUUAAA UGAUG GCAUGCACUAUGC GCG ACACAGUC | 4134 |
| 1772 | AGGUCUUU G UACUAGGA | 1626 | UCCUAGUA UGAUG GCAUGCACUAUGC GCG AAAGACCU | 4135 |
| 1785 | AGGAGGCU G UAGGCAUA | 1627 | UAUGCCUA UGAUG GCAUGCACUAUGC GCG AGCCUCCU | 4136 |
| 1801 | AAAUUGGU G UGUUCACC | 1628 | GGUGAACA UGAUG GCAUGCACUAUGC GCG ACCAAUUU | 4137 |
| 1803 | AUUGGUGU G UUCACCAG | 1629 | CUGGUGAA UGAUG GCAUGCACUAUGC GCG ACACCAAU | 4138 |
| 1850 | CAUCUCAU G UUCAUGUC | 1630 | GACAUGAA UGAUG GCAUGCACUAUGC GCG AUGAGAUG | 4139 |
| 1856 | AUGUUCAU G UCCUACUG | 1631 | CAGUAGGA UGAUG GCAUGCACUAUGC GCG AUGAACAU | 4140 |
| 1864 | GUCCUACU G UUCAAGCC | 1632 | GGCUUGAA UGAUG GCAUGCACUAUGC GCG AGUAGGAC | 4141 |
| 1881 | UCCAAGCU G UGCCUUGG | 1633 | CCAAGGCA UGAUG GCAUGCACUAUGC GCG AGCUUGGA | 4142 |
| 1939 | GAGCUUCU G UGGAGUUA | 1634 | UAACUCCA UGAUG GCAUGCACUAUGC GCG AGAAGCUC | 4143 |
| 2013 | UCUGCUCU G UAUCGGGG | 1635 | CCCCGAUA UGAUG GCAUGCACUAUGC GCG AGAGCAGA | 4144 |
| 2045 | GGAACAUU G UUCACCUC | 1636 | GAGGUGAA UGAUG GCAUGCACUAUGC GCG AAUGUUCC | 4145 |
| 2082 | GCUAUUCU G UGUUGGGG | 1637 | CCCCAACA UGAUG GCAUGCACUAUGC GCG AGAAUAGC | 4146 |
| 2084 | UAUUCUGU G UUGGGGUG | 1638 | CACCCCAA UGAUG GCAUGCACUAUGC GCG ACAGAAUA | 4147 |
| 2167 | UCAGCUAU G UCAACGUU | 1639 | AACGUUGA UGAUG GCAUGCACUAUGC GCG AUAGCUGA | 4148 |
| 2205 | CAACUAUU G UGGUUUCA | 1640 | UGAAACCA UGAUG GCAUGCACUAUGC GCG AAUAGUUG | 4149 |
| 2222 | CAUUUCCU G UCUUACUU | 1641 | AAGUAAGA UGAUG GCAUGCACUAUGC GCG AGGAAAUG | 4150 |
| 2245 | GAGAAACU G UUCUUGAA | 1642 | UUCAAGAA UGAUG GCAUGCACUAUGC GCG AGUUUCUC | 4151 |
| 2262 | UAUUUGGU G UCUUUUGG | 1643 | CCAAAAGA UGAUG GCAUGCACUAUGC GCG ACCAAAUA | 4152 |
| 2274 | UUUGGAGU G UGGAUUCG | 1644 | CGAAUCCA UGAUG GCAUGCACUAUGC GCG ACUCCAAA | 4153 |
| 2344 | AAACUACU G UUGUUAGA | 1645 | UCUAACAA UGAUG GCAUGCACUAUGC GCG AGUAGUUU | 4154 |
| 2347 | CUACUGUU G UUAGACGA | 1646 | UCGUCUAA UGAUG GCAUGCACUAUGC GCG AACAGUAG | 4155 |
| 2450 | AUCUCAAU G UUAGUAUU | 1647 | AAUACUAA UGAUG GCAUGCACUAUGC GCG AUUGAGAU | 4156 |
| 2573 | AGGACAUU G UUGAUAGA | 1648 | UCUAUCAA UGAUG GCAUGCACUAUGC GCG AAUGUCCU | 4157 |
| 2583 | UGAUAGAU G UAAGCAAU | 1649 | AUUGCUUA UGAUG GCAUGCACUAUGC GCG AUCUAUCA | 4158 |
| 2594 | AGCAAUUU G UGGGGCCC | 1650 | GGGCCCCA UGAUG GCAUGCACUAUGC GCG AAAUUGCU | 4159 |
| 2663 | AUCCCAAU G UUACUAAA | 1651 | UUUAGUAA UGAUG CCAUGCACUAUGC GCG AUUGGGAU | 4160 |
| 2717 | CAGAGUAU G UAGUUAAU | 1652 | AUUAACUA UGAUG GCAUGCACUAUGC GCG AUACUCUG | 4161 |
| 2901 | AUCUUUCU G UCCCCAAU | 1653 | AUUGGGGA UGAUG GCAUGCACUAUGC GCG AGAAAGAU | 4162 |
| 3071 | GGGGGACU G UUGGGGUG | 1654 | CACCCCAA UGAUG GCAUGCACUAUGC GCG AGUCCCCC | 4163 |
| 3111 | UCACAACU G UGCCAGCA | 1655 | UGCUGGCA UGAUG GCAUGCACUAUGC GCG AGUUGUGA | 4164 |

Input Sequence = AF100308. Cut Site = YG/M or UG/U.
Stem Length = 8. Core Sequence = UGAUG GCAUGCACUAUgC GCG
AF100308 (Hepatitis B virus strain 2-18, 3215 bp)

TABLE VIII

HUMAN HBV ZINZYME AND SUBSTRATE SEQUENCE

| | | | | |
|---|---|---|---|---|
| Pos | Substrate | Seq ID | ZinZyme | Seq ID |

| | | | | |
|---|---|---|---|---|
| 61 | ACUUUCCU G CUGGUGGC | 1448 | GCCACCAG GCcgaaagGCGaGuCaaGGuCu AGGAAAGU | 4165 |
| 94 | UGAGCCCU G CUCAGAAU | 1450 | AUUCUGAG GCcgaaagGCGaGuCaaGGuCu AGGGCUCA | 4166 |
| 112 | CUGUCUCU G CCAUAUCG | 1451 | CGAUAUGG GCcgaaagGCGaGuCaaGGuCu AGAGACAG | 4167 |
| 169 | AGAACAUC G CAUCAGGA | 1454 | UCCUGAUG GCcgaaagGCGaGuCaaGGuCu GAUGUUCU | 4168 |
| 192 | GGACCCCU G CUCGUGUU | 1455 | AACACGAG GCcgaaagGCGaGuCaaGGuCu AGGGGUCC | 4169 |
| 315 | CAAAAUUC G CAGUCCCA | 1457 | UGGGACUG GCcgaaagGCGaGuCaaGGuCu GAAUUUUG | 4170 |
| 374 | UGGUUAUC G CUGGAUGU | 1458 | ACAUCCAG GCcgaaagGCGaGuCaaGGuCu GAUAACCA | 4171 |
| 387 | AUGUGUCU G CGGCGUUU | 1459 | AAACGCCG GCcgaaagGCGaGuCaaGGuCu AGACACAU | 4172 |
| 410 | CUUCCUCU G CAUCCUGC | 1460 | GCAGGAUG GCcgaaagGCGaGuCaaGGuCu AGAGGAAG | 4173 |
| 417 | UGCAUCCU G CUGCUAUG | 1461 | CAUAGCAG GCcgaaagGCGaGuCaaGGuCu AGGAUGCA | 4174 |
| 420 | AUCCUGCU G CUAUGCCU | 1462 | AGGCAUAG GCcgaaagGCGaGuCaaGGuCu AGCAGGAU | 4175 |
| 425 | GCUGCUAU G CCUCAUCU | 1463 | AGAUGAGG GCcgaaagGCGaGuCaaGGuCu AUAGCAGC | 4176 |
| 468 | GGUAUGUU G CCCGUUUG | 1464 | CAAACGGG GCcgaaagGCGaGuCaaGGuCu AACAUACC | 4177 |
| 518 | CGGACCAU G CAAAACCU | 1465 | AGGUUUUG GCcgaaagGCGaGuCaaGGuCu AUGGUCCG | 4178 |
| 527 | CAAAACCU G CACAACUC | 1466 | GAGUUGUG GCcgaaagGCGaGuCaaGGuCu AGGUUUUG | 4179 |
| 538 | CAACUCCU G CUCAAGGA | 1467 | UCCUUGAG GCcgaaagGCGaGuCaaGGuCu AGGAGUUG | 4180 |
| 569 | CUCAUGUU G CUGUACAA | 1468 | UUGUACAG GCcgaaagGCGaGuCaaGGuCu AACAUGAG | 4181 |
| 596 | CGGAAACU G CACCUGUA | 1469 | UACAGGUG GCcgaaagGCGaGuCaaGGuCu AGUUUCCG | 4182 |
| 631 | GGGCUUUC G CAAAAUAC | 1470 | GUAUUUUG GCcgaaagGCGaGuCaaGGuCu GAAAGCCC | 4183 |
| 687 | UUACUAGU G CCAUUUGU | 1471 | ACAAAUGG GCcgaaagGCGaGuCaaGGuCu ACUAGUAA | 4184 |
| 795 | CCCUUUAU G CCGCUGUU | 1474 | AACAGCGG GCcgaaagGCGaGuCaaGGuCu AUAAAGGG | 4185 |
| 798 | UUUAUGCC G CUGUUACC | 1475 | GGUAACAG GCcgaaagGCGaGuCaaGGuCu GGCAUAAA | 4186 |
| 911 | GGCACAUU G CCACAGGA | 1476 | UCCUGUGG GCcgaaagGCGaGuCaaGGuCu AAUGUGCC | 4187 |
| 1020 | UGGGGUUU G CCGCCCCU | 1479 | AGGGGCGG GCcgaaagGCGaGuCaaGGuCu AAACCCCA | 4188 |
| 1023 | GGUUUGCC G CCCCUUUC | 1480 | GAAAGGGG GCcgaaagGCGaGuCaaGGuCu GGCAAACC | 4189 |
| 1034 | CCUUUCAC G CAAUGUGG | 1481 | CCACAUUG GCcgaaagGCGaGuCaaGGuCu GUGAAAGG | 4190 |
| 1050 | GAUAUUCU G CUUUAAUG | 1482 | CAUUAAAG GCcgaaagGCGaGuCaaGGuCu AGAAUAUC | 4191 |
| 1058 | GCUUUAAU G CCUUUAUA | 1483 | UAUAAAGG GCcgaaagGCGaGuCaaGGuCu AUUAAAGC | 4192 |
| 1068 | CUUUAUAU G CAUGCAUA | 1484 | UAUGCAUG GCcgaaagGCGaGuCaaGGuCu AUAUAAAG | 4193 |
| 1072 | AUAUGCAU G CAUACAAG | 1485 | CUUGUAUG GCcgaaagGCGaGuCaaGGuCu AUGCAUAU | 4194 |
| 1103 | ACUUUCUC G CCAACUUA | 1486 | UAAGUUGG GCcgaaagGCGaGuCaaGGuCu GAGAAAGU | 4195 |
| 1155 | ACCCCGUU G CUCGGCAA | 1488 | UUGCCGAG GCcgaaagGCGaGuCaaGGuCu AACGGGGU | 4196 |
| 1177 | UGGUCUAU G CCAAGUGU | 1489 | ACACUUGG GCcgaaagGCGaGuCaaGGuCu AUAGACCA | 4197 |
| 1188 | AAGUGUUU G CUGACGCA | 1490 | UGCGUCAG GCcgaaagGCGaGuCaaGGuCu AAACACUU | 4198 |
| 1194 | UUGCUGAC G CAACCCCC | 1492 | GGGGGUUG GCcgaaagGCGaGuCaaGGuCu GUCAGCAA | 4199 |
| 1234 | CCAUCAGC G CAUGCGUG | 1493 | CACGCAUG GCcgaaagGCGaGuCaaGGuCu GCUGAUGG | 4200 |
| 1238 | CAGCGCAU G CGUGGAAC | 1494 | GUUCCACG GCcgaaagGCGaGuCaaGGuCu AUGCGCUG | 4201 |
| 1262 | UCUCCUCU G CCGAUCCA | 1495 | UGGAUCGG GCcgaaagGCGaGuCaaGGuCu AGAGGAGA | 4202 |
| 1275 | UCCAUACC G CGGAACUC | 1497 | GAGUUCCG GCcgaaagGCGaGuCaaGGuCu GGUAUGGA | 4203 |
| 1290 | UCCUAGCC G CUUGUUUU | 1498 | AAAACAAG GCcgaaagGCGaGuCaaGGuCu GGCUAGGA | 4204 |
| 1299 | CUUGUUUU G CUCGCAGC | 1499 | GCUGCGAG GCcgaaagGCGaGuCaaGGuCu AAAACAAG | 4205 |
| 1303 | UUUUGCUC G CAGCAGGU | 1500 | ACCUGCUG GCcgaaagGCGaGuCaaGGuCu GAGCAAAA | 4206 |
| 1349 | UCUGUCGU G CUCUCCCG | 1502 | CGGGAGAG GCcgaaagGCGaGuCaaGGuCu ACGACAGA | 4207 |
| 1357 | GCUCUCCC G CAAAUAUA | 1503 | UAUAUUUG GCcgaaagGCGaGuCaaGGuCu GGGAGAGC | 4208 |
| 1382 | CCAUGGCU G CUAGGCUG | 1504 | CAGCCUAG GCcgaaagGCGaGuCaaGGuCu AGCCAUGG | 4209 |
| 1392 | UAGGCUGU G CUGCCAAC | 1505 | GUUGGCAG GCcgaaagGCGaGuCaaGGuCu ACAGCCUA | 4210 |
| 1395 | GCUGUGCU G CCAACUGG | 1506 | CCAGUUGG GCcgaaagGCGaGuCaaGGuCu AGCACAGC | 4211 |
| 1411 | GAUCCUAC G CGGGACGU | 1507 | ACGUCCCG GCcgaaagGCGaGuCaaGGuCu GUAGGAUC | 4212 |
| 1442 | CCGUCGGC G CUGAAUCC | 1508 | GGAUUCAG GCcgaaagGCGaGuCaaGGuCu GCCGACGG | 4213 |
| 1452 | UGAAUCCC G CGGACGAC | 1510 | GUCGUCCG GCcgaaagGCGaGuCaaGGuCu GGGAUUCA | 4214 |
| 1474 | CCGGGGCC G CUUGGGGC | 1512 | GCCCCAAG GCcgaaagGCGaGuCaaGGuCu GGCCCCGG | 4215 |
| 1489 | GCUCUACC G CCCGCUUC | 1513 | GAAGCGGG GCcgaaagGCGaGuCaaGGuCu GGUAGAGC | 4216 |
| 1493 | UACCGCCC G CUUCUCCG | 1514 | CGGAGAAG GCcgaaagGCGaGuCaaGGuCu GGGCGGUA | 4217 |
| 1501 | GCUUCUCC G CCUAUUGU | 1515 | ACAAUAGG GCcgaaagGCGaGuCaaGGuCu GGAGAAGC | 4218 |
| 1528 | CACGGGGC G CACCUCUC | 1517 | GAGAGGUG GCcgaaagGCGaGuCaaGGuCu GCCCCGUG | 4219 |
| 1542 | CUCUUUAC G CGGACUCC | 1518 | GGAGUCCG GCcgaaagGCGaGuCaaGGuCu GUAAAGAG | 4220 |
| 1559 | CCGUCUGU G CCUUCUCA | 1519 | UGAGAAGG GCcgaaagGCGaGuCaaGGuCu ACAGACGG | 4221 |
| 1571 | UCUCAUCU G CCGGACCG | 1520 | CGGUCCGG GCcgaaagGCGaGuCaaGGuCu AGAUGAGA | 4222 |
| 1583 | GACCGUGU G CACUUCGC | 1521 | GCGAAGUG GCcgaaagGCGaGuCaaGGuCu ACACGGUC | 4223 |
| 1590 | UGCACUUC G CUUCACCU | 1522 | AGGUGAAG GCcgaaagGCGaGuCaaGGuCu GAAGUGCA | 4224 |
| 1601 | UCACCUCU G CACGUCGC | 1523 | GCGACGUG GCcgaaagGCGaGuCaaGGuCu AGAGGUGA | 4225 |
| 1608 | UGCACGUC G CAUGGAGA | 1524 | UCUCCAUG GCcgaaagGCGaGuCaaGGuCu GACGUGCA | 4226 |
| 1628 | CCGUGAAC G CCCACAGG | 1526 | CCUGUGGG GCcgaaagGCGaGuCaaGGuCu GUUCACGG | 4227 |
| 1642 | AGGAACCU G CCCAAGGU | 1527 | ACCUUGGG GCcgaaagGCGaGuCaaGGuCu AGGUUCCU | 4228 |
| 1654 | AAGGUCUU G CAUAAGAG | 1528 | CUCUUAUG GCcgaaagGCGaGuCaaGGuCu AAGACCUU | 4229 |
| 1818 | AGCACCAU G CAACUUUU | 1533 | AAAAGUUG GCcgaaagGCGaGuCaaGGuCu AUGGUGCU | 4230 |
| 1835 | UCACCUCU G CCUAAUCA | 1534 | UGAUUAGG GCcgaaagGCGaGuCaaGGuCu AGAGGUGA | 4231 |
| 1883 | CAAGCUGU G CCUUGGGU | 1535 | ACCCAAGG GCcgaaagGCGaGuCaaGGuCu ACAGCUUG | 4232 |
| 1959 | UCUUUUUU G CCUUCUGA | 1537 | UCAGAAGG GCcgaaagGCGaGuCaaGGuCu AAAAAAGA | 4233 |
| 2002 | UCGACACC G CCUCUGCU | 1541 | AGCAGAGG GCcgaaagGCGaGuCaaGGuCu GGUGUCGA | 4234 |
| 2008 | CCGCCUCU G CUCUGUAU | 1542 | AUACAGAG GCcgaaagGCGaGuCaaGGuCu AGAGGCGG | 4235 |
| 2282 | GUGGAUUC G CACUCCUC | 1548 | GAGGAGUG GCcgaaagGCGaGuCaaGGuCu GAAUCCAC | 4236 |
| 2293 | CUCCUCCU G CAUAUAGA | 1549 | UCUAUAUG GCcgaaagGCGaGuCaaGGuCu AGGAGGAG | 4237 |
| 2311 | CACCAAAU G CCCCUAUC | 1550 | GAUAGGGG GCcgaaagGCGaGuCaaGGuCu AUUUGGUG | 4238 |
| 2388 | ACUCCCUC G CCUCGCAG | 1552 | CUGCGAGG GCcgaaagGCGaGuCaaGGuCu GAGGGAGU | 4239 |
| 2393 | CUCGCCUC G CAGACGAA | 1553 | UUCGUCUG GCcgaaagGCGaGuCaaGGuCu GAGGCGAG | 4240 |
| 2412 | UCUCAAUC G CCGCGUCG | 1555 | CGACGCGG GCcgaaagGCGaGuCaaGGuCu GAUUGAGA | 4241 |
| 2415 | CAAUCGCC G CGUCGCAG | 1556 | CUGCGACG GCcgaaagGCGaGuCaaGGuCu GGCGAUUG | 4242 |
| 2420 | GCCGCGUC G CAGAAGAU | 1557 | AUCUUCUG GCcgaaagGCGaGuCaaGGuCu GACGCGGC | 4243 |
| 2514 | GGUACCUU G CUUUAAUC | 1558 | GAUUAAAG GCcgaaagGCGaGuCaaGGuCu AAGGUACC | 4244 |
| 2560 | AUUCAUUU G CAGGAGGA | 1560 | UCCUCCUG GCcgaaagGCGaGuCaaGGuCu AAAUGAAU | 4245 |
| 2641 | UUAACUAU G CCUGCUAG | 1563 | CUAGCAGG GCcgaaagGCGaGuCaaGGuCu AUAGUUAA | 4246 |
| 2645 | CUAUGCCU G CUAGGUUU | 1564 | AAACCUAG GCcgaaagGCGaGuCaaGGuCu AGGCAUAG | 4247 |
| 2677 | AAAUAUUU G CCCUUAGA | 1565 | UCUAAGGG GCcgaaagGCGaGuCaaGGuCu AAAUAUUU | 4248 |
| 2740 | UUCCAGAC G CGACAUUA | 1566 | UAAUGUCG GCcgaaagGCGaGuCaaGGuCu GUCUGGAA | 4249 |
| 2804 | CACGUAGC G CCUCAUUU | 1568 | AAAUGAGG GCcgaaagGCGaGuCaaGGuCu GCUACGUG | 4250 |
| 2814 | CUCAUUUU G CGGGUCAC | 1569 | GUGACCCG GCcgaaagGCGaGuCaaGGuCu AAAAUGAG | 4251 |
| 2946 | UGGACCCU G CAUUCAAA | 1572 | UUUGAAUG GCcgaaagGCGaGuCaaGGuCu AGGGUCCA | 4252 |
| 2990 | CUCAACCC G CACAAGGA | 1573 | UCCUUGUG GCcgaaagGCGaGuCaaGGuCu GGGUUGAG | 4253 |
| 3012 | GGCCGGAC G CCAACAAG | 1574 | CUUGUUGG GCcgaaagGCGaGuCaaGGuCu GUCCGGCC | 4254 |
| 3090 | GCCCUCAC G CUCAGGGC | 1575 | GCCCUGAG GCcgaaagGCGaGuCaaGGuCu GUGAGGGC | 4255 |
| 3113 | ACAACUGU G CCAGCAGC | 1576 | GCUGCUGG GCcgaaagGCGaGuCaaGGuCu ACAGUUGU | 4256 |
| 3132 | CUCCUCCU G CCUCCACC | 1577 | GGUGGAGG GCcgaaagGCGaGuCaaGGuCu AGGAGGAG | 4257 |
| 51 | AGGGCCCU G UACUUUCC | 1578 | GGAAAGUA GCcgaaagGCGaGuCaaGGuCu AGGGCCCU | 4258 |
| 106 | AGAAUACU G UCUCUGCC | 1579 | GGCAGAGA GCcgaaagGCGaGuCaaGGuCu AGUAUUCU | 4259 |
| 148 | GGGACCCU G UACCGAAC | 1580 | GUUCGGUA GCcgaaagGCGaGuCaaGGuCu AGGGUCCC | 4260 |
| 198 | CUGCUCGU G UUACAGGC | 1581 | GCCUGUAA GCcgaaagGCGaGuCaaGGuCu ACGAGCAG | 4261 |
| 219 | UUUUUCUU G UUGACAAA | 1582 | UUUGUCAA GCcgaaagGCGaGuCaaGGuCu AAGAAAAA | 4262 |
| 297 | ACACCCGU G UGUCUUGG | 1583 | CCAAGACA GCcgaaagGCGaGuCaaGGuCu ACGGGUGU | 4263 |
| 299 | ACCCGUGU G UCUUGGCC | 1584 | GGCCAAGA GCcgaaagGCGaGuCaaGGuCu ACACGGGU | 4264 |
| 347 | ACCAACCU G UUGUCCUC | 1585 | GAGGACAA GCcgaaagGCGaGuCaaGGuCu AGGUUGGU | 4265 |
| 350 | AACCUGUU G UCCUCCAA | 1586 | UUGGAGGA GCcgaaagGCGaGuCaaGGuCu AACAGGUU | 4266 |
| 362 | UCCAAUUU G UCCUGGUU | 1587 | AACCAGGA GCcgaaagGCGaGuCaaGGuCu AAAUUGGA | 4267 |
| 381 | CGCUGGAU G UGUCUGCG | 1588 | CGCAGACA GCcgaaagGCGaGuCaaGGuCu AUCCAGCG | 4268 |
| 383 | CUGGAUGU G UCUGCGGC | 1589 | GCCGCAGA GCcgaaagGCGaGuCaaGGuCu ACAUCCAG | 4269 |
| 438 | AUCUUCUU G UUGGUUCU | 1590 | AGAACCAA GCcgaaagGCGaGuCaaGGuCu AAGAAGAU | 4270 |
| 465 | CAAGGUAU G UUGCCCGU | 1591 | ACGGGCAA GCcgaaagGCGaGuCaaGGuCu AUACCUUG | 4271 |
| 476 | GCCCGUUU G UCCUCUAA | 1592 | UUAGAGGA GCcgaaagGCGaGuCaaGGuCu AAACGGGC | 4272 |
| 555 | ACCUCUAU G UUUCCCUC | 1593 | GAGGGAAA GCcgaaagGCGaGuCaaGGuCu AUAGAGGU | 4273 |
| 566 | UCCCUCAU G UUGCUGUA | 1594 | UACAGCAA GCcgaaagGCGaGuCaaGGuCu AUGAGGGA | 4274 |
| 572 | AUGUUGCU G UACAAAAC | 1595 | GUUUUGUA GCcgaaagGCGaGuCaaGGuCu AGCAACAU | 4275 |
| 602 | CUGCACCU G UAUUCCCA | 1596 | UGGGAAUA GCcgaaagGCGaGuCaaGGuCu AGGUGCAG | 4276 |
| 694 | UGCCAUUU G UUCAGUGG | 1597 | CCACUGAA GCcgaaagGCGaGuCaaGGuCu AAAUGGCA | 4277 |
| 724 | CCCCCACU G UCUGGCUU | 1598 | AAGCCAGA GCcgaaagGCGaGuCaaGGuCu AGUGGGGG | 4278 |
| 750 | UGGAUGAU G UGGUUUUG | 1599 | CAAAACCA GCcgaaagGCGaGuCaaGGuCu AUCAUCCA | 4279 |
| 771 | CCAAGUCU G UACAACAU | 1600 | AUGUUGUA GCcgaaagGCGaGuCaaGGuCu AGACUUGG | 4280 |
| 801 | AUGCCGCU G UUACCAAU | 1601 | AUUGGUAA GCcgaaagGCGaGuCaaGGuCu AGCGGCAU | 4281 |
| 818 | UUUCUUUU G UCUUUGGG | 1602 | CCCAAAGA GCcgaaagGCGaGuCaaGGuCu AAAAGAAA | 4282 |
| 888 | UGGGAUAU G UAAUUGGG | 1603 | CCCAAUUA GCcgaaagGCGaGuCaaGGuCu AUAUCCCA | 4283 |
| 927 | AACAUAUU G UACAAAAA | 1604 | UUUUUGUA GCcgaaagGCGaGuCaaGGuCu AAUAUGUU | 4284 |
| 944 | AUCAAAAU G UGUUUUAG | 1605 | CUAAAACA GCcgaaagGCGaGuCaaGGuCu AUUUUGAU | 4285 |
| 946 | CAAAAUGU G UUUUAGGA | 1606 | UCCUAAAA GCcgaaagGCGaGuCaaGGuCu ACAUUUUG | 4286 |
| 963 | AACUUCCU G UAAACAGG | 1607 | CCUGUUUA GCcgaaagGCGaGuCaaGGuCu AGGAAGUU | 4287 |
| 991 | GAAAGUAU G UCAACGAA | 1608 | UUCGUUGA GCcgaaagGCGaGuCaaGGuCu AUACUUUC | 4288 |
| 1002 | AACGAAUU G UGGGUCUU | 1609 | AAGACCCA GCcgaaagGCGaGuCaaGGuCu AAUUCGUU | 4289 |
| 1039 | CACGCAAU G UGGAUAUU | 1610 | AAUAUCCA GCcgaaagGCGaGuCaaGGuCu AUUGCGUG | 4290 |
| 1137 | AACAGUAU G UGAACCUU | 1611 | AAGGUUCA GCcgaaagGCGaGuCaaGGuCu AUACUGUU | 4291 |
| 1184 | UGCCAAGU G UUUGCUGA | 1612 | UCAGCAAA GCcgaaagGCGaGuCaaGGuCu ACUUGGCA | 4292 |
| 1251 | GAACCUUU G UGUCUCCU | 1613 | AGGAGACA GCcgaaagGCGaGuCaaGGuCu AAAGGUUC | 4293 |
| 1253 | ACCUUUGU G UCUCCUCU | 1614 | AGAGGAGA GCcgaaagGCGaGuCaaGGuCu ACAAAGGU | 4294 |
| 1294 | AGCCGCUU G UUUUGCUC | 1615 | GAGCAAAA GCcgaaagGCGaGuCaaGGuCu AAGCGGCU | 4295 |
| 1344 | ACAAUUCU G UCGUGCUC | 1616 | GAGCACGA GCcgaaagGCGaGuCaaGGuCu AGAAUUGU | 4296 |
| 1390 | GCUAGGCU G UGCUGCCA | 1617 | UGGCAGCA GCcgaaagGCGaGuCaaGGuCu AGCCUAGC | 4297 |
| 1425 | CGUCCUUU G UUUACGUC | 1618 | GACGUAAA GCcgaaagGCGaGuCaaGGuCu AAAGGACG | 4298 |
| 1508 | CGCCUAUU G UACCGACC | 1619 | GGUCGGUA GCcgaaagGCGaGuCaaGGuCu AAUAGGCG | 4299 |
| 1557 | CCCCGUCU G UGCCUUCU | 1620 | AGAAGGCA GCcgaaagGCGaGuCaaGGuCu AGACGGGG | 4300 |
| 1581 | CGGACCGU G UGCACUUC | 1621 | GAAGUGCA GCcgaaagGCGaGuCaaGGuCu ACGGUCCG | 4301 |
| 1684 | UCAGCAAU G UCAACGAC | 1622 | GUCGUUGA GCcgaaagGCGaGuCaaGGuCu AUUGCUGA | 4302 |
| 1719 | CAAAGACU G UGUGUUUA | 1623 | UAAACACA GCcgaaagGCGaGuCaaGGuCu AGUCUUUG | 4303 |
| 1721 | AAGACUGU G UGUUUAAU | 1624 | AUUAAACA GCcgaaagGCGaGuCaaGGuCu ACAGUCUU | 4304 |
| 1723 | GACUGUGU G UUUAAUGA | 1625 | UCAUUAAA GCcgaaagGCGaGuCaaGGuCu ACACAGUC | 4305 |
| 1772 | AGGUCUUU G UACUAGGA | 1626 | UCCUAGUA GCcgaaagGCGaGuCaaGGuCu AAAGACCU | 4306 |
| 1785 | AGGAGGCU G UAGGCAUA | 1627 | UAUGCCUA GCcgaaagGCGaGuCaaGGuCu AGCCUCCU | 4307 |
| 1801 | AAAUUGGU G UGUUCACC | 1628 | GGUGAACA GCcgaaagGCGaGuCaaGGuCu ACCAAUUU | 4308 |
| 1803 | AUUGGUGU G UUCACCAG | 1629 | CUGGUGAA GCcgaaagGCGaGuCaaGGuCu ACACCAAU | 4309 |
| 1850 | CAUCUCAU G UUCAUGUC | 1630 | GACAUGAA GCcgaaagGCGaGuCaaGGuCu AUGAGAUG | 4310 |
| 1856 | AUGUUCAU G UCCUACUG | 1631 | CAGUAGGA GCcgaaagGCGaGuCaaGGuCu AUGAACAU | 4311 |
| 1864 | GUCCUACU G UUCAAGCC | 1632 | GGCUUGAA GCcgaaagGCGaGuCaaGGuCu AGUAGGAC | 4312 |
| 1881 | UCCAAGCU G UGCCUUGG | 1633 | CCAAGGCA GCcgaaagGCGaGuCaaGGuCu AGCUUGGA | 4313 |
| 1939 | GAGCUUCU G UGGAGUUA | 1634 | UAACUCCA GCcgaaagGCGaGuCaaGGuCu AGAAGCUC | 4314 |
| 2013 | UCUGCUCU G UAUCGGGG | 1635 | CCCCGAUA GCcgaaagGCGaGuCaaGGuCu AGAGCAGA | 4315 |
| 2045 | GGAACAUU G UUCACCUC | 1636 | GAGGUGAA GCcgaaagGCGaGuCaaGGuCu AAUGUUCC | 4316 |
| 2082 | GCUAUUCU G UGUUGGGG | 1637 | CCCCAACA GCcgaaagGCGaGuCaaGGuCu AGAAUAGC | 4317 |
| 2084 | UAUUCUGU G UUGGGGUG | 1638 | CACCCCAA GCcgaaagGCGaGuCaaGGuCu ACAGAAUA | 4318 |
| 2167 | UCAGCUAU G UCAACGUU | 1639 | AACGUUGA GCcgaaagGCGaGuCaaGGuCu AUAGCUGA | 4319 |
| 2205 | CAACUAUU G UGGUUUCA | 1640 | UGAAACCA GCcgaaagGCGaGuCaaGGuCu AAUAGUUG | 4320 |
| 2222 | CAUUUCCU G UCUUACUU | 1641 | AAGUAAGA GCcgaaagGCGaGuCaaGGuCu AGGAAAUG | 4321 |
| 2245 | GAGAAACU G UUCUUGAA | 1642 | UUCAAGAA GCcgaaagGCGaGuCaaGGuCu AGUUUCUC | 4322 |
| 2262 | UAUUUGGU G UCUUUUGG | 1643 | CCAAAAGA GCcgaaagGCGaGuCaaGGuCu ACCAAAUA | 4323 |
| 2274 | UUUGGAGU G UGGAUUCG | 1644 | CGAAUCCA GCcgaaagGCGaGuCaaGGuCu ACUCCAAA | 4324 |
| 2344 | AAACUACU G UUGUUAGA | 1645 | UCUAACAA GCcgaaagGCGaGuCaaGGuCu AGUAGUUU | 4325 |
| 2347 | CUACUGUU G UUAGACGA | 1646 | UCGUCUAA GCcgaaagGCGaGuCaaGGuCu AACAGUAG | 4326 |
| 2450 | AUCUCAAU G UUAGUAUU | 1647 | AAUACUAA GCcgaaagGCGaGuCaaGGuCu AUUGAGAU | 4327 |
| 2573 | AGGACAUU G UUGAUAGA | 1648 | UCUAUCAA GCcgaaagGCGaGuCaaGGuCu AAUGUCCU | 4328 |
| 2583 | UGAUAGAU G UAAGCAAU | 1649 | AUUGCUUA GCcgaaagGCGaGuCaaGGuCu AUCUAUCA | 4329 |
| 2594 | AGCAAUUU G UGGGGCCC | 1650 | GGGCCCCA GCcgaaagGCGaGuCaaGGuCu AAAUUGCU | 4330 |
| 2663 | AUCCCAAU G UUACUAAA | 1651 | UUUAGUAA GCcgaaagGCGaGuCaaGGuCu AUUGGGAU | 4331 |
| 2717 | CAGAGUAU G UAGUUAAU | 1652 | AUUAACUA GCcgaaagGCGaGuCaaGGuCu AUACUCUG | 4332 |
| 2901 | AUCUUUCU G UCCCCAAU | 1653 | AUUGGGGA GCcgaaagGCGaGuCaaGGuCu AGAAAGAU | 4333 |
| 3071 | GGGGGACU G UUGGGGUG | 1654 | CACCCCAA GCcgaaagGCGaGuCaaGGuCu AGUCCCCC | 4334 |
| 3111 | UCACAACU G UGCCAGCA | 1655 | UGCUGGCA GCcgaaagGCGaGuCaaGGuCu AGUUGUGA | 4335 |
| 40 | AUCCCAGA G UCAGGGCC | 1656 | GGCCCUGA GCcgaaagGCGaGuCaaGGuCu UCUGGGAU | 4336 |
| 46 | GAGUCAGG G CCCUGUAC | 1657 | GUACAGGG GCcgaaagGCGaGuCaaGGuCu CCUGACUC | 4337 |
| 65 | UCCUGCUG G UGGCUCCA | 1658 | UGGAGCCA GCcgaaagGCGaGuCaaGGuCu CAGCAGGA | 4338 |
| 68 | UGCUGGUG G CUCCAGUU | 1659 | AACUGGAG GCcgaaagGCGaGuCaaGGuCu CACCAGCA | 4339 |
| 74 | UGGCUCCA G UUCAGGAA | 1660 | UUCCUGAA GCcgaaagGCGaGuCaaGGuCu UGGAGCCA | 4340 |
| 85 | CAGGAACA G UGAGCCCU | 1661 | AGGGCUCA GCcgaaagGCGaGuCaaGGuCu UGUUCCUG | 4341 |
| 89 | AACAGUGA G CCCUGCUC | 1662 | GAGCAGGG GCcgaaagGCGaGuCaaGGuCu UCACUGUU | 4342 |
| 120 | GCCAUAUC G UCAAUCUU | 1663 | AAGAUUGA GCcgaaagGCGaGuCaaGGuCu GAUAUGGC | 4343 |
| 196 | CCCUGCUC G UGUUACAG | 1664 | CUGUAACA GCcgaaagGCGaGuCaaGGuCu GAGCAGGG | 4344 |
| 205 | UGUUACAG G CGGGGUUU | 1665 | AAACCCCG GCcgaaagGCGaGuCaaGGuCu CUGUAACA | 4345 |
| 210 | CAGGCGGG G UUUUUCUU | 1666 | AAGAAAAA GCcgaaagGCGaGuCaaGGuCu CCCGCCUG | 4346 |
| 248 | ACCACAGA G UCUAGACU | 1667 | AGUCUAGA GCcgaaagGCGaGuCaaGGuCu UCUGUGGU | 4347 |
| 258 | CUAGACUC G UGGUGGAC | 1668 | GUCCACCA GCcgaaagGCGaGuCaaGGuCu GAGUCUAG | 4348 |
| 261 | GACUCGUG G UGGACUUC | 1669 | GAAGUCCA GCcgaaagGCGaGuCaaGGuCu CACGAGUC | 4349 |
| 295 | GAACACCC G UGUGUCUU | 1670 | AAGACACA GCcgaaagGCGaGuCaaGGuCu GGGUGUUC | 4350 |
| 305 | GUGUCUUG G CCAAAAUU | 1671 | AAUUUUGG GCcgaaagGCGaGuCaaGGuCu CAAGACAC | 4351 |
| 318 | AAUUCGCA G UCCCAAAU | 1672 | AUUUGGGA GCcgaaagGCGaGuCaaGGuCu UGCGAAUU | 4352 |
| 332 | AAUCUCCA G UCACUCAC | 1673 | GUGAGUGA GCcgaaagGCGaGuCaaGGuCu UGGAGAUU | 4353 |
| 368 | UUGUCCUG G UUAUCGCU | 1674 | AGCGAUAA GCcgaaagGCGaGuCaaGGuCu CAGGACAA | 4354 |
| 390 | UGUCUGCG G CGUUUUAU | 1675 | AUAAAACG GCcgaaagGCGaGuCaaGGuCu CGCAGACA | 4355 |
| 392 | UCUGCGGC G UUUUAUCA | 1676 | UGAUAAAA GCcgaaagGCGaGuCaaGGuCu GCCGCAGA | 4356 |
| 442 | UCUUGUUG G UUCUUCUG | 1677 | CAGAAGAA GCcgaaagGCGaGuCaaGGuCu CAACAAGA | 4357 |
| 461 | CUAUCAAG G UAUGUUGC | 1678 | GCAACAUA GCcgaaagGCGaGuCaaGGuCu CUUGAUAG | 4358 |
| 472 | UGUUGCCC G UUUGUCCU | 1679 | AGGACAAA GCcgaaagGCGaGuCaaGGuCu GGGCAACA | 4359 |
| 506 | AACAACCA G CACCGGAC | 1680 | GUCCGGUG GCcgaaagGCGaGuCaaGGuCu UGGUUGUU | 4360 |
| 625 | CAUCUUGG G CUUUCGCA | 1681 | UGCGAAAG GCcgaaagGCGaGuCaaGGuCu CCAAGAUG | 4361 |
| 648 | CUAUGGGA G UGGGCCUC | 1682 | GAGGCCCA GCcgaaagGCGaGuCaaGGuCu UCCCAUAG | 4362 |
| 652 | GGGAGUGG G CCUCAGUC | 1683 | GACUGAGG GCcgaaagGCGaGuCaaGGuCu CCACUCCC | 4363 |
| 658 | GGGCCUCA G UCCGUUUC | 1684 | GAAACGGA GCcgaaagGCGaGuCaaGGuCu UGAGGCCC | 4364 |
| 662 | CUCAGUCC G UUUCUCUU | 1685 | AAGAGAAA GCcgaaagGCGaGuCaaGGuCu GGACUGAG | 4365 |
| 672 | UUCUCUUG G CUCAGUUU | 1686 | AAACUGAG GCcgaaagGCGaGuCaaGGuCu CAAGAGAA | 4366 |
| 677 | UUGGCUCA G UUUACUAG | 1687 | CUAGUAAA GCcgaaagGCGaGuCaaGGuCu UGAGCCAA | 4367 |
| 685 | GUUUACUA G UGCCAUUU | 1688 | AAAUGGCA GCcgaaagGCGaGuCaaGGuCu UAGUAAAC | 4368 |
| 699 | UUUGUUCA G UGGUUCGU | 1689 | ACGAACCA GCcgaaagGCGaGuCaaGGuCu UGAACAAA | 4369 |
| 702 | GUUCAGUG G UUCGUAGG | 1690 | CCUACGAA GCcgaaagGCGaGuCaaGGuCu CACUGAAC | 4370 |
| 706 | AGUGGUUC G UAGGGCUU | 1691 | AAGCCCUA GCcgaaagGCGaGuCaaGGuCu GAACCACU | 4371 |
| 711 | UUCGUAGG G CUUUCCCC | 1692 | GGGGAAAG GCcgaaagGCGaGuCaaGGuCu CCUACGAA | 4372 |
| 729 | ACUGUCUG G CUUUCAGU | 1693 | ACUGAAAG GCcgaaagGCGaGuCaaGGuCu CAGACAGU | 4373 |
| 736 | GGCUUUCA G UUAUAUGG | 1694 | CCAUAUAA GCcgaaagGCGaGuCaaGGuCu UGAAAGCC | 4374 |
| 753 | AUGAUGUG G UUUUGGGG | 1695 | CCCCAAAA GCcgaaagGCGaGuCaaGGuCu CACAUCAU | 4375 |
| 762 | UUUUGGGG G CCAAGUCU | 1696 | AGACUUGG GCcgaaagGCGaGuCaaGGuCu CCCCAAAA | 4376 |
| 767 | GGGGCCAA G UCUGUACA | 1697 | UGUACAGA GCcgaaagGCGaGuCaaGGuCu UUGGCCCC | 4377 |
| 785 | CAUCUUGA G UCCCUUUA | 1698 | UAAAGGGA GCcgaaagGCGaGuCaaGGuCu UCAAGAUG | 4378 |
| 826 | GUCUUUGG G UAUACAUU | 1699 | AAUGUAUA GCcgaaagGCGaGuCaaGGuCu CCAAAGAC | 4379 |
| 898 | AAUUGGGA G UUGGGGCA | 1700 | UGCCCCAA GCcqaaagGCGaGuCaaGGuCu UCCCAAUU | 4380 |
| 904 | GAGUUGGG G CACAUUGC | 1701 | GCAAUGUG GCcgaaagGCGaGuCaaGGuCu CCCAACUC | 4381 |
| 971 | GUAAACAG G CCUAUUGA | 1702 | UCAAUAGG GCcgaaagGCGaGuCaaGGuCu CUGUUUAC | 4382 |
| 987 | AUUGGAAA G UAUGUCAA | 1703 | UUGACAUA GCcgaaagGCGaGuCaaGGuCu UUUCCAU | 4383 |
| 1006 | AAUUGUGG G UCUUUUGG | 1704 | CCAAAAGA GCcgaaagGCGaGuCaaGGuCu CCACAAUU | 4384 |
| 1016 | CUUUUGGG G UUUGCCGC | 1705 | GCGGCAAA GCcgaaagGCGaGuCaaGGuCu CCCAAAAG | 4385 |
| 1080 | GCAUACAA G CAAAACAG | 1706 | CUGUUUUG GCcgaaagGCGaGuCaaGGuCu UUGUAUGC | 4386 |
| 1089 | CAAAACAG G CUUUUACU | 1707 | AGUAAAAG GCcgaaagGCGaGuCaaGGuCu CUGUUUUG | 4387 |
| 1116 | CUUACAAG G CCUUUCUA | 1708 | UAGAAAGG GCcgaaagGCGaGuCaaGGuCu CUUGUAAG | 4388 |
| 1126 | CUUUCUAA G UAAACAGU | 1709 | ACUGUUUA GCcgaaagGCGaGuCaaGGuCu UUAGAAAG | 4389 |
| 1133 | AGUAAACA G UAUGUGAA | 1710 | UUCACAUA GCcgaaagGCGaGuCaaGGuCu UGUUUACU | 4390 |
| 1152 | UUUACCCC G UUGCUCGG | 1711 | CCGAGCAA GCcgaaagGCGaGuCaaGGuCu GGGGUAAA | 4391 |
| 1160 | GUUGCUCG G CAACGGCC | 1712 | GGCCGUUG GCcgaaagGCGaGuCaaGGuCu CGAGCAAC | 4392 |
| 1166 | CGGCAACG G CCUGGUCU | 1713 | AGACCAGG GCcgaaagGCGaGuCaaGGuCu CGUUGCCG | 4393 |
| 1171 | ACGGCCUG G UCUAUGCC | 1714 | GGCAUAGA GCcgaaagGCGaGuCaaGGuCu CAGGCCGU | 4394 |
| 1182 | UAUGCCAA G UGUUUGCU | 1715 | AGCAAACA GCcgaaagGCGaGuCaaGGuCu UUGGCAUA | 4395 |
| 1207 | CCCCACUG G UUGGGGCU | 1716 | AGCCCCAA GCcgaaagGCGaGuCaaGGuCu CAGUGGGG | 4396 |
| 1213 | UGGUUGGG G CUUGGCCA | 1717 | UGGCCAAG GCcgaaagGCGaGuCaaGGuCu CCCAACCA | 4397 |
| 1218 | GGGGCUUG G CCAUAGGC | 1718 | GCCUAUGG GCcgaaagGCGaGuCaaGGuCu CAAGCCCC | 4398 |
| 1225 | GGCCAUAG G CCAUCAGC | 1719 | GCUGAUGG GCcgaaagGCGaGuCaaGGuCu CUAUGGCC | 4399 |
| 1232 | GGCCAUCA G CGCAUGCG | 1720 | CGCAUGCG GCcgaaagGCGaGuCaaGGuCu UGAUGGCC | 4400 |
| 1240 | GCGCAUGC G UGGAACCU | 1721 | AGGUUCCA GCcgaaagGCGaGuCaaGGuCu GCAUGCGC | 4401 |
| 1287 | AACUCCUA G CCGCUUGU | 1722 | ACAAGCGG GCcgaaagGCGaGuCaaGGuCu UAGGAGUU | 4402 |
| 1306 | UGCUCGCA G CAGGUCUG | 1723 | CAGACCUG GCcgaaagGCGaGuCaaGGuCu UGCGAGCA | 4403 |
| 1310 | CGCAGCAG G UCUGGGGC | 1724 | GCCCCAGA GCcgaaagGCGaGuCaaGGuCu CUGCUGCG | 4404 |
| 1317 | GGUCUGGG G CAAAACUC | 1725 | GAGUUUUG GCcgaaagGCGaGuCaaGGuCu CCCAGACC | 4405 |
| 1347 | AUUCUGUC G UGCUCUCC | 1726 | GGAGAGCA GCcgaaagGCGaGuCaaGGuCu GACAGAAU | 4406 |
| 1379 | UUUCCAUG G CUGCUAGG | 1727 | CCUAGCAG GCcgaaagGCGaGuCaaGGuCu CAUGGAAA | 4407 |
| 1387 | GCUGCUAG G CUGUGCUG | 1728 | CAGCACAG GCcgaaagGCGaGuCaaGGuCu CUAGCAGC | 4408 |
| 1418 | CGCGGGAC G UCCUUUGU | 1729 | ACAAAGGA GCcgaaagGCGaGuCaaGGuCu GUCCCGCG | 4409 |
| 1431 | UUGUUUAC G UCCCGUCG | 1730 | CGACGGGA GCcgaaagGCGaGuCaaGGuCu GUAAACAA | 4410 |
| 1436 | UACGUCCC G UCGGCGCU | 1731 | AGCGCCGA GCcgaaagGCGaGuCaaGGuCu GGGACGUA | 4411 |
| 1440 | UCCCGUCG G CGCUGAAU | 1732 | AUUCAGCG GCcgaaagGCGaGuCaaGGuCu CGACGGGA | 4412 |
| 1471 | CUCCCGGG G CCGCUUGG | 1733 | CCAAGCGG GCcgaaagGCGaGuCaaGGuCu CCCGGGAG | 4413 |
| 1481 | CGCUUGGG G CUCUACCG | 1734 | CGGUAGAG GCcgaaagGCGaGuCaaGGuCu CCCAAGCG | 4414 |
| 1517 | UACCGACC G UCCACGGG | 1735 | CCCGUGGA GCcgaaagGCGaGuCaaGGuCu GGUCGGUA | 4415 |
| 1526 | UCCACGGG G CGCACCUC | 1736 | GAGGUGCG GCcgaaagGCGaGuCaaGGuCu CCCGUGGA | 4416 |
| 1553 | GACUCCCC G UCUGUGCC | 1737 | GGCACAGA GCcgaaagGCGaGuCaaGGuCu GGGGAGUC | 4417 |
| 1579 | GCCGGACC G UGUGCACU | 1738 | AGUGCACA GCcgaaagGCGaGuCaaGGuCu GGUCCGGC | 4418 |
| 1605 | CUCUGCAC G UCGCAUGG | 1739 | CCAUGCGA GCcgaaagGCGaGuCaaGGuCu GUGCAGAG | 4419 |
| 1622 | AGACCACC G UGAACGCC | 1740 | GGCGUUCA GCcgaaagGCGaGuCaaGGuCu GGUGGUCU | 4420 |
| 1649 | UGCCCAAG G UCUUGCAU | 1741 | AUGCAAGA GCcgaaagGCGaGuCaaGGuCu CUUGGGCA | 4421 |
| 1679 | GACUUUCA G CAAUGUCA | 1742 | UGACAUUG GCcgaaagGCGaGuCaaGGuCu UGAAAGUC | 4422 |
| 1703 | ACCUUGAG G CAUACUUC | 1743 | GAAGUAUG GCcgaaagGCGaGuCaaGGuCu CUCAAGGU | 4423 |
| 1732 | UUUAAUGA G UGGGAGGA | 1744 | UCCUCCCA GCcgaaagGCGaGuCaaGGuCu UCAUUAAA | 4424 |
| 1741 | UGGGAGGA G UUGGGGGA | 1745 | UCCCCCAA GCcgaaagGCGaGuCaaGGuCu UCCUCCCA | 4425 |
| 1754 | GGGAGGAG G UUAGGUUA | 1746 | UAACCUAA GCcgaaagGCGaGuCaaGGuCu CUCCUCCC | 4426 |
| 1759 | GAGGUUAG G UUAAAGGU | 1747 | ACCUUUAA GCcgaaagGCGaGuCaaGGuCu CUAACCUC | 4427 |
| 1766 | GGUUAAAG G UCUUUGUA | 1748 | UACAAAGA GCcgaaagGCGaGuCaaGGuCu CUUUAACC | 4428 |
| 1782 | ACUAGGAG G CUGUAGGC | 1749 | GCCUACAG GCcgaaagGCGaGuCaaGGuCu CUCCUAGU | 4429 |
| 1789 | GGCUGUAG G CAUAAAUU | 1750 | AAUUUAUG GCcgaaagGCGaGuCaaGGuCu CUACAGCC | 4430 |
| 1799 | AUAAAUUG G UGUGUUCA | 1751 | UGAACACA GCcgaaagGCGaGuCaaGGuCu CAAUUUAU | 4431 |
| 1811 | GUUCACCA G CACCAUGC | 1752 | GCAUGGUG GCcgaaagGCGaGuCaaGGuCu UGGUGAAC | 4432 |
| 1870 | CUGUUCAA G CCUCCAAG | 1753 | CUUGGAGG GCcgaaagGCGaGuCaaGGuCu UUGAACAG | 4433 |
| 1878 | GCCUCCAA G CUGUGCCU | 1754 | AGGCACAG GCcgaaagGCGaGuCaaGGuCu UUGGAGGC | 4434 |
| 1890 | UGCCUUGG G UGGCUUUG | 1755 | CAAAGCCA GCcgaaagGCGaGuCaaGGuCu CCAAGGCA | 4435 |
| 1893 | CUUGGGUG G CUUUGGGG | 1756 | CCCCAAAG GCcgaaagGCGaGuCaaGGuCu CACCCAAG | 4436 |
| 1901 | GCUUUGGG G CAUGGACA | 1757 | UGUCCAUG GCcgaaagGCGaGuCaaGGuCu CCCAAAGC | 4437 |
| 1917 | AUUGACCC G UAUAAAGA | 1758 | UCUUUAUA GCcgaaagGCGaGuCaaGGuCu GGGUCAAU | 4438 |
| 1933 | AAUUUGGA G CUUCUGUG | 1759 | CACAGAAG GCcgaaagGCGaGuCaaGGuCu UCCAAAUU | 4439 |
| 1944 | UCUGUGGA G UUACUCUC | 1760 | GAGAGUAA GCcgaaagGCGaGuCaaGGuCu UCCACAGA | 4440 |
| 2023 | AUCGGGGG G CCUUAGAG | 1761 | CUCUAAGG GCcgaaagGCGaGuCaaGGuCu CCCCCCAU | 4441 |
| 2031 | GCCUUAGA G UCUCCGGA | 1762 | UCCGGAGA GCcgaaagGCGaGuCaaGGuCu UCUAAGGC | 4442 |
| 2062 | ACCAUACG G CACUCAGG | 1763 | CCUGAGUG GCcgaaagGCGaGuCaaGGuCu CGUAUGGU | 4443 |
| 2070 | GCACUCAG G CAAGCUAU | 1764 | AUAGCUUG GCcgaaagGCGaGuCaaGGuCu CUGAGUGC | 4444 |
| 2074 | UCAGGCAA G CUAUUCUG | 1765 | CAGAAUAG GCcgaaagGCGaGuCaaGGuCu UUGCCUGA | 4445 |
| 2090 | GUGUUGGG G UGAGUUGA | 1766 | UCAACUCA GCcgaaagGCGaGuCaaGGuCu CCCAACAC | 4446 |
| 2094 | UGGGGUGA G UUGAUGAA | 1767 | UUCAUCAA GCcgaaagGCGaGuCaaGGuCu UCACCCCA | 4447 |
| 2107 | UGAAUCUA G CCACCUGG | 1768 | CCAGGUGG GCcgaaagGCGaGuCaaGGuCu UAGAUUCA | 4448 |
| 2116 | CCACCUGG G UGGGAAGU | 1769 | ACUUCCCA GCcgaaagGCGaGuCaaGGuCu CCAGGUGG | 4449 |
| 2123 | GGUGGGAA G UAAUUUGG | 1770 | CCAAAUUA GCcgaaagGCGaGuCaaGGuCu UUCCCACC | 4450 |
| 2140 | AAGAUCCA G CAUCCAGG | 1771 | CCUGGAUG GCcgaaagGCGaGuCaaGGuCu UGGAUCUU | 4451 |
| 2155 | GGGAAUUA G UAGUCAGC | 1772 | GCUGACUA GCcgaaagGCGaGuCaaGGuCu UAAUUCCC | 4452 |
| 2158 | AAUUAGUA G UCAGCUAU | 1773 | AUAGCUGA GCcgaaagGCGaGuCaaGGuCu UACUAAUU | 4453 |
| 2162 | AGUAGUCA G CUAUGUCA | 1774 | UGACAUAG GCcgaaagGCGaGuCaaGGuCu UGACUACU | 4454 |
| 2173 | AUGUCAAC G UUAAUAUG | 1775 | CAUAUUAA GCcgaaagGCGaGuCaaGGuCu GUUGACAU | 4455 |
| 2183 | UAAUAUGG G CCUAAAAA | 1776 | UUUUUAGG GCcgaaagGCGaGuCaaGGuCu CCAUAUUA | 4456 |
| 2208 | CUAUUGUG G UUUCACAU | 1777 | AUGUGAAA GCcgaaagGCGaGuCaaGGuCu CACAAUAG | 4457 |
| 2235 | ACUUUUGG G CGAGAAAC | 1778 | GUUUCUCG GCcgaaagGCGaGuCaaGGuCu CCAAAACU | 4458 |
| 2260 | AAUAUUUG G UGUCUUUU | 1779 | AAAAGACA GCcgaaagGCGaGuCaaGGuCu CAAAUAUU | 4459 |
| 2272 | CUUUUGGA G UGUGGAUU | 1780 | AAUCCACA GCcgaaagGCGaGuCaaGGuCu UCCAAAAG | 4460 |
| 2360 | ACGAAGAG G CAGGUCCC | 1781 | GGGACCUG GCcgaaagGCGaGuCaaGGuCu CUCUUCGU | 4461 |
| 2364 | AGAGGCAG G UCCCCUAG | 1782 | CUAGGGGA GCcgaaagGCGaGuCaaGGuCu CUGCCUCU | 4462 |
| 2403 | AGACGAAG G UCUCAAUC | 1783 | GAUUGAGA GCcgaaagGCGaGuCaaGGuCu CUUCGUCU | 4463 |
| 2417 | AUCGCCGC G UCGCAGAA | 1784 | UUCUGCGA GCcgaaagGCGaGuCaaGGuCu GCGGCGAU | 4464 |
| 2454 | CAAUGUUA G UAUUCCUU | 1785 | AAGGAAUA GCcgaaagGCGaGuCaaGGuCu UAACAUUG | 4465 |
| 2474 | CACAUAAG G UGGGAAAC | 1786 | GUUUCCCA GCcgaaagGCGaGuCaaGGuCu CUUAUGUG | 4466 |
| 2491 | UUUACGGG G CUUUAUUC | 1787 | GAAUAAAG GCcgaaagGCGaGuCaaGGuCu CCCGUAAA | 4467 |
| 2507 | CUUCUACG G UACCUUGC | 1788 | GCAAGGUA GCcgaaagGCGaGuCaaGGuCu CGUAGAAG | 4468 |
| 2530 | CCUAAAUG G CAAACUCC | 1789 | GGAGUUUG GCcgaaagGCGaGuCaaGGuCu CAUUUAGG | 4469 |
| 2587 | AGAUGUAA G CAAUUUGU | 1790 | ACAAAUUG GCcgaaagGCGaGuCaaGGuCu UUACAUCU | 4470 |
| 2599 | UUUGUGGG G CCCCUUAC | 1791 | GUAAGGGG GCcgaaagGCGaGuCaaGGuCu CCCACAAA | 4471 |
| 2609 | CCCUUACA G UAAAUGAA | 1792 | UUCAUUUA GCcgaaagGCGaGuCaaGGuCu UGUAAGGG | 4472 |
| 2650 | CCUGCUAG G UUUUAUCC | 1793 | GGAUAAAA GCcgaaagGCGaGuCaaGGuCu CUAGCAGG | 4473 |
| 2701 | AUCAAACC G UAUUAUCC | 1794 | GGAUAAUA GCcgaaagGCGaGuCaaGGuCu GGUUUGAU | 4474 |
| 2713 | UAUCCAGA G UAUGUAGU | 1795 | ACUACAUA GCcgaaagGCGaGuCaaGGuCu UCUGGAUA | 4475 |
| 2720 | AGUAUGUA G UUAAUCAU | 1796 | AUGAUUAA GCcgaaagGCGaGuCaaGGuCu UACAUACU | 4476 |
| 2768 | UUUGGAAG G CGGGGAUC | 1797 | GAUCCCCG GCcgaaagGCGaGuCaaGGuCu CUUCCAAA | 4477 |
| 2791 | AAAAGAGA G UCCACACG | 1798 | CGUGUGGA GCcgaaagGCGaGuCaaGGuCu UCUCUUUU | 4478 |
| 2799 | GUCCACAC G UAGCGCCU | 1799 | AGGCGCUA GCcgaaagGCGaGuCaaGGuCu GUGUGGAC | 4479 |
| 2802 | CACACGUA G CGCCUCAU | 1800 | AUGAGGCG GCcgaaagGCGaGuCaaGGuCu UACGUGUG | 4480 |
| 2818 | UUUUGCGG G UCACCAUA | 1801 | UAUGGUGA GCcgaaagGCGaGuCaaGGuCu CCGCAAAA | 4481 |
| 2848 | GAUCUACA G CAUGGGAG | 1802 | CUCCCAUG GCcgaaagGCGaGuCaaGGuCu UGUAGAUC | 4482 |
| 2857 | CAUGGGAG G UUGGUCUU | 1803 | AAGACCAA GCcgaaagGCGaGuCaaGGuCu CUCCCAUG | 4483 |
| 2861 | GGAGGUUG G UCUUCCAA | 1804 | UUGGAAGA GCcgaaagGCGaGuCaaGGuCu CAACCUCC | 4484 |
| 2881 | UCGAAAAG G CAUGGGGA | 1805 | UCCCCAUG GCcgaaagGCGaGuCaaGGuCu CUUUUCGA | 4485 |
| 2936 | GAUCAUCA G UUGGACCC | 1806 | GGGUCCAA GCcgaaagGCGaGuCaaGGuCu UGAUGAUC | 4486 |
| 2955 | CAUUCAAA G CCAACUCA | 1807 | UGAGUUGG GCcgaaagGCGaGuCaaGGuCu UUUGAAUG | 4487 |
| 2964 | CCAACUCA G UAAAUCCA | 1808 | UGGAUUUA GCcgaaagGCGaGuCaaGGuCu UGAGUUGG | 4488 |
| 3005 | GACAACUG G CCGGACGC | 1809 | GCGUCCGG GCcgaaagGCGaGuCaaGGuCu CAGUUGUC | 4489 |
| 3021 | CCAACAAG G UGGGAGUG | 1810 | CACUCCCA GCcgaaagGCGaGuCaaGGuCu CUUGUUGG | 4490 |
| 3027 | AGGUGGGA G UGGGAGCA | 1811 | UGCUCCCA GCcgaaagGCGaGuCaaGGuCu UCCCACCU | 4491 |
| 3033 | GAGUGGGA G CAUUCGGG | 1812 | CCCGAAUG GCcgaaagGCGaGuCaaGGuCu UCCCACUC | 4492 |
| 3041 | GCAUUCGG G CCAGGGUU | 1813 | AACCCUGG GCcgaaagGCGaGuCaaGGuCu CCGAAUGC | 4493 |
| 3047 | GGGCCAGG G UUCACCCC | 1814 | GGGGUGAA GCcgaaagGCGaGuCaaGGuCu CCUGGCCC | 4494 |
| 3077 | CUGUUGGG G UGGAGCCC | 1815 | GGGCUCCA GCcgaaagGCGaGuCaaGGuCu CCCAACAG | 4495 |
| 3082 | GGGGUGGA G CCCUCACG | 1816 | CGUGAGGG GCcgaaagGCGaGuCaaGGuCu UCCACCCC | 4496 |
| 3097 | CGCUCAGG G CCUACUCA | 1817 | UGAGUAGG GCcgaaagGCGaGuCaaGGuCu CCUGAGCG | 4497 |
| 3117 | CUGUGCCA G CAGCUCCU | 1818 | AGGAGCUG GCcgaaagGCGaGuCaaGGuCu UGGCACAG | 4498 |
| 3120 | UGCCAGCA G CUCCUCCU | 1819 | AGGAGGAG GCcgaaagGCGaGuCaaGGuCu UGCUGGCA | 4499 |
| 3146 | ACCAAUCG G CAGUCAGG | 1820 | CCUGACUG GCcgaaagGCGaGuCaaGGuCu CGAUUGGU | 4500 |
| 3149 | AAUCGGCA G UCAGGAAG | 1821 | CUUCCUGA GCcgaaagGCGaGuCaaGGuCu UGCCGAUU | 4501 |
| 3158 | UCAGGAAG G CAGCCUAC | 1822 | GUAGGCUG GCcgaaagGCGaGuCaaGGuCu CUUCCUGA | 4502 |
| 3161 | GGAAGGCA G CCUACUCC | 1823 | GGAGUAGG GCcgaaagGCGaGuCaaGGuCu UGCCUUCC | 4503 |
| 3204 | AUCCUCAG G CCAUGCAG | 1824 | CUGCAUGG GCcgaaagGCGaGuCaaGGuCu CUGAGGAU | 4504 |

Input Sequence = AF100308. Cut Site = YG/M or UG/U.
Stem Length = 8. Core Sequence = GCcgaaagGCGaGuCaaGGuCu
AF100308 (Hepatitis B virus strain 2-18, 3215 bp)

TABLE IX

HUMAN HBV DNAZYME AND SUBSTRATE SEQUENCE

| | | | | |
|---|---|---|---|---|
| Pos | Substrate | Seq ID | DNAzyme | Seq ID |

| | | | | |
|---|---|---|---|---|
| 508 | CAACCAGC A CCGGACCA | 833 | TGGTCCGG GGCTAGCTACAACGA GCTGGTTG | 4505 |
| 1632 | GAACGCCC A CAGGAACC | 1096 | GGTTCCTG GGCTAGCTACAACGA GGGCGTTC | 4506 |
| 2992 | CAACCCGC A CAAGGACA | 1376 | TGTCCTTG GGCTAGCTACAACGA GCGGGTTG | 4507 |
| 61 | ACUUUCCU G CUGGUGGC | 1448 | GCCACCAG GGCTAGCTACAACGA AGGAAAGT | 4508 |
| 94 | UGAGCCCU G CUCAGAAU | 1450 | ATTCTGAG GGCTAGCTACAACGA AGGGCTCA | 4509 |
| 112 | CUGUCUCU G CCAUAUCG | 1451 | CGATATGG GGCTAGCTACAACGA AGAGACAG | 4510 |
| 169 | AGAACAUC G CAUCAGGA | 1454 | TCCTGATG GGCTAGCTACAACGA GATGTTCT | 4511 |
| 192 | GGACCCCU G CUCGUGUU | 1455 | AACACGAG GGCTAGCTACAACGA AGGGGTCC | 4512 |
| 315 | CAAAAUUC G CAGUCCCA | 1457 | TGGGACTG GGCTAGCTACAACGA GAATTTTG | 4513 |
| 374 | UGGUUAUC G CUGGAUGU | 1458 | ACATCCAG GGCTAGCTACAACGA GATAACCA | 4514 |
| 387 | AUGUGUCU G CGGCGUUU | 1459 | AAACGCCG GGCTAGCTACAACGA AGACACAT | 4515 |
| 410 | CUUCCUCU G CAUCCUGC | 1460 | GCAGGATG GGCTAGCTACAACGA AGAGGAAG | 4516 |
| 417 | UGCAUCCU G CUGCUAUG | 1461 | CATAGCAG GGCTAGCTACAACGA AGGATGCA | 4517 |
| 420 | AUCCUGCU G CUAUGCCU | 1462 | AGGCATAG GGCTAGCTACAACGA AGCAGGAT | 4518 |
| 425 | GCUGCUAU G CCUCAUCU | 1463 | AGATGAGG GGCTAGCTACAACGA ATAGCAGC | 4519 |
| 468 | GGUAUGUU G CCCGUUUG | 1464 | CAAACGGG GGCTAGCTACAACGA AACATACC | 4520 |
| 518 | CGGACCAU G CAAAACCU | 1465 | AGGTTTTG GGCTAGCTACAACGA ATGGTCCG | 4521 |
| 527 | CAAAACCU G CACAACUC | 1466 | GAGTTGTG GGCTAGCTACAACGA AGGTTTTG | 4522 |
| 538 | CAACUCCU G CUCAAGGA | 1467 | TCCTTGAG GGCTAGCTACAACGA AGGAGTTG | 4523 |
| 569 | CUCAUGUU G CUGUACAA | 1468 | TTGTACAG GGCTAGCTACAACGA AACATGAG | 4524 |
| 596 | CGGAAACU G CACCUGUA | 1469 | TACAGGTG GGCTAGCTACAACGA AGTTTCCG | 4525 |
| 631 | GGGCUUUC G CAAAAUAC | 1470 | GTATTTTG GGCTAGCTACAACGA GAAAGCCC | 4526 |
| 687 | UUACUAGU G CCAUUUGU | 1471 | ACAAATGG GGCTAGCTACAACGA ACTAGTAA | 4527 |
| 795 | CCCUUUAU G CCGCUGUU | 1474 | AACAGCGG GGCTAGCTACAACGA ATAAAGGG | 4528 |
| 798 | UUUAUGCC G CUGUUACC | 1475 | GGTAACAG GGCTAGCTACAACGA GGCATAAA | 4529 |
| 911 | GGCACAUU G CCACAGGA | 1476 | TCCTGTGG GGCTAGCTACAACGA AATGTGCC | 4530 |
| 1020 | UGGGGUUU G CCGCCCCU | 1479 | AGGGGCGG GGCTAGCTACAACGA AAACCCCA | 4531 |
| 1023 | GGUUUGCC G CCCCUUUC | 1480 | GAAAGGGG GGCTAGCTACAACGA GGCAAACC | 4532 |
| 1034 | CCUUUCAC G CAAUGUGG | 1481 | CCACATTG GGCTAGCTACAACGA GTGAAAGG | 4533 |
| 1050 | GAUAUUCU G CUUUAAUG | 1482 | CATTAAAG GGCTAGCTACAACGA AGAATATC | 4534 |
| 1058 | GCUUUAAU G CCUUUAUA | 1483 | TATAAAGG GGCTAGCTACAACGA ATTAAAGC | 4535 |
| 1068 | CUUUAUAU G CAUGCAUA | 1484 | TATGCATG GGCTAGCTACAACGA ATATAAAG | 4536 |
| 1072 | AUAUGCAU G CAUACAAG | 1485 | CTTGTATG GGCTAGCTACAACGA ATGCATAT | 4537 |
| 1103 | ACUUUCUC G CCAACUUA | 1486 | TAAGTTGG GGCTAGCTACAACGA GAGAAAGT | 4538 |
| 1155 | ACCCCGUU G CUCGGCAA | 1488 | TTGCCGAG GGCTAGCTACAACGA AACGGGGT | 4539 |
| 1177 | UGGUCUAU G CCAAGUGU | 1489 | ACACTTGG GGCTAGCTACAACGA ATAGACCA | 4540 |
| 1188 | AAGUGUUU G CUGACGCA | 1490 | TGCGTCAG GGCTAGCTACAACGA AAACACTT | 4541 |
| 1194 | UUGCUGAC G CAACCCCC | 1492 | GGGGGTTG GGCTAGCTACAACGA GTCAGCAA | 4542 |
| 1234 | CCAUCAGC G CAUGCGUG | 1493 | CACGCATG GGCTAGCTACAACGA GCTGATGG | 4543 |
| 1238 | CAGCGCAU G CGUGGAAC | 1494 | GTTCCACG GGCTAGCTACAACGA ATGCGCTG | 4544 |
| 1262 | UCUCCUCU G CCGAUCCA | 1495 | TGGATCGG GGCTAGCTACAACGA AGAGGAGA | 4545 |
| 1275 | UCCAUACC G CGGAACUC | 1497 | GAGTTCCG GGCTAGCTACAACGA GGTATGGA | 4546 |
| 1290 | UCCUAGCC G CUUGUUUU | 1498 | AAAACAAG GGCTAGCTACAACGA GGCTAGGA | 4547 |
| 1299 | CUUGUUUU G CUCGCAGC | 1499 | GCTGCGAG GGCTAGCTACAACGA AAAACAAG | 4548 |
| 1303 | UUUUGCUC G CAGCAGGU | 1500 | ACCTGCTG GGCTAGCTACAACGA GAGCAAAA | 4549 |
| 1349 | UCUGUCGU G CUCUCCCG | 1502 | CGGGAGAG GGCTAGCTACAACGA ACGACAGA | 4550 |
| 1357 | GCUCUCCC G CAAAUAUA | 1503 | TATATTTG GGCTAGCTACAACGA GGGAGAGC | 4551 |
| 1382 | CCAUGGCU G CUAGGCUG | 1504 | CAGCCTAG GGCTAGCTACAACGA AGCCATGG | 4552 |
| 1392 | UAGGCUGU G CUGCCAAC | 1505 | GTTGGCAG GGCTAGCTACAACGA ACAGCCTA | 4553 |
| 1395 | GCUGUGCU G CCAACUGG | 1506 | CCAGTTGG GGCTAGCTACAACGA AGCACAGC | 4554 |
| 1411 | GAUCCUAC G CGGGACGU | 1507 | ACGTCCCG GGCTAGCTACAACGA GTAGGATC | 4555 |
| 1442 | CCGUCGGC G CUGAAUCC | 1508 | GGATTCAG GGCTAGCTACAACGA GCCGACGG | 4556 |
| 1452 | UGAAUCCC G CGGACGAC | 1510 | GTCGTCCG GGCTAGCTACAACGA GGGATTCA | 4557 |
| 1474 | CCGGGGCC G CUUGGGGC | 1512 | GCCCCAAG GGCTAGCTACAACGA GGCCCCGG | 4558 |
| 1489 | GCUCUACC G CCCGCUUC | 1513 | GAAGCGGG GGCTAGCTACAACGA GGTAGAGC | 4559 |
| 1493 | UACCGCCC G CUUCUCCG | 1514 | CGGAGAAG GGCTAGCTACAACGA GGGCGGTA | 4560 |
| 1501 | GCUUCUCC G CCUAUUGU | 1515 | ACAATAGG GGCTAGCTACAACGA GGAGAAGC | 4561 |
| 1528 | CACGGGGC G CACCUCUC | 1517 | GAGAGGTG GGCTAGCTACAACGA GCCCCGTG | 4562 |
| 1542 | CUCUUUAC G CGGACUCC | 1518 | GGAGTCCG GGCTAGCTACAACGA GTAAAGAG | 4563 |
| 1559 | CCGUCUGU G CCUUCUCA | 1519 | TGAGAAGG GGCTAGCTACAACGA ACAGACGG | 4564 |
| 1571 | UCUCAUCU G CCGGACCG | 1520 | CGGTCCGG GGCTAGCTACAACGA AGATGAGA | 4565 |
| 1583 | GACCGUGU G CACUUCGC | 1521 | GCGAAGTG GGCTAGCTACAACGA ACACGGTC | 4566 |
| 1590 | UGCACUUC G CUUCACCU | 1522 | AGGTGAAG GGCTAGCTACAACGA GAAGTGCA | 4567 |
| 1601 | UCACCUCU G CACGUCGC | 1523 | GCGACGTG GGCTAGCTACAACGA AGAGGTGA | 4568 |
| 1608 | UGCACGUC G CAUGGAGA | 1524 | TCTCCATG GGCTAGCTACAACGA GACGTGCA | 4569 |
| 1628 | CCGUGAAC G CCCACAGG | 1526 | CCTGTGGG GGCTAGCTACAACGA GTTCACGG | 4570 |
| 1642 | AGGAACCU G CCCAAGGU | 1527 | ACCTTGGG GGCTAGCTACAACGA AGGTTCCT | 4571 |
| 1654 | AAGGUCUU G CAUAAGAG | 1528 | CTCTTATG GGCTAGCTACAACGA AAGACCTT | 4572 |
| 1818 | AGCACCAU G CAACUUUU | 1533 | AAAAGTTG GGCTAGCTACAACGA ATGGTGCT | 4573 |
| 1835 | UCACCUCU G CCUAAUCA | 1534 | TGATTAGG GGCTAGCTACAACGA AGAGGTGA | 4574 |
| 1883 | CAAGCUGU G CCUUGGGU | 1535 | ACCCAAGG GGCTAGCTACAACGA ACAGCTTG | 4575 |
| 1959 | UCUUUUUU G CCUUCUGA | 1537 | TCAGAAGG GGCTAGCTACAACGA AAAAAAGA | 4576 |
| 2002 | UCGACACC G CCUCUGCU | 1541 | AGCAGAGG GGCTAGCTACAACGA GGTGTCGA | 4577 |
| 2008 | CCGCCUCU G CUCUGUAU | 1542 | ATACAGAG GGCTAGCTACAACGA AGAGGCGG | 4578 |
| 2282 | GUGGAUUC G CACUCCUC | 1548 | GAGGAGTG GGCTAGCTACAACGA GAATCCAC | 4579 |
| 2293 | CUCCUCCU G CAUAUAGA | 1549 | TCTATATG GGCTAGCTACAACGA AGGAGGAG | 4580 |
| 2311 | CACCAAAU G CCCCUAUC | 1550 | GATAGGGG GGCTAGCTACAACGA ATTTGGTG | 4581 |
| 2388 | ACUCCCUC G CCUCGCAG | 1552 | CTGCGAGG GGCTAGCTACAACGA GAGGGAGT | 4582 |
| 2393 | CUCGCCUC G CAGACGAA | 1553 | TTCGTCTG GGCTAGCTACAACGA GAGGCGAG | 4583 |
| 2412 | UCUCAAUC G CCGCGUCG | 1555 | CGACGCGG GGCTAGCTACAACGA GATTGAGA | 4584 |
| 2415 | CAAUCGCC G CGUCGCAG | 1556 | CTGCGACG GGCTAGCTACAACGA GGCGATTG | 4585 |
| 2420 | GCCGCGUC G CAGAAGAU | 1557 | ATCTTCTG GGCTAGCTACAACGA GACGCGGC | 4586 |
| 2514 | GGUACCUU G CUUUAAUC | 1558 | GATTAAAG GGCTAGCTACAACGA AAGGTACC | 4587 |
| 2560 | AUUCAUUU G CAGGAGGA | 1560 | TCCTCCTG GGCTAGCTACAACGA AAATGAAT | 4588 |
| 2641 | UUAACUAU G CCUGCUAG | 1563 | CTAGCAGG GGCTAGCTACAACGA ATAGTTAA | 4589 |
| 2645 | CUAUGCCU G CUAGGUUU | 1564 | AAACCTAG GGCTAGCTACAACGA AGGCATAG | 4590 |
| 2677 | AAAUAUUU G CCCUUAGA | 1565 | TCTAAGGG GGCTAGCTACAACGA AAATATTT | 4591 |
| 2740 | UUCCAGAC G CGACAUUA | 1566 | TAATGTCG GGCTAGCTACAACGA GTCTGGAA | 4592 |
| 2804 | CACGUAGC G CCUCAUUU | 1568 | AAATGAGG GGCTAGCTACAACGA GCTACGTG | 4593 |
| 2814 | CUCAUUUU G CGGGUCAC | 1569 | GTGACCCG GGCTAGCTACAACGA AAAATGAG | 4594 |
| 2946 | UGGACCCU G CAUUCAAA | 1572 | TTTGAATG GGCTAGCTACAACGA AGGGTCCA | 4595 |
| 2990 | CUCAACCC G CACAAGGA | 1573 | TCCTTGTG GGCTAGCTACAACGA GGGTTGAG | 4596 |
| 3012 | GGCCGGAC G CCAACAAG | 1574 | CTTGTTGG GGCTAGCTACAACGA GTCCGGCC | 4597 |
| 3090 | GCCCUCAC G CUCAGGGC | 1575 | GCCCTGAG GGCTAGCTACAACGA GTGAGGGC | 4598 |
| 3113 | ACAACUGU G CCAGCAGC | 1576 | GCTGCTGG GGCTAGCTACAACGA ACAGTTGT | 4599 |
| 3132 | CUCCUCCU G CCUCCACC | 1577 | GGTGGAGG GGCTAGCTACAACGA AGGAGGAG | 4600 |
| 51 | AGGGCCCU G UACUUUCC | 1578 | GGAAAGTA GGCTAGCTACAACGA AGGGCCCT | 4601 |
| 106 | AGAAUACU G UCUCUGCC | 1579 | GGCAGAGA GGCTAGCTACAACGA AGTATTCT | 4602 |
| 148 | GGGACCCU G UACCGAAC | 1580 | GTTCGGTA GGCTAGCTACAACGA AGGGTCCC | 4603 |
| 198 | CUGCUCGU G UUACAGGC | 1581 | GCCTGTAA GGCTAGCTACAACGA ACGAGCAG | 4604 |
| 219 | UUUUUCUU G UUGACAAA | 1582 | TTTGTCAA GGCTAGCTACAACGA AAGAAAAA | 4605 |
| 297 | ACACCCGU G UGUCUUGG | 1583 | CCAAGACA GGCTAGCTACAACGA ACGGGTGT | 4606 |
| 299 | ACCCGUGU G UCUUGGCC | 1584 | GGCCAAGA GGCTAGCTACAACGA ACACGGGT | 4607 |
| 347 | ACCAACCU G UUGUCCUC | 1585 | GAGGACAA GGCTAGCTACAACGA AGGTTGGT | 4608 |
| 350 | AACCUGUU G UCCUCCAA | 1586 | TTGGAGGA GGCTAGCTACAACGA AACAGGTT | 4609 |
| 362 | UCCAAUUU G UCCUGGUU | 1587 | AACCAGGA GGCTAGCTACAACGA AAATTGGA | 4610 |
| 381 | CGCUGGAU G UGUCUGCG | 1588 | CGCAGACA GGCTAGCTACAACGA ATCCAGCG | 4611 |
| 383 | CUGGAUGU G UCUGCGGC | 1589 | GCCGCAGA GGCTAGCTACAACGA ACATCCAG | 4612 |
| 438 | AUCUUCUU G UUGGUUCU | 1590 | AGAACCAA GGCTAGCTACAACGA AAGAAGAT | 4613 |
| 465 | CAAGGUAU G UUGCCCGU | 1591 | ACGGGCAA GGCTAGCTACAACGA ATACCTTG | 4614 |
| 476 | GCCCGUUU G UCCUCUAA | 1592 | TTAGAGGA GGCTAGCTACAACGA AAACGGGC | 4615 |
| 555 | ACCUCUAU G UUUCCCUC | 1593 | GAGGGAAA GGCTAGCTACAACGA ATAGAGGT | 4616 |
| 566 | UCCCUCAU G UUGCUGUA | 1594 | TACAGCAA GGCTAGCTACAACGA ATGAGGGA | 4617 |
| 572 | AUGUUGCU G UACAAAAC | 1595 | GTTTTGTA GGCTAGCTACAACGA AGCAACAT | 4618 |
| 602 | CUGCACCU G UAUUCCCA | 1596 | TGGGAATA GGCTAGCTACAACGA AGGTGCAG | 4619 |
| 694 | UGCCAUUU G UUCAGUGG | 1597 | CCACTGAA GGCTAGCTACAACGA AAATGGCA | 4620 |
| 724 | CCCCCACU G UCUGGCUU | 1598 | AAGCCAGA GGCTAGCTACAACGA AGTGGGGG | 4621 |
| 750 | UGGAUGAU G UGGUUUUG | 1599 | CAAAACCA GGCTAGCTACAACGA ATCATCCA | 4622 |
| 771 | CCAAGUCU G UACAACAU | 1600 | ATGTTGTA GGCTAGCTACAACGA AGACTTGG | 4623 |
| 801 | AUGCCGCU G UUACCAAU | 1601 | ATTGGTAA GGCTAGCTACAACGA AGCGGCAT | 4624 |
| 818 | UUUCUUUU G UCUUUGGG | 1602 | CCCAAAGA GGCTAGCTACAACGA AAAAGAAA | 4625 |
| 888 | UGGGAUAU G UAAUUGGG | 1603 | CCCAATTA GGCTAGCTACAACGA ATATCCCA | 4626 |
| 927 | AACAUAUU G UACAAAAA | 1604 | TTTTTGTA GGCTAGCTACAACGA AATATGTT | 4627 |
| 944 | AUCAAAAU G UGUUUUAG | 1605 | CTAAAACA GGCTAGCTACAACGA ATTTTGAT | 4628 |
| 946 | CAAAAUGU G UUUUAGGA | 1606 | TCCTAAAA GGCTAGCTACAACGA ACATTTTG | 4629 |
| 963 | AACUUCCU G UAAACAGG | 1607 | CCTGTTTA GGCTAGCTACAACGA AGGAAGTT | 4630 |
| 991 | GAAAGUAU G UCAACGAA | 1608 | TTCGTTGA GGCTAGCTACAACGA ATACTTTC | 4631 |
| 1002 | AACGAAUU G UGGGUCUU | 1609 | AAGACCCA GGCTAGCTACAACGA AATTCGTT | 4632 |
| 1039 | CACGCAAU G UGGAUAUU | 1610 | AATATCCA GGCTAGCTACAACGA ATTGCGTG | 4633 |
| 1137 | AACAGUAU G UGAACCUU | 1611 | AAGGTTCA GGCTAGCTACAACGA ATACTGTT | 4634 |
| 1184 | UGCCAAGU G UUUGCUGA | 1612 | TCAGCAAA GGCTAGCTACAACGA ACTTGGCA | 4635 |
| 1251 | GAACCUUU G UGUCUCCU | 1613 | AGGAGACA GGCTAGCTACAACGA AAAGGTTC | 4636 |
| 1253 | ACCUUUGU G UCUCCUCU | 1614 | AGAGGAGA GGCTAGCTACAACGA ACAAAGGT | 4637 |
| 1294 | AGCCGCUU G UUUUGCUC | 1615 | GAGCAAAA GGCTAGCTACAACGA AAGCGGCT | 4638 |
| 1344 | ACAAUUCU G UCGUGCUC | 1616 | GAGCACGA GGCTAGCTACAACGA AGAATTGT | 4639 |
| 1390 | GCUAGGCU G UGCUGCCA | 1617 | TGGCAGCA GGCTAGCTACAACGA AGCCTAGC | 4640 |
| 1425 | CGUCCUUU G UUUACGUC | 1618 | GACGTAAA GGCTAGCTACAACGA AAAGGACG | 4641 |
| 1508 | CGCCUAUU G UACCGACC | 1619 | GGTCGGTA GGCTAGCTACAACGA AATAGGCG | 4642 |
| 1557 | CCCCGUCU G UGCCUUCU | 1620 | AGAAGGCA GGCTAGCTACAACGA AGACGGGG | 4643 |
| 1581 | CGGACCGU G UGCACUUC | 1621 | GAAGTGCA GGCTAGCTACAACGA ACGGTCCG | 4644 |
| 1684 | UCAGCAAU G UCAACGAC | 1622 | GTCGTTGA GGCTAGCTACAACGA ATTGCTGA | 4645 |
| 1719 | CAAAGACU G UGUGUUUA | 1623 | TAAACACA GGCTAGCTACAACGA AGTCTTTG | 4646 |
| 1721 | AAGACUGU G UGUUUAAU | 1624 | ATTAAACA GGCTAGCTACAACGA ACAGTCTT | 4647 |
| 1723 | GACUGUGU G UUUAAUGA | 1625 | TCATTAAA GGCTAGCTACAACGA ACACAGTC | 4648 |
| 1772 | AGGUCUUU G UACUAGGA | 1626 | TCCTAGTA GGCTAGCTACAACGA AAAGACCT | 4649 |
| 1785 | AGGAGGCU G UAGGCAUA | 1627 | TATGCCTA GGCTAGCTACAACGA AGCCTCCT | 4650 |
| 1801 | AAAUUGGU G UGUUCACC | 1628 | GGTGAACA GGCTAGCTACAACGA ACCAATTT | 4651 |
| 1803 | AUUGGUGU G UUCACCAG | 1629 | CTGGTGAA GGCTAGCTACAACGA ACACCAAT | 4652 |
| 1850 | CAUCUCAU G UUCAUGUC | 1630 | GACATGAA GGCTAGCTACAACGA ATGAGATG | 4653 |
| 1856 | AUGUUCAU G UCCUACUG | 1631 | CAGTAGGA GGCTAGCTACAACGA ATGAACAT | 4654 |
| 1864 | GUCCUACU G UUCAAGCC | 1632 | GGCTTGAA GGCTAGCTACAACGA AGTAGGAC | 4655 |
| 1881 | UCCAAGCU G UGCCUUGG | 1633 | CCAAGGCA GGCTAGCTACAACGA AGCTTGGA | 4656 |
| 1939 | GAGCUUCU G UGGAGUUA | 1634 | TAACTCCA GGCTAGCTACAACGA AGAAGCTC | 4657 |
| 2013 | UCUGCUCU G UAUCGGGG | 1635 | CCCCGATA GGCTAGCTACAACGA AGAGCAGA | 4658 |
| 2045 | GGAACAUU G UUCACCUC | 1636 | GAGGTGAA GGCTAGCTACAACGA AATGTTCC | 4659 |
| 2082 | GCUAUUCU G UGUUGGGG | 1637 | CCCCAACA GGCTAGCTACAACGA AGAATAGC | 4660 |
| 2084 | UAUUCUGU G UUGGGGUG | 1638 | CACCCCAA GGCTAGCTACAACGA ACAGAATA | 4661 |
| 2167 | UCAGCUAU G UCAACGUU | 1639 | AACGTTGA GGCTAGCTACAACGA ATAGCTGA | 4662 |
| 2205 | CAACUAUU G UGGUUUCA | 1640 | TGAAACCA GGCTAGCTACAACGA AATAGTTG | 4663 |
| 2222 | CAUUUCCU G UCUUACUU | 1641 | AAGTAAGA GGCTAGCTACAACGA AGGAAATG | 4664 |
| 2245 | GAGAAACU G UUCUUGAA | 1642 | TTCAAGAA GGCTAGCTACAACGA AGTTTCTC | 4665 |
| 2262 | UAUUUGGU G UCUUUUGG | 1643 | CCAAAAGA GGCTAGCTACAACGA ACCAAATA | 4666 |
| 2274 | UUUGGAGU G UGGAUUCG | 1644 | CGAATCCA GGCTAGCTACAACGA ACTCCAAA | 4667 |
| 2344 | AAACUACU G UUGUUAGA | 1645 | TCTAACAA GGCTAGCTACAACGA AGTAGTTT | 4668 |
| 2347 | CUACUGUU G UUAGACGA | 1646 | TCGTCTAA GGCTAGCTACAACGA AACAGTAG | 4669 |
| 2450 | AUCUCAAU G UUAGUAUU | 1647 | AATACTAA GGCTAGCTACAACGA ATTGAGAT | 4670 |
| 2573 | AGGACAUU G UUGAUAGA | 1648 | TCTATCAA GGCTAGCTACAACGA AATGTCCT | 4671 |
| 2583 | UGAUAGAU G UAAGCAAU | 1649 | ATTGCTTA GGCTAGCTACAACGA ATCTATCA | 4672 |
| 2594 | AGCAAUUU G UGGGGCCC | 1650 | GGGCCCCA GGCTAGCTACAACGA AAATTGCT | 4673 |
| 2663 | AUCCCAAU G UUACUAAA | 1651 | TTTAGTAA GGCTAGCTACAACGA ATTGGGAT | 4674 |
| 2717 | CAGAGUAU G UAGUUAAU | 1652 | ATTAACTA GGCTAGCTACAACGA ATACTCTG | 4675 |
| 2901 | AUCUUUCU G UCCCCAAU | 1653 | ATTGGGGA GGCTAGCTACAACGA AGAAAGAT | 4676 |
| 3071 | GGGGGACU G UUGGGGUG | 1654 | CACCCCAA GGCTAGCTACAACGA AGTCCCCC | 4677 |
| 3111 | UCACAACU G UGCCAGCA | 1655 | TGCTGGCA GGCTAGCTACAACGA AGTTGTGA | 4678 |
| 40 | AUCCCAGA G UCAGGGCC | 1656 | GGCCCTGA GGCTAGCTACAACGA TCTGGGAT | 4679 |
| 46 | GAGUCAGG G CCCUGUAC | 1657 | GTACAGGG GGCTAGCTACAACGA CCTGACTC | 4680 |
| 65 | UCCUGCUG G UGGCUCCA | 1658 | TGGAGCCA GGCTAGCTACAACGA CAGCAGGA | 4681 |
| 68 | UGCUGGUG G CUCCAGUU | 1659 | AACTGGAG GGCTAGCTACAACGA CACCAGCA | 4682 |
| 74 | UGGCUCCA G UUCAGGAA | 1660 | TTCCTGAA GGCTAGCTACAACGA TGGAGCCA | 4683 |
| 85 | CAGGAACA G UGAGCCCU | 1661 | AGGGCTCA GGCTAGCTACAACGA TGTTCCTG | 4684 |
| 89 | AACAGUGA G CCCUGCUC | 1662 | GAGCAGGG GGCTAGCTACAACGA TCACTGTT | 4685 |
| 120 | GCCAUAUC G UCAAUCUU | 1663 | AAGATTGA GGCTAGCTACAACGA GATATGGC | 4686 |
| 196 | CCCUGCUC G UGUUACAG | 1664 | CTGTAACA GGCTAGCTACAACGA GAGCAGGG | 4687 |
| 205 | UGUUACAG G CGGGGUUU | 1665 | AAACCCCG GGCTAGCTACAACGA CTGTAACA | 4688 |
| 210 | CAGGCGGG G UUUUUCUU | 1666 | AAGAAAAA GGCTAGCTACAACGA CCCGCCTG | 4689 |
| 248 | ACCACAGA G UCUAGACU | 1667 | AGTCTAGA GGCTAGCTACAACGA TCTGTGGT | 4690 |
| 258 | CUAGACUC G UGGUGGAC | 1668 | GTCCACCA GGCTAGCTACAACGA GAGTCTAG | 4691 |
| 261 | GACUCGUG G UGGACUUC | 1669 | GAAGTCCA GGCTAGCTACAACGA CACGAGTC | 4692 |
| 295 | GAACACCC G UGUGUCUU | 1670 | AAGACACA GGCTAGCTACAACGA GGGTGTTC | 4693 |
| 305 | GUGUCUUG G CCAAAAUU | 1671 | AATTTTGG GGCTAGCTACAACGA CAAGACAC | 4694 |
| 318 | AAUUCGCA G UCCCAAAU | 1672 | ATTTGGGA GGCTAGCTACAACGA TGCGAATT | 4695 |
| 332 | AAUCUCCA G UCACUCAC | 1673 | GTGAGTGA GGCTAGCTACAACGA TGGAGATT | 4696 |
| 368 | UUGUCCUG G UUAUCGCU | 1674 | AGCGATAA GGCTAGCTACAACGA CAGGACAA | 4697 |
| 390 | UGUCUGCG G CGUUUUAU | 1675 | ATAAAACG GGCTAGCTACAACGA CGCAGACA | 4698 |
| 392 | UCUGCGGC G UUUUAUCA | 1676 | TGATAAAA GGCTAGCTACAACGA GCCGCAGA | 4699 |
| 442 | UCUUGUUG G UUCUUCUG | 1677 | CAGAAGAA GGCTAGCTACAACGA CAACAAGA | 4700 |
| 461 | CUAUCAAG G UAUGUUGC | 1678 | GCAACATA GGCTAGCTACAACGA CTTGATAG | 4701 |
| 472 | UGUUGCCC G UUUGUCCU | 1679 | AGGACAAA GGCTAGCTACAACGA GGGCAACA | 4702 |
| 506 | AACAACCA G CACCGGAC | 1680 | GTCCGGTG GGCTAGCTACAACGA TGGTTGTT | 4703 |
| 625 | CAUCUUGG G CUUUCGCA | 1681 | TGCGAAAG GGCTAGCTACAACGA CCAAGATG | 4704 |
| 648 | CUAUGGGA G UGGGCCUC | 1682 | GAGGCCCA GGCTAGCTACAACGA TCCCATAG | 4705 |
| 652 | GGGAGUGG G CCUCAGUC | 1683 | GACTGAGG GGCTAGCTACAACGA CCACTCCC | 4706 |
| 658 | GGGCCUCA G UCCGUUUC | 1684 | GAAACGGA GGCTAGCTACAACGA TGAGGCCC | 4707 |
| 662 | CUCAGUCC G UUUCUCUU | 1685 | AAGAGAAA GGCTAGCTACAACGA GGACTGAG | 4708 |
| 672 | UUCUCUUG G CUCAGUUU | 1686 | AAACTGAG GGCTAGCTACAACGA CAAGAGAA | 4709 |
| 677 | UUGGCUCA G UUUACUAG | 1687 | CTAGTAAA GGCTAGCTACAACGA TGAGCCAA | 4710 |
| 685 | GUUUACUA G UGCCAUUU | 1688 | AAATGGCA GGCTAGCTACAACGA TAGTAAAC | 4711 |
| 699 | UUUGUUCA G UGGUUCGU | 1689 | ACGAACCA GGCTAGCTACAACGA TGAACAAA | 4712 |
| 702 | GUUCAGUG G UUCGUAGG | 1690 | CCTACGAA GGCTAGCTACAACGA CACTGAAC | 4713 |
| 706 | AGUGGUUC G UAGGGCUU | 1691 | AAGCCCTA GGCTAGCTACAACGA GAACCACT | 4714 |
| 711 | UUCGUAGG G CUUUCCCC | 1692 | GGGGAAAG GGCTAGCTACAACGA CCTACGAA | 4715 |
| 729 | ACUGUCUG G CUUUCAGU | 1693 | ACTGAAAG GGCTAGCTACAACGA CAGACAGT | 4716 |
| 736 | GGCUUUCA G UUAUAUGG | 1694 | CCATATAA GGCTAGCTACAACGA TGAAAGCC | 4717 |
| 753 | AUGAUGUG G UUUUGGGG | 1695 | CCCCAAAA GGCTAGCTACAACGA CACATCAT | 4718 |
| 762 | UUUUGGGG G CCAAGUCU | 1696 | AGACTTGG GGCTAGCTACAACGA CCCCAAAA | 4719 |
| 767 | GGGGCCAA G UCUGUACA | 1697 | TGTACAGA GGCTAGCTACAACGA TTGGCCCC | 4720 |
| 785 | CAUCUUGA G UCCCUUUA | 1698 | TAAAGGGA GGCTAGCTACAACGA TCAAGATG | 4721 |
| 826 | GUCUUUGG G UAUACAUU | 1699 | AATGTATA GGCTAGCTACAACGA CCAAAGAC | 4722 |
| 898 | AAUUGGGA G UUGGGGCA | 1700 | TGCCCCAA GGCTAGCTACAACGA TCCCAATT | 4723 |
| 904 | GAGUUGGG G CACAUUGC | 1701 | GCAATGTG GGCTAGCTACAACGA CCCAACTC | 4724 |
| 971 | GUAAACAG G CCUAUUGA | 1702 | TCAATAGG CGCTAGCTACAACGA CTGTTTAC | 4725 |
| 987 | AUUGGAAA G UAUGUCAA | 1703 | TTGACATA GGCTAGCTACAACGA TTTCCAAT | 4726 |
| 1006 | AAUUGUGG G UCUUUUGG | 1704 | CCAAAAGA GGCTAGCTACAACGA CCACAATT | 4727 |
| 1016 | CUUUUGGG G UUUGCCGC | 1705 | GCGGCAAA GGCTAGCTACAACGA CCCAAAAG | 4728 |
| 1080 | GCAUACAA G CAAAACAG | 1706 | CTGTTTTG GGCTAGCTACAACGA TTGTATGC | 4729 |
| 1089 | CAAAACAG G CUUUUACU | 1707 | AGTAAAAG GGCTAGCTACAACGA CTGTTTTG | 4730 |
| 1116 | CUUACAAG G CCUUUCUA | 1708 | TAGAAAGG GGCTAGCTACAACGA CTTGTAAG | 4731 |
| 1126 | CUUUCUAA G UAAACAGU | 1709 | ACTGTTTA GGCTAGCTACAACGA TTAGAAAG | 4732 |
| 1133 | AGUAAACA G UAUGUGAA | 1710 | TTCACATA GGCTAGCTACAACGA TGTTTACT | 4733 |
| 1152 | UUUACCCC G UUGCUCGG | 1711 | CCGAGCAA GGCTAGCTACAACGA GGGGTAAA | 4734 |
| 1160 | GUUGCUCG G CAACGGCC | 1712 | GGCCGTTG GGCTAGCTACAACGA CGAGCAAC | 4735 |
| 1166 | CGGCAACG G CCUGGUCU | 1713 | AGACCAGG GGCTAGCTACAACGA CGTTGCCG | 4736 |
| 1171 | ACGGCCUG G UCUAUGCC | 1714 | GGCATAGA GGCTAGCTACAACGA CAGGCCGT | 4737 |
| 1182 | UAUGCCAA G UGUUUGCU | 1715 | AGCAAACA GGCTAGCTACAACGA TTGGCATA | 4738 |
| 1207 | CCCCACUG G UUGGGGCU | 1716 | AGCCCCAA GGCTAGCTACAACGA CAGTGGGG | 4739 |
| 1213 | UGGUUGGG G CUUGGCCA | 1717 | TGGCCAAG GGCTAGCTACAACGA CCCAACCA | 4740 |
| 1218 | GGGGCUUG G CCAUAGGC | 1718 | GCCTATGG GGCTAGCTACAACGA CAAGCCCC | 4741 |
| 1225 | GGCCAUAG G CCAUCAGC | 1719 | GCTGATGG GGCTAGCTACAACGA CTATGGCC | 4742 |
| 1232 | GGCCAUCA G CGCAUGCG | 1720 | CGCATGCG GGCTAGCTACAACGA TGATGGCC | 4743 |
| 1240 | GCGCAUGC G UGGAACCU | 1721 | AGGTTCCA GGCTAGCTACAACGA GCATGCGC | 4744 |
| 1287 | AACUCCUA G CCGCUUGU | 1722 | ACAAGCGG GGCTAGCTACAACGA TAGGAGTT | 4745 |
| 1306 | UGCUCGCA G CAGGUCUG | 1723 | CAGACCTG GGCTAGCTACAACGA TGCGAGCA | 4746 |
| 1310 | CGCAGCAG G UCUGGGGC | 1724 | GCCCCAGA GGCTAGCTACAACGA CTGCTGCG | 4747 |
| 1317 | GGUCUGGG G CAAAACUC | 1725 | GAGTTTTG GGCTAGCTACAACGA CCCAGACC | 4748 |
| 1347 | AUUCUGUC G UGCUCUCC | 1726 | GGAGAGCA GGCTAGCTACAACGA GACAGAAT | 4749 |
| 1379 | UUUCCAUG G CUGCUAGG | 1727 | CCTAGCAG GGCTAGCTACAACGA CATGGAAA | 4750 |
| 1387 | GCUGCUAG G CUGUGCUG | 1728 | CAGCACAG GGCTAGCTACAACGA CTAGCAGC | 4751 |
| 1418 | CGCGGGAC G UCCUUUGU | 1729 | ACAAAGGA GGCTAGCTACAACGA GTCCCGCG | 4752 |
| 1431 | UUGUUUAC G UCCCGUCG | 1730 | CGACGGGA GGCTAGCTACAACGA GTAAACAA | 4753 |
| 1436 | UACGUCCC G UCGGCGCU | 1731 | AGCGCCGA GGCTAGCTACAACGA GGGACGTA | 4754 |
| 1440 | UCCCGUCG G CGCUGAAU | 1732 | ATTCAGCG GGCTAGCTACAACGA CGACGGGA | 4755 |
| 1471 | CUCCCGGG G CCGCUUGG | 1733 | CCAAGCGG GGCTAGCTACAACGA CCCGGGAG | 4756 |
| 1481 | CGCUUGGG G CUCUACCG | 1734 | CGGTAGAG GGCTAGCTACAACGA CCCAAGCG | 4757 |
| 1517 | UACCGACC G UCCACGGG | 1735 | CCCGTGGA GGCTAGCTACAACGA GGTCGGTA | 4758 |
| 1526 | UCCACGGG G CGCACCUC | 1736 | GAGGTGCG GGCTAGCTACAACGA CCCGTGGA | 4759 |
| 1553 | GACUCCCC G UCUGUGCC | 1737 | GGCACAGA GGCTAGCTACAACGA GGGGAGTC | 4760 |
| 1579 | GCCGGACC G UGUGCACU | 1738 | AGTGCACA GGCTAGCTACAACGA GGTCCGGC | 4761 |
| 1605 | CUCUGCAC G UCGCAUGG | 1739 | CCATGCGA GGCTAGCTACAACGA GTGCAGAG | 4762 |
| 1622 | AGACCACC G UGAACGCC | 1740 | GGCGTTCA GGCTAGCTACAACGA GGTGGTCT | 4763 |
| 1649 | UGCCCAAG G UCUUGCAU | 1741 | ATGCAAGA GGCTAGCTACAACGA CTTGGGCA | 4764 |
| 1679 | GACUUUCA G CAAUGUCA | 1742 | TGACATTG GGCTAGCTACAACGA TGAAAGTC | 4765 |
| 1703 | ACCUUGAG G CAUACUUC | 1743 | GAAGTATG GGCTAGCTACAACGA CTCAAGGT | 4766 |
| 1732 | UUUAAUGA G UGGGAGGA | 1744 | TCCTCCCA GGCTAGCTACAACGA TCATTAAA | 4767 |
| 1741 | UGGGAGGA G UUGGGGGA | 1745 | TCCCCCAA GGCTAGCTACAACGA TCCTCCCA | 4768 |
| 1754 | GGGAGGAG G UUAGGUUA | 1746 | TAACCTAA GGCTAGCTACAACGA CTCCTCCC | 4769 |
| 1759 | GAGGUUAG G UUAAAGGU | 1747 | ACCTTTAA GGCTAGCTACAACGA CTAACCTC | 4770 |
| 1766 | GGUUAAAG G UCUUUGUA | 1748 | TACAAAGA GGCTAGCTACAACGA CTTTAACC | 4771 |
| 1782 | ACUAGGAG G CUGUAGGC | 1749 | GCCTACAG GGCTAGCTACAACGA CTCCTAGT | 4772 |
| 1789 | GGCUGUAG G CAUAAAUU | 1750 | AATTTATG GGCTAGCTACAACGA CTACAGCC | 4773 |
| 1799 | AUAAAUUG G UGUGUUCA | 1751 | TGAACACA GGCTAGCTACAACGA CAATTTAT | 4774 |
| 1811 | GUUCACCA G CACCAUGC | 1752 | GCATGGTG GGCTAGCTACAACGA TGGTGAAC | 4775 |
| 1870 | CUGUUCAA G CCUCCAAG | 1753 | CTTGGAGG GGCTAGCTACAACGA TTGAACAG | 4776 |
| 1878 | GCCUCCAA G CUGUGCCU | 1754 | AGGCACAG GGCTAGCTACAACGA TTGGAGGC | 4777 |
| 1890 | UGCCUUGG G UGGCUUUG | 1755 | CAAAGCCA GGCTAGCTACAACGA CCAAGGCA | 4778 |
| 1893 | CUUGGGUG G CUUUGGGG | 1756 | CCCCAAAG GGCTAGCTACAACGA CACCCAAG | 4779 |
| 1901 | GCUUUGGG G CAUGGACA | 1757 | TGTCCATG GGCTAGCTACAACGA CCCAAAGC | 4780 |
| 1917 | AUUGACCC G UAUAAAGA | 1758 | TCTTTATA GGCTAGCTACAACGA GGGTCAAT | 4781 |
| 1933 | AAUUUGGA G CUUCUGUG | 1759 | CACAGAAG GGCTAGCTACAACGA TCCAAATT | 4782 |
| 1944 | UCUGUGGA G UUACUCUC | 1760 | GAGAGTAA GGCTAGCTACAACGA TCCACAGA | 4783 |
| 2023 | AUCGGGGG G CCUUAGAG | 1761 | CTCTAAGG GGCTAGCTACAACGA CCCCCGAT | 4784 |
| 2031 | GCCUUAGA G UCUCCGGA | 1762 | TCCGGAGA GGCTAGCTACAACGA TCTAAGGC | 4785 |
| 2062 | ACCAUACG G CACUCAGG | 1763 | CCTGAGTG GGCTAGCTACAACGA CGTATGGT | 4786 |
| 2070 | GCACUCAG G CAAGCUAU | 1764 | ATAGCTTG GGCTAGCTACAACGA CTGAGTGC | 4787 |
| 2074 | UCAGGCAA G CUAUUCUG | 1765 | CAGAATAG GGCTAGCTACAACGA TTGCCTGA | 4788 |
| 2090 | GUGUUGGG G UGAGUUGA | 1766 | TCAACTCA GGCTAGCTACAACGA CCCAACAC | 4789 |
| 2094 | UGGGGUGA G UUGAUGAA | 1767 | TTCATCAA GGCTAGCTACAACGA TCACCCCA | 4790 |
| 2107 | UGAAUCUA G CCACCUGG | 1768 | CCAGGTGG GGCTAGCTACAACGA TAGATTCA | 4791 |
| 2116 | CCACCUGG G UGGGAAGU | 1769 | ACTTCCCA GGCTAGCTACAACGA CCAGGTGG | 4792 |
| 2123 | GGUGGGAA G UAAUUUGG | 1770 | CCAAATTA GGCTAGCTACAACGA TTCCCACC | 4793 |
| 2140 | AAGAUCCA G CAUCCAGG | 1771 | CCTGGATG GGCTAGCTACAACGA TGGATCTT | 4794 |
| 2155 | GGGAAUUA G UAGUCAGC | 1772 | GCTGACTA GGCTAGCTACAACGA TAATTCCC | 4795 |
| 2158 | AAUUAGUA G UCAGCUAU | 1773 | ATAGCTGA GGCTAGCTACAACGA TACTAATT | 4796 |
| 2162 | AGUAGUCA G CUAUGUCA | 1774 | TGACATAG GGCTAGCTACAACGA TGACTACT | 4797 |
| 2173 | AUGUCAAC G UUAAUAUG | 1775 | CATATTAA GGCTAGCTACAACGA GTTGACAT | 4798 |
| 2183 | UAAUAUGG G CCUAAAAA | 1776 | TTTTTAGG GGCTAGCTACAACGA CCATATTA | 4799 |
| 2208 | CUAUUGUG G UUUCACAU | 1777 | ATGTGAAA GGCTAGCTACAACGA CACAATAG | 4800 |
| 2235 | ACUUUUGG G CGAGAAAC | 1778 | GTTTCTCG GGCTAGCTACAACGA CCAAAAGT | 4801 |
| 2260 | AAUAUUUG G UGUCUUUU | 1779 | AAAAGACA GGCTAGCTACAACGA CAAATATT | 4802 |
| 2272 | CUUUUGGA G UGUGGAUU | 1780 | AATCCACA GGCTAGCTACAACGA TCCAAAAG | 4803 |
| 2360 | ACGAAGAG G CAGGUCCC | 1781 | GGGACCTG GGCTAGCTACAACGA CTCTTCGT | 4804 |
| 2364 | AGAGGCAG G UCCCCUAG | 1782 | CTAGGGGA GGCTAGCTACAACGA CTGCCTCT | 4805 |
| 2403 | AGACGAAG G UCUCAAUC | 1783 | GATTGAGA GGCTAGCTACAACGA CTTCGTCT | 4806 |
| 2417 | AUCGCCGC G UCGCAGAA | 1784 | TTCTGCGA GGCTAGCTACAACGA GCGGCGAT | 4807 |
| 2454 | CAAUGUUA G UAUUCCUU | 1785 | AAGGAATA GGCTAGCTACAACGA TAACATTG | 4808 |
| 2474 | CACAUAAG G UGGGAAAC | 1786 | GTTTCCCA GGCTAGCTACAACGA CTTATGTG | 4809 |
| 2491 | UUUACGGG G CUUUAUUC | 1787 | GAATAAAG GGCTAGCTACAACGA CCCGTAAA | 4810 |
| 2507 | CUUCUACG G UACCUUGC | 1788 | GCAAGGTA GGCTAGCTACAACGA CGTAGAAG | 4811 |
| 2530 | CCUAAAUG G CAAACUCC | 1789 | GGAGTTTG GGCTAGCTACAACGA CATTTAGG | 4812 |
| 2587 | AGAUGUAA G CAAUUUGU | 1790 | ACAAATTG GGCTAGCTACAACGA TTACATCT | 4813 |
| 2599 | UUUGUGGG G CCCCUUAC | 1791 | GTAAGGGG GGCTAGCTACAACGA CCCACAAA | 4814 |
| 2609 | CCCUUACA G UAAAUGAA | 1792 | TTCATTTA GGCTAGCTACAACGA TGTAAGGG | 4815 |
| 2650 | CCUGCUAG G UUUUAUCC | 1793 | GGATAAAA GGCTAGCTACAACGA CTAGCAGG | 4816 |
| 2701 | AUCAAACC G UAUUAUCC | 1794 | GGATAATA GGCTAGCTACAACGA GGTTTGAT | 4817 |
| 2713 | UAUCCAGA G UAUGUAGU | 1795 | ACTACATA GGCTAGCTACAACGA TCTGGATA | 4818 |
| 2720 | AGUAUGUA G UUAAUCAU | 1796 | ATGATTAA GGCTAGCTACAACGA TACATACT | 4819 |
| 2768 | UUUGGAAG G CGGGGAUC | 1797 | GATCCCCG GGCTAGCTACAACGA CTTCCAAA | 4820 |
| 2791 | AAAAGAGA G UCCACACG | 1798 | CGTGTGGA GGCTAGCTACAACGA TCTCTTTT | 4821 |
| 2799 | GUCCACAC G UAGCGCCU | 1799 | AGGCGCTA GGCTAGCTACAACGA GTGTGGAC | 4822 |
| 2802 | CACACGUA G CGCCUCAU | 1800 | ATGAGGCG GGCTAGCTACAACGA TACGTGTG | 4823 |
| 2818 | UUUUGCGG G UCACCAUA | 1801 | TATGGTGA GGCTAGCTACAACGA CCGCAAAA | 4824 |
| 2848 | GAUCUACA G CAUGGGAG | 1802 | CTCCCATG GGCTAGCTACAACGA TGTAGATC | 4825 |
| 2857 | CAUGGGAG G UUGGUCUU | 1803 | AAGACCAA GGCTAGCTACAACGA CTCCCATG | 4826 |
| 2861 | GGAGGUUG G UCUUCCAA | 1804 | TTGGAAGA GGCTAGCTACAACGA CAACCTCC | 4827 |
| 2881 | UCGAAAAG G CAUGGGGA | 1805 | TCCCCATG GGCTAGCTACAACGA CTTTTCGA | 4828 |
| 2936 | GAUCAUCA G UUGGACCC | 1806 | GGGTCCAA GGCTAGCTACAACGA TGATGATC | 4829 |
| 2955 | CAUUCAAA G CCAACUCA | 1807 | TGAGTTGG GGCTAGCTACAACGA TTTGAATG | 4830 |
| 2964 | CCAACUCA G UAAAUCCA | 1808 | TGGATTTA GGCTAGCTACAACGA TGAGTTGG | 4831 |
| 3005 | GACAACUG G CCGGACGC | 1809 | GCGTCCGG GGCTAGCTACAACGA CAGTTGTC | 4832 |
| 3021 | CCAACAAG G UGGGAGUG | 1810 | CACTCCCA GGCTAGCTACAACGA CTTGTTGG | 4833 |
| 3027 | AGGUGGGA G UGGGAGCA | 1811 | TGCTCCCA GGCTAGCTACAACGA TCCCACCT | 4834 |
| 3033 | GAGUGGGA G CAUUCGGG | 1812 | CCCGAATG GGCTAGCTACAACGA TCCCACTC | 4835 |
| 3041 | GCAUUCGG G CCAGGGUU | 1813 | AACCCTGG GGCTAGCTACAACGA CCGAATGC | 4836 |
| 3047 | GGGCCAGG G UUCACCCC | 1814 | GGGGTGAA GGCTAGCTACAACGA CCTGGCCC | 4837 |
| 3077 | CUGUUGGG G UGGAGCCC | 1815 | GGGCTCCA GGCTAGCTACAACGA CCCAACAG | 4838 |
| 3082 | GGGGUGGA G CCCUCACG | 1816 | CGTGAGGG GGCTAGCTACAACGA TCCACCCC | 4839 |
| 3097 | CGCUCAGG G CCUACUCA | 1817 | TGAGTAGG GGCTAGCTACAACGA CCTGAGCG | 4840 |
| 3117 | CUGUGCCA G CAGCUCCU | 1818 | AGGAGCTG GGCTAGCTACAACGA TGGCACAG | 4841 |
| 3120 | UGCCAGCA G CUCCUCCU | 1819 | AGGAGGAG GGCTAGCTACAACGA TGCTGGCA | 4842 |
| 3146 | ACCAAUCG G CAGUCAGG | 1820 | CCTGACTG GGCTAGCTACAACGA CGATTGGT | 4843 |
| 3149 | AAUCGGCA G UCAGGAAG | 1821 | CTTCCTGA GGCTAGCTACAACGA TGCCGATT | 4844 |
| 3158 | UCAGGAAG G CAGCCUAC | 1822 | GTAGGCTG GGCTAGCTACAACGA CTTCCTGA | 4845 |
| 3161 | GGAAGGCA G CCUACUCC | 1823 | GGAGTAGG GGCTAGCTACAACGA TGCCTTCC | 4846 |
| 3204 | AUCCUCAG G CCAUGCAG | 1824 | CTGCATGG GGCTAGCTACAACGA CTGAGGAT | 4847 |
| 10 | ACUCCACC A CUUUCCAC | 1825 | GTGGAAAG GGCTAGCTACAACGA GGTGGAGT | 4848 |
| 17 | CACUUUCC A CCAAACUC | 1826 | GAGTTTGG GGCTAGCTACAACGA GGAAAGTG | 4849 |
| 22 | UCCACCAA A CUCUUCAA | 1827 | TTGAAGAG GGCTAGCTACAACGA TTGGTGGA 4850 | |
| 32 | UCUUCAAG A UCCCAGAG | 1828 | CTCTGGGA GGCTAGCTACAACGA CTTGAAGA | 4851 |
| 53 | GGCCCUGU A CUUUCCUG | 1829 | CAGGAAAG GGCTAGCTACAACGA ACAGGGCC 4852 | |
| 82 | GUUCAGGA A CAGUGAGC | 1830 | GCTCACTG GGCTAGCTACAACGA TCCTGAAC | 4853 |
| 101 | UGCUCAGA A UACUGUCU | 1831 | AGACAGTA GGCTAGCTACAACGA TCTGAGCA | 4854 |
| 103 | CUCAGAAU A CUGUCUCU | 1832 | AGAGACAG GGCTAGCTACAACGA ATTCTGAG | 4855 |
| 115 | UCUCUGCC A UAUCGUCA | 1833 | TGACGATA GGCTAGCTACAACGA GGCAGAGA | 4856 |
| 117 | UCUGCCAU A UCGUCAAU | 1834 | ATTGACGA GGCTAGCTACAACGA ATGGCAGA | 4857 |
| 124 | UAUCGUCA A UCUUAUCG | 1835 | CGATAAGA GGCTAGCTACAACGA TGACGATA | 4858 |
| 129 | UCAAUCUU A UCGAAGAC | 1836 | GTCTTCGA GGCTAGCTACAACGA AAGATTGA | 4859 |
| 136 | UAUCGAAG A CUGGGGAC | 1837 | GTCCCCAG GGCTAGCTACAACGA CTTCGATA | 4860 |
| 143 | GACUGGGG A CCCUGUAC | 1838 | GTACAGGG GGCTAGCTACAACGA CCCCAGTC | 4861 |
| 150 | GACCCUGU A CCGAACAU | 1839 | ATGTTCGG GGCTAGCTACAACGA ACAGGGTC | 4862 |
| 155 | UGUACCGA A CAUGGAGA | 1840 | TCTCCATG GGCTAGCTACAACGA TCGGTACA | 4863 |
| 157 | UACCGAAC A UGGAGAAC | 1841 | GTTCTCCA GGCTAGCTACAACGA GTTCGGTA | 4864 |
| 164 | CAUGGAGA A CAUCGCAU | 1842 | ATGCGATG GGCTAGCTACAACGA TCTCCATG | 4865 |
| 166 | UGGAGAAC A UCGCAUCA | 1843 | TGATGCGA GGCTAGCTACAACGA GTTCTCCA | 4866 |
| 171 | AACAUCGC A UCAGGACU | 1844 | AGTCCTGA GGCTAGCTACAACGA GCGATGTT | 4867 |
| 177 | GCAUCAGG A CUCCUAGG | 1845 | CCTAGGAG GGCTAGCTACAACGA CCTGATGC | 4868 |
| 186 | CUCCUAGG A CCCCUGCU | 1846 | AGCAGGGG GGCTAGCTACAACGA CCTAGGAG | 4869 |
| 201 | CUCGUGUU A CAGGCGGG | 1847 | CCCGCCTG GGCTAGCTACAACGA AACACGAG | 4870 |
| 223 | UCUUGUUG A CAAAAAUC | 1848 | GATTTTTG GGCTAGCTACAACGA CAACAAGA | 4871 |
| 229 | UGACAAAA A UCCUCACA | 1849 | TGTGAGGA GGCTAGCTACAACGA TTTTGTCA | 4872 |
| 235 | AAAUCCUC A CAAUACCA | 1850 | TGGTATTG GGCTAGCTACAACGA GAGGATTT | 4873 |
| 238 | UCCUCACA A UACCACAG | 1851 | CTGTGGTA GGCTAGCTACAACGA TGTGAGGA | 4874 |
| 240 | CUCACAAU A CCACAGAG | 1852 | CTCTGTGG GGCTAGCTACAACGA ATTGTGAG | 4875 |
| 243 | ACAAUACC A CAGAGUCU | 1853 | AGACTCTG GGCTAGCTACAACGA GGTATTGT | 4876 |
| 254 | GAGUCUAG A CUCGUGGU | 1854 | ACCACGAG GGCTAGCTACAACGA CTAGACTC | 4877 |
| 265 | CGUGGUGG A CUUCUCUC | 1855 | GAGAGAAG GGCTAGCTACAACGA CCACCACG | 4878 |
| 275 | UUCUCUCA A UUUUCUAG | 1856 | CTAGAAAA GGCTAGCTACAACGA TGAGAGAA | 4879 |
| 289 | UAGGGGGA A CACCCGUG | 1857 | CACGGGTG GGCTAGCTACAACGA TCCCCCTA | 4880 |
| 291 | GGGGGAAC A CCCGUGUG | 1858 | CACACGGG GGCTAGCTACAACGA GTTCCCCC | 4881 |
| 311 | UGGCCAAA A UUCGCAGU | 1859 | ACTGCGAA GGCTAGCTACAACGA TTTGGCCA | 4882 |
| 325 | AGUCCCAA A UCUCCAGU | 1860 | ACTGGAGA GGCTAGCTACAACGA TTGGGACT | 4883 |
| 335 | CUCCAGUC A CUCACCAA | 1861 | TTGGTGAG GGCTAGCTACAACGA GACTGGAG | 4884 |
| 339 | AGUCACUC A CCAACCUG | 1862 | CAGGTTGG GGCTAGCTACAACGA GAGTGACT | 4885 |
| 343 | ACUCACCA A CCUGUUGU | 1863 | ACAACAGG GGCTAGCTACAACGA TGGTGAGT | 4886 |
| 358 | GUCCUCCA A UUUGUCCU | 1864 | AGGACAAA GGCTAGCTACAACGA TGGAGGAC | 4887 |
| 371 | UCCUGGUU A UCGCUGGA | 1865 | TCCAGCGA GGCTAGCTACAACGA AACCAGGA | 4888 |
| 379 | AUCGCUGG A UGUGUCUG | 1866 | CAGACACA GGCTAGCTACAACGA CCAGCGAT | 4889 |
| 397 | GGCGUUUU A UCAUCUUC | 1867 | GAAGATGA GGCTAGCTACAACGA AAAACGCC | 4890 |
| 400 | GUUUUAUC A UCUUCCUC | 1868 | GAGGAAGA GGCTAGCTACAACGA GATAAAAC | 4891 |
| 412 | UCCUCUGC A UCCUGCUG | 1869 | CAGCAGGA GGCTAGCTACAACGA GCAGAGGA | 4892 |
| 423 | CUGCUGCU A UGCCUCAU | 1870 | ATGAGGCA GGCTAGCTACAACGA AGCAGCAG | 4893 |
| 430 | UAUGCCUC A UCUUCUUG | 1871 | CAAGAAGA GGCTAGCTACAACGA GAGGCATA | 4894 |
| 452 | UCUUCUGG A CUAUCAAG | 1872 | CTTGATAG GGCTAGCTACAACGA CCAGAAGA | 4895 |
| 455 | UCUGGACU A UCAAGGUA | 1873 | TACCTTGA GGCTAGCTACAACGA AGTCCAGA | 4896 |
| 463 | AUCAAGGU A UGUUGCCC | 1874 | GGGCAACA GGCTAGCTACAACGA ACCTTGAT | 4897 |
| 484 | GUCCUCUA A UUCCAGGA | 1875 | TCCTGGAA GGCTAGCTACAACGA TAGAGGAC | 4898 |
| 492 | AUUCCAGG A UCAUCAAC | 1876 | GTTGATGA GGCTAGCTACAACGA CCTGGAAT | 4899 |
| 495 | CCAGGAUC A UCAACAAC | 1877 | GTTGTTGA GGCTAGCTACAACGA GATCCTGG | 4900 |
| 499 | GAUCAUCA A CAACCAGC | 1878 | GCTGGTTG GGCTAGCTACAACGA TGATGATC | 4901 |
| 502 | CAUCAACA A CCAGCACC | 1879 | GGTGCTGG GGCTAGCTACAACGA TGTTGATG | 4902 |
| 513 | AGCACCGG A CCAUGCAA | 1880 | TTGCATGG GGCTAGCTACAACGA CCGGTGCT | 4903 |
| 516 | ACCGGACC A UGCAAAAC | 1881 | GTTTTGCA GGCTAGCTACAACGA GGTCCGGT | 4904 |
| 523 | CAUGCAAA A CCUGCACA | 1882 | TGTGCAGG GGCTAGCTACAACGA TTTGCATG | 4905 |
| 529 | AAACCUGC A CAACUCCU | 1883 | AGGAGTTG GGCTAGCTACAACGA GCAGGTTT | 4906 |
| 532 | CCUGCACA A CUCCUGCU | 1884 | AGCAGGAG GGCTAGCTACAACGA TGTGCAGG | 4907 |
| 547 | CUCAAGGA A CCUCUAUG | 1885 | CATAGAGG GGCTAGCTACAACGA TCCTTGAG | 4908 |
| 553 | GAACCUCU A UGUUUCCC | 1886 | GGGAAACA GGCTAGCTACAACGA AGAGGTTC | 4909 |
| 564 | UUUCCCUC A UGUUGCUG | 1887 | CAGCAACA GGCTAGCTACAACGA GAGGGAAA | 4910 |
| 574 | GUUGCUGU A CAAAACCU | 1888 | AGGTTTTG GGCTAGCTACAACGA ACAGCAAC | 4911 |
| 579 | UGUACAAA A CCUACGGA | 1889 | TCCGTAGG GGCTAGCTACAACGA TTTGTACA | 4912 |
| 583 | CAAAACCU A CGGACGGA | 1890 | TCCGTCCG GGCTAGCTACAACGA AGGTTTTG | 4913 |
| 587 | ACCUACGG A CGGAAACU | 1891 | AGTTTCCG GGCTAGCTACAACGA CCGTAGGT | 4914 |
| 593 | GGACGGAA A CUGCACCU | 1892 | AGGTGCAG GGCTAGCTACAACGA TTCCGTCC | 4915 |
| 598 | GAAACUGC A CCUGUAUU | 1893 | AATACAGG GGCTAGCTACAACGA GCAGTTTC | 4916 |
| 604 | GCACCUGU A UUCCCAUC | 1894 | GATGGGAA GGCTAGCTACAACGA ACAGGTGC | 4917 |
| 610 | GUAUUCCC A UCCCAUCA | 1895 | TGATGGGA GGCTAGCTACAACGA GGGAATAC | 4918 |
| 615 | CCCAUCCC A UCAUCUUG | 1896 | CAAGATGA GGCTAGCTACAACGA GGGATGGG | 4919 |
| 618 | AUCCCAUC A UCUUGGGC | 1897 | GCCCAAGA GGCTAGCTACAACGA GATGGGAT | 4920 |
| 636 | UUCGCAAA A UACCUAUG | 1898 | CATAGGTA GGCTAGCTACAACGA TTTGCGAA | 4921 |
| 638 | CGCAAAAU A CCUAUGGG | 1899 | CCCATAGG GGCTAGCTACAACGA ATTTTGCG | 4922 |
| 642 | AAAUACCU A UGGGAGUG | 1900 | CACTCCCA GGCTAGCTACAACGA AGGTATTT | 4923 |
| 681 | CUCAGUUU A CUAGUGCC | 1901 | GGCACTAG GGCTAGCTACAACGA AAACTGAG | 4924 |
| 690 | CUAGUGCC A UUUGUUCA | 1902 | TGAACAAA GGCTAGCTACAACGA GGCACTAG | 4925 |
| 721 | UUUCCCCC A CUGUCUGG | 1903 | CCAGACAG GGCTAGCTACAACGA GGGGGAAA | 4926 |
| 739 | UUUCAGUU A UAUGGAUG | 1904 | CATCCATA GGCTAGCTACAACGA AACTGAAA | 4927 |
| 741 | UCAGUUAU A UGGAUGAU | 1905 | ATCATCCA GGCTAGCTACAACGA ATAACTGA | 4928 |
| 745 | UUAUAUGG A UGAUGUGG | 1906 | CCACATCA GGCTAGCTACAACGA CCATATAA | 4929 |
| 748 | UAUGGAUG A UGUGGUUU | 1907 | AAACCACA GGCTAGCTACAACGA CATCCATA | 4930 |
| 773 | AAGUCUGU A CAACAUCU | 1908 | AGATGTTG GGCTAGCTACAACGA ACAGACTT | 4931 |
| 776 | UCUGUACA A CAUCUUGA | 1909 | TCAAGATG GGCTAGCTACAACGA TGTACAGA | 4932 |
| 778 | UGUACAAC A UCUUGAGU | 1910 | ACTCAAGA GGCTAGCTACAACGA GTTGTACA | 4933 |
| 793 | GUCCCUUU A UGCCGCUG | 1911 | CAGCGGCA GGCTAGCTACAACGA AAAGGGAC | 4934 |
| 804 | CCGCUGUU A CCAAUUUU | 1912 | AAAATTGG GGCTAGCTACAACGA AACAGCGG | 4935 |
| 808 | UGUUACCA A UUUUCUUU | 1913 | AAAGAAAA GGCTAGCTACAACGA TGGTAACA | 4936 |
| 828 | CUUUGGGU A UACAUUUA | 1914 | TAAATGTA GGCTAGCTACAACGA ACCCAAAG | 4937 |
| 830 | UUGGGUAU A CAUUUAAA | 1915 | TTTAAATG GGCTAGCTACAACGA ATACCCAA | 4938 |
| 832 | GGGUAUAC A UUUAAACC | 1916 | GGTTTAAA GGCTAGCTACAACGA GTATACCC | 4939 |
| 838 | ACAUUUAA A CCCUCACA | 1917 | TGTGAGGG GGCTAGCTACAACGA TTAAATGT | 4940 |
| 844 | AAACCCUC A CAAAACAA | 1918 | TTGTTTTG GGCTAGCTACAACGA GAGGGTTT | 4941 |
| 849 | CUCACAAA A CAAAAAGA | 1919 | TCTTTTTG GGCTAGCTACAACGA TTTGTGAG | 4942 |
| 857 | ACAAAAAG A UGGGGAUA | 1920 | TATCCCCA GGCTAGCTACAACGA CTTTTTGT | 4943 |
| 863 | AGAUGGGG A UAUUCCCU | 1921 | AGGGAATA GGCTAGCTACAACGA CCCCATCT | 4944 |
| 865 | AUGGGGAU A UUCCCUUA | 1922 | TAAGGGAA GGCTAGCTACAACGA ATCCCCAT | 4945 |
| 874 | UUCCCUUA A CUUCAUGG | 1923 | CCATGAAG GGCTAGCTACAACGA TAAGGGAA | 4946 |
| 879 | UUAACUUC A UGGGAUAU | 1924 | ATATCCCA GGCTAGCTACAACGA GAAGTTAA | 4947 |
| 884 | UUCAUGGG A UAUGUAAU | 1925 | ATTACATA GGCTAGCTACAACGA CCCATGAA | 4948 |
| 886 | CAUGGGAU A UGUAAUUG | 1926 | CAATTACA GGCTAGCTACAACGA ATCCCATG | 4949 |
| 891 | GAUAUGUA A UUGGGAGU | 1927 | ACTCCCAA GGCTAGCTACAACGA TACATATC | 4950 |
| 906 | GUUGGGGC A CAUUGCCA | 1928 | TGGCAATG GGCTAGCTACAACGA GCCCCAAC | 4951 |
| 908 | UGGGGCAC A UUGCCACA | 1929 | TGTGGCAA GGCTAGCTACAACGA GTGCCCCA | 4952 |
| 914 | ACAUUGCC A CAGGAACA | 1930 | TGTTCCTG GGCTAGCTACAACGA GGCAATGT | 4953 |
| 920 | CCACAGGA A CAUAUUGU | 1931 | ACAATATG GGCTAGCTACAACGA TCCTGTGG | 4954 |
| 922 | ACAGGAAC A UAUUGUAC | 1932 | GTACAATA GGCTAGCTACAACGA GTTCCTGT | 4955 |
| 924 | AGGAACAU A UUGUACAA | 1933 | TTGTACAA GGCTAGCTACAACGA ATGTTCCT | 4956 |
| 929 | CAUAUUGU A CAAAAAAU | 1934 | ATTTTTTG GGCTAGCTACAACGA ACAATATG | 4957 |
| 936 | UACAAAAA A UCAAAAUG | 1935 | CATTTTGA GGCTAGCTACAACGA TTTTTGTA | 4958 |
| 942 | AAAUCAAA A UGUGUUUU | 1936 | AAAACACA GGCTAGCTACAACGA TTTGATTT | 4959 |
| 956 | UUUAGGAA A CUUCCUGU | 1937 | ACAGGAAG GGCTAGCTACAACGA TTCCTAAA | 4960 |
| 967 | UCCUGUAA A CAGGCCUA | 1938 | TAGGCCTG GGCTAGCTACAACGA TTACAGGA | 4961 |
| 975 | ACAGGCCU A UUGAUUGG | 1939 | CCAATCAA GGCTAGCTACAACGA AGGCCTGT | 4962 |
| 979 | GCCUAUUG A UUGGAAAG | 1940 | CTTTCCAA GGCTAGCTACAACGA CAATAGGC | 4963 |
| 989 | UGGAAAGU A UGUCAACG | 1941 | CGTTGACA GGCTAGCTACAACGA ACTTTCCA | 4964 |
| 995 | GUAUGUCA A CGAAUUGU | 1942 | ACAATTCG GGCTAGCTACAACGA TGACATAC | 4965 |
| 999 | GUCAACGA A UUGUGGGU | 1943 | ACCCACAA GGCTAGCTACAACGA TCGTTGAC | 4966 |
| 1032 | CCCCUUUC A CGCAAUGU | 1944 | ACATTGCG GGCTAGCTACAACGA GAAAGGGG | 4967 |
| 1037 | UUCACGCA A UGUGGAUA | 1945 | TATCCACA GGCTAGCTACAACGA TGCGTGAA | 4968 |
| 1043 | CAAUGUGG A UAUUCUGC | 1946 | GCAGAATA GGCTAGCTACAACGA CCACATTG | 4969 |
| 1045 | AUGUGGAU A UUCUGCUU | 1947 | AAGCAGAA GGCTAGCTACAACGA ATCCACAT | 4970 |
| 1056 | CUGCUUUA A UGCCUUUA | 1948 | TAAAGGCA GGCTAGCTACAACGA TAAAGCAG | 4971 |
| 1064 | AUGCCUUU A UAUGCAUG | 1949 | CATGCATA GGCTAGCTACAACGA AAAGGCAT | 4972 |
| 1066 | GCCUUUAU A UGCAUGCA | 1950 | TGCATGCA GGCTAGCTACAACGA ATAAAGGC | 4973 |
| 1070 | UUAUAUGC A UGCAUACA | 1951 | TGTATGCA GGCTAGCTACAACGA GCATATAA | 4974 |
| 1074 | AUGCAUGC A UACAAGCA | 1952 | TGCTTGTA GGCTAGCTACAACGA GCATGCAT | 4975 |
| 1076 | GCAUGCAU A CAAGCAAA | 1953 | TTTGCTTG GGCTAGCTACAACGA ATGCATGC | 4976 |
| 1085 | CAAGCAAA A CAGGCUUU | 1954 | AAAGCCTG GGCTAGCTACAACGA TTTGCTTG | 4977 |
| 1095 | AGGCUUUU A CUUUCUCG | 1955 | CGAGAAAG GGCTAGCTACAACGA AAAAGCCT | 4978 |
| 1107 | UCUCGCCA A CUUACAAG | 1956 | CTTGTAAG GGCTAGCTACAACGA TGGCGAGA | 4979 |
| 1111 | GCCAACUU A CAAGGCCU | 1957 | AGGCCTTG GGCTAGCTACAACGA AAGTTGGC | 4980 |
| 1130 | CUAAGUAA A CAGUAUGU | 1958 | ACATACTG GGCTAGCTACAACGA TTACTTAG | 4981 |
| 1135 | UAAACAGU A UGUGAACC | 1959 | GGTTCACA GGCTAGCTACAACGA ACTGTTTA | 4982 |
| 1141 | GUAUGUGA A CCUUUACC | 1960 | GGTAAAGG GGCTAGCTACAACGA TCACATAC | 4983 |
| 1147 | GAACCUUU A CCCCGUUG | 1961 | CAACGGGG GGCTAGCTACAACGA AAAGGTTC | 4984 |
| 1163 | GCUCGGCA A CGGCCUGG | 1962 | CCAGGCCG GGCTAGCTACAACGA TGCCGAGC | 4985 |
| 1175 | CCUGGUCU A UGCCAAGU | 1963 | ACTTGGCA GGCTAGCTACAACGA AGACCAGG | 4986 |
| 1192 | GUUUGCUG A CGCAACCC | 1964 | GGGTTGCG GGCTAGCTACAACGA CAGCAAAC | 4987 |
| 1197 | CUGACGCA A CCCCCACU | 1965 | AGTGGGGG GGCTAGCTACAACGA TGCGTCAG | 4988 |
| 1203 | CAACCCCC A CUGGUUGG | 1966 | CCAACCAG GGCTAGCTACAACGA GGGGGTTG | 4989 |
| 1221 | GCUUGGCC A UAGGCCAU | 1967 | ATGGCCTA GGCTAGCTACAACGA GGCCAAGC | 4990 |
| 1228 | CAUAGGCC A UCAGCGCA | 1968 | TGCGCTGA GGCTAGCTACAACGA GGCCTATG | 4991 |
| 1236 | AUCAGCGC A UGCGUGGA | 1969 | TCCACGCA GGCTAGCTACAACGA GCGCTGAT | 4992 |
| 1245 | UGCGUGGA A CCUUUGUG | 1970 | CACAAAGG GGCTAGCTACAACGA TCCACGCA | 4993 |
| 1266 | CUCUGCCG A UCCAUACC | 1971 | GGTATGGA GGCTAGCTACAACGA CGGCAGAG | 4994 |
| 1270 | GCCGAUCC A UACCGCGG | 1972 | CCGCGGTA GGCTAGCTACAACGA GGATCGGC | 4995 |
| 1272 | CGAUCCAU A CCGCGGAA | 1973 | TTCCGCGG GGCTAGCTACAACGA ATGGATCG | 4996 |
| 1280 | ACCGCGGA A CUCCUAGC | 1974 | GCTAGGAG GGCTAGCTACAACGA TCCGCGGT | 4997 |
| 1322 | GGGGCAAA A CUCAUCGG | 1975 | CCGATGAG GGCTAGCTACAACGA TTTGCCCC | 4998 |
| 1326 | CAAAACUC A UCGGGACU | 1976 | AGTCCCGA GGCTAGCTACAACGA GAGTTTTG | 4999 |
| 1332 | UCAUCGGG A CUGACAAU | 1977 | ATTGTCAG GGCTAGCTACAACGA CCCGATGA | 5000 |
| 1336 | CGGGACUG A CAAUUCUG | 1978 | CAGAATTG GGCTAGCTACAACGA CAGTCCCG | 5001 |
| 1339 | GACUGACA A UUCUGUCG | 1979 | CGACAGAA GGCTAGCTACAACGA TGTCAGTC | 5002 |
| 1361 | UCCCGCAA A UAUACAUC | 1980 | GATGTATA GGCTAGCTACAACGA TTGCGGGA | 5003 |
| 1363 | CCGCAAAU A UACAUCAU | 1981 | ATGATGTA GGCTAGCTACAACGA ATTTGCGG | 5004 |
| 1365 | GCAAAUAU A CAUCAUUU | 1982 | AAATGATG GGCTAGCTACAACGA ATATTTGC | 5005 |
| 1367 | AAAUAUAC A UCAUUUCC | 1983 | GGAAATGA GGCTAGCTACAACGA GTATATTT | 5006 |
| 1370 | UAUACAUC A UUUCCAUG | 1984 | CATGGAAA GGCTAGCTACAACGA GATGTATA | 5007 |
| 1376 | UCAUUUCC A UGGCUGCU | 1985 | AGCAGCCA GGCTAGCTACAACGA GGAAATGA | 5008 |
| 1399 | UGCUGCCA A CUGGAUCC | 1986 | GGATCCAG GGCTAGCTACAACGA TGGCAGCA | 5009 |
| 1404 | CCAACUGG A UCCUACGC | 1987 | GCGTAGGA GGCTAGCTACAACGA CCAGTTGG | 5010 |
| 1409 | UGGAUCCU A CGCGGGAC | 1988 | GTCCCGCG GGCTAGCTACAACGA AGGATCCA | 5011 |
| 1416 | UACGCGGG A CGUCCUUU | 1989 | AAAGGACG GGCTAGCTACAACGA CCCGCGTA | 5012 |
| 1429 | CUUUGUUU A CGUCCCGU | 1990 | ACGGGACG GGCTAGCTACAACGA AAACAAAG | 5013 |
| 1447 | GGCGCUGA A UCCCGCGG | 1991 | CCGCGGGA GGCTAGCTACAACGA TCAGCGCC | 5014 |
| 1456 | UCCCGCGG A CGACCCCU | 1992 | AGGGGTCG GGCTAGCTACAACGA CCGCGGGA | 5015 |
| 1459 | CGCGGACG A CCCCUCCC | 1993 | GGGAGGGG GGCTAGCTACAACGA CGTCCGCG | 5016 |
| 1486 | GGGGCUCU A CCGCCCGC | 1994 | GCGGGCGG GGCTAGCTACAACGA AGAGCCCC | 5017 |
| 1505 | CUCCGCCU A UUGUACCG | 1995 | CGGTACAA GGCTAGCTACAACGA AGGCGGAG | 5018 |
| 1510 | CCUAUUGU A CCGACCGU | 1996 | ACGGTCGG GGCTAGCTACAACGA ACAATAGG | 5019 |
| 1514 | UUGUACCG A CCGUCCAC | 1997 | GTGGACGG GGCTAGCTACAACGA CGGTACAA | 5020 |
| 1521 | GACCGUCC A CGGGGCGC | 1998 | GCGCCCCG GGCTAGCTACAACGA GGACGGTC | 5021 |
| 1530 | CGGGGCGC A CCUCUCUU | 1999 | AAGAGAGG GGCTAGCTACAACGA GCGCCCCG | 5022 |
| 1540 | CUCUCUUU A CGCGGACU | 2000 | AGTCCGCG GGCTAGCTACAACGA AAAGAGAG | 5023 |
| 1546 | UUACGCGG A CUCCCCGU | 2001 | ACGGGGAG GGCTAGCTACAACGA CCGCGTAA | 5024 |
| 1567 | GCCUUCUC A UCUGCCGG | 2002 | CCGGCAGA GGCTAGCTACAACGA GAGAAGGC | 5025 |
| 1576 | UCUGCCGG A CCGUGUGC | 2003 | GCACACGG GGCTAGCTACAACGA CCGGCAGA | 5026 |
| 1585 | CCGUGUGC A CUUCGCUU | 2004 | AAGCGAAG GGCTAGCTACAACGA GCACACGG | 5027 |
| 1595 | UUCGCUUC A CCUCUGCA | 2005 | TGCAGAGG GGCTAGCTACAACGA GAAGCGAA | 5028 |
| 1603 | ACCUCUGC A CGUCGCAU | 2006 | ATGCGACG GGCTAGCTACAACGA GCAGAGGT | 5029 |
| 1610 | CACGUCGC A UGGAGACC | 2007 | GGTCTCCA GGCTAGCTACAACGA GCGACGTG | 5030 |
| 1616 | GCAUGGAG A CCACCGUG | 2008 | CACGGTGG GGCTAGCTACAACGA CTCCATGC | 5031 |
| 1619 | UGGAGACC A CCGUGAAC | 2009 | GTTCACGG GGCTAGCTACAACGA GGTCTCCA | 5032 |
| 1626 | CACCGUGA A CGCCCACA | 2010 | TGTGGGCG GGCTAGCTACAACGA TCACGGTG | 5033 |
| 1638 | CCACAGGA A CCUGCCCA | 2011 | TGGGCAGG GGCTAGCTACAACGA TCCTGTGG | 5034 |
| 1656 | GGUCUUGC A UAAGAGGA | 2012 | TCCTCTTA GGCTAGCTACAACGA GCAAGACC | 5035 |
| 1664 | AUAAGAGG A CUCUUGGA | 2013 | TCCAAGAG GGCTAGCTACAACGA CCTCTTAT | 5036 |
| 1672 | ACUCUUGG A CUUUCAGC | 2014 | GCTGAAAG GGCTAGCTACAACGA CCAAGAGT | 5037 |
| 1682 | UUUCAGCA A UGUCAACG | 2015 | CGTTGACA GGCTAGCTACAACGA TGCTGAAA | 5038 |
| 1688 | CAAUGUCA A CGACCGAC | 2016 | GTCGGTCG GGCTAGCTACAACGA TGACATTG | 5039 |
| 1691 | UGUCAACG A CCGACCUU | 2017 | AAGGTCGG GGCTAGCTACAACGA CGTTGACA | 5040 |
| 1695 | AACGACCG A CCUUGAGG | 2018 | CCTCAAGG GGCTAGCTACAACGA CGGTCGTT | 5041 |
| 1705 | CUUGAGGC A UACUUCAA | 2019 | TTGAAGTA GGCTAGCTACAACGA GCCTCAAG | 5042 |
| 1707 | UGAGGCAU A CUUCAAAG | 2020 | CTTTGAAG GGCTAGCTACAACGA ATGCCTCA | 5043 |
| 1716 | CUUCAAAG A CUGUGUGU | 2021 | ACACACAG GGCTAGCTACAACGA CTTTGAAG | 5044 |
| 1728 | UGUGUUUA A UGAGUGGG | 2022 | CCCACTCA GGCTAGCTACAACGA TAAACACA | 5045 |
| 1774 | GUCUUUGU A CUAGGAGG | 2023 | CCTCCTAG GGCTAGCTACAACGA ACAAAGAC | 5046 |
| 1791 | CUGUAGGC A UAAAUUGG | 2024 | CCAATTTA GGCTAGCTACAACGA GCCTACAG | 5047 |
| 1795 | AGGCAUAA A UUGGUGUG | 2025 | CACACCAA GGCTAGCTACAACGA TTATGCCT | 5048 |
| 1807 | GUGUGUUC A CCAGCACC | 2026 | GGTGCTGG GGCTAGCTACAACGA GAACACAC | 5049 |
| 1813 | UCACCAGC A CCAUGCAA | 2027 | TTGCATGG GGCTAGCTACAACGA GCTGGTGA | 5050 |
| 1816 | CCAGCACC A UGCAACUU | 2028 | AAGTTGCA GGCTAGCTACAACGA GGTGCTGG | 5051 |
| 1821 | ACCAUGCA A CUUUUUCA | 2029 | TGAAAAAG GGCTAGCTACAACGA TGCATGGT | 5052 |
| 1829 | ACUUUUUC A CCUCUGCC | 2030 | GGCAGAGG GGCTAGCTACAACGA GAAAAAGT | 5053 |
| 1840 | UCUGCCUA A UCAUCUCA | 2031 | TGAGATGA GGCTAGCTACAACGA TAGGCAGA | 5054 |
| 1843 | GCCUAAUC A UCUCAUGU | 2032 | ACATGAGA GGCTAGCTACAACGA GATTAGGC | 5055 |
| 1848 | AUCAUCUC A UGUUCAUG | 2033 | CATGAACA GGCTAGCTACAACGA GAGATGAT | 5056 |
| 1854 | UCAUGUUC A UGUCCUAC | 2034 | GTAGGACA GGCTAGCTACAACGA GAACATGA | 5057 |
| 1861 | CAUGUCCU A CUGUUCAA | 2035 | TTGAACAG GGCTAGCTACAACGA AGGACATG | 5058 |
| 1903 | UUUGGGGC A UGGACAUU | 2036 | AATGTCCA GGCTAGCTACAACGA GCCCCAAA | 5059 |
| 1907 | GGGCAUGG A CAUUGACC | 2037 | GGTCAATG GGCTAGCTACAACGA CCATGCCC | 5060 |
| 1909 | GCAUGGAC A UUGACCCG | 2038 | CGGGTCAA GGCTAGCTACAACGA GTCCATGC | 5061 |
| 1913 | GGACAUUG A CCCGUAUA | 2039 | TATACGGG GGCTAGCTACAACGA CAATGTCC | 5062 |
| 1919 | UGACCCGU A UAAAGAAU | 2040 | ATTCTTTA GGCTAGCTACAACGA ACGGGTCA | 5063 |
| 1926 | UAUAAAGA A UUUGGAGC | 2041 | GCTCCAAA GGCTAGCTACAACGA TCTTTATA | 5064 |
| 1947 | GUGGAGUU A CUCUCUUU | 2042 | AAAGAGAG GGCTAGCTACAACGA AACTCCAC | 5065 |
| 1967 | GCCUUCUG A CUUCUUUC | 2043 | GAAAGAAG GGCTAGCTACAACGA CAGAAGGC | 5066 |
| 1981 | UUCCUUCU A UUCGAGAU | 2044 | ATCTCGAA GGCTAGCTACAACGA AGAAGGAA | 5067 |
| 1988 | UAUUCGAG A UCUCCUCG | 2045 | CGAGGAGA GGCTAGCTACAACGA CTCGAATA | 5068 |
| 1997 | UCUCCUCG A CACCGCCU | 2046 | AGGCGGTG GGCTAGCTACAACGA CGAGGAGA | 5069 |
| 1999 | UCCUCGAC A CCGCCUCU | 2047 | AGAGGCGG GGCTAGCTACAACGA GTCGAGGA | 5070 |
| 2015 | UGCUCUGU A UCGGGGGG | 2048 | CCCCCCGA GGCTAGCTACAACGA ACAGAGCA | 5071 |
| 2040 | UCUCCGGA A CAUUGUUC | 2049 | GAACAATG GGCTAGCTACAACGA TCCGGAGA | 5072 |
| 2042 | UCCGGAAC A UUGUUCAC | 2050 | GTGAACAA GGCTAGCTACAACGA GTTCCGGA | 5073 |
| 2049 | CAUUGUUC A CCUCACCA | 2051 | TGGTGAGG GGCTAGCTACAACGA GAACAATG | 5074 |
| 2054 | UUCACCUC A CCAUACGG | 2052 | CCGTATGG GGCTAGCTACAACGA GAGGTGAA | 5075 |
| 2057 | ACCUCACC A UACGGCAC | 2053 | GTGCCGTA GGCTAGCTACAACGA GGTGAGGT | 5076 |
| 2059 | CUCACCAU A CGGCACUC | 2054 | GAGTGCCG GGCTAGCTACAACGA ATGGTGAG | 5077 |
| 2064 | CAUACGGC A CUCAGGCA | 2055 | TGCCTGAG GGCTAGCTACAACGA GCCGTATG | 5078 |
| 2077 | GGCAAGCU A UUCUGUGU | 2056 | ACACAGAA GGCTAGCTACAACGA AGCTTGCC | 5079 |
| 2098 | GUGAGUUG A UGAAUCUA | 2057 | TAGATTCA GGCTAGCTACAACGA CAACTCAC | 5080 |
| 2102 | GUUGAUGA A UCUAGCCA | 2058 | TGGCTAGA GGCTAGCTACAACGA TCATCAAC | 5081 |
| 2110 | AUCUAGCC A CCUGGGUG | 2059 | CACCCAGG GGCTAGCTACAACGA GGCTAGAT | 5082 |
| 2126 | GGGAAGUA A UUUGGAAG | 2060 | CTTCCAAA GGCTAGCTACAACGA TACTTCCC | 5083 |
| 2135 | UUUGGAAG A UCCAGCAU | 2061 | ATGCTGGA GGCTAGCTACAACGA CTTCCAAA | 5084 |
| 2142 | GAUCCAGC A UCCAGGGA | 2062 | TCCCTGGA GGCTAGCTACAACGA GCTGGATC | 5085 |
| 2151 | UCCAGGGA A UUAGUAGU | 2063 | ACTACTAA GGCTAGCTACAACGA TCCCTGGA | 5086 |
| 2165 | AGUCAGCU A UGUCAACG | 2064 | CGTTGACA GGCTAGCTACAACGA AGCTGACT | 5087 |
| 2171 | CUAUGUCA A CGUUAAUA | 2065 | TATTAACG GGCTAGCTACAACGA TGACATAG | 5088 |
| 2177 | CAACGUUA A UAUGGGCC | 2066 | GGCCCATA GGCTAGCTACAACGA TAACGTTG | 5089 |
| 2179 | ACGUUAAU A UGGGCCUA | 2067 | TAGGCCCA GGCTAGCTACAACGA ATTAACGT | 5090 |
| 2191 | GCCUAAAA A UCAGACAA | 2068 | TTGTCTGA GGCTAGCTACAACGA TTTTAGGC | 5091 |
| 2196 | AAAAUCAG A CAACUAUU | 2069 | AATAGTTG GGCTAGCTACAACGA CTGATTTT | 5092 |
| 2199 | AUCAGACA A CUAUUGUG | 2070 | CACAATAG GGCTAGCTACAACGA TGTCTGAT | 5093 |
| 2202 | AGACAACU A UUGUGGUU | 2071 | AACCACAA GGCTAGCTACAACGA AGTTGTCT | 5094 |
| 2213 | GUGGUUUC A CAUUUCCU | 2072 | AGGAAATG GGCTAGCTACAACGA GAAACCAC | 5095 |
| 2215 | GGUUUCAC A UUUCCUGU | 2073 | ACAGGAAA GGCTAGCTACAACGA GTGAAACC | 5096 |
| 2227 | CCUGUCUU A CUUUUGGG | 2074 | CCCAAAAG GGCTAGCTACAACGA AAGACAGG | 5097 |
| 2242 | GGCGAGAA A CUGUUCUU | 2075 | AAGAACAG GGCTAGCTACAACGA TTCTCGCC | 5098 |
| 2253 | GUUCUUGA A UAUUUGGU | 2076 | ACCAAATA GGCTAGCTACAACGA TCAAGAAC | 5099 |
| 2255 | UCUUGAAU A UUUGGUGU | 2077 | ACACCAAA GGCTAGCTACAACGA ATTCAAGA | 5100 |
| 2278 | GAGUGUGG A UUCGCACU | 2078 | AGTGCGAA GGCTAGCTACAACGA CCACACTC | 5101 |
| 2284 | GGAUUCGC A CUCCUCCU | 2079 | AGGAGGAG GGCTAGCTACAACGA GCGAATCC | 5102 |
| 2295 | CCUCCUGC A UAUAGACC | 2080 | GGTCTATA GGCTAGCTACAACGA GCAGGAGG | 5103 |
| 2297 | UCCUGCAU A UAGACCAC | 2081 | GTGGTCTA GGCTAGCTACAACGA ATGCAGGA | 5104 |
| 2301 | GCAUAUAG A CCACCAAA | 2082 | TTTGGTGG GGCTAGCTACAACGA CTATATGC | 5105 |
| 2304 | UAUAGACC A CCAAAUGC | 2083 | GCATTTGG GGCTAGCTACAACGA GGTCTATA | 5106 |
| 2309 | ACCACCAA A UGCCCCUA | 2084 | TAGGGGCA GGCTAGCTACAACGA TTGGTGGT | 5107 |
| 2317 | AUGCCCCU A UCUUAUCA | 2085 | TGATAAGA GGCTAGCTACAACGA AGGGGCAT | 5108 |
| 2322 | CCUAUCUU A UCAACACU | 2086 | AGTGTTGA GGCTAGCTACAACGA AAGATAGG | 5109 |
| 2326 | UCUUAUCA A CACUUCCG | 2087 | CGGAAGTG GGCTAGCTACAACGA TGATAAGA | 5110 |
| 2328 | UUAUCAAC A CUUCCGGA | 2088 | TCCGGAAG GGCTAGCTACAACGA GTTGATAA | 5111 |
| 2338 | UUCCGGAA A CUACUGUU | 2089 | AACAGTAG GGCTAGCTACAACGA TTCCGGAA | 5112 |
| 2341 | CGGAAACU A CUGUUGUU | 2090 | AACAACAG GGCTAGCTACAACGA AGTTTCCG | 5113 |
| 2352 | GUUGUUAG A CGAAGAGG | 2091 | CCTCTTCG GGCTAGCTACAACGA CTAACAAC | 5114 |
| 2380 | GAAGAAGA A CUCCCUCG | 2092 | CGAGGGAG GGCTAGCTACAACGA TCTTCTTC | 5115 |
| 2397 | CCUCGCAG A CGAAGGUC | 2093 | GACCTTCG GGCTAGCTACAACGA CTGCGAGG | 5116 |
| 2409 | AGGUCUCA A UCGCCGCG | 2094 | CGCGGCGA GGCTAGCTACAACGA TGAGACCT | 5117 |
| 2427 | CGCAGAAG A UCUCAAUC | 2095 | GATTGAGA GGCTAGCTACAACGA CTTCTGCG | 5118 |
| 2433 | AGAUCUCA A UCUCGGGA | 2096 | TCCCGAGA GGCTAGCTACAACGA TGAGATCT | 5119 |
| 2442 | UCUCGGGA A UCUCAAUG | 2097 | CATTGAGA GGCTAGCTACAACGA TCCCGAGA | 5120 |
| 2448 | GAAUCUCA A UGUUAGUA | 2098 | TACTAACA GGCTAGCTACAACGA TGAGATTC | 5121 |
| 2456 | AUGUUAGU A UUCCUUGG | 2099 | CCAAGGAA GGCTAGCTACAACGA ACTAACAT | 5122 |
| 2465 | UUCCUUGG A CACAUAAG | 2100 | CTTATGTG GGCTAGCTACAACGA CCAAGGAA | 5123 |
| 2467 | CCUUGGAC A CAUAAGGU | 2101 | ACCTTATG GGCTAGCTACAACGA GTCCAAGG | 5124 |
| 2469 | UUGGACAC A UAAGGUGG | 2102 | CCACCTTA GGCTAGCTACAACGA GTGTCCAA | 5125 |
| 2481 | GGUGGGAA A CUUUACGG | 2103 | CCGTAAAG GGCTAGCTACAACGA TTCCCACC | 5126 |
| 2486 | GAAACUUU A CGGGGCUU | 2104 | AAGCCCCG GGCTAGCTACAACGA AAAGTTTC | 5127 |
| 2496 | GGGGCUUU A UUCUUCUA | 2105 | TAGAAGAA GGCTAGCTACAACGA AAAGCCCC | 5128 |
| 2504 | AUUCUUCU A CGGUACCU | 2106 | AGGTACCG GGCTAGCTACAACGA AGAAGAAT | 5129 |
| 2509 | UCUACGGU A CCUUGCUU | 2107 | AAGCAAGG GGCTAGCTACAACGA ACCGTAGA | 5130 |
| 2520 | UUGCUUUA A UCCUAAAU | 2108 | ATTTAGGA GGCTAGCTACAACGA TAAAGCAA | 5131 |
| 2527 | AAUCCUAA A UGGCAAAC | 2109 | GTTTGCCA GGCTAGCTACAACGA TTAGGATT | 5132 |
| 2534 | AAUGGCAA A CUCCUUCU | 2110 | AGAAGGAG GGCTAGCTACAACGA TTGCCATT | 5133 |
| 2550 | UUUUCCUG A CAUUCAUU | 2111 | AATGAATG GGCTAGCTACAACGA CAGGAAAA | 5134 |
| 2552 | UUCCUGAC A UUCAUUUG | 2112 | CAAATGAA GGCTAGCTACAACGA GTCAGGAA | 5135 |
| 2556 | UGACAUUC A UUUGCAGG | 2113 | CCTGCAAA GGCTAGCTACAACGA GAATGTCA | 5136 |
| 2568 | GCAGGAGG A CAUUGUUG | 2114 | CAACAATG GGCTAGCTACAACGA CCTCCTGC | 5137 |
| 2570 | AGGAGGAC A UUGUUGAU | 2115 | ATCAACAA GGCTAGCTACAACGA GTCCTCCT | 5138 |
| 2577 | CAUUGUUG A UAGAUGUA | 2116 | TACATCTA GGCTAGCTACAACGA CAACAATG | 5139 |
| 2581 | GUUGAUAG A UGUAAGCA | 2117 | TGCTTACA GGCTAGCTACAACGA CTATCAAC | 5140 |
| 2590 | UGUAAGCA A UUUGUGGG | 2118 | CCCACAAA GGCTAGCTACAACGA TGCTTACA | 5141 |
| 2606 | GGCCCCUU A CAGUAAAU | 2119 | ATTTACTG GGCTAGCTACAACGA AAGGGGCC | 5142 |
| 2613 | UACAGUAA A UGAAAACA | 2120 | TGTTTTCA GGCTAGCTACAACGA TTACTGTA | 5143 |
| 2619 | AAAUGAAA A CAGGAGAC | 2121 | GTCTCCTG GGCTAGCTACAACGA TTTCATTT | 5144 |
| 2626 | AACAGGAG A CUUAAAUU | 2122 | AATTTAAG GGCTAGCTACAACGA CTCCTGTT | 5145 |
| 2632 | AGACUUAA A UUAACUAU | 2123 | ATAGTTAA GGCTAGCTACAACGA TTAAGTCT | 5146 |
| 2636 | UUAAAUUA A CUAUGCCU | 2124 | AGGCATAG GGCTAGCTACAACGA TAATTTAA | 5147 |
| 2639 | AAUUAACU A UGCCUGCU | 2125 | AGCAGGCA GGCTAGCTACAACGA AGTTAATT | 5148 |
| 2655 | UAGGUUUU A UCCCAAUG | 2126 | CATTGGGA GGCTAGCTACAACGA AAAACCTA | 5149 |
| 2661 | UUAUCCCA A UGUUACUA | 2127 | TAGTAACA GGCTAGCTACAACGA TGGGATAA | 5150 |
| 2666 | CCAAUGUU A CUAAAUAU | 2128 | ATATTTAG GGCTAGCTACAACGA AACATTGG | 5151 |
| 2671 | GUUACUAA A UAUUUGCC | 2129 | GGCAAATA GGCTAGCTACAACGA TTAGTAAC | 5152 |
| 2673 | UACUAAAU A UUUGCCCU | 2130 | AGGGCAAA GGCTAGCTACAACGA ATTTAGTA | 5153 |
| 2685 | GCCCUUAG A UAAAGGGA | 2131 | TCCCTTTA GGCTAGCTACAACGA CTAAGGGC | 5154 |
| 2693 | AUAAAGGG A UCAAACCG | 2132 | CGGTTTGA GGCTAGCTACAACGA CCCTTTAT | 5155 |
| 2698 | GGGAUCAA A CCGUAUUA | 2133 | TAATACGG GGCTAGCTACAACGA TTGATCCC | 5156 |
| 2703 | CAAACCGU A UUAUCCAG | 2134 | CTGGATAA GGCTAGCTACAACGA ACGGTTTG | 5157 |
| 2706 | ACCGUAUU A UCCAGAGU | 2135 | ACTCTGGA GGCTAGCTACAACGA AATACGGT | 5158 |
| 2715 | UCCAGAGU A UGUAGUUA | 2136 | TAACTACA GGCTAGCTACAACGA ACTCTGGA | 5159 |
| 2724 | UGUAGUUA A UCAUUACU | 2137 | AGTAATGA GGCTAGCTACAACGA TAACTACA | 5160 |
| 2727 | AGUUAAUC A UUACUUCC | 2138 | GGAAGTAA GGCTAGCTACAACGA GATTAACT | 5161 |
| 2730 | UAAUCAUU A CUUCCAGA | 2139 | TCTGGAAG GGCTAGCTACAACGA AATGATTA | 5162 |
| 2738 | ACUUCCAG A CGCGACAU | 2140 | ATGTCGCG GGCTAGCTACAACGA CTGGAAGT | 5163 |
| 2743 | CAGACGCG A CAUUAUUU | 2141 | AAATAATG GGCTAGCTACAACGA CGCGTCTG | 5164 |
| 2745 | GACGCGAC A UUAUUUAC | 2142 | GTAAATAA GGCTAGCTACAACGA GTCGCGTC | 5165 |
| 2748 | GCGACAUU A UUUACACA | 2143 | TGTGTAAA GGCTAGCTACAACGA AATGTCGC | 5166 |
| 2752 | CAUUAUUU A CACACUCU | 2144 | AGAGTGTG GGCTAGCTACAACGA AAATAATG | 5167 |
| 2754 | UUAUUUAC A CACUCUUU | 2145 | AAAGAGTG GGCTAGCTACAACGA GTAAATAA | 5168 |
| 2756 | AUUUACAC A CUCUUUGG | 2146 | CCAAAGAG GGCTAGCTACAACGA GTGTAAAT | 5169 |
| 2774 | AGGCGGGG A UCUUAUAU | 2147 | ATATAAGA GGCTAGCTACAACGA CCCCGCCT | 5170 |
| 2779 | GGGAUCUU A UAUAAAAG | 2148 | CTTTTATA GGCTAGCTACAACGA AAGATCCC | 5171 |
| 2781 | GAUCUUAU A UAAAAGAG | 2149 | CTCTTTTA GGCTAGCTACAACGA ATAAGATC | 5172 |
| 2795 | GAGAGUCC A CACGUAGC | 2150 | GCTACGTG GGCTAGCTACAACGA GGACTCTC | 5173 |
| 2797 | GAGUCCAC A CGUAGCGC | 2151 | GCGCTACG GGCTAGCTACAACGA GTGGACTC | 5174 |
| 2809 | AGCGCCUC A UUUUGCGG | 2152 | CCGCAAAA GGCTAGCTACAACGA GAGGCGCT | 5175 |
| 2821 | UGCGGGUC A CCAUAUUC | 2153 | GAATATGG GGCTAGCTACAACGA GACCCGCA | 5176 |
| 2824 | GGGUCACC A UAUUCUUG | 2154 | CAAGAATA GGCTAGCTACAACGA GGTGACCC | 5177 |
| 2826 | GUCACCAU A UUCUUGGG | 2155 | CCCAAGAA GGCTAGCTACAACGA ATGGTGAC | 5178 |
| 2836 | UCUUGGGA A CAAGAUCU | 2156 | AGATCTTG GGCTAGCTACAACGA TCCCAAGA | 5179 |
| 2841 | GGAACAAG A UCUACAGC | 2157 | GCTGTAGA GGCTAGCTACAACGA CTTGTTCC | 5180 |
| 2845 | CAAGAUCU A CAGCAUGG | 2158 | CCATGCTG GGCTAGCTACAACGA AGATCTTG | 5181 |
| 2850 | UCUACAGC A UGGGAGGU | 2159 | ACCTCCCA GGCTAGCTACAACGA GCTGTAGA | 5182 |
| 2870 | UCUUCCAA A CCUCGAAA | 2160 | TTTCGAGG GGCTAGCTACAACGA TTGGAAGA | 5183 |
| 2883 | GAAAAGGC A UGGGGACA | 2161 | TGTCCCCA GGCTAGCTACAACGA GCCTTTTC | 5184 |
| 2889 | GCAUGGGG A CAAAUCUU | 2162 | AAGATTTG GGCTAGCTACAACGA CCCCATGC | 5185 |
| 2893 | GGGGACAA A UCUUUCUG | 2163 | CAGAAAGA GGCTAGCTACAACGA TTGTCCCC | 5186 |
| 2908 | UGUCCCCA A UCCCCUGG | 2164 | CCAGGGGA GGCTAGCTACAACGA TGGGGACA | 5187 |
| 2918 | CCCCUGGG A UUCUUCCC | 2165 | GGGAAGAA GGCTAGCTACAACGA CCCAGGGG | 5188 |
| 2929 | CUUCCCCG A UCAUCAGU | 2166 | ACTGATGA GGCTAGCTACAACGA CGGGGAAG | 5189 |
| 2932 | CCCCGAUC A UCAGUUGG | 2167 | CCAACTGA GGCTAGCTACAACGA GATCGGGG | 5190 |
| 2941 | UCAGUUGG A CCCUGCAU | 2168 | ATGCAGGG GGCTAGCTACAACGA CCAACTGA | 5191 |
| 2948 | GACCCUGC A UUCAAAGC | 2169 | GCTTTGAA GGCTAGCTACAACGA GCAGGGTC | 5192 |
| 2959 | CAAAGCCA A CUCAGUAA | 2170 | TTACTGAG GGCTAGCTACAACGA TGGCTTTG | 5193 |
| 2968 | CUCAGUAA A UCCAGAUU | 2171 | AATCTGGA GGCTAGCTACAACGA TTACTGAG | 5194 |
| 2974 | AAAUCCAG A UUGGGACC | 2172 | GGTCCCAA GGCTAGCTACAACGA CTGGATTT | 5195 |
| 2980 | AGAUUGGG A CCUCAACC | 2173 | GGTTGAGG GGCTAGCTACAACGA CCCAATCT | 5196 |
| 2986 | GGACCUCA A CCCGCACA | 2174 | TGTGCGGG GGCTAGCTACAACGA TGAGGTCC | 5197 |
| 2998 | GCACAAGG A CAACUGGC | 2175 | GCCAGTTG GGCTAGCTACAACGA CCTTGTGC | 5198 |
| 3001 | CAAGGACA A CUGGCCGG | 2176 | CCGGCCAG GGCTAGCTACAACGA TGTCCTTG | 5199 |
| 3010 | CUGGCCGG A CGCCAACA | 2177 | TGTTGGCG GGCTAGCTACAACGA CCGGCCAG | 5200 |
| 3016 | GGACGCCA A CAAGGUGG | 2178 | CCACCTTG GGCTAGCTACAACGA TGGCGTCC | 5201 |
| 3035 | GUGGGAGC A UUCGGGCC | 2179 | GGCCCGAA GGCTAGCTACAACGA GCTCCCAC | 5202 |
| 3051 | CAGGGUUC A CCCCUCCC | 2180 | GGGAGGGG GGCTAGCTACAACGA GAACCCTG | 5203 |
| 3061 | CCCUCCCC A UGGGGGAC | 2181 | GTCCCCCA GGCTAGCTACAACGA GGGGAGGG | 5204 |
| 3068 | CAUGGGGG A CUGUUGGG | 2182 | CCCAACAG GGCTAGCTACAACGA CCCCCATG | 5205 |
| 3088 | GAGCCCUC A CGCUCAGG | 2183 | CCTGAGCG GGCTAGCTACAACGA GAGGGCTC | 5206 |
| 3101 | CAGGGCCU A CUCACAAC | 2184 | GTTGTGAG GGCTAGCTACAACGA AGGCCCTG | 5207 |
| 3105 | GCCUACUC A CAACUGUG | 2185 | CACAGTTG GGCTAGCTACAACGA GAGTAGGC | 5208 |
| 3108 | UACUCACA A CUGUGCCA | 2186 | TGGCACAG GGCTAGCTACAACGA TGTGAGTA | 5209 |
| 3138 | CUGCCUCC A CCAAUCGG | 2187 | CCGATTGG GGCTAGCTACAACGA GGAGGCAG | 5210 |
| 3142 | CUCCACCA A UCGGCAGU | 2188 | ACTGCCGA GGCTAGCTACAACGA TGGTGGAG | 5211 |
| 3165 | GGCAGCCU A CUCCCUUA | 2189 | TAAGGGAG GGCTAGCTACAACGA AGGCTGCC | 5212 |
| 3173 | ACUCCCUU A UCUCCACC | 2190 | GGTGGAGA GGCTAGCTACAACGA AAGGGAGT | 5213 |
| 3179 | UUAUCUCC A CCUCUAAG | 2191 | CTTAGAGG GGCTAGCTACAACGA GGAGATAA | 5214 |
| 3190 | UCUAAGGG A CACUCAUC | 2192 | GATGAGTG GGCTAGCTACAACGA CCCTTAGA | 5215 |
| 3192 | UAAGGGAC A CUCAUCCU | 2193 | AGGATGAG GGCTAGCTACAACGA GTCCCTTA | 5216 |
| 3196 | GGACACUC A UCCUCAGG | 2194 | CCTGAGGA GGCTAGCTACAACGA GAGTGTCC | 5217 |
| 3207 | CUCAGGCC A UGCAGUGG | 2195 | CCACTGCA GGCTAGCTACAACGA GGCCTGAG | 5218 |

Input Sequence = AF100308. Cut Site = YG/M or UG/U.
Stem Length = 8. Core Sequence = GGCTAGCTACAACGA
AF100308 (Hepatitis B virus strain 2-18, 3215 bp)

TABLE X
HUMAN HBV AMBERZYME AND SUBSTRATE SEQUENCE

| Pos | Substrate | Seq ID | Amberzyme | Seq ID |
|---|---|---|---|---|
| 61 | ACUUUCCU G CUGGUGGC | 1448 | GCCACCAG GGAGGAAACUCC CU UCAAGGACAUCGUCCGGG AGGAAAGU | 5219 |
| 87 | GGAACAGU G AGCCCUGC | 1449 | GCAGGGCU GGAGGAAACUCC CU UCAAGGACAUCGUCCGGG ACUGUUCC | 5220 |
| 94 | UGAGCCCU G CUCAGAAU | 1450 | AUUCUGAG GGAGGAAACUCC CU UCAAGGACAUCGUCCGGG AGGGCUCA | 5221 |
| 112 | CUGUCUCU G CCAUAUCG | 1451 | CGAUAUGG GGAGGAAACUCC CU UCAAGGACAUCGUCCGGG AGAGACAG | 5222 |
| 132 | AUCUUAUC G AAGACUGG | 1452 | CCAGUCUU GGAGGAAACUCC CU UCAAGGACAUCGUCCGGG GAUAAGAU | 5223 |
| 153 | CCUGUACC G AACAUGGA | 1453 | UCCAUGUU GGAGGAAACUCC CU UCAAGGACAUCGUCCGGG GGUACAGG | 5224 |
| 169 | AGAACAUC G CAUCAGGA | 1454 | UCCUGAUG GGAGGAAACUCC CU UCAAGGACAUCGUCCGGG GAUGUUCU | 5225 |
| 192 | GGACCCCU G CUCGUGUU | 1455 | AACACGAG GGAGGAAACUCC CU UCAAGGACAUCGUCCGGG AGGGGUCC | 5226 |
| 222 | UUCUUGUU G ACAAAAAU | 1456 | AUUUUUGU GGAGGAAACUCC CU UCAAGGACAUCGUCCGGG AACAAGAA | 5227 |
| 315 | CAAAAUUC G CAGUCCCA | 1457 | UGGGACUG GGAGGAAACUCC CU UCAAGGACAUCGUCCGGG GAAUUUUG | 5228 |
| 374 | UGGUUAUC G CUGGAUGU | 1458 | ACAUCCAG GGAGGAAACUCC CU UCAAGGACAUCGUCCGGG GAUAACCA | 5229 |
| 387 | AUGUGUCU G CGGCGUUU | 1459 | AAACGCCG GGAGGAAACUCC CU UCAAGGACAUCGUCCGGG AGACACAU | 5230 |
| 410 | CUUCCUCU G CAUCCUGC | 1460 | GCAGGAUG GGAGGAAACUCC CU UCAAGGACAUCGUCCGGG AGAGGAAG | 5231 |
| 417 | UGCAUCCU G CUGCUAUG | 1461 | CAUAGCAG GGAGGAAACUCC CU UCAAGGACAUCGUCCGGG AGGAUGCA | 5232 |
| 420 | AUCCUGCU G CUAUGCCU | 1462 | AGGCAUAG GGAGGAAACUCC CU UCAAGGACAUCGUCCGGG AGCAGGAU | 5233 |
| 425 | GCUGCUAU G CCUCAUCU | 1463 | AGAUGAGG GGAGGAAACUCC CU UCAAGGACAUCGUCCGGG AUAGCAGC | 5234 |
| 468 | GGUAUGUU G CCCGUUUG | 1484 | CAAACGGG GGAGGAAACUCC CU UCAAGGACAUCGUCCGGG AACAUACC | 5235 |
| 518 | CGGACCAU G CAAAACCU | 1465 | AGGUUUUG GGAGGAAACUCC CU UCAAGGACAUCGUCCGGG AUGGUCCG | 5236 |
| 527 | CAAAACCU G CACAACUC | 1466 | GAGUUGUG GGAGGAAACUCC CU UCAAGGACAUCGUCCGGG AGGUUUUG | 5237 |
| 538 | CAACUCCU G CUCAAGGA | 1467 | UCCUUGAG GGAGGAAACUCC CU UCAAGGACAUCGUCCGGG AGGAGUUG | 5238 |
| 569 | CUCAUGUU G CUGUACAA | 1468 | UUGUACAG GGAGGAAACUCC CU UCAAGGACAUCGUCCGGG AACAUGAG | 5239 |
| 596 | CGGAAACU G CACCUGUA | 1469 | UACAGGUG GGAGGAAACUCC CU UCAAGGACAUCGUCCGGG AGUUUCCG | 5240 |
| 631 | GGGCUUUC G CAAAAUAC | 1470 | GUAUUUUG GGAGGAAACUCC CU UCAAGGACAUCGUCCGGG GAAAGCCC | 5241 |
| 687 | UUACUAGU G CCAUUUGU | 1471 | ACAAAUGG GGAGGAAACUCC CU UCAAGGACAUCGUCCGGG ACUAGUAA | 5242 |
| 747 | AUAUGGAU G AUGUGGUU | 1472 | AACCACAU GGAGGAAACUCC CU UCAAGGACAUCGUCCGGG AUCCAUAU | 5243 |
| 783 | AACAUCUU G AGUCCCUU | 1473 | AAGGGACU GGAGGAAACUCC CU UCAAGGACAUCGUCCGGG AAGAUGUU | 5244 |
| 795 | CCCUUUAU G CCGCUGUU | 1474 | AACAGCGG GGAGGAAACUCC CU UCAAGGACAUCGUCCGGG AUAAAGGG | 5245 |
| 798 | UUUAUGCC G CUGUUACC | 1475 | GGUAACAG GGAGGAAACUCC CU UCAAGGACAUCGUCCGGG GGCAUAAA | 5246 |
| 911 | GGCACAUU G CCACAGGA | 1476 | UCCUGUGG GGAGGAAACUCC CU UCAAGGACAUCGUCCGGG AAUGUGCC | 5247 |
| 978 | GGCCUAUU G AUUGGAAA | 1477 | UUUCCAAU GGAGGAAACUCC CU UCAAGGACAUCGUCCGGG AAUAGGCC | 5248 |
| 997 | AUGUCAAC G AAUUGUGG | 1478 | CCACAAUU GGAGGAAACUCC CU UCAAGGACAUCGUCCGGG GUUGACAU | 5249 |
| 1020 | UGGGGUUU G CCGCCCCU | 1479 | AGGGGCGG GGAGGAAACUCC CU UCAAGGACAUCGUCCGGG AAACCCCA | 5250 |
| 1023 | GGUUUGCC G CCCCUUUC | 1480 | GAAAGGGG GGAGGAAACUCC CU UCAAGGACAUCGUCCGGG GGCAAACC | 5251 |
| 1034 | CCUUUCAC G CAAUGUGG | 1481 | CCACAUUG GGAGGAAACUCC CU UCAAGGACAUCGUCCGGG GUGAAAGG | 5252 |
| 1050 | GAUAUUCU G CUUUAAUG | 1482 | CAUUAAAG GGAGGAAACUCC CU UCAAGGACAUCGUCCGGG AGAAUAUC | 5253 |
| 1058 | GCUUUAAU G CCUUUAUA | 1483 | UAUAAAGG GGAGGAAACUCC CU UCAAGGACAUCGUCCGGG AUUAAAGC | 5254 |
| 1068 | CUUUAUAU G CAUGCAUA | 1484 | UAUGCAUG GGAGGAAACUCC CU UCAAGGACAUCGUCCGGG AUAUAAAG | 5255 |
| 1072 | AUAUGCAU G CAUACAAG | 1485 | CUUGUAUG GGAGGAAACUCC CU UCAAGGACAUCGUCCGGG AUGCAUAU | 5256 |
| 1103 | ACUUUCUC G CCAACUUA | 1486 | UAAGUUGG GGAGGAAACUCC CU UCAAGGACAUCGUCCGGG GAGAAAGU | 5257 |
| 1139 | CAGUAUGU G AACCUUUA | 1487 | UAAAGGUU GGAGGAAACUCC CU UCAAGGACAUCGUCCGGG ACAUACUG | 5258 |
| 1155 | ACCCCGUU G CUCGGCAA | 1488 | UUGCCGAG GGAGGAAACUCC CU UCAAGGACAUCGUCCGGG AACGGGGU | 5259 |
| 1177 | UGGUCUAU G CCAAGUGU | 1489 | ACACUUGG GGAGGAAACUCC CU UCAAGGACAUCGUCCGGG AUAGACCA | 5260 |
| 1188 | AAGUGUUU G CUGACGCA | 1490 | UGCGUCAG GGAGGAAACUCC CU UCAAGGACAUCGUCCGGG AAACACUU | 5261 |
| 1191 | UGUUUGCU G ACGCAACC | 1491 | GGUUGCGU GGAGGAAACUCC CU UCAAGGACAUCGUCCGGG AGCAAACA | 5262 |
| 1194 | UUGCUGAC G CAACCCCC | 1492 | GGGGGUUG GGAGGAAACUCC CU UCAAGGACAUCGUCCGGG GUCAGCAA | 5263 |
| 1234 | CCAUCAGC G CAUGCGUG | 1493 | CACGCAUG GGAGGAAACUCC CU UCAAGGACAUCGUCCGGG GCUGAUGG | 5264 |
| 1238 | CAGCGCAU G CGUGGAAC | 1494 | GUUCCACG GGAGGAAACUCC CU UCAAGGACAUCGUCCGGG AUGCGCUG | 5265 |
| 1262 | UCUCCUCU G CCGAUCCA | 1495 | UGGAUCGG GGAGGAAACUCC CU UCAAGGACAUCGUCCGGG AGAGGAGA | 5266 |
| 1265 | CCUCUGCC G AUCCAUAC | 1496 | GUAUGGAU GGAGGAAACUCC CU UCAAGGACAUCGUCCGGG GGCAGAGG | 5267 |
| 1275 | UCCAUACC G CGGAACUC | 1497 | GAGUUCCG GGAGGAAACUCC CU UCAAGGACAUCGUCCGGG GGUAUGGA | 5268 |
| 1290 | UCCUAGCC G CUUGUUUU | 1498 | AAAACAAG GGAGGAAACUCC CU UCAAGGACAUCGUCCGGG GGCUAGGA | 5269 |
| 1299 | CUUGUUUU G CUCGCAGC | 1499 | GCUGCGAG GGAGGAAACUCC CU UCAAGGACAUCGUCCGGG AAAACAAG | 5270 |
| 1303 | UUUUGCUC G CAGCAGGU | 1500 | ACCUGCUG GGAGGAAACUCC CU UCAAGGACAUCGUCCGGG GAGCAAAA | 5271 |
| 1335 | UCGGGACU G ACAAUUCU | 1501 | AGAAUUGU GGAGGAAACUCC CU UCAAGGACAUCGUCCGGG AGUCCCGA | 5272 |
| 1349 | UCUGUCGU G CUCUCCCG | 1502 | CGGGAGAG GGAGGAAACUCC CU UCAAGGACAUCGUCCGGG ACGACAGA | 5273 |
| 1357 | GCUCUCCC G CAAAUAUA | 1503 | UAUAUUUG GGAGGAAACUCC CU UCAAGGACAUCGUCCGGG GGGAGAGC | 5274 |
| 1382 | CCAUGGCU G CUAGGCUG | 1504 | CAGCCUAG GGAGGAAACUCC CU UCAAGGACAUCGUCCGGG AGCCAUGG | 5275 |
| 1392 | UAGGCUGU G CUGCCAAC | 1505 | GUUGGCAG GGAGGAAACUCC CU UCAAGGACAUCGUCCGGG ACAGCCUA | 5276 |
| 1395 | GCUGUGCU G CCAACUGG | 1506 | CCAGUUGG GGAGGAAACUCC CU UCAAGGACAUCGUCCGGG AGCACAGC | 5277 |
| 1411 | GAUCCUAC G CGGGACGU | 1507 | ACGUCCCG GGAGGAAACUCC CU UCAAGGACAUCGUCCGGG GUAGGAUC | 5278 |
| 1442 | CCGUCGGC G CUGAAUCC | 1508 | GGAUUCAG GGAGGAAACUCC CU UCAAGGACAUCGUCCGGG GCCGACGG | 5279 |
| 1445 | UCGGCGCU G AAUCCCGC | 1509 | GCGGGAUU GGAGGAAACUCC CU UCAAGGACAUCGUCCGGG AGCGCCGA | 5280 |
| 1452 | UGAAUCCC G CGGACGAC | 1510 | GUCGUCCG GGAGGAAACUCC CU UCAAGGACAUCGUCCGGG GGGAUUCA | 5281 |
| 1458 | CCGCGGAC G ACCCCUCC | 1511 | GGAGGGGU GGAGGAAACUCC CU UCAAGGACAUCGUCCGGG GUCCGCGG | 5282 |
| 1474 | CCGGGGCC G CUUGGGGC | 1512 | GCCCCAAG GGAGGAAACUCC CU UCAAGGACAUCGUCCGGG GGCCCCGG | 5283 |
| 1489 | GCUCUACC G CCCGCUUC | 1513 | GAAGCGGG GGAGGAAACUCC CU UCAAGGACAUCGUCCGGG GGUAGAGC | 5284 |
| 1493 | UACCGCCC G CUUCUCCG | 1514 | CGGAGAAG GGAGGAAACUCC CU UCAAGGACAUCGUCCGGG GGGCGGUA | 5285 |
| 1501 | GCUUCUCC G CCUAUUGU | 1515 | ACAAUAGG GGAGGAAACUCC CU UCAAGGACAUCGUCCGGG GGAGAAGC | 5286 |
| 1513 | AUUGUACC G ACCGUCCA | 1516 | UGGACGGU GGAGGAAACUCC CU UCAAGGACAUCGUCCGGG GGUACAAU | 5287 |
| 1528 | CACGGGGC G CACCUCUC | 1517 | GAGAGGUG GGAGGAAACUCC CU UCAAGGACAUCGUCCGGG GCCCCGUG | 5288 |
| 1542 | CUCUUUAC G CGGACUCC | 1518 | GGAGUCCG GGAGGAAACUCC CU UCAAGGACAUCGUCCGGG GUAAAGAG | 5289 |
| 1559 | CCGUCUGU G CCUUCUCA | 1519 | UGAGAAGG GGAGGAAACUCC CU UCAAGGACAUCGUCCGGG ACAGACGG | 5290 |
| 1571 | UCUCAUCU G CCGGACCG | 1520 | CGGUCCGG GGAGGAAACUCC CU UCAAGGACAUCGUCCGGG AGAUGAGA | 5291 |
| 1583 | GACCGUGU G CACUUCGC | 1521 | GCGAAGUG GGAGGAAACUCC CU UCAAGGACAUCGUCCGGG ACACGGUC | 5292 |
| 1590 | UGCACUUC G CUUCACCU | 1522 | AGGUGAAG GGAGGAAACUCC CU UCAAGGACAUCGUCCGGG GAAGUGCA | 5293 |
| 1601 | UCACCUCU G CACGUCGC | 1523 | GCGACGUG GGAGGAAACUCC CU UCAAGGACAUCGUCCGGG AGAGGUGA | 5294 |
| 1608 | UGCACGUC G CAUGGAGA | 1524 | UCUCCAUG GGAGGAAACUCC CU UCAAGGACAUCGUCCGGG GACGUGCA | 5295 |
| 1624 | ACCACCGU G AACGCCCA | 1525 | UGGGCGUU GGAGGAAACUCC CU UCAAGGACAUCGUCCGGG ACGGUGGU | 5296 |
| 1628 | CCGUGAAC G CCCACAGG | 1526 | CCUGUGGG GGAGGAAACUCC CU UCAAGGACAUCGUCCGGG GUUCACGG | 5297 |
| 1642 | AGGAACCU G CCCAAGGU | 1527 | ACCUUGGG GGAGGAAACUCC CU UCAAGGACAUCGUCCGGG AGGUUCCU | 5298 |
| 1654 | AAGGUCUU G CAUAAGAG | 1528 | CUCUUAUG GGAGGAAACUCC CU UCAAGGACAUCGUCCGGG AAGACCUU | 5299 |
| 1690 | AUGUCAAC G ACCGACCU | 1529 | AGGUCGGU GGAGGAAACUCC CU UCAAGGACAUCGUCCGGG GUUGACAU | 5300 |
| 1694 | CAACGACC G ACCUUGAG | 1530 | CUCAAGGU GGAGGAAACUCC CU UCAAGGACAUCGUCCGGG GGUCGUUG | 5301 |
| 1700 | CCGACCUU G AGGCAUAC | 1531 | GUAUGCCU GGAGGAAACUCC CU UCAAGGACAUCGUCCGGG AAGGUCGG | 5302 |
| 1730 | UGUUUAAU G AGUGGGAG | 1532 | CUCCCACU GGAGGAAACUCC CU UCAAGGACAUCGUCCGGG AUUAAACA | 5303 |
| 1818 | AGCACCAU G CAACUUUU | 1533 | AAAAGUUG GGAGGAAACUCC CU UCAAGGACAUCGUCCGGG AUGGUGCU | 5304 |
| 1835 | UCACCUCU G CCUAAUCA | 1534 | UGAUUAGG GGAGGAAACUCC CU UCAAGGACAUCGUCCGGG AGAGGUGA | 5305 |
| 1883 | CAAGCUGU G CCUUGGGU | 1535 | ACCCAAGG GGAGGAAACUCC CU UCAAGGACAUCGUCCGGG ACAGCUUG | 5306 |
| 1912 | UGGACAUU G ACCCGUAU | 1536 | AUACGGGU GGAGGAAACUCC CU UCAAGGACAUCGUCCGGG AAUGUCCA | 5307 |
| 1959 | UCUUUUUU G CCUUCUGA | 1537 | UCAGAAGG GGAGGAAACUCC CU UCAAGGACAUCGUCCGGG AAAAAAGA | 5308 |
| 1966 | UGCCUUCU G ACUUCUUU | 1538 | AAAGAAGU GGAGGAAACUCC CU UCAAGGACAUCGUCCGGG AGAAGGCA | 5309 |
| 1985 | UUCUAUUC G AGAUCUCC | 1539 | GGAGAUCU GGAGGAAACUCC CU UCAAGGACAUCGUCCGGG GAAUAGAA | 5310 |
| 1996 | AUCUCCUC G ACACCGCC | 1540 | GGCGGUGU GGAGGAAACUCC CU UCAAGGACAUCGUCCGGG GAGGAGAU | 5311 |
| 2002 | UCGACACC G CCUCUGCU | 1541 | AGCAGAGG GGAGGAAACUCC CU UCAAGGACAUCGUCCGGG GGUGUCGA | 5312 |
| 2008 | CCGCCUCU G CUCUGUAU | 1542 | AUACAGAG GGAGGAAACUCC CU UCAAGGACAUCGUCCGGG AGAGGCGG | 5313 |
| 2092 | GUUGGGGU G AGUUGAUG | 1543 | CAUCAACU GGAGGAAACUCC CU UCAAGGACAUCGUCCGGG ACCCCAAC | 5314 |
| 2097 | GGUGAGUU G AUGAAUCU | 1544 | AGAUUCAU GGAGGAAACUCC CU UCAAGGACAUCGUCCGGG AACUCACC | 5315 |
| 2100 | GAGUUGAU G AAUCUAGC | 1545 | GCUAGAUU GGAGGAAACUCC CU UCAAGGACAUCGUCCGGG AUCAACUC | 5316 |
| 2237 | UUUUGGGC G AGAAACUG | 1546 | CAGUUUCU GGAGGAAACUCC CU UCAAGGACAUCGUCCGGG GCCCAAAA | 5317 |
| 2251 | CUGUUCUU G AAUAUUUG | 1547 | CAAAUAUU GGAGGAAACUCC CU UCAAGGACAUCGUCCGGG AAGAACAG | 5318 |
| 2282 | GUGGAUUC G CACUCCUC | 1548 | GAGGAGUG GGAGGAAACUCC CU UCAAGGACAUCGUCCGGG GAAUCCAC | 5319 |
| 2293 | CUCCUCCU G CAUAUAGA | 1549 | UCUAUAUG GGAGGAAACUCC CU UCAAGGACAUCGUCCGGG AGGAGGAG | 5320 |
| 2311 | CACCAAAU G CCCCUAUC | 1550 | GAUAGGGG GGAGGAAACUCC CU UCAAGGACAUCGUCCGGG AUUUGGUG | 5321 |
| 2354 | UGUUAGAC G AAGAGGCA | 1551 | UGCCUCUU GGAGGAAACUCC CU UCAAGGACAUCGUCCGGG GUCUAACA | 5322 |
| 2388 | ACUCCCUC G CCUCGCAG | 1552 | CUGCGAGG GGAGGAAACUCC CU UCAAGGACAUCGUCCGGG GAGGGAGU | 5323 |
| 2393 | CUCGCCUC G CAGACGAA | 1553 | UUCGUCUG GGAGGAAACUCC CU UCAAGGACAUCGUCCGGG GAGGCGAG | 5324 |
| 2399 | UCGCAGAC G AAGGUCUC | 1554 | GAGACCUU GGAGGAAACUCC CU UCAAGGACAUCGUCCGGG GUCUGCGA | 5325 |
| 2412 | UCUCAAUC G CCGCGUCG | 1555 | CGACGCGG GGAGGAAACUCC CU UCAAGGACAUCGUCCGGG GAUUGAGA | 5326 |
| 2415 | CAAUCGCC G CGUCGCAG | 1556 | CUGCGACG GGAGGAAACUCC CU UCAAGGACAUCGUCCGGG GGCGAUUG | 5327 |
| 2420 | GCCGCGUC G CAGAAGAU | 1557 | AUCUUCUG GGAGGAAACUCC CU UCAAGGACAUCGUCCGGG GACGCGGC | 5328 |
| 2514 | GGUACCUU G CUUUAAUC | 1558 | GAUUAAAG GGAGGAAACUCC CU UCAAGGACAUCGUCCGGG AAGGUACC | 5329 |
| 2549 | CUUUUCCU G ACAUUCAU | 1559 | AUGAAUGU GGAGGAAACUCC CU UCAAGGACAUCGUCCGGG AGGAAAAG | 5330 |
| 2560 | AUUCAUUU G CAGGAGGA | 1560 | UCCUCCUG GGAGGAAACUCC CU UCAAGGACAUCGUCCGGG AAAUGAAU | 5331 |
| 2576 | ACAUUGUU G AUAGAUGU | 1561 | ACAUCUAU GGAGGAAACUCC CU UCAAGGACAUCGUCCGGG AACAAUGU | 5332 |
| 2615 | CAGUAAAU G AAAACAGG | 1562 | CCUGUUUU GGAGGAAACUCC CU UCAAGGACAUCGUCCGGG AUUUACUG | 5333 |
| 2641 | UUAACUAU G CCUGCUAG | 1562 | CUAGCAGG GGAGGAAACUCC CU UCAAGGACAUCGUCCGGG AUAGUUAA | 5334 |
| 2645 | CUAUGCCU G CUAGGUUU | 1564 | AAACCUAG GGAGGAAACUCC CU UCAAGGACAUCGUCCGGG AGGCAUAG | 5335 |
| 2677 | AAAUAUUU G CCCUUAGA | 1565 | UCUAAGGG GGAGGAAACUCC CU UCAAGGACAUCGUCCGGG AAAUAUUU | 5336 |
| 2740 | UUCCAGAC G CGACAUUA | 1566 | UAAUGUCG GGAGGAAACUCC CU UCAAGGACAUCGUCCGGG GUCUGGAA | 5337 |
| 2742 | CCAGACGC G ACAUUAUU | 1567 | AAUAAUGU GGAGGAAACUCC CU UCAAGGACAUCGUCCGGG GCGUCUGG | 5338 |
| 2804 | CACGUAGC G CCUCAUUU | 1568 | AAAUGAGG GGAGGAAACUCC CU UCAAGGACAUCGUCCGGG GCUACGUG | 5339 |
| 2814 | CUCAUUUU G CGGGUCAC | 1569 | GUGACCCG GGAGGAAACUCC CU UCAAGGACAUCGUCCGGG AAAAUGAG | 5340 |
| 2875 | CAAACCUC G AAAAGGCA | 1570 | UGCCUUUU GGAGGAAACUCC CU UCAAGGACAUCGUCCGGG GAGGUUUG | 5341 |
| 2928 | UCUUCCCC G AUCAUCAG | 1571 | CUGAUGAU GGAGGAAACUCC CU UCAAGGACAUCGUCCGGG GGGGAAGA | 5342 |
| 2946 | UGGACCCU G CAUUCAAA | 1572 | UUUGAAUG GGAGGAAACUCC CU UCAAGGACAUCGUCCGGG AGGGUCCA | 5343 |
| 2990 | CUCAACCC G CACAAGGA | 1573 | UCCUGGUG GGAGGAAACUCC CU UCAAGGACAUCGUCCGGG GGGUUGAG | 5344 |
| 3012 | GGCCGGAC G CCAACAAG | 1574 | CUUGUUGG GGAGGAAACUCC CU UCAAGGACAUCGUCCGGG GUCCGGCC | 5345 |
| 3090 | GCCCUCAC G CUCAGGGC | 1575 | GCCCUGAG GGAGGAAACUCC CU UCAAGGACAUCGUCCGGG GUGAGGGC | 5346 |
| 3113 | ACAACUGU G CCAGCAGC | 1576 | GCUGCUGG GGAGGAAACUCC CU UCAAGGACAUCGUCCGGG ACAGUUGU | 5347 |
| 3132 | CUCCUCCU G CCUCCACC | 1577 | GGUGGAGG GGAGGAAACUCC CU UCAAGGACAUCGUCCGGG AGGAGGAG | 5348 |
| 51A | AGGGCCCU G UACUUUCC | 1578 | GGAAAGUA GGAGGAAACUCC CU UCAAGGACAUCGUCCGGG AGGGCCCU | 5349 |
| 106 | AGAAUACU G UCUCUGCC | 1579 | GGCAGAGA GGAGGAAACUCC CU UCAAGGACAUCGUCCGGG AGUAUUCU | 5350 |
| 148 | GGGACCCU G UACCGAAC | 1580 | GUUCGGUA GGAGGAAACUCC CU UCAAGGACAUCGUCCGGG AGGGUCCC | 5351 |
| 198 | CUGCUCGU G UUACAGGC | 1581 | GCCUGUAA GGAGGAAACUCC CU UCAAGGACAUCGUCCGGG ACGAGCAG | 5352 |
| 219 | UUUUUCUU G UUGACAAA | 1582 | UUUGUCAA GGAGGAAACUCC CU UCAAGGACAUCGUCCGGG AAGAAAAA | 5353 |
| 297 | ACACCCGU G UGUCUUGG | 1583 | CCAAGACA GGAGGAAACUCC CU UCAAGGACAUCGUCCGGG ACGGGUGU | 5354 |
| 299 | ACCCGUGU G UCUUGGCC | 1584 | GGCCAAGA GGAGGAAACUCC CU UCAAGGACAUCGUCCGGG ACACGGGU | 5355 |
| 347 | ACCAACCU G UUGUCCUC | 1585 | GAGGACAA GGAGGAAACUCC CU UCAAGGACAUCGUCCGGG AGGUUGGU | 5356 |
| 350 | AACCUGUU G UCCUCCAA | 1586 | UUGGAGGA GGAGGAAACUCC CU UCAAGGACAUCGUCCGGG AACAGGUU | 5357 |
| 362 | UCCAAUUU G UCCUGGUU | 1587 | AACCAGGA GGAGGAAACUCC CU UCAAGGACAUCGUCCGGG AAAUUGGA | 5358 |
| 381 | CGCUGGAU G UGUCUGCG | 1588 | CGCAGACA GGAGGAAACUCC CU UCAAGGACAUCGUCCGGG AUCCAGCG | 5359 |
| 383 | CUGGAUGU G UCUGCGGC | 1589 | GCCGCAGA GGAGGAAACUCC CU UCAAGGACAUCGUCCGGG ACAUCCAG | 5360 |
| 438 | AUCUUCUU G UUGGUUCU | 1590 | AGAACCAA GGAGGAAACUCC CU UCAAGGACAUCGUCCGGG AAGAAGAU | 5361 |
| 465 | CAAGGUAU G UUGCCCGU | 1591 | ACGGGCAA GGAGGAAACUCC CU UCAAGGACAUCGUCCGGG AUACCUUG | 5362 |
| 476 | GCCCGUUU G UCCUCUAA | 1592 | UUAGAGGA GGAGGAAACUCC CU UCAAGGACAUCGUCCGGG AAACGGGC | 5363 |
| 555 | ACCUCUAU G UUUCCCUC | 1593 | GAGGGAAA GGAGGAAACUCC CU UCAAGGACAUCGUCCGGG AUAGAGGU | 5364 |
| 566 | UCCCUCAU G UUGCUGUA | 1594 | UACAGCAA GGAGGAAACUCC CU UCAAGGACAUCGUCCGGG AUGAGGGA | 5365 |
| 572 | AUGUUGCU G UACAAAAC | 1595 | GUUUUGUA GGAGGAAACUCC CU UCAAGGACAUCGUCCGGG AGCAACAU | 5366 |
| 602 | CUGCACCU G UAUUCCCA | 1596 | UGGGAAUA GGAGGAAACUCC CU UCAAGGACAUCGUCCGGG AGGUGCAG | 5367 |
| 694 | UGCCAUUU G UUCAGUGG | 1597 | CCACUGAA GGAGGAAACUCC CU UCAAGGACAUCGUCCGGG AAAUGGCA | 5368 |
| 724 | CCCCCACU G UCUGGCUU | 1598 | AAGCCAGA GGAGGAAACUCC CU UCAAGGACAUCGUCCGGG AGUGGGGG | 5369 |
| 750 | UGGAUGAU G UGGUUUUG | 1599 | CAAAACCA GGAGGAAACUCC CU UCAAGGACAUCGUCCGGG AUCAUCCA | 5370 |
| 771 | CCAAGUCU G UACAACAU | 1600 | AUGUUGUA GGAGGAAACUCC CU UCAAGGACAUCGUCCGGG AGACUUGG | 5371 |
| 801 | AUGCCGCU G UUACCAAU | 1601 | AUUGGUAA GGAGGAAACUCC CU UCAAGGACAUCGUCCGGG AGCGGCAU | 5372 |
| 818 | UUUCUUUU G UCUUUGGG | 1602 | CCCAAAGA GGAGGAAACUCC CU UCAAGGACAUCGUCCGGG AAAAGAAA | 5373 |
| 888 | UGGGAUAU G UAAUUGGG | 1603 | CCCAAUUA GGAGGAAACUCC CU UCAAGGACAUCGUCCGGG AUAUCCCA | 5374 |
| 927 | AACAUAUU G UACAAAAA | 1604 | UUUUUGUA GGAGGAAACUCC CU UCAAGGACAUCGUCCGGG AAUAUGUU | 5375 |
| 944 | AUCAAAAU G UGUUUUAG | 1605 | CUAAAACA GGAGGAAACUCC CU UCAAGGACAUCGUCCGGG AUUUUGAU | 5376 |
| 946 | CAAAAUGU G UUUUAGGA | 1606 | UCCUAAAA GGAGGAAACUCC CU UCAAGGACAUCGUCCGGG ACAUUUUG | 5377 |
| 963 | AACUUCCU G UAAACAGG | 1607 | CCUGUUUA GGAGGAAACUCC CU UCAAGGACAUCGUCCGGG AGGAAGUU | 5378 |
| 991 | GAAAGUAU G UCAACGAA | 1608 | UUCGUUGA GGAGGAAACUCC CU UCAAGGACAUCGUCCGGG AUACUUUC | 5379 |
| 1002 | AACGAAUU G UGGGUCUU | 1609 | AAGACCCA GGAGGAAACUCC CU UCAAGGACAUCGUCCGGG AAUUCGUU | 5380 |
| 1039 | CACGCAAU G UGGAUAUU | 1610 | AAUAUCCA GGAGGAAACUCC CU UCAAGGACAUCGUCCGGG AUUGCGUG | 5381 |
| 1137 | AACAGUAU G UGAACCUU | 1611 | AAGGUUCA GGAGGAAACUCC CU UCAAGGACAUCGUCCGGG AUACUGUU | 5382 |
| 1184 | UGCCAAGU G UUUGCUGA | 1612 | UCAGCAAA GGAGGAAACUCC CU UCAAGGACAUCGUCCGGG ACUUGGCA | 5383 |
| 1251 | GAACCUUU G UGUCUCCU | 1613 | AGGAGACA GGAGGAAACUCC CU UCAAGGACAUCGUCCGGG AAAGGUUC | 5384 |
| 1253 | ACCUUUGU G UCUCCUCU | 1614 | AGAGGAGA GGAGGAAACUCC CU UCAAGGACAUCGUCCGGG ACAAAGGU | 5385 |
| 1294 | AGCCGCUU G UUUUGCUC | 1615 | GAGCAAAA GGAGGAAACUCC CU UCAAGGACAUCGUCCGGG AAGCGGCU | 5386 |
| 1344 | ACAAUUCU G UCGUGCUC | 1616 | GAGCACGA GGAGGAAACUCC CU UCAAGGACAUCGUCCGGG AGAAUUGU | 5387 |
| 1390 | GCUAGGCU G UGCUGCCA | 1617 | UGGCAGCA GGAGGAAACUCC CU UCAAGGACAUCGUCCGGG AGCCUAGC | 5388 |
| 1425 | CGUCCUUU G UUUACGUC | 1618 | GACGUAAA GGAGGAAACUCC CU UCAAGGACAUCGUCCGGG AAAGGACG | 5389 |
| 1508 | CGCCUAUU G UACCGACC | 1619 | GGUCGGUA GGAGGAAACUCC CU UCAAGGACAUCGUCCGGG AAUAGGCG | 5390 |
| 1557 | CCCCGUCU G UGCCUUCU | 1620 | AGAAGGCA GGAGGAAACUCC CU UCAAGGACAUCGUCCGGG AGACGGGG | 5391 |
| 1581 | CGGACCGU G UGCACUUC | 1621 | GAAGUGCA GGAGGAAACUCC CU UCAAGGACAUCGUCCGGG ACGGUCCG | 5392 |
| 1684 | UCAGCAAU G UCAACGAC | 1622 | GUCGUUGA GGAGGAAACUCC CU UCAAGGACAUCGUCCGGG AUUGCUGA | 5393 |
| 1719 | CAAAGACU G UGUGUUUA | 1623 | UAAACACA GGAGGAAACUCC CU UCAAGGACAUCGUCCGGG AGUCUUUG | 5394 |
| 1721 | AAGACUGU G UGUUUAAU | 1624 | AUUAAACA GGAGGAAACUCC CU UCAAGGACAUCGUCCGGG ACAGUCUU | 5395 |
| 1723 | GACUGUGU G UUUAAUGA | 1625 | UCAUUAAA GGAGGAAACUCC CU UCAAGGACAUCGUCCGGG ACACAGUC | 5396 |
| 1772 | AGGUCUUU G UACUAGGA | 1626 | UCCUAGUA GGAGGAAACUCC CU UCAAGGACAUCGUCCGGG AAAGACCU | 5397 |
| 1785 | AGGAGGCU G UAGGCAUA | 1627 | UAUGCCUA GGAGGAAACUCC CU UCAAGGACAUCGUCCGGG AGCCUCCU | 5398 |
| 1801 | AAAUUGGU G UGUUCACC | 1628 | GGUGAACA GGAGGAAACUCC CU UCAAGGACAUCGUCCGGG ACCAAUUU | 5399 |
| 1803 | AUUGGUGU G UUCACCAG | 1629 | CUGGUGAA GGAGGAAACUCC CU UCAAGGACAUCGUCCGGG ACACCAAU | 5400 |
| 1850 | CAUCUCAU G UUCAUGUC | 1630 | GACAUGAA GGAGGAAACUCC CU UCAAGGACAUCGUCCGGG AUGAGAUG | 5401 |
| 1856 | AUGUUCAU G UCCUACUG | 1631 | CAGUAGGA GGAGGAAACUCC CU UCAAGGACAUCGUCCGGG AUGAACAU | 5402 |
| 1864 | GUCCUACU G UUCAAGCC | 1632 | GGCUUGAA GGAGGAAACUCC CU UCAAGGACAUCGUCCGGG AGUAGGAC | 5403 |
| 1881 | UCCAAGCU G UGCCUUGG | 1633 | CCAAGGCA GGAGGAAACUCC CU UCAAGGACAUCGUCCGGG AGCUUGGA | 5404 |
| 1939 | GAGCUUCU G UGGAGUUA | 1634 | UAACUCCA GGAGGAAACUCC CU UCAAGGACAUCGUCCGGG AGAAGCUC | 5405 |
| 2013 | UCUGCUCU G UAUCGGGG | 1635 | CCCCGAUA GGAGGAAACUCC CU UCAAGGACAUCGUCCGGG AGAGCAGA | 5406 |
| 2045 | GGAACAUU G UUCACCUC | 1636 | GAGGUGAA GGAGGAAACUCC CU UCAAGGACAUCGUCCGGG AAUGUUCC | 5407 |
| 2082 | GCUAUUCU G UGUUGGGG | 1637 | CCCCAACA GGAGGAAACUCC CU UCAAGGACAUCGUCCGGG AGAAUAGC | 5408 |
| 2084 | UAUUCUGU G UUGGGGUG | 1638 | CACCCCAA GGAGGAAACUCC CU UCAAGGACAUCGUCCGGG ACAGAAUA | 5409 |
| 2167 | UCAGCUAU G UCAACGUU | 1639 | AACGUUGA GGAGGAAACUCC CU UCAAGGACAUCGUCCGGG AUAGCUGA | 5410 |
| 2205 | CAACUAUU G UGGUUUCA | 1640 | UGAAACCA GGAGGAAACUCC CU UCAAGGACAUCGUCCGGG AAUAGUUG | 5411 |
| 2222 | CAUUUCCU G UCUUACUU | 1641 | AAGUAAGA GGAGGAAACUCC CU UCAAGGACAUCGUCCGGG AGGAAAUG | 5412 |
| 2245 | GAGAAACU G UUCUUGAA | 1642 | UUCAAGAA GGAGGAAACUCC CU UCAAGGACAUCGUCCGGG AGUUUCUC | 5413 |
| 2262 | UAUUUGGU G UCUUUUGG | 1643 | CCAAAAGA GGAGGAAACUCC CU UCAAGGACAUCGUCCGGG ACCAAAUA | 5414 |
| 2274 | UUUGGAGU G UGGAUUCG | 1644 | CGAAUCCA GGAGGAAACUCC CU UCAAGGACAUCGUCCGGG ACUCCAAA | 5415 |
| 2344 | AAACUACU G UUGUUAGA | 1645 | UCUAACAA GGAGGAAACUCC CU UCAAGGACAUCGUCCGGG AGUAGUUU | 5416 |
| 2347 | CUACUGUU G UUAGACGA | 1646 | UCGUCUAA GGAGGAAACUCC CU UCAAGGACAUCGUCCGGG AACAGUAG | 5417 |
| 2450 | AUCUCAAU G UUAGUAUU | 1647 | AAUACUAA GGAGGAAACUCC CU UCAAGGACAUCGUCCGGG AUUGAGAU | 5418 |
| 2573 | AGGACAUU G UUGAUAGA | 1648 | UCUAUCAA GGAGGAAACUCC CU UCAAGGACAUCGUCCGGG AAUGUCCU | 5419 |
| 2583 | UGAUAGAU G UAAGCAAU | 1649 | AUUGCUUA GGAGGAAACUCC CU UCAAGGACAUCGUCCGGG AUCUAUCA | 5420 |
| 2594 | AGCAAUUU G UGGGGCCC | 1650 | GGGCCCCA GGAGGAAACUCC CU UCAAGGACAUCGUCCGGG AAAUUGCU | 5421 |
| 2663 | AUCCCAAU G UUACUAAA | 1651 | UUUAGUAA GGAGGAAACUCC CU UCAAGGACAUCGUCCGGG AUUGGGAU | 5422 |
| 2717 | CAGAGUAU G UAGUUAAU | 1652 | AUUAACUA GGAGGAAACUCC CU UCAAGGACAUCGUCCGGG AUACUCUG | 5423 |
| 2901 | AUCUUUCU G UCCCCAAU | 1653 | AUUGGGGA GGAGGAAACUCC CU UCAAGGACAUCGUCCGGG AGAAAGAU | 5424 |
| 3071 | GGGGGACU G UUGGGGUG | 1654 | CACCCCAA GGAGGAAACUCC CU UCAAGGACAUCGUCCGGG AGUCCCCC | 5425 |
| 3111 | UCACAACU G UGCCAGCA | 1655 | UGCUGGCA GGAGGAAACUCC CU UCAAGGACAUCGUCCGGG AGUUGUGA | 5426 |
| 40 | AUCCCAGA G UCAGGGCC | 1656 | GGCCCUGA GGAGGAAACUCC CU UCAAGGACAUCGUCCGGG UCUGGGAU | 5427 |
| 46 | GAGUCAGG G CCCUGUAC | 1657 | GUACAGGG GGAGGAAACUCC CU UCAAGGACAUCGUCCGGG CCUGACUC | 5428 |
| 65 | UCCUGCUG G UGGCUCCA | 1658 | UGGAGCCA GGAGGAAACUCC CU UCAAGGACAUCGUCCGGG CAGCAGGA | 5429 |
| 68 | UGCUGGUG G CUCCAGUU | 1659 | AACUGGAG GGAGGAAACUCC CU UCAAGGACAUCGUCCGGG CACCAGCA | 5430 |
| 74 | UGGCUCCA G UUCAGGAA | 1660 | UUCCUGAA GGAGGAAACUCC CU UCAAGGACAUCGUCCGGG UGGAGCCA | 5431 |
| 85 | CAGGAACA G UGAGCCCU | 1661 | AGGGCUCA GGAGGAAACUCC CU UCAAGGACAUCGUCCGGG UGUUCCUG | 5432 |
| 89 | AACAGUGA G CCCUGCUC | 1662 | GAGCAGGG GGAGGAAACUCC CU UCAAGGACAUCGUCCGGG UCACUGUU | 5433 |
| 120 | GCCAUAUC G UCAAUCUU | 1663 | AAGAUUGA CCAGGAAACUCC CU UCAAGGACAUCGUCCGGG GAUAUGGC | 5434 |
| 196 | CCCUGCUC G UGUUACAG | 1664 | CUGUAACA GGAGGAAACUCC CU UCAAGGACAUCGUCCGGG GAGCAGGG | 5435 |
| 205 | UGUUACAG G CGGGGUUU | 1665 | AAACCCCG GGAGGAAACUCC CU UCAAGGACAUCGUCCGGG CUGUAACA | 5436 |
| 210 | CAGGCGGG G UUUUUCUU | 1666 | AAGAAAAA GGAGGAAACUCC CU UCAAGGACAUCGUCCGGG CCCGCCUG | 5437 |
| 248 | ACCACAGA G UCUAGACU | 1667 | AGUCUAGA GGAGGAAACUCC CU UCAAGGACAUCGUCCGGG UCUGUGGU | 5438 |
| 258 | CUAGACUC G UGGUGGAC | 1668 | GUCCACCA GGAGGAAACUCC CU UCAAGGACAUCGUCCGGG GAGUCUAG | 5439 |
| 261 | GACUCGUG G UGGACUUC | 1669 | GAAGUCCA GGAGGAAACUCC CU UCAAGGACAUCGUCCGGG CACGAGUC | 5440 |
| 295 | GAACACCC G UGUGUCUU | 1670 | AAGACACA GGAGGAAACUCC CU UCAAGGACAUCGUCCGGG GGGUGUUC | 5441 |
| 305 | GUGUCUUG G CCAAAAUU | 1671 | AAUUUUGG GGAGGAAACUCC CU UCAAGGACAUCGUCCGGG CAAGACAC | 5442 |
| 318 | AAUUCGCA G UCCCAAAU | 1672 | AUUUGGGA GGAGGAAACUCC CU UCAAGGACAUCGUCCGGG UGCGAAUU | 5443 |
| 332 | AAUCUCCA G UCACUCAC | 1673 | GUGAGUGA GGAGGAAACUCC CU UCAAGGACAUCGUCCGGG UGGAGAUU | 5444 |
| 368 | UUGUCCUG G UUAUCGCU | 1674 | AGCGAUAA GGAGGAAACUCC CU UCAAGGACAUCGUCCGGG CAGGACAA | 5445 |
| 390 | UGUCUGCG G CGUUUUAU | 1675 | AUAAAACG GGAGGAAACUCC CU UCAAGGACAUCGUCCGGG CGCAGACA | 5446 |
| 392 | UCUGCGGC G UUUUAUCA | 1676 | UGAUAAAA GGAGGAAACUCC CU UCAAGGACAUCGUCCGGG GCCGCAGA | 5447 |
| 442 | UCUUGUUG G UUCUUCUG | 1677 | CAGAAGAA GGAGGAAACUCC CU UCAAGGACAUCGUCCGGG CAACAAGA | 5448 |
| 461 | CUAUCAAG G UAUGUUGC | 1678 | GCAACAUA GGAGGAAACUCC CU UCAAGGACAUCGUCCGGG CUUGAUAG | 5449 |
| 472 | UGUUGCCC G UUUGUCCU | 1679 | AGGACAAA GGAGGAAACUCC CU UCAAGGACAUCGUCCGGG GGGCAACA | 5450 |
| 506 | AACAACCA G CACCGGAC | 1680 | GUCCGGUG GGAGGAAACUCC CU UCAAGGACAUCGUCCGGG UGGUUGUU | 5451 |
| 625 | CAUCUUGG G CUUUCGCA | 1681 | UGCGAAAG GGAGGAAACUCC CU UCAAGGACAUCGUCCGGG CCAAGAUG | 5452 |
| 648 | CUAUGGGA G UGGGCCUC | 1682 | GAGGCCCA GGAGGAAACUCC CU UCAAGGACAUCGUCCGGG UCCCAUAG | 5453 |
| 652 | GGGAGUGG G CCUCAGUC | 1683 | GACUGAGG GGAGGAAACUCC CU UCAAGGACAUCGUCCGGG CCACUCCC | 5454 |
| 658 | GGGCCUCA G UCCGUUUC | 1684 | GAAACGGA GGAGGAAACUCC CU UCAAGGACAUCGUCCGGG UGAGGCCC | 5455 |
| 662 | CUCAGUCC G UUUCUCUU | 1685 | AAGAGAAA GGAGGAAACUCC CU UCAAGGACAUCGUCCGGG GGACUGAG | 5456 |
| 672 | UUCUCUUG G CUCAGUUU | 1686 | AAACUGAG GGAGGAAACUCC CU UCAAGGACAUCGUCCGGG CAAGAGAA | 5457 |
| 677 | UUGGCUCA G UUUACUAG | 1687 | CUAGUAAA GGAGGAAACUCC CU UCAAGGACAUCGUCCGGG UGAGCCAA | 5458 |
| 685 | GUUUACUA G UGCCAUUU | 1688 | AAAUGGCA GGAGGAAACUCC CU UCAAGGACAUCGUCCGGG UAGUAAAC | 5459 |
| 699 | UUUGUUCA G UGGUUCGU | 1689 | ACGAACCA GGAGGAAACUCC CU UCAAGGACAUCGUCCGGG UGAACAAA | 5460 |
| 702 | GUUCAGUG G UUCGUAGG | 1690 | CCUACGAA GGAGGAAACUCC CU UCAAGGACAUCGUCCGGG CACUGAAC | 5461 |
| 706 | AGUGGUUC G UAGGGCUU | 1691 | AAGCCCUA GGAGGAAACUCC CU UCAAGGACAUCGUCCGGG GAACCACU | 5462 |
| 711 | UUCGUAGG G CUUUCCCC | 1692 | GGGGAAAG GGAGGAAACUCC CU UCAAGGACAUCGUCCGGG CCUACGAA | 5463 |
| 729 | ACUGUCUG G CUUUCAGU | 1693 | ACUGAAAG GGAGGAAACUCC CU UCAAGGACAUCGUCCGGG CAGACAGU | 5464 |
| 736 | GGCUUUCA G UUAUAUGG | 1694 | CCAUAUAA GGAGGAAACUCC CU UCAAGGACAUCGUCCGGG UGAAAGCC | 5465 |
| 753 | AUGAUGUG G UUUUGGGG | 1695 | CCCCAAAA GGAGGAAACUCC CU UCAAGGACAUCGUCCGGG CACAUCAU | 5466 |
| 762 | UUUUGGGG G CCAAGUCU | 1696 | AGACUUGG GGAGGAAACUCC CU UCAAGGACAUCGUCCGGG CCCCAAAA | 5467 |
| 767 | GGGGCCAA G UCUGUACA | 1697 | UGUACAGA GGAGGAAACUCC CU UCAAGGACAUCGUCCGGG UUGGCCCC | 5468 |
| 785 | CAUCUUGA G UCCCUUUA | 1698 | UAAAGGGA GGAGGAAACUCC CU UCAAGGACAUCGUCCGGG UCAAGAUG | 5469 |
| 826 | GUCUUUGG G UAUACAUU | 1699 | AAUGUAUA GGAGGAAACUCC CU UCAAGGACAUCGUCCGGG CCAAAGAC | 5470 |
| 898 | AAUUGGGA G UUGGGGCA | 1700 | UGCCCCAA GGAGGAAACUCC CU UCAAGGACAUCGUCCGGG UCCCAAUU | 5471 |
| 904 | GAGUUGGG G CACAUUGC | 1701 | GCAAUGUG GGAGGAAACUCC CU UCAAGGACAUCGUCCGGG CCCAACUC | 5472 |
| 971 | GUAAACAG G CCUAUUGA | 1702 | UCAAUAGG GGAGGAAACUCC CU UCAAGGACAUCGUCCGGG CUGUUUAC | 5473 |
| 987 | AUUGGAAA G UAUGUCAA | 1703 | UUGACAUA GGAGGAAACUCC CU UCAAGGACAUCGUCCGGG UUUCCAAU | 5474 |
| 1006 | AAUUGUGG G UCUUUUGG | 1704 | CCAAAAGA GGAGGAAACUCC CU UCAAGGACAUCGUCCGGG CCACAAUU | 5475 |
| 1016 | CUUUUGGG G UUUGCCGC | 1705 | GCGGCAAA GGAGGAAACUCC CU UCAAGGACAUCGUCCGGG CCCAAAAG | 5476 |
| 1080 | GCAUACAA G CAAAACAG | 1706 | CUGUUUUG GGAGGAAACUCC CU UCAAGGACAUCGUCCGGG UUGUAUGC | 5477 |
| 1089 | CAAAACAG G CUUUUACU | 1707 | AGUAAAAG GGAGGAAACUCC CU UCAAGGACAUCGUCCGGG CUGUUUUG | 5478 |
| 1116 | CUUACAAG G CCUUUCUA | 1708 | UAGAAAGG GGAGGAAACUCC CU UCAAGGACAUCGUCCGGG CUUGUAAG | 5479 |
| 1126 | CUUUCUAA G UAAACAGU | 1709 | ACUGUUUA GGAGGAAACUCC CU UCAAGGACAUCGUCCGGG UUAGAAAG | 5480 |
| 1132 | AGUAAACA G UAUGUGAA | 1710 | UUCACAUA GGAGGAAACUCC CU UCAAGGACAUCGUCCGGG UGUUUACU | 5481 |
| 1152 | UUUACCCC G UUGCUCGG | 1711 | CCGAGCAA GGAGGAAACUCC CU UCAAGGACAUCGUCCGGG GGGGUAAA | 5482 |
| 1160 | GUUGCUCG G CAACGGCC | 1712 | GGCCGUUG GGAGGAAACUCC CU UCAAGGACAUCGUCCGGG CGAGCAAC | 5483 |
| 1166 | CGGCAACG G CCUGGUCU | 1713 | AGACCAGG GGAGGAAACUCC CU UCAAGGACAUCGUCCGGG CGUUGCCG | 5484 |
| 1171 | ACGGCCUG G UCUAUGCC | 1714 | GGCAUAGA GGAGGAAACUCC CU UCAAGGACAUCGUCCGGG CAGGCCGU | 5485 |
| 1182 | UAUGCCAA G UGUUUGCU | 1715 | AGCAAACA GGAGGAAACUCC CU UCAAGGACAUCGUCCGGG UUGGCAUA | 5486 |
| 1207 | CCCCACUG G UUGGGGCU | 1716 | AGCCCCAA GGAGGAAACUCC CU UCAAGGACAUCGUCCGGG CAGUGGGG | 5487 |
| 1213 | UGGUUGGG G CUUGGCCA | 1717 | UGGCCAAG GGAGGAAACUCC CU UCAAGGACAUCGUCCGGG CCCAACCA | 5488 |
| 1218 | GGGGCUUG G CCAUAGGC | 1718 | GCCUAUGG GGAGGAAACUCC CU UCAAGGACAUCGUCCGGG CAAGCCCC | 5489 |
| 1225 | GGCCAUAG G CCAUCAGC | 1719 | GCUGAUGG GGAGGAAACUCC CU UCAAGGACAUCGUCCGGG CUAUGGCC | 5490 |
| 1232 | GGCCAUCA G CGCAUGCG | 1720 | CGCAUGCG GGAGGAAACUCC CU UCAAGGACAUCGUCCGGG UGAUGGCC | 5491 |
| 1240 | GCGCAUGC G UGGAACCU | 1721 | AGGUUCCA GGAGGAAACUCC CU UCAAGGACAUCGUCCGGG GCAUGCGC | 5492 |
| 1287 | AACUCCUA G CCGCUUGU | 1722 | ACAAGCGG GGAGGAAACUCC CU UCAAGGACAUCGUCCGGG UAGGAGUU | 5493 |
| 1306 | UGCUCGCA G CAGGUCUG | 1723 | CAGACCUG GGAGGAAACUCC CU UCAAGGACAUCGUCCGGG UGCGAGCA | 5494 |
| 1310 | CGCAGCAG G UCUGGGGC | 1724 | GCCCCAGA GGAGGAAACUCC CU UCAAGGACAUCGUCCGGG CUGCUGCG | 5495 |
| 1317 | GGUCUGGG G CAAAACUC | 1725 | GAGUUUUG GGAGGAAACUCC CU UCAAGGACAUCGUCCGGG CCCAGACC | 5496 |
| 1347 | AUUCUGUC G UGCUCUCC | 1726 | GGAGAGCA GGAGGAAACUCC CU UCAAGGACAUCGUCCGGG GACAGAAU | 5497 |
| 1379 | UUUCCAUG G CUGCUAGG | 1727 | CCUAGCAG GGAGGAAACUCC CU UCAAGGACAUCGUCCGGG CAUGGAAA | 5498 |
| 1387 | GCUGCUAG G CUGUGCUG | 1728 | CAGCACAG GGAGGAAACUCC CU UCAAGGACAUCGUCCGGG CUAGCAGC | 5499 |
| 1418 | CGCGGGAC G UCCUUUGU | 1729 | ACAAAGGA GGAGGAAACUCC CU UCAAGGACAUCGUCCGGG GUCCCGCG | 5500 |
| 1431 | UUGUUUAC G UCCCGUCG | 1730 | CGACGGGA GGAGGAAACUCC CU UCAAGGACAUCGUCCGGG GUAAACAA | 5501 |
| 1436 | UACGUCCC G UCGGCGCU | 1731 | AGCGCCGA GGAGGAAACUCC CU UCAAGGACAUCGUCCGGG GGGACGUA | 5502 |
| 1440 | UCCCGUCG G CGCUGAAU | 1732 | AUUCAGCG GGAGGAAACUCC CU UCAAGGACAUCGUCCGGG CGACGGGA | 5503 |
| 1471 | CUCCCGGG G CCGCUUGG | 1733 | CCAAGCGG GGAGGAAACUCC CU UCAAGGACAUCGUCCGGG CCCGGGAG | 5504 |
| 1481 | CGCUUGGG G CUCUACCG | 1734 | CGGUAGAG GGAGGAAACUCC CU UCAAGGACAUCGUCCGGG CCCAAGCG | 5505 |
| 1517 | UACCGACC G UCCACGGG | 1735 | CCCGUGGA GGAGGAAACUCC CU UCAAGGACAUCGUCCGGG GGUCGGUA | 5506 |
| 1526 | UCCACGGG G CGCACCUC | 1736 | GAGGUGCG GGAGGAAACUCC CU UCAAGGACAUCGUCCGGG CCCGUGGA | 5507 |
| 1553 | GACUCCCC G UCUGUGCC | 1737 | GGCACAGA GGAGGAAACUCC CU UCAAGGACAUCGUCCGGG GGGGAGUC | 5508 |
| 1579 | GCCGGACC G UGUGCACU | 1738 | AGUGCACA GGAGGAAACUCC CU UCAAGGACAUCGUCCGGG GGUCCGGC | 5509 |
| 1605 | CUCUGCAC G UCGCAUGG | 1739 | CCAUGCGA GGAGGAAACUCC CU UCAAGGACAUCGUCCGGG GUGCAGAG | 5510 |
| 1622 | AGACCACC G UGAACGCC | 1740 | GGCGUUCA GGAGGAAACUCC CU UCAAGGACAUCGUCCGGG GGUGGUCU | 5511 |
| 1649 | UGCCCAAG G UCUUGCAU | 1741 | AUGCAAGA GGAGGAAACUCC CU UCAAGGACAUCGUCCGGG CUUGGGCA | 5512 |
| 1679 | GACUUUCA G CAAUGUCA | 1742 | UGACAUUG GGAGGAAACUCC CU UCAAGGACAUCGUCCGGG UGAAAGUC | 5513 |
| 1703 | ACCUUGAG G CAUACUCC | 1743 | GAAGUAUG GGAGGAAACUCC CU UCAAGGACAUCGUCCGGG CUCAAGGU | 5514 |
| 1732 | UUUAAUGA G UGGGAGGA | 1744 | UCCUCCCA GGAGGAAACUCC CU UCAAGGACAUCGUCCGGG UCAUUAAA | 5515 |
| 1741 | UGGGAGGA G UUGGGGGA | 1745 | UCCCCCAA GGAGGAAACUCC CU UCAAGGACAUCGUCCGGG UCCUCCCA | 5516 |
| 1754 | GGGAGGAG G UUAGGUUA | 1746 | UAACCUAA GGAGGAAACUCC CU UCAAGGACAUCGUCCGGG CUCCUCCC | 5517 |
| 1759 | GAGGUUAG G UUAAAGGU | 1747 | ACCUUUAA GGAGGAAACUCC CU UCAAGGACAUCGUCCGGG CUAACCUC | 5518 |
| 1766 | GGUUAAAG G UCUUUGUA | 1748 | UACAAAGA GGAGGAAACUCC CU UCAAGGACAUCGUCCGGG CUUUAACC | 5519 |
| 1782 | ACUAGGAG G CUGUAGGC | 1749 | GCCUACAG GGAGGAAACUCC CU UCAAGGACAUCGUCCGGG CUCCUAGU | 5520 |
| 1789 | GGCUGUAG G CAUAAAUU | 1750 | AAUUUAUG GGAGGAAACUCC CU UCAAGGACAUCGUCCGGG CUACAGCC | 5521 |
| 1799 | AUAAAUUG G UGUGUUCA | 1751 | UGAACACA GGAGGAAACUCC CU UCAAGGACAUCGUCCGGG CAAUUUAU | 5522 |
| 1811 | GUUCACCA G CACCAUGC | 1752 | GCAUGGUG GGAGGAAACUCC CU UCAAGGACAUCGUCCGGG UGGUGAAC | 5523 |
| 1870 | CUGUUCAA G CCUCCAAG | 1753 | CUUGGAGG GGAGGAAACUCC CU UCAAGGACAUCGUCCGGG UUGAACAG | 5524 |
| 1878 | GCCUCCAA G CUGUGCCU | 1754 | AGGCACAG GGAGGAAACUCC CU UCAAGGACAUCGUCCGGG UUGGAGGC | 5525 |
| 1890 | UGCCUUGG G UGGCUUUG | 1755 | CAAAGCCA GGAGGAAACUCC CU UCAAGGACAUCGUCCGGG CCAAGGCA | 5526 |
| 1893 | CUUGGGUG G CUUUGGGG | 1756 | CCCCAAAG GGAGGAAACUCC CU UCAAGGACAUCGUCCGGG CACCCAAG | 5527 |
| 1901 | GCUUUGGG G CAUGGACA | 1757 | UGUCCAUG GGAGGAAACUCC CU UCAAGGACAUCGUCCGGG CCCAAAGC | 5528 |
| 1917 | AUUGACCC G UAUAAAGA | 1758 | UCUUUAUA GGAGGAAACUCC CU UCAAGGACAUCGUCCGGG GGGUCAAU | 5529 |
| 1933 | AAUUUGGA G CUUCUGUG | 1759 | CACAGAAG GGAGGAAACUCC CU UCAAGGACAUCGUCCGGG UCCAAAUU | 5530 |
| 1944 | UCUGUGGA G UUACUCUC | 1760 | GAGAGUAA GGAGGAAACUCC CU UCAAGGACAUCGUCCGGG UCCACAGA | 5531 |
| 2023 | AUCGGGGG G CCUUAGAG | 1761 | CUCUAAGG GGAGGAAACUCC CU UCAAGGACAUCGUCCGGG CCCCCGAU | 5532 |
| 2031 | GCCUUAGA G UCUCCGGA | 1762 | UCCGGAGA GGAGGAAACUCC CU UCAAGGACAUCGUCCGGG UCUAAGGC | 5533 |
| 2062 | ACCAUACG G CACUCAGG | 1763 | CCUGAGUG GGAGGAAACUCC CU UCAAGGACAUCGUCCGGG CGUAUGGU | 5534 |
| 2070 | GCACUCAG G CAAGCUAU | 1764 | AUAGCUUG GGAGGAAACUCC CU UCAAGGACAUCGUCCGGG CUGAGUGC | 5535 |
| 2074 | UCAGGCAA G CUAUUCUG | 1765 | CAGAAUAG GGAGGAAACUCC CU UCAAGGACAUCGUCCGGG UUGCCUGA | 5536 |
| 2090 | GUGUUGGG G UGAGUUGA | 1766 | UCAACUCA GGAGGAAACUCC CU UCAAGGACAUCGUCCGGG CCCAACAC | 5537 |
| 2094 | UGGGGUGA G UUGAUGAA | 1767 | UUCAUCAA GGAGGAAACUCC CU UCAAGGACAUCGUCCGGG UCACCCCA | 5538 |
| 2107 | UGAAUCUA G CCACCUGG | 1768 | CCAGGUGG GGAGGAAACUCC CU UCAAGGACAUCGUCCGGG UAGAUUCA | 5539 |
| 2116 | CCACCUGG G UGGGAAGU | 1769 | ACUUCCCA GGAGGAAACUCC CU UCAAGGACAUCGUCCGGG CCAGGUGG | 5540 |
| 2123 | GGUGGGAA G UAAUUUGG | 1770 | CCAAAUUA GGAGGAAACUCC CU UCAAGGACAUCGUCCGGG UUCCCACC | 5541 |
| 2140 | AAGAUCCA G CAUCCAGG | 1771 | CCUGGAUG GGAGGAAACUCC CU UCAAGGACAUCGUCCGGG UGGAUCUU | 5542 |
| 2155 | GGGAAUUA G UAGUCAGC | 1772 | GCUGACUA GGAGGAAACUCC CU UCAAGGACAUCGUCCGGG UAAUUCCC | 5543 |
| 2158 | AAUUAGUA G UCAGCUAU | 1773 | AUAGCUGA GGAGGAAACUCC CU UCAAGGACAUCGUCCGGG UACUAAUU | 5544 |
| 2162 | AGUAGUCA G CUAUGUCA | 1774 | UGACAUAG GGAGGAAACUCC CU UCAAGGACAUCGUCCGGG UGACUACU | 5545 |
| 2173 | AUGUCAAC G UUAAUAUG | 1775 | CAUAUUAA GGAGGAAACUCC CU UCAAGGACAUCGUCCGGG GUUGACAU | 5546 |
| 2183 | UAAUAUGG G CCUAAAAA | 1776 | UUUUUAGG GGAGGAAACUCC CU UCAAGGACAUCGUCCGGG CCAUAUUA | 5547 |
| 2208 | CUAUUGUG G UUUCACAU | 1777 | AUGUGAAA GGAGGAAACUCC CU UCAAGGACAUCGUCCGGG CACAAUAG | 5548 |
| 2235 | ACUUUUGG G CGAGAAAC | 1778 | GUUUCUCG GGAGGAAACUCC CU UCAAGGACAUCGUCCGGG CCAAAAGU | 5549 |
| 2260 | AAUAUUUG G UGUCUUUU | 1779 | AAAAGACA GGAGGAAACUCC CU UCAAGGACAUCGUCCGGG CAAAUAUU | 5550 |
| 2272 | CUUUUGGA G UGUGGAUU | 1780 | AAUCCACA GGAGGAAACUCC CU UCAAGGACAUCGUCCGGG UCCAAAAG | 5551 |
| 2360 | ACGAAGAG G CAGGUCCC | 1781 | GGGACCUG GGAGGAAACUCC CU UCAAGGACAUCGUCCGGG CUCUUCGU | 5552 |
| 2364 | AGAGGCAG G UCCCCUAG | 1782 | CUAGGGGA GGAGGAAACUCC CU UCAAGGACAUCGUCCGGG CUGCCUCU | 5553 |
| 2403 | AGACGAAG G UCUCAAUC | 1783 | GAUUGAGA GGAGGAAACUCC CU UCAAGGACAUCGUCCGGG CUUCGUCU | 5554 |
| 2417 | AUCGCCGC G UCGCAGAA | 1784 | UUCUGCGA GGAGGAAACUCC CU UCAAGGACAUCGUCCGGG GCGGCGAU | 5555 |
| 2454 | CAAUGUUA G UAUUCCUU | 1785 | AAGGAAUA GGAGGAAACUCC CU UCAAGGACAUCGUCCGGG UAACAUUG | 5556 |
| 2474 | CACAUAAG G UGGGAAAC | 1786 | GUUUCCCA GGAGGAAACUCC CU UCAAGGACAUCGUCCGGG CUUAUGUG | 5557 |
| 2491 | UUUACGGG G CUUUAUUC | 1787 | GAAUAAAG GGAGGAAACUCC CU UCAAGGACAUCGUCCGGG CCCGUAAA | 5558 |
| 2507 | CUUCUACG G UACCUUGC | 1788 | GCAAGGUA GGAGGAAACUCC CU UCAAGGACAUCGUCCGGG CGUAGAAG | 5559 |
| 2530 | CCUAAAUG G CAAACUCC | 1789 | GGAGUUUG GGAGGAAACUCC CU UCAAGGACAUCGUCCGGG CAUUUAGG | 5560 |
| 2587 | AGAUGUAA G CAAUUUGU | 1790 | ACAAAUUG GGAGGAAACUCC CU UCAAGGACAUCGUCCGGG UUACAUCU | 5561 |
| 2599 | UUUGUGGG G CCCCUUAC | 1791 | GUAAGGGG GGAGGAAACUCC CU UCAAGGACAUCGUCCGGG CCCACAAA | 5562 |
| 2609 | CCCUUACA G UAAAUGAA | 1792 | UUCAUUUA GGAGGAAACUCC CU UCAAGGACAUCGUCCGGG UGUAAGGG | 5563 |
| 2650 | CCUGCUAG G UUUUAUCC | 1793 | GGAUAAAA GGAGGAAACUCC CU UCAAGGACAUCGUCCGGG CUAGCAGG | 5564 |
| 2701 | AUCAAACC G UAUUAUCC | 1794 | GGAUAAUA GGAGGAAACUCC CU UCAAGGACAUCGUCCGGG GGUUUGAU | 5565 |
| 2713 | UAUCCAGA G UAUGUAGU | 1795 | ACUACAUA GGAGGAAACUCC CU UCAAGGACAUCGUCCGGG UCUGGAUA | 5566 |
| 2720 | AGUAUGUA G UUAAUCAU | 1796 | AUGAUUAA GGAGGAAACUCC CU UCAAGGACAUCGUCCGGG UACAUACU | 5567 |
| 2768 | UUUGGAAG G CGGGGAUC | 1797 | GAUCCCCG GGAGGAAACUCC CU UCAAGGACAUCGUCCGGG CUUCCAAA | 5568 |
| 2791 | AAAAGAGA G UCCACACG | 1798 | CGUGUGGA GGAGGAAACUCC CU UCAAGGACAUCGUCCGGG UCUCUUUU | 5569 |
| 2799 | GUCCACAC G UAGCGCCU | 1799 | AGGCGCUA GGAGGAAACUCC CU UCAAGGACAUCGUCCGGG GUGUGGAC | 5570 |
| 2802 | CACACGUA G CGCCUCAU | 1800 | AUGAGGCG GGAGGAAACUCC CU UCAAGGACAUCGUCCGGG UACGUGUG | 5571 |
| 2818 | UUUUGCGG G UCACCAUA | 1801 | UAUGGUGA GGAGGAAACUCC CU UCAAGGACAUCGUCCGGG CCGCAAAA | 5572 |
| 2848 | GAUCUACA G CAUGGGAG | 1802 | CUCCCAUG GGAGGAAACUCC CU UCAAGGACAUCGUCCGGG UGUAGAUC | 5573 |
| 2857 | CAUGGGAG G UUGGUCUU | 1803 | AAGACCAA GGAGGAAACUCC CU UCAAGGACAUCGUCCGGG CUCCCAUG | 5574 |
| 2861 | GGAGGUUG G UCUUCCAA | 1804 | UUGGAAGA GGAGGAAACUCC CU UCAAGGACAUCGUCCGGG CAACCUCC | 5575 |
| 2881 | UCGAAAAG G CAUGGGGA | 1805 | UCCCCAUG GGAGGAAACUCC CU UCAAGGACAUCGUCCGGG CUUUUCGA | 5576 |
| 2936 | GAUCAUCA G UUGGACCC | 1806 | GGGUCCAA GGAGGAAACUCC CU UCAAGGACAUCGUCCGGG UGAUGAUC | 5577 |
| 2955 | CAUUCAAA G CCAACUCA | 1807 | UGAGUUGG GGAGGAAACUCC CU UCAAGGACAUCGUCCGGG UUUGAAUG | 5578 |
| 2964 | CCAACUCA G UAAAUCCA | 1808 | UGGAUUUA GGAGGAAACUCC CU UCAAGGACAUCGUCCGGG UGAGUUGG | 5579 |
| 3005 | GACAACUG G CCGGACGC | 1809 | GCGUCCGG GGAGGAAACUCC CU UCAAGGACAUCGUCCGGG CAGUUGUC | 5580 |
| 3021 | CCAACAAG G UGGGAGUG | 1810 | CACUCCCA GGAGGAAACUCC CU UCAAGGACAUCGUCCGGG CUUGUUGG | 5581 |
| 3027 | AGGUGGGA G UGGGAGCA | 1811 | UGCUCCCA GGAGGAAACUCC CU UCAAGGACAUCGUCCGGG UCCCACCU | 5582 |
| 3033 | GAGUGGGA G CAUUCGGG | 1812 | CCCGAAUG GGAGGAAACUCC CU UCAAGGACAUCGUCCGGG UCCCACUC | 5583 |
| 3041 | GCAUUCGG G CCAGGGUU | 1813 | AACCCUGG GGAGGAAACUCC CU UCAAGGACAUCGUCCGGG CCGAAUGC | 5584 |
| 3047 | GGGCCAGG G UUCACCCC | 1814 | GGGGUGAA GGAGGAAACUCC CU UCAAGGACAUCGUCCGGG CCUGGCCC | 5585 |
| 3077 | CUGUUGGG G UGGAGCCC | 1815 | GGGCUCCA GGAGGAAACUCC CU UCAAGGACAUCGUCCGGG CCCAACAG | 5586 |
| 3082 | GGGGUGGA G CCCUCACG | 1816 | CGUGAGGG GGAGGAAACUCC CU UCAAGGACAUCGUCCGGG UCCACCCC | 5587 |
| 3097 | CGCUCAGG G CCUACUCA | 1817 | UGAGUAGG GGAGGAAACUCC CU UCAAGGACAUCGUCCGGG CCUGAGCG | 5588 |
| 3117 | CUGUGCCA G CAGCUCCU | 1818 | AGGAGCUG GGAGGAAACUCC CU UCAAGGACAUCGUCCGGG UGGCACAG | 5589 |
| 3120 | UGCCAGCA G CUCCUCCU | 1819 | AGGAGGAG GGAGGAAACUCC CU UCAAGGACAUCGUCCGGG UGCUGGCA | 5590 |
| 3146 | ACCAAUCG G CAGUCAGG | 1820 | CCUGACUG GGAGGAAACUCC CU UCAAGGACAUCGUCCGGG CGAUUGGU | 5591 |
| 3149 | AAUCGGCA G UCAGGAAG | 1821 | CUUCCUGA GGAGGAAACUCC CU UCAAGGACAUCGUCCGGG UGCCGAUU | 5592 |
| 3158 | UCAGGAAG G CAGCCUAC | 1822 | GUAGGCUG GGAGGAAACUCC CU UCAAGGACAUCGUCCGGG CUUCCUGA | 5593 |
| 3161 | GGAAGGCA G CCUACUCC | 1823 | GGAGUAGG GGAGGAAACUCC CU UCAAGGACAUCGUCCGGG UGCCUUCC | 5594 |
| 3204 | AUCCUCAG G CCAUGCAG | 1824 | CUGCAUGG GGAGGAAACUCC CU UCAAGGACAUCGUCCGGG CUGAGGAU | 5595 |
| 31 | CUCUUCAA G AUCCCAGA | 2196 | UCUGGGAU GGAGGAAACUCC CU UCAAGGACAUCGUCCGGG UUGAAGAG | 5596 |
| 38 | AGAUCCCA G AGUCAGGG | 2197 | CCCUGACU GGAGGAAACUCC CU UCAAGGACAUCGUCCGGG UGGGAUCU | 5597 |
| 44 | CAGAGUCA G GGCCCUGU | 2198 | ACAGGGCC GGAGGAAACUCC CU UCAAGGACAUCGUCCGGG UGACUCUG | 5598 |
| 45 | AGAGUCAG G GCCCUGUA | 2199 | UACAGGGC GGAGGAAACUCC CU UCAAGGACAUCGUCCGGG CUGACUCU | 5599 |
| 64 | UUCCUGCU G GUGGCUCC | 2200 | GGAGCCAC GGAGGAAACUCC CU UCAAGGACAUCGUCCGGG AGCAGGAA | 5600 |
| 67 | CUGCUGGU G GCUCCAGU | 2201 | ACUGGAGC GGAGGAAACUCC CU UCAAGGACAUCGUCCGGG ACCAGCAG | 5601 |
| 79 | CCAGUUCA G GAACAGUG | 2202 | CACUGUUC GGAGGAAACUCC CU UCAAGGACAUCGUCCGGG UGAACUGG | 5602 |
| 80 | CAGUUCAG G AACAGUGA | 2203 | UCACUGUU GGAGGAAACUCC CU UCAAGGACAUCGUCCGGG CUGAACUG | 5603 |
| 99 | CCUGCUCA G AAUACUGU | 2204 | ACAGUAUU GGAGGAAACUCC CU UCAAGGACAUCGUCCGGG UGAGCAGG | 5604 |
| 135 | UUAUCGAA G ACUGGGGA | 2205 | UCCCCAGU GGAGGAAACUCC CU UCAAGGACAUCGUCCGGG UUCGAUAA | 5605 |
| 139 | CGAAGACU G GGGACCCU | 2206 | AGGGUCCC GGAGGAAACUCC CU UCAAGGACAUCGUCCGGG AGUCUUCG | 5606 |
| 140 | GAAGACUG G GGACCCUG | 2207 | CAGGGUCC GGAGGAAACUCC CU UCAAGGACAUCGUCCGGG CAGUCUUC | 5607 |
| 141 | AAGACUGG G GACCCUGU | 2208 | ACAGGGUC GGAGGAAACUCC CU UCAAGGACAUCGUCCGGG CCAGUCUU | 5608 |
| 142 | AGACUGGG G ACCCUGUA | 2209 | UACAGGGU GGAGGAAACUCC CU UCAAGGACAUCGUCCGGG CCCAGUCU | 5609 |
| 159 | CCGAACAU G GAGAACAU | 2210 | AUGUUCUC GGAGGAAACUCC CU UCAAGGACAUCGUCCGGG AUGUUCGG | 5610 |
| 160 | CGAACAUG G AGAACAUC | 2211 | GAUGUUCU GGAGGAAACUCC CU UCAAGGACAUCGUCCGGG CAUGUUCG | 5611 |
| 162 | AACAUGGA G AACAUCGC | 2212 | GCGAUGUU GGAGGAAACUCC CU UCAAGGACAUCGUCCGGG UCCAUGUU | 5612 |
| 175 | UCGCAUCA G GACUCCUA | 2213 | UAGGAGUC GGAGGAAACUCC CU UCAAGGACAUCGUCCGGG UGAUGCGA | 5613 |
| 176 | CGCAUCAG G ACUCCUAG | 2214 | CUAGGAGU GGAGGAAACUCC CU UCAAGGACAUCGUCCGGG CUGAUGCG | 5614 |
| 184 | GACUCCUA G GACCCCUG | 2215 | CAGGGGUC GGAGGAAACUCC CU UCAAGGACAUCGUCCGGG UAGGAGUC | 5615 |
| 185 | ACUCCUAG G ACCCCUGC | 2216 | GCAGGGGU GGAGGAAACUCC CU UCAAGGACAUCGUCCGGG CUAGGAGU | 5616 |
| 204 | GUGUUACA G GCGGGGUU | 2217 | AACCCCGC GGAGGAAACUCC CU UCAAGGACAUCGUCCGGG UGUAACAC | 5617 |
| 207 | UUACAGGC G GGGUUUUU | 2218 | AAAAACCC GGAGGAAACUCC CU UCAAGGACAUCGUCCGGG GCCUGUAA | 5618 |
| 208 | UACAGGCG G GGUUUUUC | 2219 | GAAAAACC GGAGGAAACUCC CU UCAAGGACAUCGUCCGGG CGCCUGUA | 5619 |
| 209 | ACAGGCGG G GUUUUUCU | 2220 | AGAAAAAC GGAGGAAACUCC CU UCAAGGACAUCGUCCGGG CCGCCUGU | 5620 |
| 246 | AUACCACA G AGUCUAGA | 2221 | UCUAGACU GGAGGAAACUCC CU UCAAGGACAUCGUCCGGG UGUGGUAU | 5621 |
| 253 | AGAGUCUA G ACUCGUGG | 2222 | CCACGAGU GGAGGAAACUCC CU UCAAGGACAUCGUCCGGG UAGACUCU | 5622 |
| 260 | AGACUCGU G GUGGACUU | 2223 | AAGUCCAC GGAGGAAACUCC CU UCAAGGACAUCGUCCGGG ACGAGUCU | 5623 |
| 263 | CUCGUGGU G GACUUCUC | 2224 | GAGAAGUC GGAGGAAACUCC CU UCAAGGACAUCGUCCGGG ACCACGAG | 5624 |
| 264 | UCGUGGUG G ACUUCUCU | 2225 | AGAGAAGU GGAGGAAACUCC CU UCAAGGACAUCGUCCGGG CACCACGA | 5625 |
| 283 | AUUUUCUA G GGGGAACA | 2226 | UGUUCCCC GGAGGAAACUCC CU UCAAGGACAUCGUCCGGG UAGAAAAU | 5626 |
| 284 | UUUUCUAG G GGGAACAC | 2227 | GUGUUCCC GGAGGAAACUCC CU UCAAGGACAUCGUCCGGG CUAGAAAA | 5627 |
| 285 | UUUCUAGG G GGAACACC | 2228 | GGUGUUCC GGAGGAAACUCC CU UCAAGGACAUCGUCCGGG CCUAGAAA | 5628 |
| 286 | UUCUAGGG G GAACACCC | 2229 | GGGUGUUC GGAGGAAACUCC CU UCAAGGACAUCGUCCGGG CCCUAGAA | 5629 |
| 287 | UCUAGGGG G AACACCCG | 2230 | CGGGUGUU GGAGGAAACUCC CU UCAAGGACAUCGUCCGGG CCCCUAGA | 5630 |
| 304 | UGUGUCUU G GCCAAAAU | 2231 | AUUUUGGC GGAGGAAACUCC CU UCAAGGACAUCGUCCGGG AAGACACA | 5631 |
| 367 | UUUGUCCU G GUUAUCGC | 2232 | GCGAUAAC GGAGGAAACUCC CU UCAAGGACAUCGUCCGGG AGGACAAA | 5632 |
| 377 | UUAUCGCU G GAUGUGUC | 2233 | GACACAUC GGAGGAAACUCC CU UCAAGGACAUCGUCCGGG AGCGAUAA | 5633 |
| 378 | UAUCGCUG G AUGUGUCU | 2234 | AGACACAU GGAGGAAACUCC CU UCAAGGACAUCGUCCGGG CAGCGAUA | 5634 |
| 389 | GUGUCUGC G GCGUUUUA | 2235 | UAAAACGC GGAGGAAACUCC CU UCAAGGACAUCGUCCGGG GCAGACAC | 5635 |
| 441 | UUCUUGUU G GUUCUUCU | 2236 | AGAAGAAC GGAGGAAACUCC CU UCAAGGACAUCGUCCGGG AACAAGAA | 5636 |
| 450 | GUUCUUCU G GACUAUCA | 2237 | UGAUAGUC GGAGGAAACUCC CU UCAAGGACAUCGUCCGGG AGAAGAAC | 5637 |
| 451 | UUCUUCUG G ACUAUCAA | 2238 | UUGAUAGU GGAGGAAACUCC CU UCAAGGACAUCGUCCGGG CAGAAGAA | 5638 |
| 460 | ACUAUCAA G GUAUGUUG | 2239 | CAACAUAC GGAGGAAACUCC CU UCAAGGACAUCGUCCGGG UUGAUAGU | 5639 |
| 490 | UAAUUCCA G GAUCAUCA | 2240 | UGAUGAUC GGAGGAAACUCC CU UCAAGGACAUCGUCCGGG UGGAAUUA | 5640 |
| 491 | AAUUCCAG G AUCAUCAA | 2241 | UUGAUGAU GGAGGAAACUCC CU UCAAGGACAUCGUCCGGG CUGGAAUU | 5641 |
| 511 | CCAGCACC G GACCAUGC | 2242 | GCAUGGUC GGAGGAAACUCC CU UCAAGGACAUCGUCCGGG GGUGCUGG | 5642 |
| 512 | CAGCACCG G ACCAUGCA | 2243 | UGCAUGGU GGAGGAAACUCC CU UCAAGGACAUCGUCCGGG CGGUGCUG | 5643 |
| 544 | CUGCUCAA G GAACCUCU | 2244 | AGAGGUUC GGAGGAAACUCC CU UCAAGGACAUCGUCCGGG UUGAGCAG | 5644 |
| 545 | UGCUCAAG G AACCUCUA | 2245 | UAGAGGUU GGAGGAAACUCC CU UCAAGGACAUCGUCCGGG CUUGAGCA | 5645 |
| 585 | AAACCUAC G GACGGAAA | 2246 | UUUCCGUC GGAGGAAACUCC CU UCAAGGACAUCGUCCGGG GUAGGUUU | 5646 |
| 586 | AACCUACG G ACGGAAAC | 2247 | GUUUCCGU GGAGGAAACUCC CU UCAAGGACAUCGUCCGGG CGUAGGUU | 5647 |
| 589 | CUACGGAC G GAAACUGC | 2248 | GCAGUUUC GGAGGAAACUCC CU UCAAGGACAUCGUCCGGG GUCCGUAG | 5648 |
| 590 | UACGGACG G AAACUGCA | 2249 | UGCAGUUU GGAGGAAACUCC CU UCAAGGACAUCGUCCGGG CGUCCGUA | 5649 |
| 623 | AUCAUCUU G GGCUUUCG | 2250 | CGAAAGCC GGAGGAAACUCC CU UCAAGGACAUCGUCCGGG AAGAUGAU | 5650 |
| 624 | UCAUCUUG G GCUUUCGC | 2251 | GCGAAAGC GGAGGAAACUCC CU UCAAGGACAUCGUCCGGG CAAGAUGA | 5651 |
| 644 | AUACCUAU G GGAGUGGG | 2252 | CCCACUCC GGAGGAAACUCC CU UCAAGGACAUCGUCCGGG AUAGGUAU | 5652 |
| 645 | UACCUAUG G GAGUGGGC | 2253 | GCCCACUC GGAGGAAACUCC CU UCAAGGACAUCGUCCGGG CAUAGGUA | 5653 |
| 646 | ACCUAUGG G AGUGGGCC | 2254 | GGCCCACU GGAGGAAACUCC CU UCAAGGACAUCGUCCGGG CCAUAGGU | 5654 |
| 650 | AUGGGAGU G GGCCUCAG | 2255 | CUGAGGCC GGAGGAAACUCC CU UCAAGGACAUCGUCCGGG ACUCCCAU | 5655 |
| 651 | UGGGAGUG G GCCUCAGU | 2256 | ACUGAGGC GGAGGAAACUCC CU UCAAGGACAUCGUCCGGG CACUCCCA | 5656 |
| 671 | UUUCUCUU G GCUCAGUU | 2257 | AACUGAGC GGAGGAAACUCC CU UCAAGGACAUCGUCCGGG AAGAGAAA | 5657 |
| 701 | UGUUCAGU G GUUCGUAG | 2258 | CUACGAAC GGAGGAAACUCC CU UCAAGGACAUCGUCCGGG ACUGAACA | 5658 |
| 709 | GGUUCGUA G GGCUUUCC | 2259 | GGAAAGCC GGAGGAAACUCC CU UCAAGGACAUCGUCCGGG UACGAACC | 5659 |
| 710 | GUUCGUAG G GCUUUCCC | 2260 | GGGAAAGC GGAGGAAACUCC CU UCAAGGACAUCGUCCGGG CUACGAAC | 5660 |
| 728 | CACUGUCU G GCUUUCAG | 2261 | CUGAAAGC GGAGGAAACUCC CU UCAAGGACAUCGUCCGGG AGACAGUG | 5661 |
| 743 | AGUUAUAU G GAUGAUGU | 2262 | ACAUCAUC GGAGGAAACUCC CU UCAAGGACAUCGUCCGGG AUAUAACU | 5662 |
| 744 | GUUAUAUG G AUGAUGUG | 2263 | CACAUCAU GGAGGAAACUCC CU UCAAGGACAUCGUCCGGG CAUAUAAC | 5663 |
| 752 | GAUGAUGU G GUUUUGGG | 2264 | CCCAAAAC GGAGGAAACUCC CU UCAAGGACAUCGUCCGGG ACAUCAUC | 5664 |
| 758 | GUGGUUUU G GGGGCCAA | 2265 | UUGGCCCC GGAGGAAACUCC CU UCAAGGACAUCGUCCGGG AAAACCAC | 5665 |
| 759 | UGGUUUUG G GGGCCAAG | 2266 | CUUGGCCC GGAGGAAACUCC CU UCAAGGACAUCGUCCGGG CAAAACCA | 5666 |
| 760 | GGUUUUGG G GGCCAAGU | 2267 | ACUUGGCC GGAGGAAACUCC CU UCAAGGACAUCGUCCGGG CCAAAACC | 5667 |
| 761 | GUUUUGGG G GCCAAGUC | 2268 | GACUUGGC GGAGGAAACUCC CU UCAAGGACAUCGUCCGGG CCCAAAAC | 5668 |
| 824 | UUGUCUUU G GGUAUACA | 2269 | UGUAUACC GGAGGAAACUCC CU UCAAGGACAUCGUCCGGG AAAGACAA | 5669 |
| 825 | UGUCUUUG G GUAUACAU | 2270 | AUGUAUAC GGAGGAAACUCC CU UCAAGGACAUCGUCCGGG CAAAGACA | 5670 |
| 856 | AACAAAAA G AUGGGGAU | 2271 | AUCCCCAU GGAGGAAACUCC CU UCAAGGACAUCGUCCGGG UUUUUGUU | 5671 |
| 859 | AAAAAGAU G GGGAUAUU | 2272 | AAUAUCCC GGAGGAAACUCC CU UCAAGGACAUCGUCCGGG AUCUUUUU | 5672 |
| 860 | AAAAGAUG G GGAUAUUC | 2273 | GAAUAUCC GGAGGAAACUCC CU UCAAGGACAUCGUCCGGG CAUCUUUU | 5673 |
| 861 | AAAGAUGG G GAUAUUCC | 2274 | GGAAUAUC GGAGGAAACUCC CU UCAAGGACAUCGUCCGGG CCAUCUUU | 5674 |
| 862 | AAGAUGGG G AUAUUCCC | 2275 | GGGAAUAU GGAGGAAACUCC CU UCAAGGACAUCGUCCGGG CCCAUCUU | 5675 |
| 881 | AACUUCAU G GGAUAUGU | 2276 | ACAUAUCC GGAGGAAACUCC CU UCAAGGACAUCGUCCGGG AUGAAGUU | 5676 |
| 882 | ACUUCAUG G GAUAUGUA | 2277 | UACAUAUC GGAGGAAACUCC CU UCAAGGACAUCGUCCGGG CAUGAAGU | 5677 |
| 883 | CUUCAUGG G AUAUGUAA | 2278 | UUACAUAU GGAGGAAACUCC CU UCAAGGACAUCGUCCGGG CCAUGAAG | 5678 |
| 894 | AUGUAAUU G GGAGUUGG | 2279 | CCAACUCC GGAGGAAACUCC CU UCAAGGACAUCGUCCGGG AAUUACAU | 5679 |
| 895 | UGUAAUUG G GAGUUGGG | 2280 | CCCAACUC GGAGGAAACUCC CU UCAAGGACAUCGUCCGGG CAAUUACA | 5680 |
| 896 | GUAAUUGG G AGUUGGGG | 2281 | CCCCAACU GGAGGAAACUCC CU UCAAGGACAUCGUCCGGG CCAAUUAC | 5681 |
| 901 | UGGGAGUU G GGGCACAU | 2282 | AUGUGCCC GGAGGAAACUCC CU UCAAGGACAUCGUCCGGG AACUCCCA | 5682 |
| 902 | GGGAGUUG G GGCACAUU | 2283 | AAUGUGCC GGAGGAAACUCC CU UCAAGGACAUCGUCCGGG CAACUCCC | 5683 |
| 903 | GGAGUUGG G GCACAUUG | 2284 | CAAUGUGC GGAGGAAACUCC CU UCAAGGACAUCGUCCGGG CCAACUCC | 5684 |
| 917 | UUGCCACA G GAACAUAU | 2285 | AUAUGUUC GGAGGAAACUCC CU UCAAGGACAUCGUCCGGG UGUGGCAA | 5685 |
| 918 | UGCCACAG G AACAUAUU | 2286 | AAUAUGUU GGAGGAAACUCC CU UCAAGGACAUCGUCCGGG CUGUGGCA | 5686 |
| 952 | GUGUUUUA G GAAACUUC | 2287 | GAAGUUUC GGAGGAAACUCC CU UCAAGGACAUCGUCCGGG UAAAACAC | 5687 |
| 953 | UGUUUUAG G AAACUUCC | 2288 | GGAAGUUU GGAGGAAACUCC CU UCAAGGACAUCGUCCGGG CUAAAACA | 5688 |
| 970 | UGUAAACA G GCCUAUUG | 2289 | CAAUAGGC GGAGGAAACUCC CU UCAAGGACAUCGUCCGGG UGUUUACA | 5689 |
| 982 | UAUUGAUU G GAAAGUAU | 2290 | AUACUUUC GGAGGAAACUCC CU UCAAGGACAUCGUCCGGG AAUCAAUA | 5690 |
| 983 | AUUGAUUG G AAAGUAUG | 2291 | CAUACUUU GGAGGAAACUCC CU UCAAGGACAUCGUCCGGG CAAUCAAU | 5691 |
| 1004 | CGAAUUGU G GGUCUUUU | 2292 | AAAAGACC GGAGGAAACUCC CU UCAAGGACAUCGUCCGGG ACAAUUCG | 5692 |
| 1005 | GAAUUGUG G GUCUUUUG | 2293 | CAAAAGAC GGAGGAAACUCC CU UCAAGGACAUCGUCCGGG CACAAUUC | 5693 |
| 1013 | GGUCUUUU G GGGUUUGC | 2294 | GCAAACCC GGAGGAAACUCC CU UCAAGGACAUCGUCCGGG AAAAGACC | 5694 |
| 1014 | GUCUUUUG G GGUUUGCC | 2295 | GGCAAACC GGAGGAAACUCC CU UCAAGGACAUCGUCCGGG CAAAAGAC | 5695 |
| 1015 | UCUUUUGG G GUUUGCCG | 2296 | CGGCAAAC GGAGGAAACUCC CU UCAAGGACAUCGUCCGGG CCAAAAGA | 5696 |
| 1041 | CGCAAUGU G GAUAUUCU | 2297 | AGAAUAUC GGAGGAAACUCC CU UCAAGGACAUCGUCCGGG ACAUUGCG | 5697 |
| 1042 | GCAAUGUG G AUAUUCUG | 2298 | CAGAAUAU GGAGGAAACUCC CU UCAAGGACAUCGUCCGGG CACAUUGC | 5698 |
| 1088 | GCAAAACA G GCUUUUAC | 2299 | GUAAAAGC GGAGGAAACUCC CU UCAAGGACAUCGUCCGGG UGUUUUGC | 5699 |
| 1115 | ACUUACAA G GCCUUUCU | 2300 | AGAAAGGC GGAGGAAACUCC CU UCAAGGACAUCGUCCGGG UUGUAAGU | 5700 |
| 1159 | CGUUGCUC G GCAACGGC | 2301 | GCCGUUGC GGAGGAAACUCC CU UCAAGGACAUCGUCCGGG GAGCAACG | 5701 |
| 1165 | UCGGCAAC G GCCUGGUC | 2302 | GACCAGGC GGAGGAAACUCC CU UCAAGGACAUCGUCCGGG GUUGCCGA | 5702 |
| 1170 | AACGGCCU G GUCUAUGC | 2303 | GCAUAGAC GGAGGAAACUCC CU UCAAGGACAUCGUCCGGG AGGCCGUU | 5703 |
| 1206 | CCCCCACU G GUUGGGGC | 2304 | GCCCCAAC GGAGGAAACUCC CU UCAAGGACAUCGUCCGGG AGUGGGGG | 5704 |
| 1210 | CACUGGUU G GGGCUUGG | 2305 | CCAAGCCC GGAGGAAACUCC CU UCAAGGACAUCGUCCGGG AACCAGUG | 5705 |
| 1211 | ACUGGUUG G GGCUUGGC | 2306 | GCCAAGCC GGAGGAAACUCC CU UCAAGGACAUCGUCCGGG CAACCAGU | 5706 |
| 1212 | CUGGUUGG G GCUUGGCC | 2307 | GGCCAAGC GGAGGAAACUCC CU UCAAGGACAUCGUCCGGG CCAACCAG | 5707 |
| 1217 | UGGGGCUU G GCCAUAGG | 2308 | CCUAUGGC GGAGGAAACUCC CU UCAAGGACAUCGUCCGGG AAGCCCCA | 5708 |
| 1224 | UGGCCAUA G GCCAUCAG | 2309 | CUGAUGGC GGAGGAAACUCC CU UCAAGGACAUCGUCCGGG UAUGGCCA | 5709 |
| 1242 | GCAUGCGU G GAACCUUU | 2310 | AAAGGUUC GGAGGAAACUCC CU UCAAGGACAUCGUCCGGG ACGCAUGC | 5710 |
| 1243 | CAUGCGUG G AACCUUUG | 2311 | CAAAGGUU GGAGGAAACUCC CU UCAAGGACAUCGUCCGGG CACGCAUG | 5711 |
| 1277 | CAUACCGC G GAACUCCU | 2312 | AGGAGUUC GGAGGAAACUCC CU UCAAGGACAUCGUCCGGG GCGGUAUG | 5712 |
| 1278 | AUACCGCG G AACUCCUA | 2313 | UAGGAGUU GGAGGAAACUCC CU UCAAGGACAUCGUCCGGG CGCGGUAU | 5713 |
| 1309 | UCGCAGCA G GUCUGGGG | 2314 | CCCCAGAC GGAGGAAACUCC CU UCAAGGACAUCGUCCGGG UGCUGCGA | 5714 |
| 1314 | GCAGGUCU G GGGCAAAA | 2315 | UUUUGCCC GGAGGAAACUCC CU UCAAGGACAUCGUCCGGG AGACCUGC | 5715 |
| 1315 | CAGGUCUG G GGCAAAAC | 2316 | GUUUUGCC GGAGGAAACUCC CU UCAAGGACAUCGUCCGGG CAGACCUG | 5716 |
| 1316 | AGGUCUGG G GCAAAACU | 2317 | AGUUUUGC GGAGGAAACUCC CU UCAAGGACAUCGUCCGGG CCAGACCU | 5717 |
| 1329 | AACUCAUC G GGACUGAC | 2318 | GUCAGUCC GGAGGAAACUCC CU UCAAGGACAUCGUCCGGG GAUGAGUU | 5718 |
| 1330 | ACUCAUCG G GACUGACA | 2319 | UGUCAGUC GGAGGAAACUCC CU UCAAGGACAUCGUCCGGG CGAUGAGU | 5719 |
| 1331 | CUCAUCGG G ACUGACAA | 2320 | UUGUCAGU GGAGGAAACUCC CU UCAAGGACAUCGUCCGGG CCGAUGAG | 5720 |
| 1378 | AUUUCCAU G GCUGCUAG | 2321 | CUAGCAGC GGAGGAAACUCC CU UCAAGGACAUCGUCCGGG AUGGAAAU | 5721 |
| 1386 | GGCUGCUA G GCUGUGCU | 2322 | AGCACAGC GGAGGAAACUCC CU UCAAGGACAUCGUCCGGG UAGCAGCC | 5722 |
| 1402 | UGCCAACU G GAUCCUAC | 2323 | GUAGGAUC GGAGGAAACUCC CU UCAAGGACAUCGUCCGGG AGUUGGCA | 5723 |
| 1403 | GCCAACUG G AUCCUACG | 2324 | CGUAGGAU GGAGGAAACUCC CU UCAAGGACAUCGUCCGGG CAGUUGGC | 5724 |
| 1413 | UCCUACGC G GGACGUCC | 2325 | GGACGUCC GGAGGAAACUCC CU UCAAGGACAUCGUCCGGG GCGUAGGA | 5725 |
| 1414 | CCUACGCG G GACGUCCU | 2326 | AGGACGUC GGAGGAAACUCC CU UCAAGGACAUCGUCCGGG CGCGUAGG | 5726 |
| 1415 | CUACGCGG G ACGUCCUU | 2327 | AAGGACGU GGAGGAAACUCC CU UCAAGGACAUCGUCCGGG CCGCGUAG | 5727 |
| 1439 | GUCCCGUC G GCGCUGAA | 2328 | UUCAGCGC GGAGGAAACUCC CU UCAAGGACAUCGUCCGGG GACGGGAC | 5728 |
| 1454 | AAUCCCGC G GACGACCC | 2329 | GGGUCGUC GGAGGAAACUCC CU UCAAGGACAUCGUCCGGG GCGGGAUU | 5729 |
| 1455 | AUCCCGCG G ACGACCCC | 2330 | GGGGUCGU GGAGGAAACUCC CU UCAAGGACAUCGUCCGGG CGCGGGAU | 5730 |
| 1468 | CCCCUCCC G GGGCCGCU | 2331 | AGCGGCCC GGAGGAAACUCC CU UCAAGGACAUCGUCCGGG GGGAGGGG | 5731 |
| 1469 | CCCUCCCG G GGCCGCUU | 2332 | AAGCGGCC GGAGGAAACUCC CU UCAAGGACAUCGUCCGGG CGGGAGGG | 5732 |
| 1470 | CCUCCCGG G GCCGCUUG | 2333 | CAAGCGGC GGAGGAAACUCC CU UCAAGGACAUCGUCCGGG CCGGGAGG | 5733 |
| 1478 | GGCCGCUU G GGGCUCUA | 2334 | UAGAGCCC GGAGGAAACUCC CU UCAAGGACAUCGUCCGGG AAGCGGCC | 5734 |
| 1479 | GCCGCUUG G GGCUCUAC | 2335 | GUAGAGCC GGAGGAAACUCC CU UCAAGGACAUCGUCCGGG CAAGCGGC | 5735 |
| 1480 | CCGCUUGG G GCUCUACC | 2336 | GGUAGAGC GGAGGAAACUCC CU UCAAGGACAUCGUCCGGG CCAAGCGG | 5736 |
| 1523 | CCGUCCAC G GGGCGCAC | 2337 | GUGCGCCC GGAGGAAACUCC CU UCAAGGACAUCGUCCGGG GUGGACGG | 5737 |
| 1524 | CGUCCACG G GGCGCACC | 2338 | GGUGCGCC GGAGGAAACUCC CU UCAAGGACAUCGUCCGGG GCUGGACG | 5738 |
| 1525 | GUCCACGG G GCGCACCU | 2339 | AGGUGCGC GGAGGAAACUCC CU UCAAGGACAUCGUCCGGG CCGUGGAC | 5739 |
| 1544 | CUUUACGC G GACUCCCC | 2340 | GGGGAGUC GGAGGAAACUCC CU UCAAGGACAUCGUCCGGG GCGUAAAG | 5740 |
| 1545 | UUUACGCG G ACUCCCCG | 2341 | CGGGGAGU GGAGGAAACUCC CU UCAAGGACAUCGUCCGGG CGCGUAAA | 5741 |
| 1574 | CAUCUGCC G GACCGUGU | 2342 | ACACGGUC GGAGGAAACUCC CU UCAAGGACAUCGUCCGGG GGCAGAUG | 5742 |
| 1575 | AUCUGCCG G ACCGUGUG | 2343 | CACACGGU GGAGGAAACUCC CU UCAAGGACAUCGUCCGGG CGGCAGAU | 5743 |
| 1612 | CGUCGCAU G GAGACCAC | 2344 | GUGGUCUC GGAGGAAACUCC CU UCAAGGACAUCGUCCGGG AUGCGACG | 5744 |
| 1613 | GUCGCAUG G AGACCACC | 2345 | GGUGGUCU GGAGGAAACUCC CU UCAAGGACAUCGUCCGGG CAUGCGAC | 5745 |
| 1615 | CGCAUGGA G ACCACCGU | 2346 | ACGGUGGU GGAGGAAACUCC CU UCAAGGACAUCGUCCGGG UCCAUGCG | 5746 |
| 1635 | CGCCCACA G GAACCUGC | 2347 | GCAGGUUC GGAGGAAACUCC CU UCAAGGACAUCGUCCGGG UGUGGGCG | 5747 |
| 1636 | GCCCACAG G AACCUGCC | 2348 | GGCAGGUU GGAGGAAACUCC CU UCAAGGACAUCGUCCGGG CUGUGGGC | 5748 |
| 1648 | CUGCCCAA G GUCUUGCA | 2349 | UGCAAGAC GGAGGAAACUCC CU UCAAGGACAUCGUCCGGG UUGGGCAG | 5749 |
| 1660 | UUGCAUAA G AGGACUCU | 2350 | AGAGUCCU GGAGGAAACUCC CU UCAAGGACAUCGUCCGGG UUAUGCAA | 5750 |
| 1662 | GCAUAAGA G GACUCUUG | 2351 | CAAGAGUC GGAGGAAACUCC CU UCAAGGACAUCGUCCGGG UCUUAUGC | 5751 |
| 1663 | CAUAAGAG G ACUCUUGG | 2352 | CCAAGAGU GGAGGAAACUCC CU UCAAGGACAUCGUCCGGG CUCUUAUG | 5752 |
| 1670 | GGACUCUU G GACUUUCA | 2353 | UGAAAGUC GGAGGAAACUCC CU UCAAGGACAUCGUCCGGG AAGAGUCC | 5753 |
| 1671 | GACUCUUG G ACUUUCAG | 2354 | CUGAAAGU GGAGGAAACUCC CU UCAAGGACAUCGUCCGGG CAAGAGUC | 5754 |
| 1702 | GACCUUGA G GCAUACUU | 2355 | AAGUAUGC GGAGGAAACUCC CU UCAAGGACAUCGUCCGGG UCAAGGUC | 5755 |
| 1715 | ACUUCAAA G ACUGUGUG | 2356 | CACACAGU GGAGGAAACUCC CU UCAAGGACAUCGUCCGGG UUUGAAGU | 5756 |
| 1734 | UAAUGAGU G GGAGGAGU | 2357 | ACUCCUCC GGAGGAAACUCC CU UCAAGGACAUCGUCCGGG ACUCAUUA | 5757 |
| 1735 | AAUGAGUG G GAGGAGUU | 2358 | AACUCCUC GGAGGAAACUCC CU UCAAGGACAUCGUCCGGG CACUCAUU | 5758 |
| 1736 | AUGAGUGG G AGGAGUUG | 2359 | CAACUCCU GGAGGAAACUCC CU UCAAGGACAUCGUCCGGG CCACUCAU | 5759 |
| 1738 | GAGUGGGA G GAGUUGGG | 2360 | CCCAACUC GGAGGAAACUCC CU UCAAGGACAUCGUCCGGG UCCCACUC | 5760 |
| 1739 | AGUGGGAG G AGUUGGGG | 2361 | CCCCAACU GGAGGAAACUCC CU UCAAGGACAUCGUCCGGG CUCCCACU | 5761 |
| 1744 | GAGGAGUU G GGGGAGGA | 2362 | UCCUCCCC GGAGGAAACUCC CU UCAAGGACAUCGUCCGGG AACUCCUC | 5762 |
| 1745 | AGGAGUUG G GGGAGGAG | 2363 | CUCCUCCC GGAGGAAACUCC CU UCAAGGACAUCGUCCGGG CAACUCCU | 5763 |
| 1746 | GGAGUUGG G GGAGGAGG | 2364 | CCUCCUCC GGAGGAAACUCC CU UCAAGGACAUCGUCCGGG CCAACUCC | 5764 |
| 1747 | GAGUUGGG G GAGGAGGU | 2365 | ACCUCCUC GGAGGAAACUCC CU UCAAGGACAUCGUCCGGG CCCAACUC | 5765 |
| 1748 | AGUUGGGG G AGGAGGUU | 2366 | AACCUCCU GGAGGAAACUCC CU UCAAGGACAUCGUCCGGG CCCCAACU | 5766 |
| 1750 | UUGGGGGA G GAGGUUAG | 2367 | CUAACCUC GGAGGAAACUCC CU UCAAGGACAUCGUCCGGG UCCCCCAA | 5767 |
| 1751 | UGGGGGAG G AGGUUAGG | 2368 | CCUAACCU GGAGGAAACUCC CU UCAAGGACAUCGUCCGGG CUCCCCCA | 5768 |
| 1753 | GGGGAGGA G GUUAGGUU | 2369 | AACCUAAC GGAGGAAACUCC CU UCAAGGACAUCGUCCGGG UCCUCCCC | 5769 |
| 1758 | GGAGGUUA G GUUAAAGG | 2370 | CCUUUAAC GGAGGAAACUCC CU UCAAGGACAUCGUCCGGG UAACCUCC | 5770 |
| 1765 | AGGUUAAA G GUCUUUGU | 2371 | ACAAAGAC GGAGGAAACUCC CU UCAAGGACAUCGUCCGGG UUUAACCU | 5771 |
| 1778 | UUGUACUA G GAGGCUGU | 2372 | ACAGCCUC GGAGGAAACUCC CU UCAAGGACAUCGUCCGGG UAGUACAA | 5772 |
| 1779 | UGUACUAG G AGGCUGUA | 2373 | UACAGCCU GGAGGAAACUCC CU UCAAGGACAUCGUCCGGG CUAGUACA | 5773 |
| 1781 | UACUAGGA G GCUGUAGG | 2374 | CCUACAGC GGAGGAAACUCC CU UCAAGGACAUCGUCCGGG UCCUAGUA | 5774 |
| 1788 | AGGCUGUA G GCAUAAAU | 2375 | AUUUAUGC GGAGGAAACUCC CU UCAAGGACAUCGUCCGGG UACAGCCU | 5775 |
| 1798 | CAUAAAUU G GUGUGUUC | 2376 | GAACACAC GGAGGAAACUCC CU UCAAGGACAUCGUCCGGG AAUUUAUG | 5776 |
| 1888 | UGUGCCUU G GGUGGCUU | 2377 | AAGCCACC GGAGGAAACUCC CU UCAAGGACAUCGUCCGGG AAGGCACA | 5777 |
| 1889 | GUGCCUUG G GUGGCUUU | 2378 | AAAGCCAC GGAGGAAACUCC CU UCAAGGACAUCGUCCGGG CAAGGCAC | 5778 |
| 1892 | CCUUGGGU G GCUUUGGG | 2379 | CCCAAAGC GGAGGAAACUCC CU UCAAGGACAUCGUCCGGG ACCCAAGG | 5779 |
| 1898 | GUGGCUUU G GGGCAUGG | 2380 | CCAUGCCC GGAGGAAACUCC CU UCAAGGACAUCGUCCGGG AAAGCCAC | 5780 |
| 1899 | UGGCUUUG G GGCAUGGA | 2381 | UCCAUGCC GGAGGAAACUCC CU UCAAGGACAUCGUCCGGG CAAAGCCA | 5781 |
| 1900 | GGCUUUGG G CCAUGGAC | 2382 | GUCCAUGC GGAGGAAACUCC CU UCAAGGACAUCGUCCGGG CCAAAGCC | 5782 |
| 1905 | UGGGGCAU G GACAUUGA | 2383 | UCAAUGUC GGAGGAAACUCC CU UCAAGGACAUCGUCCGGG AUGCCCCA | 5783 |
| 1906 | GGGGCAUG G ACAUUGAC | 2384 | GUCAAUGU GGAGGAAACUCC CU UCAAGGACAUCGUCCGGG CAUGCCCC | 5784 |
| 1924 | CGUAUAAA G AAUUUGGA | 2385 | UCCAAAUU GGAGGAAACUCC CU UCAAGGACAUCGUCCGGG UUUAUACG | 5785 |
| 1930 | AAGAAUUU G GAGCUUCU | 2386 | AGAAGCUC GGAGGAAACUCC CU UCAAGGACAUCGUCCGGG AAAUUCUU | 5786 |
| 1931 | AGAAUUUG G AGCUUCUG | 2387 | CAGAAGCU GGAGGAAACUCC CU UCAAGGACAUCGUCCGGG CAAAUUCU | 5787 |
| 1941 | GCUUCUGU G GAGUUACU | 2388 | AGUAACUC GGAGGAAACUCC CU UCAAGGACAUCGUCCGGG ACAGAAGC | 5788 |
| 1942 | CUUCUGUG G ACUUACUC | 2389 | GAGUAACU GGAGGAAACUCC CU UCAAGGACAUCGUCCGGG CACAGAAG | 5789 |
| 1987 | CUAUUCGA G AUCUCCUC | 2390 | GAGGAGAU GGAGGAAACUCC CU UCAAGGACAUCGUCCGGG UCGAAUAG | 5790 |
| 2018 | UCUGUAUC G GGGGGCCU | 2391 | AGGCCCCC GGAGGAAACUCC CU UCAAGGACAUCGUCCGGG GAUACAGA | 5791 |
| 2019 | CUGUAUCG G GGGGCCUU | 2392 | AAGGCCCC GGAGGAAACUCC CU UCAAGGACAUCGUCCGGG CGAUACAG | 5792 |
| 2020 | UGUAUCGG G GGGCCUUA | 2393 | UAAGGCCC GGAGGAAACUCC CU UCAAGGACAUCGUCCGGG CCGAUACA | 5793 |
| 2021 | GUAUCGGG G GGCCUUAG | 2394 | CUAAGGCC GGAGGAAACUCC CU UCAAGGACAUCGUCCGGG CCCGAUAC | 5794 |
| 2022 | UAUCGGGG G GCCUUAGA | 2395 | UCUAAGGC GGAGGAAACUCC CU UCAAGGACAUCGUCCGGG CCCCGAUA | 5795 |
| 2029 | GGGCCUUA G AGUCUCCG | 2396 | CGGAGACU GGAGGAAACUCC CU UCAAGGACAUCGUCCGGG UAAGGCCC | 5796 |
| 2037 | GAGUCUCC G GAACAUUG | 2397 | CAAUGUUC GGAGGAAACUCC CU UCAAGGACAUCGUCCGGG GGAGACUC | 5797 |
| 2038 | AGUCUCCG G AACAUUGU | 2398 | ACAAUGUU GGAGGAAACUCC CU UCAAGGACAUCGUCCGGG CGGAGACU | 5798 |
| 2061 | CACCAUAC G GCACUCAG | 2399 | CUGAGUGC GGAGGAAACUCC CU UCAAGGACAUCGUCCGGG GUAUGGUG | 5799 |
| 2069 | GGCACUCA G GCAAGCUA | 2400 | UAGCUUGC GGAGGAAACUCC CU UCAAGGACAUCGUCCGGG UGACUGCC | 5800 |
| 2087 | UCUGUGUU G GGGUGAGU | 2401 | ACUCACCC GGAGGAAACUCC CU UCAAGGACAUCGUCCGGG AACACAGA | 5801 |
| 2088 | CUGUGUUG G GGUGAGUU | 2402 | AACUCACC GGAGGAAACUCC CU UCAAGGACAUCGUCCGGG CAACACAG | 5802 |
| 2089 | UGUGUUGG G GUGAGUUG | 2403 | CAACUCAC GGAGGAAACUCC CU UCAAGGACAUCGUCCGGG CCAACACA | 5803 |
| 2114 | AGCCACCU G GGUGGGAA | 2404 | UUCCCACC GGAGGAAACUCC CU UCAAGGACAUCGUCCGGG AGGUGGCU | 5804 |
| 2115 | GCCACCUC G GUGGGAAG | 2405 | CUUCCCAC GGAGGAAACUCC CU UCAAGGACAUCGUCCGGG CAGGUGGC | 5805 |
| 2118 | ACCUGGGU G GGAAGUAA | 2406 | UUACUUCC GGAGGAAACUCC CU UCAAGGACAUCGUCCGGG ACCCAGGU | 5806 |
| 2119 | CCUGGGUG G GAAGUAAU | 2407 | AUUACUUC GGAGGAAACUCC CU UCAAGGACAUCGUCCGGG CACCCAGG | 5807 |
| 2120 | CUGGGUGG G AAGUAAUU | 2408 | AAUUACUU GGAGGAAACUCC CU UCAAGGACAUCGUCCGGG CCACCCAG | 5808 |
| 2130 | AGUAAUUU G GAAGAUCC | 2409 | GGAUCUUC GGAGGAAACUCC CU UCAAGGACAUCGUCCGGG AAAUUACU | 5809 |
| 2131 | GUAAUUUG G AAGAUCCA | 2410 | UGGAUCUU GGAGGAAACUCC CU UCAAGGACAUCGUCCGGG CAAAUUAC | 5810 |
| 2134 | AUUUGGAA G AUCCAGCA | 2411 | UGCUGGAU GGAGGAAACUCC CU UCAAGGACAUCGUCCGGG UUCCAAAU | 5811 |
| 2147 | AGCAUCCA G GGAAUUAG | 2412 | CUAAUUCC GGAGGAAACUCC CU UCAAGGACAUCGUCCGGG UGGAUGCU | 5812 |
| 2148 | GCAUCCAG G GAAUUAGU | 2413 | ACUAAUUC GGAGGAAACUCC CU UCAAGGACAUCGUCCGGG CUGGAUGC | 5813 |
| 2149 | CAUCCAGG G AAUUAGUA | 2414 | UACUAAUU GGAGGAAACUCC CU UCAAGGACAUCGUCCGGG CCUGGAUG | 5814 |
| 2181 | GUUAAUAU G GGCCUAAA | 2415 | UUUAGGCC GGAGGAAACUCC CU UCAAGGACAUCGUCCGGG AUAUUAAC | 5815 |
| 2182 | UUAAUAUG G GCCUAAAA | 2416 | UUUUAGGC GGAGGAAACUCC CU UCAAGGACAUCGUCCGGG CAUAUUAA | 5816 |
| 2195 | AAAAAUCA G ACAACUAU | 2417 | AUAGUUGU GGAGGAAACUCC CU UCAAGGACAUCGUCCGGG UGAUUUUU | 5817 |
| 2207 | ACUAUUGU G GUUUCACA | 2418 | UGUGAAAC GGAGGAAACUCC CU UCAAGGACAUCGUCCGGG ACAAUAGU | 5818 |
| 2233 | UUACUUUU G GGCGAGAA | 2419 | UUCUCGCC GGAGGAAACUCC CU UCAAGGACAUCGUCCGGG AAAAGUAA | 5819 |
| 2234 | UACUUUUG G GCGAGAAA | 2420 | UUUCUCGC GGAGGAAACUCC CU UCAAGGACAUCGUCCGGG CAAAAGUA | 5820 |
| 2239 | UUGGGCGA G AAACUGUU | 2421 | AACAGUUU GGAGGAAACUCC CU UCAAGGACAUCGUCCGGG UCGCCCAA | 5821 |
| 2259 | GAAUAUUU G GUGUCUUU | 2422 | AAAGACAC GGAGGAAACUCC CU UCAAGGACAUCGUCCGGG AAAUAUUC | 5822 |
| 2269 | UGUCUUUU G GAGUGUGG | 2423 | CCACACUC GGAGGAAACUCC CU UCAAGGACAUCGUCCGGG AAAAGACA | 5823 |
| 2270 | GUCUUUUG G AGUGUGGA | 2424 | UCCACACU GGAGGAAACUCC CU UCAAGGACAUCGUCCGGG CAAAAGAC | 5824 |
| 2276 | UGGAGUGU G GAUUCGCA | 2425 | UGCGAAUC GGAGGAAACUCC CU UCAAGGACAUCGUCCGGG ACACUCCA | 5825 |
| 2277 | GGAGUGUG G AUUCGCAC | 2426 | GUGCGAAU GGAGGAAACUCC CU UCAAGGACAUCGUCCGGG CACACUCC | 5826 |
| 2300 | UGCAUAUA G ACCACCAA | 2427 | UUGGUGGU GGAGGAAACUCC CU UCAAGGACAUCGUCCGGG UAUAUGCA | 5827 |
| 2334 | ACACUUCC G GAAACUAC | 2428 | GUAGUUUC GGAGGAAACUCC CU UCAAGGACAUCGUCCGGG GGAAGUGU | 5828 |
| 2335 | CACUUCCG G AAACUACU | 2429 | AGUAGUUU GGAGGAAACUCC CU UCAAGGACAUCGUCCGGG CGGAAGUG | 5829 |
| 2351 | UGUUGUUA G ACGAAGAG | 2430 | CUCUUCGU GGAGGAAACUCC CU UCAAGGACAUCGUCCGGG UAACAACA | 5830 |
| 2357 | UAGACGAA G AGGCAGGU | 2431 | ACCUGCCU GGAGGAAACUCC CU UCAAGGACAUCGUCCGGG UUCGUCUA | 5831 |
| 2359 | GACGAAGA G GCAGGUCC | 2432 | GGACCUGC GGAGGAAACUCC CU UCAAGGACAUCGUCCGGG UCUUCGUC | 5832 |
| 2363 | AAGAGGCA G GUCCCCUA | 2433 | UAGGGGAC GGAGGAAACUCC CU UCAAGGACAUCGUCCGGG UGCCUCUU | 5833 |
| 2372 | GUCCCCUA G AAGAAGAA | 2434 | UUCUUCUU GGAGGAAACUCC CU UCAAGGACAUCGUCCGGG UAGGGGAC | 5834 |
| 2375 | CCCUAGAA G AAGAACUC | 2435 | GAGUUCUU GGAGGAAACUCC CU UCAAGGACAUCGUCCGGG UUCUAGGG | 5835 |
| 2378 | UAGAAGAA G AACUCCCU | 2436 | AGGGAGUU GGAGGAAACUCC CU UCAAGGACAUCGUCCGGG UUCUUCUA | 5836 |
| 2396 | GCCUCGCA G ACGAAGGU | 2437 | ACCUUCGU GGAGGAAACUCC CU UCAAGGACAUCGUCCGGG UGCGAGGC | 5837 |
| 2402 | CAGACGAA G GUCUCAAU | 2438 | AUUGAGAC GGAGGAAACUCC CU UCAAGGACAUCGUCCGGG UUCGUCUG | 5838 |
| 2423 | GCGUCGCA G AAGAUCUC | 2439 | GAGAUCUU GGAGGAAACUCC CU UCAAGGACAUCGUCCGGG UGCGACGC | 5839 |
| 2426 | UCGCAGAA G AUCUCAAU | 2440 | AUUGAGAU GGAGGAAACUCC CU UCAAGGACAUCGUCCGGG UUCUGCGA | 5840 |
| 2438 | UCAAUCUC G GGAAUCUC | 2441 | GAGAUUCC GGAGGAAACUCC CU UCAAGGACAUCGUCCGGG GAGAUUGA | 5841 |
| 2439 | CAAUCUCG G GAAUCUCA | 2442 | UGAGAUUC GGAGGAAACUCC CU UCAAGGACAUCGUCCGGG CGAGAUUG | 5842 |
| 2440 | AAUCUCGG G AAUCUCAA | 2443 | UUGAGAUU GGAGGAAACUCC CU UCAAGGACAUCGUCCGGG CCGAGAUU | 5843 |
| 2463 | UAUUCCUU G GACACAUA | 2444 | UAUGUGUC GGAGGAAACUCC CU UCAAGGACAUCGUCCGGG AAGGAAUA | 5844 |
| 2464 | AUUCCUUG G ACACAUAA | 2445 | UUAUGUGU GGAGGAAACUCC CU UCAAGGACAUCGUCCGGG CAAGGAAU | 5845 |
| 2473 | ACACAUAA G GUGGGAAA | 2446 | UUUCCCAC GGAGGAAACUCC CU UCAAGGACAUCGUCCGGG UUAUGUGU | 5846 |
| 2476 | CAUAAGGU G GGAAACUU | 2447 | AAGUUUCC GGAGGAAACUCC CU UCAAGGACAUCGUCCGGG ACCUUAUG | 5847 |
| 2477 | AUAAGGUG G GAAACUUU | 2448 | AAAGUUUC GGAGGAAACUCC CU UCAAGGACAUCGUCCGGG CACCUUAU | 5848 |
| 2478 | UAAGGUGG G AAACUUUA | 2449 | UAAAGUUU GGAGGAAACUCC CU UCAAGGACAUCGUCCGGG CCACCUUA | 5849 |
| 2488 | AACUUUAC G GGGCUUUA | 2450 | UAAAGCCC GGAGGAAACUCC CU UCAAGGACAUCGUCCGGG GUAAAGUU | 5850 |
| 2489 | ACUUUACG G GGCUUUAU | 2452 | AUAAAGCC GGAGGAAACUCC CU UCAAGGACAUCGUCCGGG CGUAAAGU | 5851 |
| 2490 | CUUUACGG G GCUUUAUU | 2452 | AAUAAAGC GGAGGAAACUCC CU UCAAGGACAUCGUCCGGG CCGUAAAG | 5852 |
| 2506 | UCUUCUAC G GUACCUUG | 2453 | CAAGGUAC GGAGGAAACUCC CU UCAAGGACAUCGUCCGGG GUAGAAGA | 5853 |
| 2529 | UCCUAAAU G GCAAACUC | 2454 | GAGUUUGC GGAGGAAACUCC CU UCAAGGACAUCGUCCGGG AUUUAGGA | 5854 |
| 2563 | CAUUUGCA G GAGGACAU | 2455 | AUGUCCUC GGAGGAAACUCC CU UCAAGGACAUCGUCCGGG UGCAAAUG | 5855 |
| 2564 | AUUUGCAG G AGGACAUU | 2456 | AAUGUCCU GGAGGAAACUCC CU UCAAGGACAUCGUCCGGG CUGCAAAU | 5856 |
| 2566 | UUGCAGGA G GACAUUGU | 2457 | ACAAUGUC GGAGGAAACUCC CU UCAAGGACAUCGUCCGGG UCCUGCAA | 5857 |
| 2567 | UGCAGGAG G ACAUUGUU | 2458 | AACAAUGU GGAGGAAACUCC CU UCAAGGACAUCGUCCGGG CUCCUGCA | 5858 |
| 2580 | UGUUGAUA G AUGUAAGC | 2459 | GCUUACAU GGAGGAAACUCC CU UCAAGGACAUCGUCCGGG UAUCAACA | 5859 |
| 2596 | CAAUUUGU G GGGCCCCU | 2460 | AGGGGCCC GGAGGAAACUCC CU UCAAGGACAUCGUCCGGG ACAAAUUG | 5860 |
| 2597 | AAUUUGUG G GGCCCCUU | 2461 | AAGGGGCC GGAGGAAACUCC CU UCAAGGACAUCGUCCGGG CACAAAUU | 5861 |
| 2598 | AUUUGUGG G GCCCCUUA | 2462 | UAAGGGGC GGAGGAAACUCC CU UCAAGGACAUCGUCCGGG CCACAAAU | 5862 |
| 2622 | UGAAAACA G GAGACUUA | 2463 | UAAGUCUC GGAGGAAACUCC CU UCAAGGACAUCGUCCGGG UGUUUUCA | 5863 |
| 2623 | GAAAACAG G AGACUUAA | 2464 | UUAAGUCU GGAGGAAACUCC CU UCAAGGACAUCGUCCGGG CUGUUUUC | 5864 |
| 2625 | AAACAGGA G ACUUAAAU | 2465 | AUUUAAGU GGAGGAAACUCC CU UCAAGGACAUCGUCCGGG UCCUGUUU | 5865 |
| 2649 | GCCUGCUA G GUUUUAUC | 2466 | GAUAAAAC GGAGGAAACUCC CU UCAAGGACAUCGUCCGGG UAGCAGGC | 5866 |
| 2684 | UGCCCUUA G AUAAAGGG | 2467 | CCCUUUAU GGAGGAAACUCC CU UCAAGGACAUCGUCCGGG UAAGGGCA | 5867 |
| 2690 | UAGAUAAA G GGAUCAAA | 2468 | UUUGAUCC GGAGGAAACUCC CU UCAAGGACAUCGUCCGGG UUUAUCUA | 5868 |
| 2691 | AGAUAAAG G GAUCAAAC | 2469 | GUUUGAUC GGAGGAAACUCC CU UCAAGGACAUCGUCCGGG CUUUAUCU | 5869 |
| 2692 | GAUAAAGG G AUCAAACC | 2470 | GGUUUGAU GGAGGAAACUCC CU UCAAGGACAUCGUCCGGG CCUUUAUC | 5870 |
| 2711 | AUUAUCCA G AGUAUGUA | 2471 | UACAUACU GGAGGAAACUCC CU UCAAGGACAUCGUCCGGG UGGAUAAU | 5871 |
| 2737 | UACUUCCA G ACGCGACA | 2472 | UGUCGCGU GGAGGAAACUCC CU UCAAGGACAUCGUCCGGG UGGAAGUA | 5872 |
| 2763 | CACUCUUU G GAAGGCGG | 2473 | CCGCCUUC GGAGGAAACUCC CU UCAAGGACAUCGUCCGGG AAAGAGUG | 5873 |
| 2764 | ACUCUUUG G AAGCCGGG | 2474 | CCCGCCUU GGAGGAAACUCC CU UCAAGGACAUCGUCCGGG CAAAGAGU | 5874 |
| 2767 | CUUUGGAA G CCGCCCAU | 2475 | AUCCCCGC GGAGGAAACUCC CU UCAAGGACAUCGUCCGGG UUCCAAAG | 5875 |
| 2770 | UGGAAGGC G GGGAUCUU | 2476 | AAGAUCCC GGAGGAAACUCC CU UCAAGGACAUCGUCCGGG GCCUUCCA | 5876 |
| 2771 | GGAAGGCG G GGAUCUUA | 2477 | UAAGAUCC GGAGGAAACUCC CU UCAAGGACAUCGUCCGGG CGCCUUCC | 5877 |
| 2772 | GAAGGCGG G GAUCUUAU | 2478 | AUAAGAUC GGAGGAAACUCC CU UCAAGGACAUCGUCCGGG CCGCCUUC | 5878 |
| 2773 | AAGGCGGG G AUCUUAUA | 2479 | UAUAAGAU GGAGGAAACUCC CU UCAAGGACAUCGUCCGGG CCCGCCUU | 5879 |
| 2787 | AUAUAAAA G AGAGUCCA | 2480 | UGGACUCU GGAGGAAACUCC CU UCAAGGACAUCGUCCGGG UUUUAUAU | 5880 |
| 2789 | AUAAAAGA G AGUCCACA | 2481 | UGUGGACU GGAGGAAACUCC CU UCAAGGACAUCGUCCGGG UCUUUUAU | 5881 |
| 2816 | CAUUUUGC G GGUCACCA | 2482 | UGGUGACC GGAGGAAACUCC CU UCAAGGACAUCGUCCGGG GCAAAAUG | 5882 |
| 2817 | AUUUUGCG G GUCACCAU | 2483 | AUGGUGAC GGAGGAAACUCC CU UCAAGGACAUCGUCCGGG CGCAAAAU | 5883 |
| 2832 | AUAUUCUU G GGAACAAG | 2484 | CUUGUUCC GGAGGAAACUCC CU UCAAGGACAUCGUCCGGG AAGAAUAU | 5884 |
| 2833 | UAUUCUUG G GAACAAGA | 2485 | UCUUGUUC GGAGGAAACUCC CU UCAAGGACAUCGUCCGGG CAAGAAUA | 5885 |
| 2834 | AUUCUUGG G AACAAGAU | 2486 | AUCUUGUU GGAGGAAACUCC CU UCAAGGACAUCGUCCGGG CCAAGAAU | 5886 |
| 2840 | GGGAACAA G AUCUACAG | 2487 | CUGUAGAU GGAGGAAACUCC CU UCAAGGACAUCGUCCGGG UUGUUCCC | 5887 |
| 2852 | UACAGCAU G GGAGGUUG | 2488 | CAACCUCC GGAGGAAACUCC CU UCAAGGACAUCGUCCGGG AUGCUGUA | 5888 |
| 2853 | ACAGCAUG G GAGGUUGG | 2489 | CCAACCUC GGAGGAAACUCC CU UCAAGGACAUCGUCCGGG CAUGCUGU | 5889 |
| 2854 | CAGCAUGG G AGGUUGGU | 2490 | ACCAACCU GGAGGAAACUCC CU UCAAGGACAUCGUCCGGG CCAUGCUG | 5890 |
| 2856 | GCAUGGGA G GUUGGUCU | 2491 | AGACCAAC GGAGGAAACUCC CU UCAAGGACAUCGUCCGGG UCCCAUGC | 5891 |
| 2860 | GGGAGGUU G GUCUUCCA | 2492 | UGGAAGAC GGAGGAAACUCC CU UCAAGGACAUCGUCCGGG AACCUCCC | 5892 |
| 2880 | CUCGAAAA G GCAUGGGG | 2493 | CCCCAUGC GGAGGAAACUCC CU UCAAGGACAUCGUCCGGG UUUUCGAG | 5893 |
| 2885 | AAAGGCAU G GGGACAAA | 2494 | UUUGUCCC GGAGGAAACUCC CU UCAAGGACAUCGUCCGGG AUGCCUUU | 5894 |
| 2886 | AAGGCAUG G GGACAAAU | 2495 | AUUUGUCC GGAGGAAACUCC CU UCAAGGACAUCGUCCGGG CAUGCCUU | 5894 |
| 2887 | AGGCAUGG G GACAAAUC | 2496 | GAUUUGUC GGAGGAAACUCC CU UCAAGGACAUCGUCCGGG CCAUGCCU | 5896 |
| 2888 | GGCAUGGG G ACAAAUCU | 2497 | AGAUUUGU GGAGGAAACUCC CU UCAAGGACAUCGUCCGGG CCCAUGCC | 5897 |
| 2915 | AAUCCCCU G GGAUUCUU | 2498 | AAGAAUCC GGAGGAAACUCC CU UCAAGGACAUCGUCCGGG AGGGGAUU | 5898 |
| 2916 | AUCCCCUG G GAUUCUUC | 2499 | GAAGAAUC GGAGGAAACUCC CU UCAAGGACAUCGUCCGGG CAGGGGAU | 5899 |
| 2917 | UCCCCUGG G AUUCUUCC | 2500 | GGAAGAAU GGAGGAAACUCC CU UCAAGGACAUCGUCCGGG CCAGGGGA | 5900 |
| 2939 | CAUCAGUU G GACCCUGC | 2501 | GCAGGGUC GGAGGAAACUCC CU UCAAGGACAUCGUCCGGG AACUGAUG | 5901 |
| 2940 | AUCAGUUG G ACCCUGCA | 2502 | UGCAGGGU GGAGGAAACUCC CU UCAAGGACAUCGUCCGGG CAACUGAU | 5902 |
| 2973 | UAAAUCCA G AUUGGGAC | 2503 | GUCCCAAU GGAGGAAACUCC CU UCAAGGACAUCGUCCGGG UGGAUUUA | 5903 |
| 2977 | UCCAGAUU G GGACCUCA | 2504 | UGAGGUCC GGAGGAAACUCC CU UCAAGGACAUCGUCCGGG AAUCUGGA | 5904 |
| 2978 | CCAGAUUG G GACCUCAA | 2505 | UUGAGGUC GGAGGAAACUCC CU UCAAGGACAUCGUCCGGG CAAUCUGG | 5905 |
| 2979 | CAGAUUGG G ACCUCAAC | 2506 | GUUGAGGU GGAGGAAACUCC CU UCAAGGACAUCGUCCGGG CCAAUCUG | 5906 |
| 2996 | CCGCACAA G GACAACUG | 2507 | CAGUUGUC GGAGGAAACUCC CU UCAAGGACAUCGUCCGGG UUGUGCGG | 5907 |
| 2997 | CGCACAAG G ACAACUGG | 2508 | CCAGUUGU GGAGGAAACUCC CU UCAAGGACAUCGUCCGGG CUUGUGCG | 5908 |
| 3004 | GGACAACU G GCCGGACG | 2509 | CGUCCGGC GGAGGAAACUCC CU UCAAGGACAUCGUCCGGG AGUUGUCC | 5909 |
| 3008 | AACUGGCC G GACGCCAA | 2510 | UUGGCGUC GGAGGAAACUCC CU UCAAGGACAUCGUCCGGG GGCCAGUU | 5910 |
| 3009 | ACUGGCCG G ACGCCAAC | 2511 | GUUGGCGU GGAGGAAACUCC CU UCAAGGACAUCGUCCGGG CGGCCAGU | 5911 |
| 3020 | GCCAACAA G GUGGGAGU | 2512 | ACUCCCAC GGAGGAAACUCC CU UCAAGGACAUCGUCCGGG UUGUUGGC | 5912 |
| 3023 | AACAAGGU G GGAGUGGG | 2513 | CCCACUCC GGAGGAAACUCC CU UCAAGGACAUCGUCCGGG ACCUUGUU | 5913 |
| 3024 | ACAAGGUG G GAGUGGGA | 2514 | UCCCACUC GGAGGAAACUCC CU UCAAGGACAUCGUCCGGG CACCUUGU | 5914 |
| 3025 | CAAGGUGG G AGUGGGAG | 2515 | CUCCCACU GGAGGAAACUCC CU UCAAGGACAUCGUCCGGG CCACCUUG | 5915 |
| 3029 | GUGGGAGU G GGAGCAUU | 2516 | AAUGCUCC GGAGGAAACUCC CU UCAAGGACAUCGUCCGGG ACUCCCAC | 5916 |
| 3030 | UGGGAGUG G GAGCACUC | 2517 | GAAUGCUC GGAGGAAACUCC CU UCAAGGACAUCGUCCGGG CACUCCCA | 5917 |
| 3031 | GGGAGUGG G AGCAUUCG | 2518 | CGAAUGCU GGAGGAAACUCC CU UCAAGGACAUCGUCCGGG CCACUCCC | 5918 |
| 3039 | GAGCAUUC G GGCCAGGG | 2519 | CCCUGGCC GGAGGAAACUCC CU UCAAGGACAUCGUCCGGG GAAUGCUC | 5919 |
| 3040 | AGCAUUCG G GCCAGGGU | 2520 | ACCCUGGC GGAGGAAACUCC CU UCAAGGACAUCGUCCGGG CGAAUGCU | 5920 |
| 3045 | UCGGGCCA G GGUUCACC | 2521 | GGUGAACC GGAGGAAACUCC CU UCAAGGACAUCGUCCGGG UGGCCCGA | 5921 |
| 3046 | CGGGCCAG G GUUCACCC | 2522 | GGGUGAAC GGAGGAAACUCC CU UCAAGGACAUCGUCCGGG CUGGCCCG | 5922 |
| 3063 | CUCCCCAU G GGGGACUG | 2523 | CAGUCCCC GGAGGAAACUCC CU UCAAGGACAUCGUCCGGG AUGGGGAG | 5923 |
| 3064 | UCCCCAUG G GGGACUGU | 2524 | ACAGUCCC GGAGGAAACUCC CU UCAAGGACAUCGUCCGGG CAUGGGGA | 5924 |
| 3065 | CCCCAUGG G GGACUGUU | 2525 | AACAGUCC GGAGGAAACUCC CU UCAAGGACAUCGUCCGGG CCAUGGGG | 5925 |
| 3066 | CCCAUGGG G GACUGUUG | 2526 | CAACAGUC GGAGGAAACUCC CU UCAAGGACAUCGUCCGGG CCCAUGGG | 5926 |
| 3067 | CCAUGGGG G ACUGUUGG | 2527 | CCAACAGU GGAGGAAACUCC CU UCAAGGACAUCGUCCGGG CCCCAUGG | 5927 |
| 3074 | GGACUGUU G GGGUGGAG | 2528 | CUCCACCC GGAGGAAACUCC CU UCAAGGACAUCGUCCGGG AACAGUCC | 5928 |
| 3075 | GACUGUUG G GGUGGAGC | 2529 | GCUCCACC GGAGGAAACUCC CU UCAAGGACAUCGUCCGGG CAACAGUC | 5929 |
| 3076 | ACUGUUGG G GUGGAGCC | 2530 | GGCUCCAC GGAGGAAACUCC CU UCAAGGACAUCGUCCGGG CCAACAGU | 5930 |
| 3079 | GUUGGGGU G GAGCCCUC | 2531 | GAGGGCUC GGAGGAAACUCC CU UCAAGGACAUCGUCCGGG ACCCCAAC | 5931 |
| 3080 | UUGGGGUG G AGCCCUCA | 2532 | UGAGGGCU GGAGGAAACUCC CU UCAAGGACAUCGUCCGGG CACCCCAA | 5932 |
| 3095 | CACGCUCA G GGCCUACU | 2533 | AGUAGGCC GGAGGAAACUCC CU UCAAGGACAUCGUCCGGG UGAGCGUG | 5933 |
| 3096 | ACGCUCAG G GCCUACUC | 2534 | GAGUAGGC GGAGGAAACUCC CU UCAAGGACAUCGUCCGGG CUGAGCGU | 5934 |
| 3145 | CACCAAUC G GCAGUCAG | 2535 | CUGACUGC GGAGGAAACUCC CU UCAAGGACAUCGUCCGGG GAUUGGUG | 5935 |
| 3153 | GGCAGUCA G GAAGGCAG | 2536 | CUGCCUUC GGAGGAAACUCC CU UCAAGGACAUCGUCCGGG UGACUGCC | 5936 |
| 3154 | GCAGUCAG G AAGGCAGC | 2537 | GCUGCCUU GGAGGAAACUCC CU UCAAGGACAUCGUCCGGG CUGACUGC | 5937 |
| 3157 | GUCAGGAA G GCAGCCUA | 2538 | UAGGCUGC GGAGGAAACUCC CU UCAAGGACAUCGUCCGGG UUCCUGAC | 5938 |
| 3187 | ACCUCUAA G GGACACUC | 2539 | GAGUGUCC GGAGGAAACUCC CU UCAAGGACAUCGUCCGGG UUAGAGGU | 5939 |
| 3188 | CCUCUAAG G GACACUCA | 2540 | UGAGUGUC GGAGGAAACUCC CU UCAAGGACAUCGUCCGGG CUUAGAGG | 5940 |
| 3189 | CUCUAAGG G ACACUCAU | 2541 | AUGAGUGU GGAGGAAACUCC CU UCAAGGACAUCGUCCGGG CCUUAGAG | 5941 |
| 3203 | CAUCCUCA G GCCAUGCA | 2542 | UGCAUGGC GGAGGAAACUCC CU UCAAGGACAUCGUCCGGG UGAGGAUG | 5942 |

Input Sequence = AF100308. Cut Site = YG/M or UG/U.
Stem Length = 8. Core Sequence = GGAGGAAACUCC CU UCAAGGACAUCGUCCGGG
AF100308 (Hepatitis B virus strain 2-18, 3215 bp)

TABLE XI
Human HBV Enzymatic Nucleic Acid and Target Sequence

| | | Seq | | | | Seq |
|---|---|---|---|---|---|---|
| Pos | SUBSTRATE | ID | RPI # | Ribozyme Alias | ENZYMATIC NUCLEIC ACID | ID |
| 313 | CCAAAAU U CGCAGUC | 5943 | 18157 | HBV-313 Rz-7 RNA | GACUGCG CUGAUGAGGCCGUUAGGCCGAA AUUUUGG B | 6293 |
| 327 | CCCAAAU C UCCAGUC | 5944 | 18158 | HBV-327 Rz-7 RNA | GACUGGA CUGAUGAGGCCGUUAGGCCGAA AUUUGGG B | 6294 |
| 334 | CUCCAGU C ACUCACC | 5945 | 18159 | HBV-334 Rz-7 RNA | GGUGAGU CUGAUGAGGCCGUUAGGCCGAA ACUGGAG B | 6295 |
| 408 | UCUUCCU C UGCAUCC | 5946 | 18160 | HBV-408 Rz-7 RNA | GGAUGCA CUGAUGAGGCCGUUAGGCCGAA AGGAAGA B | 6296 |
| 557 | UCUAUGU U UCCCUCA | 5947 | 18161 | HBV-557 Rz-7 RNA | UGAGGGA CUGAUGAGGCCGUUAGGCCGAA ACAUAGA B | 6297 |
| 1255 | UUUGUGU C UCCUCUG | 5948 | 18162 | HBV-1255 Rz-7 RNA | CAGAGGA CUGAUGAGGCCGUUAGGCCGAA ACACAAA B | 6298 |
| 1538 | CCUCUCU U UACGCGG | 5949 | 18163 | HBV-1538 Rz-7 RNA | CCGCGUA CUGAUGAGGCCGUUAGGCCGAA AGAGAGG B | 6299 |
| 1756 | AGGAGGU U AGGUUAA | 5950 | 18164 | HBV-1756 Rz-7 RNA | UUAACCU CUGAUGAGGCCGUUAGGCCGAA ACCUCCU B | 6300 |
| 1861 | AUGUCCU A CUGUUCA | 5951 | 18165 | HBV-1861 Rz-7 RNA | UGAACAG CUGAUGAGGCCGUUAGGCCGAA AGGACAU B | 6301 |
| 2504 | UUCUUCU A CGGUACC | 5952 | 18166 | HBV-2504 Rz-7 RNA | GGUACCG CUGAUGAGGCCGUUAGGCCGAA AGAAGAA B | 6302 |
| 10 | CUCCACC A CUUUCCA | 5953 | 18197 | HBV-10 CHz-7 RNA | UGGAAAG CUGAUGAGGCCGUUAGGCCGAA GGUGGAG B | 6303 |
| 335 | UCCAGUC A CUCACCA | 5954 | 18198 | HBV-335 CHz-7 RNA | UGGUGAG CUGAUGAGGCCGUUAGGCCGAA GACUGGA B | 6304 |
| 1258 | GUGUCUC C UCUGCCG | 5955 | 18199 | HBV-1258 CHz-7 RNA | CGGCAGA CUGAUGAGGCCGUUAGGCCGAA GAGACAC B | 6305 |
| 2307 | GACCACC A AAUGCCC | 5956 | 18200 | HBV-2307 CHz-7 RNA | GGGCAUU CUGAUGAGGCCGUUAGGCCGAA GGUGGUC B | 6306 |
| 347 | UCACCAACCU G UUGUC | 5957 | 18216 | HBV-347 GCI.Rz-5/10 RNA | GACAA UGAUGGCAUGCACUAUGCGCG AGGUUGGUGA B | 6307 |
| 350 | CCAACCUGUU G UCCUC | 5958 | 18217 | HBV-350 GCI.Rz-5/10 RNA | GAGGA UGAUGGCAUGCACUAUGCGCG AACAGGUUGG B | 6308 |
| 1508 | UCCGCCUAUU G UACCG | 5959 | 18218 | HBV-1508 GCI.Rz-5/10 RNA | CGGUA UGAUGGCAUGCACUAUGCGCG AAUAGGCGGA B | 6309 |
| 234 | AAUCCU C ACAAUA | 5960 | 18334 | HBV-234 Rz-6 allyl stab1 | uₛaₛuₛuₛgu cUGAuGaggccguuaggccGaa Aggauu B | 6310 |
| 252 | GAGUCU A GACUCG | 5961 | 18335 | HBV-252 Rz-6 allyl stab1 | cₛgₛaₛgₛuc cUGAuGaggccguuaggccGaa Agacuc B | 6311 |
| 268 | UGGACU U CUCUCA | 5962 | 18337 | HBV-268 Rz-6 allyl stab1 | uₛgₛaₛgₛag cUGAuGaggccguuaggccGaa Agucca B | 6312 |
| 280 | AAUUUU C UAGGGG | 5963 | 18345 | HBV-280 Rz-6 allyl stab1 | cₛcₛcₛcₛua cUGAuGaggccguuaggccGaa Aaaauu B | 6313 |
| 313 | CAAAAU U CGCAGU | 5964 | 18346 | HBV-313 Rz-6 allyl stab1 | aₛcₛuₛgₛcg cUGAuGaggccguuaggccGaa Auuuug B | 6314 |
| 395 | GGCGUU U UAUCAU | 5965 | 18350 | HBV-395 Rz-6 allyl stab1 | aₛuₛgₛaₛua cUGAuGaggccguuaggccGaa Aacgcc B | 6315 |
| 402 | UAUCAU C UUCCUC | 5966 | 18351 | HBV-402 Rz-6 ailyl stab1 | gₛaₛgₛgₛaa cUGAuGaggccguuaggccGaa Augaua B | 6316 |
| 607 | UGUAUU C CCAUCC | 5967 | 18355 | HBV-607 Rz-6 aliyl stab1 | gₛgₛaₛuₛgg cUGAuGaggccguuaggccGaa Aauaca B | 6317 |
| 697 | UUUGUU C AGUGGU | 5968 | 18362 | HBV-697 Rz-6 allyl stab1 | aₛcₛcₛaₛcu cUGAuGaggccguuaggccGaa Aacaaa B | 6318 |
| 1539 | UCUCUU U ACGCGG | 5969 | 18366 | HBV-1539 Rz-6 ailyl stab1 | cₛcₛgₛcₛgu cUGAuGaggccguuaggccGaa Aagaga B | 6319 |
| 1599 | UCACCU C UGCACG | 5970 | 18367 | HBV-1599 Rz-6 allyl stab1 | cₛgₛuₛgₛca cUGAuGaggccguuaggccGaa Agguga B | 6320 |
| 1607 | GCACGU C GCAUGG | 5971 | 18368 | HBV-1607 Rz-6 aliyl stab1 | cₛcₛaₛuₛgc cUGAuGaggccguuaggccGaa Acgugc B | 6321 |
| 1833 | UCACCU C UGCCUA | 5972 | 18371 | HBV-1833 Rz-6 allyl stab1 | uₛaₛgₛgₛca cUGAuGaggccguuaggccGaa Agguga B | 6322 |
| 2383 | AGAACU C CCUCGC | 5973 | 18374 | HBV-2383 Rz-6 allyl stab1 | gₛcₛgₛaₛgg cUGAuGaggccguuaggccGaa Aguucu B | 6323 |
| 2429 | GAAGAU C UCAAUC | 5974 | 18376 | HBV-2429 Rz-6 allyl stab1 | gₛaₛuₛuₛga cUGAuGaggccguuaggccGaa Aucuuc B | 6324 |
| 2831 | UAUUCU U GGGAAC | 5975 | 18379 | HBV-2831 Rz-6 allyl stab1 | gₛuₛuₛcₛcc cUGAuGaggccguuaggccGaa Agaaua B | 6325 |
| 430 | UGCCUC A UCUUCU | 5976 | 18391 | HBV-430 CHz-6 allyl stab1 | aₛgₛaₛaₛga cUGAuGaggccguuaggccGaa laggca B | 6326 |
| 676 | UGGCUC A GUUUAC | 5977 | 18396 | HBV-676 CHz-6 allyl stab1 | gₛuₛaₛaₛac cUGAuGaggccguuaggccGaa lagcca B | 6327 |
| 683 | GUUUAC U AGUGCC | 5978 | 18397 | HBV-683 CHz-6 allyl stab1 | gₛgₛcₛaₛcu cUGAuGaggccguuaggccGaa luaaac B | 6328 |
| 1150 | UUUACC C CGUUGC | 5979 | 18402 | HBV-1150 CHz-6 allyl stab1 | gₛcₛaₛaₛcg cUGAuGaggccguuaggccGaa lguaaa B | 6329 |
| 1200 | GCAACC C CCACUG | 5980 | 18403 | HBV-1200 CHz-6 allyl stab1 | cₛaₛgₛuₛgg cUGAuGaggccguuaggccGaa lguugc B | 6330 |
| 1201 | CAACCC C CACUGG | 5981 | 18404 | HBV-1201 CHz-6 allyl stab1 | cₛcₛaₛgₛug cUGAuGaggccguuaggccGaa lgguug B | 6331 |
| 1444 | CGGCGC U GAAUCC | 5982 | 18405 | HBV-1444 CHz-6 allyl stab1 | gₛgₛaₛuₛuc cUGAuGaggccguuaggccGaa lcgccg B | 6332 |
| 1451 | GAAUCC C GCGGAC | 5983 | 18406 | HBV-1451 CHz-6 allyl stab1 | gₛuₛcₛcₛgc cUGAuGaggccguuaggccGaa lgauuc B | 6333 |
| 1533 | CGCACC U CUCUUU | 5984 | 18407 | HBV-1533 CHz-6 allyl stab1 | aₛaₛaₛgₛag cUGAuGaggccguuaggccGaa lgugcg B | 6334 |
| 1600 | CACCUC U GCACGU | 5985 | 18410 | HBV-1600 CHz-6 allyl stab1 | aₛcₛgₛuₛgc cUGAuGaggccguuaggccGaa laggug B | 6335 |
| 1698 | CCGACC U UGAGGC | 5986 | 18411 | HBV-1698 CHz-6 allyl stab1 | gₛcₛcₛuₛca cUGAuGaggccguuaggccGaa lgucgg B | 6336 |
| 1784 | GGAGGC U GUAGGC | 5987 | 18412 | HBV-1784 CHz-6 allyl stab1 | gₛcₛcₛuₛac cUGAuGaggccguuaggccGaa lccucc B | 6337 |
| 1829 | UUUUUC A CCUCUG | 5988 | 18414 | HBV-1829 CHz-6 allyl stab1 | cₛaₛgₛaₛgg cUGAuGaggccguuaggccGaa laaaaa B | 6338 |
| 1876 | GCCUCC A AGCUGU | 5989 | 18420 | HBV-1876 CHz-6 allyl stab1 | aₛcₛaₛgₛcu cUGAuGaggccguuaggccGaa lgaggc B | 6339 |
| 1880 | CCAAGC U GUGCCU | 5990 | 18422 | HBV-1880 CHz-6 allyl stab1 | aₛgₛgₛcₛac cUGAuGaggccguuaggccGaa lcuugg B | 6340 |
| 218 | UUUUUCU U GUUGACA | 5991 | 18333 | HBV-218 Rz-7 allyl stab1 | uₛgₛuₛcₛaac cUGAuGaggccguuaggccGaa Agaaaaa B | 6341 |
| 257 | CUAGACU C GUGGUGG | 5992 | 18336 | HBV-257 Rz-7 allyl stab1 | cₛcₛaₛcₛcac cUGAuGaggccguuaggccGaa Agucuag B | 6342 |
| 268 | GUGGACU U CUCUCAA | 5993 | 18338 | HBV-268 Rz-7 allyl stab1 | uₛuₛgₛaₛgag cUGAuGaggccguuaggccGaa Aguccac B | 6343 |
| 269 | UGGACUU C UCUCAAU | 5994 | 18339 | HBV-269 Rz-7 allyl stab1 | aₛuₛuₛgₛaga cUGAuGaggccguuaggccGaa Aagucca B | 6344 |
| 271 | GACUUCU C UCAAUUU | 5995 | 18340 | HBV-271 Rz-7 allyl stab1 | aₛaₛaₛuₛuga cUGAuGaggccguuaggccGaa Agaaguc B | 6345 |
| 273 | CUUCUCU C AAUUUUC | 5996 | 18341 | HBV-273 Rz-7 allyl stab1 | gₛaₛaₛaₛauu cUGAuGaggccguuaggccGaa Agagaag B | 6346 |
| 277 | UCUCAAU U UUCUAGG | 5997 | 18342 | HBV-277 Rz-7 allyl stab1 | cₛcₛuₛaₛgaa cUGAuGaggccguuaggccGaa Auugaga B | 6347 |
| 278 | CUCAAUU U UCUAGGG | 5998 | 18343 | HBV-278 Rz-7 allyl stab1 | cₛcₛcₛuₛaga cUGAuGaggccguuaggccGaa Aauugag B | 6348 |
| 279 | UCAAUUU U CUAGGGG | 5999 | 18344 | HBV-279 Rz-7 allyl stab1 | cₛcₛcₛcₛuag cUGAuGaggccguuaggccGaa Aaauuga B | 6349 |
| 314 | CAAAAUU C GCAGUCC | 6000 | 18347 | HBV-314 Rz-7 allyl stab1 | gₛgₛaₛcₛugc cUGAuGaggccguuaggccGaa Aauuuug B | 6350 |
| 385 | GAUGUGU C UGCGGCG | 6001 | 18348 | HBV-385 Rz-7 allyl stab1 | cₛgₛcₛcₛgca cUGAuGaggccguuaggccGaa Acacauc B | 6351 |
| 394 | GCGGCGU U UUAUCAU | 6002 | 18349 | HBV-394 Rz-7 allyl stab1 | aₛuₛgₛaₛuaa cUGAuGaggccguuaggccGaa Acgccgc B | 6352 |
| 402 | UUAUCAU C UUCCUCU | 6003 | 18352 | HBV-402 Rz-7 allyl stab1 | aₛgₛaₛgₛgaa cUGAuGaggccguuaggccGaa Augauaa B | 6353 |
| 423 | UGCUGCU A UGCCUCA | 6004 | 18353 | HBV-423 Rz-7 allyl stab1 | uₛgₛaₛgₛgca cUGAuGaggccguuaggccGaa Agcagca B | 6354 |
| 429 | UAUGCCU C AUCUUCU | 6005 | 18354 | HBV-429 Rz-7 allyl stab1 | aₛgₛaₛaₛgau cUGAuGaggccguuaggccGaa Aggcaua B | 6355 |
| 679 | GCUCAGU U UACUAGU | 6006 | 18356 | HBV-679 Rz-7 allyl stab1 | aₛcₛuₛaₛgua cUGAuGaggccguuaggccGaa Acugagc B | 6356 |
| 680 | CUCAGUU U ACUAGUG | 6007 | 18357 | HBV-680 Rz-7 allyl stab1 | cₛaₛcₛuₛagu cUGAuGaggccguuaggccGaa Aacugag B | 6357 |
| 681 | UCAGUUU A CUAGUGC | 6008 | 18358 | HBV-681 Rz-7 allyl stab1 | gₛcₛaₛcₛuag cUGAuGaggccguuaggccGaa Aaacuga B | 6358 |
| 684 | GUUUACU A GUGCCAU | 6009 | 18359 | HBV-684 Rz-7 allyl stab1 | aₛuₛgₛgₛcac cUGAuGaggccguuaggccGaa Aguaaac B | 6359 |
| 692 | GUGCCAU U UGUUCAG | 6010 | 18360 | HBV-692 Rz-7 allyl stab1 | cₛuₛgₛaₛaca cUGAuGaggccguuaggccGaa Auggcac B | 6360 |
| 693 | UGCCAUU U GUUCAGU | 6011 | 18361 | HBV-693 Rz-7 allyl stab1 | aₛcₛuₛgₛaac cUGAuGaggccguuaggccGaa Aauggca B | 6361 |
| 1534 | CGCACCU C UCUUUAC | 6012 | 18363 | HBV-1534 Rz-7 allyl stab1 | gₛuₛaₛaₛaga cUGAuGaggccguuaggccGaa Aggugcg B | 6362 |
| 1536 | CACCUCU C UUUACGC | 6013 | 18364 | HBV-1536 Rz-7 allyl stab1 | gₛcₛgₛuₛaaa cUGAuGaggccguuaggccGaa Agaggug B | 6363 |
| 1538 | CCUCUCU U UACGCGG | 6014 | 18365 | HBV-1538 Rz-7 allyl stab1 | cₛcₛgₛcₛgua cUGAuGaggccguuaggccGaa Agagagg B | 6364 |
| 1787 | AGGCUGU A GGCAUAA | 6015 | 18369 | HBV-1787 Rz-7 allyl stab1 | uₛuₛaₛuₛgcc cUGAuGaggccguuaggccGaa Acagccu B | 6365 |
| 1793 | UAGGCAU A AAUUGGU | 6016 | 18370 | HBV-1793 Rz-7 allyl stab1 | aₛcₛcₛaₛauu cUGAuGaggccguuaggccGaa Augccua B | 6366 |
| 1874 | CAAGCCU C CAAGCUG | 6017 | 18372 | HBV-1874 Rz-7 allyl stab1 | cₛaₛgₛcₛuug cUGAuGaggccguuaggccGaa Aggcuug B | 6367 |
| 1887 | UGUGCCU U GGGUGGC | 6018 | 18373 | HBV-1887 Rz-7 allyl stab1 | gₛcₛcₛaₛccc cUGAuGaggccguuaggccGaa Aggcaca B | 6368 |
| 2383 | AAGAACU C CCUCGCC | 6019 | 18375 | HBV-2383 Rz-7 allyl stab1 | gₛgₛcₛgₛagg cUGAuGaggccguuaggccGaa Aguucuu B | 6369 |
| 2828 | ACCAUAU U CUUGGGA | 6020 | 18377 | HBV-2828 Rz-7 allyl stab1 | uₛcₛcₛcₛaag cUGAuGaggccguuaggccGaa Auauggu B | 6370 |
| 2829 | CCAUAUU C UUGGGAA | 6021 | 18378 | HBV-2829 Rz-7 allyl stab1 | uₛuₛcₛcₛcaa cUGAuGaggccguuaggccGaa Aauaugg B | 6371 |
| 2831 | AUAUUCU U GGGAACA | 6022 | 18380 | HBV-2831 Rz-7 allyl stab1 | uₛgₛuₛuₛccc cUGAuGaggccguuaggccGaa Agaauau B | 6372 |
| 256 | UCUAGAC U CGUGGUG | 6023 | 18381 | HBV-256 CHz-7 allyl stab1 | cₛaₛcₛcₛacg cUGAuGaggccguuaggccGaa lucuaga B | 6373 |
| 267 | GGUGGAC U UCUCUCA | 6024 | 18382 | HBV-267 CHz-7 allyl stab1 | uₛgₛaₛgₛaga cUGAuGaggccguuaggccGaa luccacc B | 6374 |
| 270 | GGACUUC U CUCAAUU | 6025 | 18383 | HBV-270 CHz-7 allyl stab1 | aₛaₛuₛuₛgag cUGAuGaggccguuaggccGaa laagucc B | 6375 |
| 272 | ACUUCUC U CAAUUUU | 6026 | 18384 | HBV-272 CHz-7 allyl stab1 | aₛaₛaₛaₛuug cUGAuGaggccguuaggccGaa lagaagu B | 6376 |
| 274 | UUCUCUC A AUUUUCU | 6027 | 18385 | HBV-274 CHz-7 allyl stab1 | aₛgₛaₛaₛaau cUGAuGaggccguuaggccGaa lagagaa B | 6377 |
| 386 | AUGUGUC U GCGGCGU | 6028 | 18386 | HBV-386 CHz-7 allyl stab1 | aₛcₛgₛcₛcgc cUGAuGaggccguuaggccGaa lacacau B | 6378 |
| 419 | AUCCUGC U GCUAUGC | 6029 | 18387 | HBV-419 CHz-7 allyl stab1 | gₛcₛaₛuₛagc cUGAuGaggccguuaggccGaa lcaggau B | 6379 |
| 422 | CUGCUGC U AUGCCUC | 6030 | 18388 | HBV-422 CHz-7 allyl stab1 | gₛaₛgₛgₛcau cUGAuGaggccguuaggccGaa lcagcag B | 6380 |
| 427 | GCUAUGC C UCAUCUU | 6031 | 18389 | HBV-427 CHz-7 allyl stab1 | aₛaₛgₛaₛuga cUGAuGaggccguuaggccGaa lcauagc B | 6381 |
| 428 | CUAUGCC U CAUCUUC | 6032 | 18390 | HBV-428 CHz-7 allyl stab1 | gₛaₛaₛgₛaug cUGAuGaggccguuaggccGaa lgcauag B | 6382 |
| 430 | AUGCCUC A UCUUCUU | 6033 | 18392 | HBV-430 CHz-7 allyl stab1 | aₛaₛgₛaₛaga cUGAuGaggccguuaggccGaa laggcau B | 6383 |
| 608 | UGUAUUC C CAUCCCA | 6034 | 18393 | HBV-608 CHz-7 allyl stab1 | uₛgₛgₛgₛaug cUGAuGaggccguuaggccGaa laauaca B | 6384 |
| 609 | GUAUUCC C AUCCCAU | 6035 | 18394 | HBV-609 CHz-7 allyl stab1 | aₛuₛgₛgₛgau cUGAuGaggccguuaggccGaa lgaauac B | 6385 |
| 669 | GUUUCUC U UGGCUCA | 6036 | 18395 | HBV-669 CHz-7 allyl stab1 | uₛgₛaₛgₛcca cUGAuGaggccguuaggccGaa lagaaac B | 6386 |
| 689 | CUAGUGC C AUUUGUU | 6037 | 18398 | HBV-689 CHz-7 allyl stab1 | aₛaₛcₛaₛaau cUGAuGaggccguuaggccGaa lcacuag B | 6387 |
| 690 | UAGUGCC A UUUGUUC | 6038 | 18399 | HBV-690 CHz-7 allyl stab1 | gₛaₛaₛcₛaaa cUGAuGaggccguuaggccGaa lgcacua B | 6388 |
| 718 | GCUUUCC C CCACUGU | 6039 | 18400 | HBV-718 CHz-7 allyl stab1 | aₛcₛaₛgₛugg cUGAuGaggccguuaggccGaa lgaaagc B | 6389 |
| 1149 | CCUUUAC C CCGUUGC | 6040 | 18401 | HBV-1149 CHz-7 allyl stab1 | gₛcₛaₛaₛcgg cUGAuGaggccguuaggccGaa luaaagg B | 6390 |
| 1535 | GCACCUC U CUUUACG | 6041 | 18408 | HBV-1535 CHz-7 allyl stab1 | cₛgₛuₛaₛaag cUGAuGaggccguuaggccGaa laggugc B | 6391 |
| 1537 | ACCUCUC U UUACGCG | 6042 | 18409 | HBV-1537 CHz-7 allyl stab1 | cₛgₛcₛgₛuaa cUGAuGaggccguuaggccGaa lagaggu B | 6392 |
| 1791 | UGUAGGC A UAAAUUG | 6043 | 18413 | HBV-1791 CHz-7 allyl stab1 | cₛaₛaₛuₛuua cUGAuGaggccguuaggccGaa lccuaca B | 6393 |
| 1831 | UUUUCAC C UCUGCCU | 6044 | 18415 | HBV-1831 CHz-7 allyl stab1 | aₛgₛgₛcₛaga cUGAuGaggccguuaggccGaa lugaaaa B | 6394 |
| 1832 | UUUCACC U CUGCCUA | 6045 | 18416 | HBV-1832 CHz-7 allyl stab1 | uₛaₛgₛgₛcag cUGAuGaggccguuaggccGaa lgugaaa B | 6395 |
| 1872 | UUCAAGC C UCCAAGC | 6046 | 18417 | HBV-1872 CHz-7 allyl stab1 | gₛcₛuₛuₛgga cUGAuGaggccguuaggccGaa lcuugaa B | 6396 |
| 1873 | UCAAGCC U CCAAGCU | 6047 | 18418 | HBV-1873 CHz-7 allyl stab1 | aₛgₛcₛuₛugg cUGAuGaggccguuaggccGaa lgcuuga B | 6397 |
| 1875 | AAGCCUC C AAGCUGU | 6048 | 18419 | HBV-1875 CHz-7 allyl stab1 | aₛcₛaₛgₛcuu cUGAuGaggccguuaggccGaa laggcuu B | 6398 |
| 1876 | AGCCUCC A AGCUGUG | 6049 | 18421 | HBV-1876 CHz-7 allyl stab1 | cₛaₛcₛaₛgcu cUGAuGaggccguuaggccGaa lgaggcu B | 6399 |
| 1880 | UCCAAGC U GUGCCUU | 6050 | 18423 | HBV-1880 CHz-7 allyl stab1 | aₛaₛgₛgₛcac cUGAuGaggccguuaggccGaa lcuugga B | 6400 |
| 2382 | GAAGAAC U CCCUCGC | 6051 | 18424 | HBV-2382 CHz-7 allyl stab1 | gₛcₛgₛaₛggg cUGAuGaggccguuaggccGaa luucuuc B | 6401 |
| 2384 | AGAACUC C CUCGCCU | 6052 | 18425 | HBV-2384 CHz-7 allyl stab1 | aₛgₛgₛcₛgag cUGAuGaggccguuaggccGaa laguucu B | 6402 |
| 2385 | GAACUCC C UCGCCUC | 6053 | 18426 | HBV-2385 CHz-7 allyl stab1 | gₛaₛgₛgₛcga cUGAuGaggccguuaggccGaa lgaguuc B | 6403 |
| 2422 | GCGUCGC A GAAGAUC | 6054 | 18427 | HBV-2422 CHz-7 allyl stab1 | gₛaₛuₛcₛuuc cUGAuGaggccguuaggccGaa lcgacgc B | 6404 |
| 2830 | CAUAUUC U UGGGAAC | 6055 | 18428 | HBV-2830 CHz-7 allyl stab1 | gₛuₛuₛcₛcca cUGAuGaggccguuaggccGaa laauaug B | 6405 |
| 234 | AAUCCU C ACAAUA | 6056 | 19179 | HBV-234 Rz-6 amino stab1 | uₛaₛuₛuₛgu cUGAUGaggccguuaggccGaa Aggauu B | 6406 |
| 252 | GAGUCU A GACUCG | 6057 | 19180 | HBV-252 Rz-6 amino stab1 | cₛgₛaₛgₛuc cUGAUGaggccguuaggccGaa Agacuc B | 6407 |
| 268 | UGGACU U CUCUCA | 6058 | 19182 | HBV-268 Rz-6 amino stab1 | uₛgₛaₛgₛag cUGAUGaggccguuaggccGaa Agucca B | 6408 |
| 280 | AAUUUU C UAGGGG | 6059 | 19190 | HBV-280 Rz-6 amino stab1 | cₛcₛcₛcₛua cUGAUGaggccguuaggccGaa Aaaauu B | 6409 |
| 313 | CAAAAU U CGCAGU | 6060 | 19191 | HBV-313 Rz-6 amino stab1 | aₛcₛuₛgₛcg cUGAUGaggccguuaggccGaa Auuuug B | 6410 |
| 395 | GGCGUU U UAUCAU | 6061 | 19195 | HBV-395 Rz-6 amino stab1 | aₛuₛgₛaₛua cUGAUGaggccguuaggccGaa Aacgcc B | 6411 |
| 402 | UAUCAU C UUCCUC | 6062 | 19196 | HBV-402 Rz-6 amino stab1 | gₛaₛgₛgₛaa cUGAUGaggccguuaggccGaa Augaua B | 6412 |
| 607 | UGUAUU C CCAUCC | 6063 | 19200 | HBV-607 Rz-6 amino stab1 | gₛgₛaₛuₛgg cUGAUGaggccguuaggccGaa Aauaca B | 6413 |
| 697 | UUUGUU C AGUGGU | 6064 | 19207 | HBV-697 Rz-6 amino stab1 | aₛcₛcₛaₛcu cUGAUGaggccguuaggccGaa Aacaaa B | 6414 |
| 1539 | UCUCUU U ACGCGG | 6065 | 19211 | HBV-1539 Rz-6 amino stab1 | cₛcₛgₛcₛgu cUGAUGaggccguuaggccGaa Aagaga B | 6415 |
| 1599 | UCACCU C UGCACG | 6066 | 19212 | HBV-1599 Rz-6 amino stab1 | cₛgₛuₛgₛca cUGAUGaggccguuaggccGaa Agguga B | 6416 |
| 1607 | GCACGU C GCAUGG | 6067 | 19213 | HBV-1607 Rz-6 amino stab1 | cₛcₛaₛuₛgc cUGAUGaggccguuaggccGaa Acgugc B | 6417 |
| 1833 | UCACCU C UGCCUA | 6068 | 19216 | HBV-1833 Rz-6 amino stab1 | uₛaₛgₛgₛca cUGAUGaggccguuaggccGaa Agguga B | 6418 |
| 2383 | AGAACU C CCUCGC | 6069 | 19219 | HBV-2383 Rz-6 amino stab1 | gₛcₛgₛaₛgg cUGAUGaggccguuaggccGaa Aguucu B | 6419 |
| 2429 | GAAGAU C UCAAUC | 6070 | 19221 | HBV-2429 Rz-6 amino stab1 | gₛaₛuₛuₛga cUGAUGaggccguuaggccGaa Aucuuc B | 6420 |
| 2831 | UAUUCU U GGGAAC | 6071 | 19224 | HBV-2831 Rz-6 amino stab1 | gₛuₛuₛcₛcc cUGAUGaggccguuaggccGaa Agaaua B | 6421 |
| 430 | UGCCUC A UCUUCU | 6072 | 19236 | HBV-430 CHz-6 amino stab1 | aₛgₛaₛaₛga cUGAUGaggccguuaggccGaa laggca B | 6422 |
| 676 | UGGCUC A GUUUAC | 6073 | 19241 | HBV-676 CHz-6 amino stab1 | gₛuₛaₛaₛac cUGAUGaggccguuaggccGaa lagcca B | 6423 |
| 683 | GUUUAC U AGUGOC | 6074 | 19242 | HBV-683 CHz-6 amino stab1 | gₛgₛcₛaₛcu cUGAUGaggccguuaggccGaa luaaac B | 6424 |
| 1150 | UUUACC C CGUUGC | 6075 | 19247 | HBV-1150 CHz-6 amino stab1 | gₛcₛaₛaₛcg cUGAUGaggccguuaggccGaa lguaaa B | 6425 |
| 1200 | GCAACC C CCACUG | 6076 | 19248 | HBV-1200 CHz-6 amino stab1 | cₛaₛgₛuₛgg cUGAUGaggccguuaggccGaa lguugc B | 6426 |
| 1201 | CAACCC C CACUGG | 6077 | 19249 | HBV-1201 CHz-6 amino stab1 | cₛcₛaₛgₛug cUGAUGaggccguuaggccGaa lgguug B | 6427 |
| 1444 | CGGCGC U GAAUCC | 6078 | 19250 | HBV-1444 CHz-6 amino stab1 | gₛgₛaₛuₛuc cUGAUGaggccguuaggccGaa lcgccg B | 6428 |
| 1451 | GAAUCC C GCGGAC | 6079 | 19251 | HBV-1451 CHz-6 amino stab1 | gₛuₛcₛcₛgc cUGAUGaggccguuaggccGaa lgauuc B | 6429 |
| 1533 | CGCACC U CUCUUU | 6080 | 19252 | HBV-1533 CHz-6 amino stab1 | aₛaₛaₛgₛag cUGAUGaggccguuaggccGaa lgugcg B | 6430 |
| 1600 | CACCUC U GCACGU | 6081 | 19255 | HBV-1600 CHz-6 amino stab1 | aₛcₛgₛuₛgc cUGAUGaggccguuaggccGaa laggug B | 6431 |
| 1698 | CCGACC U UGAGGC | 6082 | 19256 | HBV-1698 CHz-6 amino stab1 | gₛcₛcₛuₛca cUGAUGaggccguuaggccGaa lgucgg B | 6432 |
| 1784 | GGAGGC U GUAGGC | 6083 | 19257 | HBV-1784 CHz-6 amino stab1 | gₛcₛcₛuₛac cUGAUGaggccguuaggccGaa lccucc B | 6433 |
| 1829 | UUUUUC A CCUCUG | 6084 | 19259 | HBV-1829 CHz-6 amino stab1 | cₛaₛgₛaₛgg cUGAUGaggccguuaggccGaa laaaaa B | 6434 |
| 1876 | GCCUCC A AGCUGU | 6085 | 19265 | HBV-1876 CHz-6 amino stab1 | aₛcₛaₛgₛcu cUGAUGaggccguuaggccGaa lgaggc B | 6435 |
| 1880 | CCAAGC U GUGCCU | 6086 | 19267 | HBV-1880 CHz-6 amino stab1 | aₛgₛgₛcₛac cUGAUGaggccguuaggccGaa lcuugg B | 6436 |
| 218 | UUUUUCU U GUUGACA | 6087 | 19178 | HBV-218 Rz-7 amino stab1 | uₛgₛuₛcₛaac cUGAUGaggccguuaggccGaa Agaaaaa B | 6437 |
| 257 | CUAGACU C GUGGUGG | 6088 | 19181 | HBV-257 Rz-7 amino stab1 | cₛcₛaₛcₛcac cUGAUGaggccguuaggccGaa Agucuag B | 6438 |
| 268 | GUGGACU U CUCUCAA | 6089 | 19183 | HBV-268 Rz-7 amino stab1 | uₛuₛgₛaₛgag cUGAUGaggccguuaggccGaa Aguccac B | 6439 |
| 269 | UGGACUU C UCUCAAU | 6090 | 19184 | HBV-269 Rz-7 amino stab1 | aₛuₛuₛgₛaga cUGAUGaggccguuaggccGaa Aagucca B | 6440 |
| 271 | GACUUCU C UCAAUUU | 6091 | 19185 | HBV-271 Rz-7 amino stab1 | aₛaₛaₛuₛuga cUGAUGaggccguuaggccGaa Agaaguc B | 6441 |
| 273 | CUUCUCU C AAUUUUC | 6092 | 19186 | HBV-273 Rz-7 amino stab1 | gₛaₛaₛaₛauu cUGAUGaggccguuaggccGaa Agagaag B | 6442 |
| 277 | UCUCAAU U UUCUAGG | 6093 | 19187 | HBV-277 Rz-7 amino stab1 | cₛcₛuₛaₛgaa cUGAUGaggccguuaggccGaa Auugaga B | 6443 |
| 278 | CUCAAUU U UCUAGGG | 6094 | 19188 | HBV-278 Rz-7 amino stab1 | cₛcₛcₛuₛaga cUGAUGaggccguuaggccGaa Aauugag B | 6444 |
| 279 | UCAAUUU U CUAGGGG | 6095 | 19189 | HBV-279 Rz-7 amino stab1 | cₛcₛcₛcₛuag cUGAUGaggccguuaggccGaa Aaauuga B | 6445 |
| 314 | CAAAAUU C GCAGUCC | 6096 | 19192 | HBV-314 Rz-7 amino stab1 | gₛgₛaₛcₛugc cUGAUGaggccguuaggccGaa Aauuuug B | 6446 |
| 385 | GAUGUGU C UGCGGCG | 6097 | 19193 | HBV-385 Rz-7 amino stab1 | cₛgₛcₛcₛgca cUGAUGaggccguuaggccGaa Acacauc B | 6447 |
| 394 | GCGGCGU U UUAUCAU | 6098 | 19194 | HBV-394 Rz-7 amino stab1 | aₛuₛgₛaₛuaa cUGAUGaggccguuaggccGaa Acgccgc B | 6448 |
| 402 | UUAUCAU C UUCCUCU | 6099 | 19197 | HBV-402 Rz-7 amino stab1 | aₛgₛaₛgₛgaa cUGAUGaggccguuaggccGaa Augauaa B | 6449 |
| 423 | UGCUGCU A UGCCUCA | 6100 | 19198 | HBV-423 Rz-7 amino stab1 | uₛgₛaₛgₛgca cUGAUGaggccguuaggccGaa Agcagca B | 6450 |
| 429 | UAUGCCU C AUCUUCU | 6101 | 19199 | HBV-429 Rz-7 amino stab1 | aₛgₛaₛaₛgau cUGAUGaggccguuaggccGaa Aggcaua B | 6451 |
| 679 | GCUCAGU U UACUAGU | 6102 | 19201 | HBV-679 Rz-7 amino stab1 | aₛcₛuₛaₛgua cUGAUGaggccguuaggccGaa Acugagc B | 6452 |
| 680 | CUCAGUU U ACUAGUG | 6103 | 19202 | HBV-680 Rz-7 amino stab1 | cₛaₛcₛuₛagu cUGAUGaggccguuaggccGaa Aacugag B | 6453 |
| 681 | UCAGUUU A CUAGUGC | 6104 | 19203 | HBV-681 Rz-7 amino stab1 | gₛcₛaₛcₛuag cUGAUGaggccguuaggccGaa Aaacuga B | 6454 |
| 684 | GUUUACU A GUGCCAU | 6105 | 19204 | HBV-684 Rz-7 amino stab1 | aₛuₛgₛgₛcac cUGAUGaggccguuaggccGaa Aguaaac B | 6455 |
| 692 | GUGCCAU U UGUUCAG | 6106 | 19205 | HBV-692 Rz-7 amino stab1 | cₛuₛgₛaₛaca cUGAUGaggccguuaggccGaa Auggcac B | 6456 |
| 693 | UGCCAUU U GUUCAGU | 6107 | 19206 | HBV-693 Rz-7 amino stab1 | aₛcₛuₛgₛaac cUGAUGaggccguuaggccGaa Aauggca B | 6457 |
| 1534 | CGCACCU C UCUUUAC | 6108 | 19208 | HBV-1534 Rz-7 amino stab1 | gₛuₛaₛaₛaga cUGAUGaggccguuaggccGaa Aggugcg B | 6458 |
| 1536 | CACCUCU C UUUACGC | 6109 | 19209 | HBV-1536 Rz-7 amino stab1 | gₛcₛgₛuₛaaa cUGAUGaggccguuaggccGaa Agaggug B | 5459 |
| 1538 | CCUCUCU U UACGCGG | 6110 | 19210 | HBV-1538 Rz-7 amino stab1 | cₛcₛgₛcₛgua cUGAUGaggccguuaggccGaa Agagagg B | 5460 |
| 1787 | AGGCUGU A GGCAUAA | 6111 | 19214 | HBV-1787 Rz-7 amino stab1 | uₛuₛaₛuₛgcc CUGAUGaggccguuaggccGaa Acagccu B | 6461 |
| 1793 | UAGGCAU A AAUUGGU | 6112 | 19215 | HBV-1793 Rz-7 amino stab1 | aₛcₛcₛaₛauu cUGAUGaggccguuaggccGaa Augccua B | 6462 |
| 1874 | CAAGCCU C CAAGCUG | 6113 | 19217 | HBV-1874 Rz-7 amino stab1 | cₛaₛgₛcₛuug cUGAUGaggccguuaggccGaa Aggcuug B | 6463 |
| 1887 | UGUGCCU U GGGUGGC | 6114 | 19218 | HBV-1887 Rz-7 amino stab1 | gₛcₛcₛaₛccc cUGAUGaggccguuaggccGaa Aggcaca B | 6464 |
| 2383 | AAGAACU C CCUCGCC | 6115 | 19220 | HBV-2383 Rz-7 amino stab1 | gₛgₛcₛgₛagg cUGAUGaggccguuaggccGaa Aguucuu B | 6465 |
| 2828 | ACCAUAU U CUUGGGA | 6116 | 19222 | HBV-2828 Rz-7 amino stab1 | uₛcₛcₛcₛaag cUGAUGaggccguuaggccGaa Auauggu B | 6466 |
| 2829 | CCAUAUU C UUGGGAA | 6117 | 19223 | HBV-2829 Rz-7 amino stab1 | uₛuₛcₛcₛcaa cUGAUGaggccguuaggccGaa Aauaugg B | 6467 |
| 2831 | AUAUUCU U GGGAACA | 6118 | 19225 | HBV-2831 Rz-7 amino stab1 | uₛgₛuₛuₛccc cUGAUGaggccguuaggccGaa Agaauau B | 6468 |
| 256 | UCUAGAC U CGUGGUG | 6119 | 19226 | HBV-256 CHz-7 amino stab1 | cₛaₛcₛcₛacg cUGAUGaggccguuaggccGaa lucuaga B | 6469 |
| 267 | GGUGGAC U UCUCUCA | 6120 | 19227 | HBV-267 CHz-7 amino stab1 | uₛgₛaₛgₛaga cUGAUGaggccguuaggccGaa luccacc B | 6470 |
| 270 | GGACUUC U CUCAAUU | 6121 | 19228 | HBV-270 CHz-7 amino stab1 | aₛaₛuₛuₛgag cUGAUGaggccguuaggccGaa laagucc B | 6471 |
| 272 | ACUUCUC U CAAUUUU | 6122 | 19229 | HBV-272 CHz-7 amino stab1 | aₛaₛaₛaₛuug cUGAUGaggccguuaggccGaa lagaagu B | 6472 |
| 274 | UUUUGUC A AUUUUCU | 6123 | 19230 | HBV-274 CHz-7 amino stab1 | aₛgₛaₛaₛaau cUGAUGaggccguuaggccGaa lagagaa B | 6473 |
| 386 | AUGUGUC U GCGGCGU | 6124 | 19231 | HBV-386 CHz-7 amino stab1 | aₛcₛgₛcₛcgc cUGAUGaggccguuaggccGaa lacacau B | 6474 |
| 419 | AUCCUGC U GCUAUGC | 6125 | 19232 | HBV-419 CHz-7 amino stab1 | gₛcₛaₛuₛagc cUGAUGaggccguuaggccGaa lcaggau B | 6475 |
| 422 | CUGCUGC U AUGCCUC | 6126 | 19233 | HBV-422 CHz-7 amino stab1 | gₛaₛgₛgₛcau cUGAUGaggccguuaggccGaa lcagcag B | 6476 |
| 427 | GCUAUGC C UCAUCUU | 6127 | 19234 | HBV-427 CHz-7 amino stab1 | aₛaₛgₛaₛuga cUGAUGaggccguuaggccGaa lcauagc B | 6477 |
| 428 | CUAUGCC U CAUCUUC | 6128 | 19235 | HBV-428 CHz-7 amino stab1 | gₛaₛaₛgₛaug cUGAUGaggccguuaggccGaa lgcauag B | 6478 |
| 430 | AUGCCUC A UCUUCUU | 6129 | 19237 | HBV-430 CHz-7 amino stab1 | aₛaₛgₛaₛaga cUGAUGaggccguuaggccGaa laggcau B | 6479 |
| 608 | UGUAUUC C CAUCCCA | 6130 | 19238 | HBV-608 CHz-7 amino stab1 | uₛgₛgₛgₛaug cUGAUGaggccguuaggccGaa laauaca B | 6480 |
| 609 | GUAUUCC C AUCCCAU | 6131 | 19239 | HBV-609 CHz-7 amino stab1 | aₛuₛgₛgₛgau cUGAUGaggccguuaggccGaa lgaauac B | 6481 |
| 669 | GUUUCUC U UGGCUCA | 6132 | 19240 | HBV-669 CHz-7 amino stab1 | uₛgₛaₛgₛcca cUGAUGaggccguuaggccGaa lagaaac B | 6482 |
| 689 | CUAGUGC C AUUUGUU | 6133 | 19243 | HBV-689 CHz-7 amino stab1 | aₛaₛcₛaₛaau cUGAUGaggccguuaggccGaa lcacuag B | 6483 |
| 690 | UAGUGCC A UUUGUUC | 6134 | 19244 | HBV-690 CHz-7 amino stab1 | gₛaₛaₛcₛaaa cUGAUGaggccguuaggccGaa lgcacua B | 6484 |
| 718 | GCUUUCC C CCACUGU | 6135 | 19245 | HBV-718 CHz-7 amino stab1 | aₛcₛaₛgₛugg cUGAUGaggccguuaggccGaa lgaaagc B | 6485 |
| 1149 | CCUUUAC C CCGUUGC | 6136 | 19246 | HBV-1149 CHz-7 amino stab1 | gₛcₛaₛaₛcgg cUGAUGaggccguuaggccGaa luaaagg B | 6486 |
| 1535 | GOACCUC U CUUUACG | 6137 | 19253 | HBV-1535 CHz-7 amino stab1 | cₛgₛuₛaₛaag cUGAUGaggccguuaggccGaa laggugc B | 6487 |
| 1537 | ACCUCUC U UUACGCG | 6138 | 19254 | HBV-1537 CHz-7 amino stab1 | cₛgₛcₛgₛuaa cUGAUGaggccguuaggccGaa lagaggu B | 6488 |
| 1791 | UGUAGGC A UAAAUUG | 6139 | 19258 | HBV-1791 CHz-7 amino stab1 | cₛaₛaₛuₛuua cUGAUGaggccguuaggccGaa lccuaca B | 6489 |
| 1831 | UUUUCAC C UCUGCCU | 6140 | 19260 | HBV-1831 CHz-7 amino stab1 | aₛgₛgₛcₛaga cUGAUGaggccguuaggccGaa lugaaaa B | 6490 |
| 1832 | UUUCACC U CUGCCUA | 6141 | 19261 | HBV-1832 CHz-7 amino stab1 | uₛaₛgₛgₛcag cUGAUGaggccguuaggccGaa lgugaaa B | 6491 |
| 1872 | UUCAAGC C UCCAAGC | 6142 | 19262 | HBV-1872 CHz-7 amino stab1 | gₛcₛuₛuₛgga cUGAUGaggccguuaggccGaa lcuugaa B | 6492 |
| 1873 | UCAAGCC U CCAAGCU | 6143 | 19263 | HBV-1873 CHz-7 amino stab1 | aₛgₛcₛuₛugg cUGAUGaggccguuaggccGaa lgcuuga B | 6493 |
| 1875 | AAGCCUC C AAGCUGU | 6144 | 19264 | HBV-1875 CHz-7 amino stab1 | aₛcₛaₛgₛcuu cUGAUGaggccguuaggccGaa laggcuu B | 6494 |
| 1876 | AGCCUCC A AGCUGUG | 6145 | 19266 | HBV-1876 CHz-7 amino stab1 | cₛaₛcₛaₛgcu cUGAUGaggccguuaggccGaa lgaggcu B | 6495 |
| 1880 | UCCAAGC U GUGCCUU | 6146 | 19268 | HBV-1880 CHz-7 amino stab1 | aₛaₛgₛgₛcac cUGAUGaggccguuaggccGaa lcuugga B | 6496 |
| 2382 | GAAGAAC U CCCUCGC | 6147 | 19269 | HBV-2382 CHz-7 amino stab1 | gₛcₛgₛaₛggg cUGAUGaggccguuaggccGaa luucuuc B | 6497 |
| 2384 | AGAACUC C CUCGCCU | 6148 | 19270 | HBV-2384 CHz-7 amino stab1 | aₛgₛgₛcₛgag cUGAUGaggccguuaggccGaa laguucu B | 6498 |
| 2385 | GAACUCC C UCGCCUC | 6149 | 19271 | HBV-2385 CHz-7 amino stab1 | gₛaₛgₛgₛcga cUGAUGaggccguuaggccGaa lgaguuc B | 6499 |
| 2422 | GOGUOGO A GAAGAUC | 6150 | 19272 | HBV-2422 CHz-7 amino stab1 | gₛaₛuₛcₛuuc cUGAUGaggccguuaggccGaa lcgacgc B | 6500 |
| 2830 | CAUAUUC U UGGGAAC | 6151 | 19273 | HBV-2830 CHz-7 amino stab1 | gₛuₛuₛcₛcca cUGAUGaggccguuaggccGaa laauaug B | 6501 |
| 315 | GCCAAAAUUC G CAGUC | 6152 | 20079 | HBV-315 GCI.Rz-5/10 stab2 | gₛaₛcₛg uGAUₛg gcauGcacuaugc gcg gaauuuuggc B | 6502 |
| 381 | AUCGCUGGAU G UGUCU | 6153 | 20080 | HBV-381 GCI.Rz-5/10 stab2 | aₛgₛaₛa uGAUₛg gcauGcacuaugc gcg auccagcgau B | 6503 |
| 476 | UUGCCCGUUU G UCCUC | 6154 | 20081 | HBV-476 GCI.Rz-5/10 stab2 | gₛaₛgₛa uGAUₛg gcauGcacuaugc gcg aaacgggcaa B | 6504 |
| 694 | AGUGCCAUUU G UUCAG | 6155 | 20082 | HBV-694 GCI.Rz-5110 stab2 | cₛuₛgₛa uGAUₛg gcauGcacuaugc gcg aaauggcacu B | 6505 |
| 1265 | CUCCUCUGCC G AUCCA | 6156 | 20083 | HBV-1265 GCI.Rz-5/10 stab2 | uₛgₛgₛu uGAUₛg gcauGcacuaugc gcg ggcagaggag B | 6506 |
| 1601 | CUUCACCUCU G CACGU | 6157 | 20084 | HBV-1601 GCI.Rz-5/10 stab2 | aₛcₛgₛg uGAUₛg gcauGcacuaugc gcg agaggugaag B | 6507 |
| 1881 | CCUCCAAGCU G UGCCU | 6158 | 20085 | HBV-1881 GCI.Rz-5/10 stab2 | aₛgₛgₛa uGAUₛg gcauGcacuaugc gcg agcuuggagg B | 6508 |
| 1883 | UCCAAGCUGU G CCUUG | 6159 | 20086 | HBV-1883 GCI.Rz-5/10 stab2 | cₛaₛaₛg uGAUₛg gcauGcacuaugc gcg acagcuugga B | 6509 |
| 2388 | GAACUCCCUC G CCUCG | 6160 | 20087 | HBV-2388 GCI.Rz-5110 stab2 | cₛgₛaₛg uGAUₛg gcauGcacuaugc gcg gagggaguuc B | 6510 |
| 381 | GCUGGAU G UGUCUGC | 6161 | 20091 | HBV-381 Zin.Rz-7 amino stab2 | gₛcₛaₛgₛaca GccgaaagGCGaGugaGGuCu auccagc B | 6511 |
| 392 | CUGCGGC G UUUUAUC | 6162 | 20092 | HBV-392 Zin.Rz-7 amino stab2 | gₛaₛuₛaₛaaa GccgaaagGCGaGugaGGuCu gccgcag B | 6512 |
| 420 | UCCUGCU G CUAUGCC | 6163 | 20093 | HBV-420 Zin.Rz-7 amino stab2 | gₛgₛcₛaₛuag GccgaaagGCGaGugaGGuCu agcagga B | 6513 |
| 648 | UAUGGGA G UGGGCCU | 6164 | 20094 | HBV-648 Zin.Rz-7 amino stab2 | aₛgₛgₛcₛcca GccgaaagGCGaGugaGGuCu ucccaua B | 6514 |
| 711 | UCGUAGG G CUUUCCC | 6165 | 20095 | HBV-711 Zin.Rz-7 amino stab2 | gₛgₛgₛaₛaag GccgaaagGCGaGugaGGuCu ccuacga B | 6515 |
| 1262 | CUCCUCU G CCGAUCC | 6166 | 20096 | HBV-1262 Zin.Rz-7 amino stab2 | gₛgₛaₛuₛcgg GccgaaagGCGaGugaGGuCu agaggag B | 6516 |
| 1835 | CACCUCU G CCUAAUC | 6167 | 20097 | HBV-1835 Zin.Rz-7 amino stab2 | gₛaₛuₛuₛagg GccgaaagGCGaGugaGGuCu agaggug B | 6517 |
| 2388 | CUCCCUC G CCUCGCA | 6168 | 20098 | HBV-2388 Zin.Rz-7 amino stab2 | uₛgₛcₛgₛagg GccgaaagGCGaGugaGGuCu gagggag B | 6518 |
| 192 | GACCCCU G CUCGUGU | 6169 | 20099 | HBV-192 Zin.Rz-7 amino stab2 | aₛcₛaₛcₛgag GccgaaagGCGagugaGGuCu agggguc B | 6519 |
| 198 | UGCUCGU G UUACAGG | 6170 | 20100 | HBV-198 Zin.Rz-7 amino stab2 | cₛcₛuₛgₛuaa GccgaaagGCGaGugaGGuCu acgagca B | 6520 |
| 315 | AAAAUUC G CAGUCCC | 6171 | 20101 | HBV-315 Zin.Rz-7 amino stab2 | gₛgₛgₛaₛcug GccgaaagGCGaGugaGGuCu gaauuuu B | 6521 |
| 383 | GGAUGU G UCUGCG | 6172 | 20102 | HBV-383 Zin.Rz-6 amino stab2 | cₛgₛcₛaₛga GccgaaagGCGaGugaGGuCu acaucc B | 6522 |
| 383 | UGGAUGU G UCUGCGG | 6173 | 20103 | HBV-383 Zin.Rz-7 amino stab2 | cₛcₛgₛcₛaga GccgaaagGCGaGugaGGuCu acaucca B | 6523 |
| 387 | GUGUCU G CGGCGU | 6174 | 20104 | HBV-387 Zin.Rz-6 amino stab2 | aₛcₛgₛcₛcg GccgaaagGCGaGugaGGuCu agacac B | 6524 |
| 390 | GUCUGCG G CGUUUUA | 6175 | 20105 | HBV-390 Zin.Rz-7 amino stab2 | uₛaₛaₛaₛacg GccgaaagGCGaGugaGGuCu cgcagac B | 6525 |
| 392 | UGCGGC G UUUUAU | 6176 | 20106 | HBV-392 Zin.Rz-6 amino stab2 | aₛuₛaₛaₛaa GccgaaagGCGaGugaGGuCu gccgca B | 6526 |
| 425 | UGCUAU G CCUCAU | 6177 | 20107 | HBV-425 Zin.Rz-6 amino stab2 | aₛuₛgₛaₛgg GccgaaagGCGaGugaGGuCu auagca B | 6527 |
| 425 | CUGCUAU G CCUCAUC | 6178 | 20108 | HBV-425 Zin.Rz-7 amino stab2 | gₛaₛuₛgₛagg GccgaaagGCGaGugaGGuCu auagcag B | 6528 |
| 468 | GUAUGUU G CCCGUUU | 6179 | 20109 | HBV-468 Zin.Rz-7 amino stab2 | aₛaₛaₛcₛggg GccgaaagGCGaGugaGGuCu aacauac B | 6529 |
| 476 | CCCGUUU G UCCUCUA | 6180 | 20110 | HBV-476 Zin.Rz-7 amino stab2 | uₛaₛgₛaₛgga GccgaaagGCGaGugaGGuCu aaacggg B | 6530 |
| 648 | AUGGGA G UGGGCC | 6181 | 20111 | HBV-648 Zin.Rz-6 amino stab2 | gₛgₛcₛcₛca GccgaaagGCGaGugaGGuCu ucccau B | 6531 |
| 694 | GCCAUUU G UUCAGUG | 6182 | 20112 | HBV-694 Zin.Rz-7 amino stab2 | cₛaₛcₛuₛgaa GccgaaagGCGaGugaGGuCu aaauggc B | 6532 |
| 699 | UUGUUCA G UGGUUCG | 6183 | 20113 | HBV-699 Zin.Rz-7 amino stab2 | cₛgₛaₛaₛcca GccgaaagGCGaGugaGGuCu ugaacaa B | 6533 |
| 1262 | UCCUCU G CCGAUC | 6184 | 20114 | HBV-1262 Zin.Rz-6 amino stab2 | gₛaₛuₛcₛgg GccgaaagGCGaGugaGGuCu agagga B | 6534 |
| 1440 | CCCGUCG G CGCUGAA | 6185 | 20115 | HBV-1440 Zin.Rz-7 amino stab2 | uₛuₛcₛaₛgcg GccgaaagGCGaGugaGGuCu cgacggg B | 6535 |
| 1526 | CACGGG G CGCACC | 6186 | 20116 | HBV-1526 Zin.Rz-6 amino stab2 | gₛgₛuₛgₛcg GccgaaagGCGaGugaGGuCu cccgug B | 6536 |
| 1526 | CCACGGG G CGCACCU | 6187 | 20117 | HBV-1526 Zin.Rz-7 amino stab2 | aₛgₛgₛuₛgcg GccgaaagGCGaGugaGGuCu cccguug B | 6537 |
| 1557 | CCCGUCU G UGCCUUC | 6188 | 20118 | HBV-1557 Zin.Rz-7 amino stab2 | gₛaₛaₛgₛgca GccgaaagGCGaGugaGGuCu agacggg B | 6538 |
| 1559 | CGUCUGU G CCUUCUC | 6189 | 20119 | HBV-1559 Zin.Rz-7 amino stab2 | gₛaₛgₛaₛagg GccgaaagGCGaGugaGGuCu acagacg B | 6539 |
| 1590 | GCACUUC G CUUCACC | 6190 | 20120 | HBV-1590 Zin.Rz-7 amino stab2 | gₛgₛuₛgₛaag GccgaaagGCGaGugaGGuCu gaagugc B | 6540 |
| 1835 | ACCUCU G CCUAAU | 6191 | 20121 | HBV-1835 Zin.Rz-6 amino stab2 | aₛuₛuₛaₛgg GccgaaagGCGaGugaGGuCu agaggu B | 6541 |
| 2311 | ACCAAAU G CCCCUAU | 6192 | 20122 | HBV-2311 Zin.Rz-7 amino stab2 | aₛuₛₛgₛggg GccgaaagGCGaGugaGGuCu auuuggu B | 6542 |
| 2420 | CCGCGUC G CAGAAGA | 6193 | 20123 | HBV-2420 Zin.Rz-7 amino stab2 | uₛcₛuₛuₛcug GccgaaagGCGaGugaGGuCu gacgcgg B | 6543 |
| 65 | CCUGCUG G UGGCUCC | 6194 | 20124 | HBV-65 Zin.Rz-7 amino stab2 | gₛgₛaₛgₛcca GccgaaagGCGaGugaGGuCu cagcagg B | 6544 |
| 192 | ACCCCU G CUCGUG | 6195 | 20125 | HBV-192 Zin.Rz-6 amino stab2 | cₛaₛcₛgₛag GccgaaagGCGaGugaGGuCu aggggu B | 6545 |
| 198 | GCUCGU G UUACAG | 6196 | 20126 | HBV-198 Zin.Rz-6 amino stab2 | cₛuₛgₛuₛaa GccgaaagGCGaGugaGGuCu acgagc B | 6546 |
| 258 | UAGACUC G UGGUGGA | 6197 | 20127 | HBV-258 Zin.Rz-7 amino stab2 | uₛcₛcₛaₛcca GccgaaagGCGaGugaGGuCu gagucua B | 6547 |
| 261 | ACUCGUG G UGGACUU | 6198 | 20128 | HBV-261 Zin.Rz-7 amino stab2 | aₛaₛgₛuₛcca GccgaaagGCGaGugaGGuCu cacgagu B | 6548 |
| 315 | AAAUUC G CAGUCC | 6199 | 20129 | HBV-315 Zin.Rz-6 amino stab2 | gₛgₛaₛcₛug GccgaaagGCGaGugaGGuCu gaauuu B | 6549 |
| 381 | CUGGAU G UGUCUG | 6200 | 20130 | HBV-381 Zin.Rz-6 amino stab2 | cₛaₛgₛaₛca GccgaaagGCGaGugaGGuCu auccag B | 6550 |
| 387 | UGUGUCU G CGGCGUU | 6201 | 20131 | HBV-387 Zin.Rz-7 amino stab2 | aₛaₛcₛgₛccg GccgaaagGCGaGugaGGuCu agacaca B | 6551 |
| 390 | UCUGCG G CGUUUU | 6202 | 20132 | HBV-390 Zin.Rz-6 amino stab2 | aₛaₛaₛaₛcg GccgaaagGCGaGugaGGuCu cgcaga B | 6552 |
| 417 | CAUCCU G CUGCUA | 6203 | 20133 | HBV-417 Zin.Rz-6 amino stab2 | uₛaₛgₛcₛag GccgaaagGCGaGugaGGuCu aggaug B | 6553 |
| 420 | CCUGCU G CUAUGC | 6204 | 20134 | HBV-420 Zin.Rz-6 amino stab2 | gₛcₛaₛuₛag GccgaaagGCGaGugaGGuCu agcagg B | 6554 |
| 468 | UAUGUU G CCCGUU | 6205 | 20135 | HBV-468 Zin.Rz-6 amino stab2 | aₛaₛcₛgₛgg GccgaaagGCGaGugaGGuCu aacaua B | 6555 |
| 476 | CCGUUU G UCCUCU | 6206 | 20136 | HBV-476 Zin.Rz-6 amino stab2 | aₛgₛaₛgₛga GccgaaagGCGaGugaGGuCu aaacgg B | 6556 |
| 677 | GGCUCA G UUUACU | 6207 | 20137 | HBV-677 Zin.Rz-6 amino stab2 | aₛgₛuₛaₛaa GccgaaagGCGaGugaGGuCu ugagcc B | 6557 |
| 677 | UGGCUCA G UUUACUA | 6208 | 20138 | HBV-677 Zin.Rz-7 amino stab2 | uₛaₛgₛuₛaaa GccgaaagGCGaGugaGGuCu ugagcca B | 6558 |
| 685 | UUACUA G UGCCAU | 6209 | 20139 | HBV-685 Zin.Rz-6 amino stab2 | aₛuₛgₛgₛca GccgaaagGCGaGugaGGuCu uaguaa B | 6559 |
| 685 | UUUACUA G UGCCAUU | 6210 | 20140 | HBV-685 Zin.Rz-7 amino stab2 | aₛaₛuₛgₛgca GccgaaagGCGaGugaGGuCu uaguaaa B | 6560 |
| 687 | UACUAGU G CCAUUUG | 6211 | 20141 | HBV-687 Zin.Rz-7 amino stab2 | cₛaₛaₛaₛugg GccgaaagGCGaGugaGGuCu acuagua B | 6561 |
| 699 | UGUUCA G UGGUUC | 6212 | 20142 | HBV-699 Zin.Rz-6 amino stab2 | gₛaₛaₛcₛca GccgaaagGCGaGugaGGuCu ugaaca B | 6562 |
| 702 | UCAGUG G UUCGUA | 6213 | 20143 | HBV-702 Zin.Rz-6 amino stab2 | uₛaₛcₛgₛaa GccgaaagGCGaGugaGGuCu cacuga B | 6563 |
| 702 | UUCAGUG G UUCGUAG | 6214 | 20144 | HBV-702 Zin.Rz-7 amino stab2 | cₛuₛaₛcₛgaa GccgaaagGCGaGugaGGuCu cacugaa B | 6564 |
| 711 | CGUAGG G CUUUCC | 6215 | 20145 | HBV-711 Zin.Rz-6 amino stab2 | gₛgₛaₛaₛag GccgaaagGCGaGugaGGuCu ccuacg B | 6565 |
| 1006 | UUGUGG G UCUUUU | 6216 | 20146 | HBV-1006 Zin.Rz-6 amino stab2 | aₛaₛaₛaₛga GccgaaagGCGaGugaGGuCu ccacaa B | 6566 |
| 1103 | UUUCUC G CCAACU | 6217 | 20147 | HBV-1103 Zin.Rz-6 amino stab2 | aₛgₛuₛuₛgg GccgaaagGCGaGugaGGuCu gagaaa B | 6567 |
| 1103 | CUUUCUC G CCAACUU | 6218 | 20148 | HBV-1103 Zin.Rz-7 amino stab2 | aₛaₛgₛuₛugg GccgaaagGCGaGugaGGuCu gagaaag B | 6568 |
| 1184 | GCCAAGU G UUUGCUG | 6219 | 20149 | HBV-1184 Zin.Rz-7 amino stab2 | cₛaₛgₛcₛaaa GccgaaagGCGaGugaGGuCu acuuggc B | 6569 |
| 1440 | CCGUCG G CGCUGA | 6220 | 20150 | HBV-1440 Zin.Rz-6 amino stab2 | uₛcₛaₛgₛcg GccgaaagGCGaGugaGGuCu cgacgg B | 6570 |
| 1442 | GUCGGC G CUGAAU | 6221 | 20151 | HBV-1442 Zin.Rz-6 amino stab2 | aₛuₛuₛcₛag GccgaaagGCGaGugaGGuCu gccgac B | 6571 |
| 1442 | CGUCGGC G CUGAAUC | 6222 | 20152 | HBV-1442 Zin.Rz-7 amino stab2 | gₛaₛuₛuₛcag GccgaaagGCGaGugaGGuCu gccgacg B | 6572 |
| 1553 | CUCCCC G UCUGUG | 6223 | 20153 | HBV-1553 Zin.Rz-6 amino stab2 | cₛaₛcₛaₛga GccgaaagGCGaGugaGGuCu ggggag B | 6573 |
| 1557 | CCGUCU G UGCCUU | 6224 | 20154 | HBV-1557 Zin.Rz-6 amino stab2 | aₛaₛgₛgₛca GccgaaagGCGaGugaGGuCu agacgg B | 6574 |
| 1559 | GUCUGU G CCUUCU | 6225 | 20155 | HBV-1559 Zin.Rz-6 amino stab2 | aₛgₛaₛaₛgg GccgaaagGCGaGugaGGuCu acagac B | 6575 |
| 1583 | CCGUGU G CACUUC | 6226 | 20156 | HBV-1583 Zin.Rz-6 amino stab2 | gₛaₛaₛgₛug GccgaaagGCGaGugaGGuCu acacgg B | 6576 |
| 1590 | CACUUC G CUUCAC | 6227 | 20157 | HBV-1590 Zin.Rz-6 amino stab2 | gₛuₛgₛaₛag GccgaaagGCGaGugaGGuCu gaagug B | 6577 |
| 1622 | ACCACC G UGAACG | 6228 | 20158 | HBV-1622 Zin.Rz-6 amino stab2 | cₛgₛuₛuₛca GccgaaagGCGaGugaGGuCu gguggu B | 6578 |
| 1870 | UGUUCAA G CCUCCAA | 6229 | 20159 | HBV-1870 Zin.Rz-7 amino stab2 | uₛuₛgₛgₛagg GccgaaagGCGaGugaGGuCu uugaaca B | 6579 |
| 1881 | CCAAGCU G UGCCUUG | 6230 | 20160 | HBV-1881 Zin.Rz-7 amino stab2 | cₛaₛaₛgₛgca GccgaaagGCGaGugaGGuCu agcuugg B | 6580 |
| 1883 | AGCUGU G CCUUGG | 6231 | 20161 | HBV-1883 Zin.Rz-6 amino stab2 | cₛcₛaₛaₛgg GccgaaagGCGaGugaGGuCu aca gcu B | 6581 |
| 1883 | AAGCUGU G CCUUGGG | 6232 | 20162 | HBV-1883 Zin.Rz-7 amino stab2 | cₛcₛcₛaₛagg GccgaaagGCGaGugaGGuCu acagcuu B | 6582 |
| 2311 | CCAAAU G CCCCUA | 6233 | 20163 | HBV-2311 Zin.Rz-6 amino stab2 | uₛaₛgₛgₛgg GccgaaagGCGaGugaGGuCu auuugg B | 6583 |
| 2347 | ACUGUU G UUAGAC | 6234 | 20164 | HBV-2347 Zin.Rz-6 amino stab2 | gₛuₛcₛuₛaa GccgaaagGCGaGugaGGuCu aacagu B | 6584 |
| 2364 | AGGCAG G UCCCCU | 6235 | 20165 | HBV-2364 Zin.Rz-6 amino stab2 | aₛgₛgₛgₛga GccgaaagGCGaGugaGGuCu cugccu B | 6585 |
| 2364 | GAGGCAG G UCCCCUA | 6236 | 20166 | HBV-2364 Zin.Rz-7 amino stab2 | uₛaₛgₛgₛgga GccgaaagGCGaGugaGGuCu cugccuc B | 6586 |
| 2388 | UCCCUC G CCUCGC | 6237 | 20167 | HBV-2388 Zin.Rz-6 amino stab2 | gₛcₛgₛaₛgg GccgaaagGCGaGugaGGuCu gaggga B | 6587 |
| 2393 | CGCCUC G CAGACG | 6238 | 20168 | HBV-2393 Zin.Rz-6 amino stab2 | cₛgₛuₛcₛug GccgaaagGCGaGugaGGuCu gaggcg B | 6588 |
| 2417 | CGCCGC G UCGCAG | 6239 | 20169 | HBV-2417 Zin.Rz-6 amino stab2 | cₛuₛgₛcₛga GccgaaagGCGaGugaGGuCu gcggcg B | 6589 |
| 2420 | CGCGUC G CAGAAG | 6240 | 20170 | HBV-2420 Zin.Rz-6 amino stab2 | cₛuₛuₛcₛug GccgaaagGCGaGugaGGuCu gacgcg B | 6590 |
| 2474 | CAUAAG G UGGGAA | 6241 | 20171 | HBV-2474 Zin.Rz-6 amino stab2 | uₛuₛcₛcₛca GccgaaagGCGaGugaGGuCu cuuaug B | 6591 |
| 381 | GCUGGAU G UGUCUGC | 6242 | 20172 | HBV-381 Amb.Rz-7 stab2 | gₛcₛaₛgₛaca gga L ucCCUUCaagga L ucCGGG auccagc B | 6592 |
| 648 | UAUGGGA G UGGGCCU | 6243 | 20173 | HBV-648 Amb.Rz-7 stab2 | aₛgₛgₛcₛcca gga L ucCCUUCaagga L ucCGGG ucccaua B | 6593 |
| 198 | UGCUCGU G UUACAGG | 6244 | 20174 | HBV-198 Amb.Rz-7 stab2 | cₛcₛuₛgₛuaa gga L ucCCUUCaagga L ucCGGG acgagca B | 6594 |
| 377 | UAUCGCU G GAUGUGU | 6245 | 20175 | HBV-377 Amb.Rz-7 stab2 | aₛcₛaₛcₛauc gga L ucCCUUCaagga L ucCGGG agcgaua B | 6595 |
| 378 | AUCGCUG G AUGUGUC | 6246 | 20176 | HBV-378 Amb.Rz-7 stab2 | gₛaₛcₛaₛcau gga L ucCCUUCaagga L ucCGGG cagcgau B | 6596 |
| 383 | UGGAUGU G UCUGCGG | 6247 | 20177 | HBV-383 Amb.Rz-7 stab2 | cₛcₛgₛcₛaga gga L ucCCUUCaagga L ucCGGG acaucca B | 6597 |
| 383 | GGAUGU G UCUGCG | 6248 | 20178 | HBV-383 Amb.Rz-6 stab2 | cₛgₛcₛaₛga gga L ucCCUUCaagga L ucCGGG acaucc B | 6598 |
| 648 | AUGGGA G UGGGCC | 6249 | 20179 | HBV-648 Amb.Rz-6 stab2 | gₛgₛcₛcₛca gga L ucCCUUCaagga L ucCGGG ucccau B | 6599 |
| 650 | UGGGAGU G GGCCUCA | 6250 | 20180 | HBV-650 Amb.Rz-7 stab2 | uₛgₛaₛgₛgcc gga L ucCCUUCaagga L ucCGGG acuccca B | 6600 |
| 650 | GGGAGU G GGCCUC | 6251 | 20181 | HBV-650 Amb.Rz-6 stab2 | gₛaₛgₛgₛcc gga L ucCCUUCaagga L ucCGGG acuccc B | 6601 |
| 694 | GCCAUUU G UUCAGUG | 6252 | 20182 | HBV-694 Amb.Rz-7 stab2 | cₛaₛcₛuₛgaa gga L ucCCUUCaagga L ucCGGG aaauggc B | 6602 |
| 699 | UUGUUCA G UGGUUCG | 6253 | 20183 | HBV-699 Amb.Rz-7 stab2 | cₛgₛaₛaₛcca gga L ucCCUUCaagga L ucCGGG ugaacaa B | 6603 |
| 701 | GUUCAGU G GUUCGUA | 6254 | 20184 | HBV-701 Amb.Rz-7 stab2 | uₛaₛcₛgₛaac gga L ucCCUUCaagga L ucCGGG acugaac B | 6604 |
| 710 | UUCGUAG G GCUUUCC | 6255 | 20185 | HBV-710 Amb.Rz-7 stab2 | gₛgₛaₛaₛagc gga L ucCCUUCaagga L ucCGGG cuacgaa B | 6605 |
| 1525 | CCACGG G GCGCAC | 6256 | 20186 | HBV-1525 Amb.Rz-6 stab2 | gₛuₛgₛcₛgc gga L ucCCUUCaagga L ucCGGG ccgugg B | 6606 |
| 1624 | CACCGU G AACGCC | 6257 | 20187 | HBV-1624 Amb.Rz-6 stab2 | gₛgₛcₛgₛuu gga L ucCCUUCaagga L ucCGGG acggug B | 6607 |
| 2069 | CACUCA G GCAAGC | 6258 | 20188 | HBV-2069 Amb.Rz-6 stab2 | gₛcₛuₛuₛgc gga L ucCCUUCaagga L ucCGGG ugagug B | 6608 |
| 2375 | CCUAGAA G AAGAACU | 6259 | 20189 | HBV-2375 Amb.Rz-7 stab2 | aₛgₛuₛuₛcuu gga L ucCCUUCaagga L ucCGGG uucuagg B | 6609 |
| 2476 | AUAAGGU G GGAAACU | 6260 | 20190 | HBV-2476 Amb.Rz-7 stab2 | aₛgₛuₛuₛucc gga L ucCCUUCaagga L ucCGGG accuuau B | 6610 |
| 65 | CCUGCUG G UGGCUCC | 6261 | 20191 | HBV-65 Amb.Rz-7 stab2 | gₛgₛaₛgₛcca gga L ucCCUUCaagga L ucCGGG cagcagg B | 6611 |
| 67 | GCUGGU G GCUCCA | 6262 | 20192 | HBV-67 Amb.Rz-6 stab2 | uₛgₛgₛaₛgc gga L ucCCUUCaagga L ucCGGG accagc B | 6612 |
| 198 | GCUCGU G UUACAG | 6263 | 20193 | HBV-198 Amb.Rz-6 stab2 | cₛuₛgₛuₛaa gga L ucCCUUCaagga L ucCGGG acgagc B | 6613 |
| 260 | GACUCGU G GUGGACU | 6264 | 20194 | HBV-260 Amb.Rz-7 stab2 | aₛgₛuₛcₛcac gga L ucCCUUCaagga L ucCGGG acgaguc B | 6614 |
| 263 | UCGUGGU G GACUUCU | 6265 | 20195 | HBV-263 Amb.Rz-7 stab2 | aₛgₛaₛaₛguc gga L ucCCUUCaagga L ucCGGG accacga B | 6615 |
| 377 | AUCGCU G GAUGUG | 6266 | 20196 | HBV-377 Amb.Rz-6 stab2 | cₛaₛcₛaₛuc gga L ucCCUUCaagga L ucCGGG agcgau B | 6616 |
| 378 | UCGCUG G AUGUGU | 6267 | 20197 | HBV-378 Amb.Rz-6 stab2 | aₛcₛaₛcₛau gga L ucCCUUCaagga L ucCGGG cagcga B | 6617 |
| 476 | CCGUUU G UCCUCU | 6268 | 20198 | HBV-476 Amb.Rz-6 stab2 | aₛgₛaₛgₛga gga L ucCCUUCaagga L ucCGGG aaacgg B | 6618 |
| 651 | GGGAGUG G GCCUCAG | 6269 | 20199 | HBV-651 Amb.Rz-7 stab2 | cₛuₛgₛaₛggc gga L ucCCUUCaagga L ucCGGG cacuccc B | 6619 |
| 677 | UGGCUCA G UUUACUA | 6270 | 20200 | HBV-677 Amb.Rz-7 stab2 | uₛaₛgₛuₛaaa gga L ucCCUUCaagga L ucCGGG ugagcca B | 6620 |
| 685 | UUUACUA G UGCCAUU | 6271 | 20201 | HBV-685 Amb.Rz-7 stab2 | aₛaₛuₛgₛgca gga L ucCCUUCaagga L ucCGGG uaguaaa B | 6621 |
| 702 | UUCAGUG G UUCGUAG | 6272 | 20202 | HBV-702 Amb.Rz-7 stab2 | cₛuₛaₛcₛgaa gga L ucCCUUCaagga L ucCGGG cacugaa B | 6622 |
| 709 | GUUCGUA G GGCUUUC | 6273 | 20203 | HBV-709 Amb.Rz-7 stab2 | gₛaₛaₛaₛgcc gga L ucCCUUCaagga L ucCGGG uacgaac B | 6623 |
| 710 | UCGUAG G GCUUUC | 6274 | 20204 | HBV-710 Amb.Rz-6 stab2 | gₛaₛaₛaₛgc gga L ucCCUUCaagga L ucCGGG cuacga B | 6624 |
| 747 | UAUGGAU G AUGUGGU | 6275 | 20205 | HBV-747 Amb.Rz-7 stab2 | aₛcₛcₛaₛcau gga L ucCCUUCaagga L ucCGGG auccaua B | 6625 |
| 1557 | CCGUCU G UGCCUU | 6276 | 20206 | HBV-1557 Amb.Rz-6 stab2 | aₛaₛgₛgₛca gga L ucCCUUCaagga L ucCGGG agacgg B | 6626 |
| 1881 | CCAAGCU G UGCCUUG | 6277 | 20207 | HBV-1881 Amb.Rz-7 stab2 | cₛaₛaₛgₛgca gga L ucCCUUCaagga L ucCGGG agcuugg B | 6627 |
| 2347 | ACUGUU G UUAGAC | 6278 | 20208 | HBV-2347 Amb.Rz-6 stab2 | gₛuₛcₛuₛaa gga L ucCCUUCaagga L ucCGGG aacagu B | 6628 |
| 2375 | CUAGAA G AAGAAC | 6279 | 20209 | HBV-2375 Amb.Rz-6 stab2 | gₛuₛuₛcₛuu gga L ucCCUUCaagga L ucCGGG uucuag B | 6629 |
| 2378 | GAAGAA G AACUCC | 6280 | 20210 | HBV-2378 Amb.Rz-6 stab2 | gₛgₛaₛgₛuu gga L ucCCUUCaagga L ucCGGG uucuuc B | 6630 |
| 2423 | CGUCGCA G AAGAUCU | 6281 | 20211 | HBV-2423 Amb.Rz-7 stab2 | aₛgₛaₛuₛcuu gga L ucCCUUCaagga L ucCGGG ugcgacg B | 6631 |
| 2426 | GCAGAA G AUCUCA | 6282 | 20212 | H8V-2426 Amb.Rz-6 stab2 | uₛgₛaₛgₛau gga L ucCCUUCaagga L ucCGGG uucugc B | 6632 |
| 2426 | CGCAGAA G AUCUCAA | 6283 | 20213 | HBV-2426 Amb.Rz-7 stab2 | uₛuₛgₛaₛgau gga L ucCCUUCaagga L ucCGGG uucugcg B | 6633 |
| 2476 | UAAGGU G GGAAAC | 6284 | 20214 | HBV-2476 Amb.Rz-6 stab2 | gₛuₛuₛuₛcc gga L ucCCUUCaagga L ucCGGG accuua B | 6634 |
| 2477 | UAAGGUG G GAAACUU | 6285 | 20215 | HBV-2477 Amb.Rz-7 stab2 | aₛaₛgₛuₛuuc gga L ucCCUUCaagga L ucCGGG caccuua B | 6635 |
| 2477 | AAGGUG G GAAACU | 6286 | 20216 | HBV-2477 Amb.Rz-6 stab2 | aₛgₛuₛuₛuc gga L ucCCUUCaagga L ucCGGG caccuu B | 6636 |
| 1607 | UGCACGU C GCAUGGA | 6287 | 20697 | HBV-1607 Rz-7 allyl stab1 (7/4) | uₛcₛcₛaₛugc cUGAuGaggccguuaggccGaa Acgugca B | 6637 |
| 1887 | GUGCCU U GGGUGG | 6288 | 20698 | HBV-1887 Rz-6 allyl stab1 (6/4) | cₛcₛaₛcₛcc cUGAuGaggccguuaggccGaa Aggcac B | 6638 |
| 1607 | GCACGU C GCAUGG | 6289 | 20699 | HBV-1607 Rz-6 allyl stab1 (6/3) | cₛcₛaₛuₛgc cUGAuGaggccguuaggccGaa Acgugc B | 6639 |
| 1607 | UGCACGU C GCAUGGA | 6290 | 20700 | HBV-1607 Rz-7 allyl stab1 (7/3) | uₛcₛcₛaₛugc cUGAuGaggccguuaggccGaa Acgugca B | 6640 |
| 1887 | GUGCCU U GGGUGG | 6291 | 20701 | HBV-1887 Rz-6 allyl stab1 (6/3) | cₛcₛaₛcₛcc cUGAuGaggccguuaggccGaa Aggcac B | 6641 |
| 1887 | UGUGCCU U GGGUGGC | 6292 | 20702 | HBV-1887 Rz-7 allyl stab1 (7/3) | gₛcₛcₛaₛccc cUGAuGaggccguuaggccGaa Aggcaca B | 6642 |
| 313 | CCAAAAU U CGCAGUC | 5943 | 22798 | HBV-313 Rz-7 Ome stab1 | gacugcg CUGAUGAggccguuaggccGAA Auuuugg B | 6659 |
| 408 | UCUUCCU C UGCAUCC | 5946 | 22799 | HBV-408 Rz-7 Ome stab1 | ggaugca CUGAUGAggccguuaggccGAA Aggaaga B | 6660 |
| 1756 | AGGAGGU U AGGUUAA | 5950 | 22800 | HBV-1756 Rz-7 Ome stab1 | uuaaccu CUGAUGAggccguuaggccGAA Accuccu B | 6661 |
| 10 | CUCCACC A CUUUCCA | 5953 | 22770 | HBV-10 CHz-7 Ome stab1 | uggaaag CUGAUGAggccguuaggccGAA lguggag B | 6662 |
| 335 | UCCAGUC A CUCACCA | 5954 | 22771 | HBV-335 CHz-7 Ome stab1 | uggugag CUGAUGAggccguuaggccGAA lacugga B | 6663 |
| 273 | CUUCUCU C AAUUUUC | 5996 | 22645 | HBV-273 Rz-7 allyl stab1 (7/3- | gₛaₛaₛaₛauu CUGAuGagccguuaggcGaa Agagaag B | 6664 |
| | | | GUUA) | | | |
| 273 | CUUCUCU C AAUUUUC | 5996 | 22646 | HBV-273 Rz-7 allyl stab1 (7/4- | gₛaₛaₛaₛauu cUGAuGagccguuaggcGaa Agagaag B | 6665 |
| | | | | GUUA) | | |
| 273 | CUUCUCU C AAUUUUC | 5996 | 22648 | HBV-273 Rz-7 allyl stab1 (7/3- | gₛaₛaₛaₛauu cUGAuGagccguuaggcGaa Agagaag B | 6666 |
| | | | | GAAA) | | |
| 273 | CUUCUCU C AAUUUUC | 5996 | 22650 | HBV-273 Rz-7 allyl stab1 (7/4- | gₛaₛaₛaₛauu cUGAuGagccguuaggcGaa Agagaag B | 6667 |
| | | | | GAAA) | | |
| 273 | UUCUCU C AAUUUU | 6643 | 22644 | HBV-273 Rz-6 allyl stabl (6/3- | aₛaₛaₛaₛuu cUGAuGagccguuaggcGaa Agagaa B | 6668 |
| | | | | GUUA) | | |
| 273 | UUCUCU C AAUUUU | 6643 | 22647 | HBV-273 Rz-6 allyl stabi (6/3- | aₛaₛaₛaₛuu cUGAuGagccguuaggcGaa Agagaa B | 6669 |
| | | | | GAAA) | | |
| 273 | UUCUCU C AAUUUU | 6643 | 22649 | HBV-273 Rz-6 allyl stabl (6/4- | aₛaₛaₛaₛuu cUGAuGagccguuaggcGaa Agagaa B | 6670 |
| | | | | GAAA) | | |
| 350 | ACCUGUU G UCCUCCA | 6644 | 22714 | HBV-350 GCI.Rz-7 5ribo stab3 | uggagga uGAUg gcauGcacuaugc gCg aacaggu B | 6671 |
| 1253 | CCUUUGU G UCUCCUC | 6645 | 22715 | HBV-1253 GCI.Rz-7 5ribo stab3 | gaggaga uGAUg gcauGcacuaugc gCg acaaagg B | 6672 |
| 1856 | UGUUCAU G UCCUACU | 6646 | 22716 | HBV-1856 GCI.Rz-7 5ribo stab3 | aguagga uGAUg gcauGcacuaugc gCg augaaca B | 6673 |
| 1966 | GCCUUCU G ACUUCUU | 6647 | 22717 | HBV-1966 GCI.Rz-7 5ribo stab3 | aagaagu uGAUg gcauGcacuaugc gCg agaaggc B | 6674 |
| 3132 | UCCUCCU G CCUCCAC | 6648 | 22718 | HBV-3132 GCI.Rz-7 5ribo stab3 | guggagg uGAUg gcauGcacuaugc gCg aggagga B | 6675 |
| 332 | AUCUCCA G UCACUCA | 6649 | 22742 | HBV-332 Zin.Rz-7 amino stab4 | ugaguga gccgaaaggCgagugaGGuCu uggagau B | 6676 |
| 350 | ACCUGUU G UCCUCCA | 6644 | 22743 | HBV-350 Zin.Rz-7 amino stab4 | uggagga gccgaaaggCgagugaGGuCu aacaggu B | 6677 |
| 410 | UUCCUCU G CAUCCUG | 6650 | 22744 | HBV-410 Zin.Rz-7 amino stab4 | caggaug gccgaaaggCgagugaGGuCu agaggaa B | 6678 |
| 1253 | CCUUUGU G UCUCCUC | 6645 | 22745 | HBV-1253 Zin.Rz-7 amino stab4 | gaggaga gccgaaaggCgagugaGGuCu acaaagg B | 6679 |
| 1754 | GGAGGAG G UUAGGUU | 6651 | 22746 | HBV-1754 Zin.Rz-7 amino stab4 | aaccuaa gccgaaaggCgagugaGGuCu cuccucc B | 6680 |
| 407 | AUCUUCC U CUGCAUC | 6652 | 22772 | HBV-407 CHz-7 Ome stab1 | gaugcag CUGAUGAggccguuaggccGAA lgaagau B | 6681 |
| 1848 | UCAUCUC A UGUUCAU | 6653 | 22773 | HBV-1848 CHz-7 Ome stab1 | augaaca CUGAUGAggccguuaggccGAA lagauga B | 6682 |
| 3124 | GCAGCUC C UCCUCCU | 6654 | 22774 | HBV-3124 CHz-7 Ome stab1 | aggagga CUGAUGAggccguuaggccGAA lagcugc B | 6683 |
| 2165 | GUCAGCU A UGUCAAC | 6655 | 22801 | HBV-2165 Rz-7 Ome stab1 | guugaca CUGAUGAggccguuaggccGAA Agcugac B | 6684 |
| 2706 | CCGUAUU A UCCAGAG | 6656 | 22802 | HBV-2706 Rz-7 Ome stab1 | cucugga CUGAUGAggccguuaggccGAA Aauacgg B | 6685 |
| 350 | ACCUGUU G UCCUCCA | 6644 | 22966 | HBV-350 Dz-7 stab3 | uggagga GGCTAGCTACAACGA aacaggu B | 6686 |
| 332 | AUCUCCA G UCACUCA | 6649 | 22967 | HBV-332 Dz-7 stab3 | ugaguga GGCTAGCTACAACGA uggagau B | 6687 |
| 1840 | CUGCCUA A UCAUCUC | 6657 | 22968 | HBV-1840 Dz-7 stab3 | gagauga GGCTAGCTACAACGA uaggcag B | 6688 |
| 358 | UCCUCCA A UUUGUCC | 6658 | 22969 | HBV-358 Dz-7 stab3 | ggacaaa GGCTAGCTACAACGA uggagga B | 6689 |
| 1253 | CCUUUGU G UCUCCUC | 6645 | 22970 | HBV-1253 Dz-7 stab3 | gaggaga GGCTAGCTACAACGA acaaagg B | 6690 |
| | | | 20599 | SAC | cₛgₛaₛuₛgu cUAGuGacccgaaagggGaa Aagagg B | 6691 |

UPPER CASE = RIBO
UNDERLINE = DEOXY
lower case = 2′-O-methyl
I = inosine
s = phosphorothioate linkage
B = inverted deoxyabasic residue
U = 2′-deoxy-2′-C-allyl Uridine
U = 2′-deoxy-2′-amino Uridine
C = 2′-deoxy-2′-amino Cytidine

TABLE XII
Group Designation and Dosage levels for HBV transgenic mouse study

| | | | Number of | Duration of |
|---|---|---|---|---|
| Group | Compound | Dose | Mice | Treatment |
| 1 | RPI.18341 | 100 mg/kg/day* | 10F | 14 days |
| | (site 273) | | | |
| 2 | RPI.18371 | 100 mg/kg/day* | 10F | 14 days |
| | (site 1833) | | | |
| 3 | RPI.18418 | 100 mg/kg/day* | 10F | 14 days |
| | (site 1873) | | | |
| 4 | RPI.18372 | 100 mg/kg/day* | 10F | 14 days |
| | (site 1874) | | | |
| 5 | Saline control | 100 mg/kg/day* | 10F | 14 days |
| 6 | Untreated | | 10F | 0 days |

*administered via sc infusion using Alzet ® mini-osmotic pumps

TABLE XIII
GROUP DESIGNATION AND DOSAGE LEVELS FOR HBV
TRANSGENIC MOUSE STUDY

| | | | | | Duration |
|---|---|---|---|---|---|
| | | | | Number | of |
| Group | Compound | Dose | | of Mice | Treatment |
| 1 | RPI.18341 | 100 | mg/kg/day* | 15 (M or F) | 14 days |
| | (site 273) | | | | |
| 2 | RPI.18341 | 30 | mg/kg/day* | 15 (M or F) | 14 days |
| | (site 273) | | | | |
| 3 | RPI.18341 | 10 | mg/kg/day* | 15 (M or F) | 14 days |
| | (site 273) | | | | |
| 4 | RPI.18371 | 100 | mg/kg/day* | 15 (M or F) | 14 days |
| | site 1833 | | | | |
| 5 | RPI.18371 | 30 | mg/kg/day* | 15 (M or F) | 14 days |
| | site 1833 | | | | |
| 6 | RPI.18371 | 10 | mg/kg/day* | 15 (M or F) | 14 days |
| | site 1833 | | | | |
| 7 | SAC | 100 | mg/kg/day* | 15 (M or F) | 14 days |
| | (RPI.20599) | | | | |
| 8 | SAC | 30 | mg/kg/day* | 15 (M or F) | 14 days |
| | (RPI.20599) | | | | |
| 9 | SAC | 10 | mg/kg/day* | 15 (M or F) | 14 days |
| | (RPI.20599) | | | | |
| 10 | Saline | 12 | μl/day* | 15 (M or F) | 14 days |
| | control | | | | |
| 11 | 3TC ® | 50 | mg/kg/day, PO | 15 (M or F) | 14 days |
| | control | | | | |

*administered via sc infusion using Alzet ® mini-osmotic pumps

What we claim is:

1. An enzymatic nucleic acid molecule that specifically cleaves RNA derived from hepatitis B virus (HBV), wherein said enzymatic nucleic acid molecule comprises sequence defined as Seq. ID No. 6346.

2. A method of administering to a cell an enzymatic nucleic acid molecule of claim 1 independently or in conduction with other therapeutic compounds comprising contacting said cell with the enzymatic nucleic acid molecule under conditions suitable for said administration.

3. The method of claim 2, wherein said other therapeutic compound is type I interferon.

4. The method of claim 2, wherein said other therapeutic compound is 3TC® (Lamivudine).

5. The method of claim 2, wherein said other therapeutic compound and the enzymatic nucleic acid molecule are administered simultaneously.

6. The method of claim 3, wherein said other therapeutic compound and enzymatic nucleic acid molecule are administered separately.

7. The method of claim 3, wherein said type I interferon is interferon alpha.

8. The method of claim 3, wherein said type I interferon is interferon beta.

9. The method of claim 3, wherein said type I interferon is consensus interferon.

10. The method of claim 3, wherein said type I interferon is polyethylene glycol interferon.

11. The method of claim 3, wherein said type I interferon is polyethylene glycol interferon alpha 2a.

12. The method of claim 3, wherein said type I interferon is polyethylene glycol interferon alpha 2b.

13. The method of claim 3, wherein said type I interferon is polyethylene glycol consensus interferon.

14. The method of claim 2, wherein said cell is a mammalian cell.

15. The method of claim 14, wherein said cell is a human cell.

16. The method of claim 14, wherein said administration is in the presence of a delivery reagent.

17. The method of claim 16, wherein said delivery reagent is a lipid.

18. The method of claim 17, wherein said lipid is a cationic lipid.

19. The method of claim 17, wherein said lipid is a phospholipid.

20. The method of claim 16, wherein said delivery reagent is a liposome.
